SEQUENCE LISTING (4 sequences)

SEQ ID NO: 1 - protein, 33 aa, synthetic construct
  MOD_RES 6: note = phosphorylated tyrosine (pY)
  MOD_RES 17: note = phosphorylated tyrosine (pY)
ESDGGYMDMSKDESIDYVPMLDMKGDIKYADIE

SEQ ID NO: 2 - RNA, 20 nt, synthetic construct
  source 1..20: mol_type = other RNA
ctctactatgaggtgaattg

SEQ ID NO: 3 - DNA, 21 bp, synthetic construct
  source 1..21: mol_type = other DNA
tctacagagttccctgtttgc

SEQ ID NO: 4 - DNA, 24 bp, synthetic construct
  source 1..24: mol_type = other DNA
agcactcaactatatcttgtcagt

(19) United States
(12) Patent Application Publication
(10) Pub. No.: US 2025/0263404 A1
(43) Pub. Date: Aug. 21, 2025

(54) AMINOPYRIDINES AS ACTIVATORS OF PI3 KINASE

(71) Applicants: UCL Business Ltd, London (GB); United Kingdom Research and Innovation, London (GB)

(72) Inventors: Bart Vanhaesebroeck, London (GB); Roger L. Williams, London (GB); Richard Angell, London (GB); Ben Allsop, London (GB); Trevor Askwith, London (GB); Alice Hooper, London (GB); Derek M. Yellon, London (GB); Aw Edith Chan, London (GB); Sally Oxenford, London (GB)

(21) Appl. No.: 18/690,114

(22) PCT Filed: Sep. 14, 2022
(86) PCT No.: PCT/GB2022/052323

§ 371 Date: Mar. 7, 2024

(30) Foreign Application Priority Data
Sep. 14, 2021 (GB) 2113079.4

(51) Int. Cl. C07D 413/14; A61K 31/4439; A61K 31/4545; A61K 31/4709; A61K 31/4725; A61K 31/496; A61K 31/498; A61K 31/506; A61K 31/5377; A61P 25/02; C07D 213/74; C07D 401/12; C07D 401/14; C07D 413/12; C07D 417/14; C07D 491/107

(52) U.S. Cl. CPC C07D 413/14; A61K 31/4439; A61K 31/4545; A61K 31/4709; A61K 31/4725; A61K 31/496; A61K 31/498; A61K 31/506; A61K 31/5377; A61P 25/02; C07D 213/74; C07D 401/12; C07D 401/14; C07D 413/12; C07D 417/14; C07D 491/107

(57) ABSTRACT

The present invention relates to PI3Kα activating compounds and pharmaceutical compositions comprising the same. The present invention further relates, inter alia, to the treatment of disorders susceptible to treatment by PI3Kα activation.

### CROSS-REFERENCE TO RELATED APPLICATIONS

This application is a national phase application under 35 U.S.C. § 371 that claims priority to International Application No. PCT/GB2022/052323 filed Sep. 14, 2022, which claims priority to United Kingdom Application No. 2113079.4 filed Sep. 14, 2021, all of which are incorporated herein by reference in their entirety.

The present invention relates to PI3Kα activating compounds and pharmaceutical compositions comprising the same. The present invention further relates, inter alia, to the treatment of disorders susceptible to treatment by PI3Kα activation.

### INCORPORATION OF SEQUENCE LISTING

The instant application contains a Sequence Listing, named “SeqLst_KEMPP0154US” (5,354 bytes; created Mar. 1, 2024) which has been submitted electronically in XML format and is hereby incorporated by reference in its entirety.

### BACKGROUND TO THE INVENTION

Compared to the creation of protein and lipid kinase inhibitors, efforts to generate pharmacological activators to harness beneficial activities of some of these enzymes, such as in tissue regeneration and protection, wound healing, immunostimulation and metabolic sensitization, have been limited to date. The extended family of lipid kinases, and particularly the key subgroup PI 3-kinases (PI3Ks), play important positive roles in these therapeutic contexts, with no small molecule activators reported for this class of enzymes.

Class IA PI 3 kinases (PI3Ks) signal downstream of tyrosine kinases, G protein-coupled receptors and small GTPases to regulate cell growth, proliferation and migration. These PI3Ks consist of a p110 catalytic subunit (p110α, β or δ) in complex with a p85 regulatory subunit that recruits these PI3Ks to activated receptor complexes at the plasma membrane. Whereas p110α and p110β show a broad tissue distribution, p110δ is highly enriched in white blood cells. Overactivation of PI3K signalling and its downstream effectors AKT and mTORC1 in cancer and immune dysregulation has driven extensive PI3K pathway inhibitor development efforts, with several PI3K inhibitors now having received regulatory approval.

As further discussed elsewhere herein, there is ample evidence to suggest that PI3K/AKT pathway activation could also be of therapeutic benefit, such as in disease-associated cell protection and tissue regeneration. Indeed, PI3K/AKT inhibition dampens the protective effect of growth factors and a range of other agents or treatments in models of cell/tissue damage involving neurons, cardiomyocytes, muscle, lung epithelial cells and cells from the retina (see Borges, G. A. et al. Regen Med 15, 1329-1344 (2020); Matsuda, S. et al. International journal of oncology 49, 1785-1790 (2016); Koh, S. H. & Lo, E. H. J Clin Neurol 11, 297-304 (2015); Zhang, Z. et al. Mol Med Rep 18, 3547-3554 (2018)). This includes protection from ischaemia reperfusion injury (IRI) upon re-oxygenation (such as in neurons following a stroke and in cardiomyocytes upon cardiac arrest), protection from ionising radiation, enhancement of tissue and wound healing as well as neuro-protection/regeneration. Given its key role in insulin signalling, activation of PI3Kα might also overcome insulin resistance in obesity and type 2 diabetes, as evidenced by cell-based studies using a genetically-activated PI3Kα allele and in mice with type-2 diabetes in which cardiac-selective increase in PI3Kα by adenoviral gene therapy attenuated several characteristics of diabetic cardiomyopathy. PI3K activation has also been shown to improve the success rate of in vitro fertilization by ex vivo activation of dormant follicles from cryopreserved ovarian tissue or in primary ovarian insufficiency. Genetic strategies of PI3K/AKT activation tested in tissue regeneration include expression of activated alleles of PI3Kα (Prakoso, D. et al. Am J Physiol Heart Circ Physiol 318, H840-H852 (2020)) or AKT (Chen, S. et al. Front Endocrinol (Lausanne) 8, 21 (2017)) or inactivation of PTEN, a lipid phosphatase, which downregulates PI3K signalling, by gene deletion or knockdown (Borges, G. A. et al. Regen Med 15, 1329-1344 (2020); Park, K. K. et al. Science (New York, N.Y 322, 963-966 (2008); Liu, K. et al. Nature Neuroscience 13, 1075-1081 (2010); Ohtake, Y., Hayat, U. & Li, S. Neural Regen Res 10, 1363-1368 (2015)). The positive effect of PI3K pathway activation in this context is thought to derive from enhanced cell survival and proliferation, and possible activation of tissue-resident stem cells (Koh, S. H. & Lo, E. H. J Clin Neurol 11, 297-304 (2015); Wang, G. et al. The EMBO journal 37 (2018)).

Using isoform-selective PI3K inhibitors, it has been shown that PI3Kα is the principal mediator of insulin- or ischaemic preconditioning-driven protection from ischaemia reperfusion in cardiomyocytes (Rossello, X. et al. Basic Res Cardiol 112, 66 (2017)). Genetic PI3Kα activation also mediates axonal regeneration in neurons (Nieuwenhuis, B. et al. EMBO molecular medicine 12, e11674 (2020)).

To date, little effort has been undertaken to create non-genetic PI3K/AKT activators. These include cell-permeable p85-binding phospho-peptides that activate the p85/p110 complex, the AKT-activating small molecules SC79 and MX-2043 and a range of PTEN inhibitors. All of these PI3K activating agents have poor drug characteristics, an unclear mechanism of PI3K pathway activation and do not target PI3K in an isoform-selective manner.

It is consequently particularly desirable to identify compounds that are capable of activating PI3Kα in a PI3K isoform-selective manner in cells and tissues in order to provide both useful biological probes and methods of treating disorders that are susceptible to treatment by PI3Kα activation, like those discussed above.

The inventors have discovered what they believe to be the first class of small molecule to directly and allosterically activate PI3K. Similar to the discovery of wortmannin and LY294002 that enabled for the first time to pharmacologically probe the cellular impact of PI3K inhibition, this permits the provision of chemical tools for investigating the consequences of direct PI3Kα activation in basic and translational studies. Other than enhancing the understanding of the molecular mechanisms of allosteric PI3Kα activation, the compounds of the present invention will facilitate controlled signalling studies to gain a better quantitative understanding of PI3Kα signalling and to delineate PI3Kα-specific signalling in cells. The inventors have also provided herein a proof-of-concept for PI3Kα activation as a therapeutic approach. They have therefore also provided a proof-of-concept for the use of the compounds of the present invention in therapy.

At the biochemical level, the inventors' studies indicate that disruption by compounds of the invention of inhibitory contacts between p85α and p110α is key to PI3Kα activation. The structural changes induced by compounds of the present invention have similarities with, but do not fully overlap with, the dynamic structural changes observed in PI3Kα activation by natural ligands (such as pY, representing the tyrosine-phosphorylated docking sites for PI3Ka on receptor and associated molecules) or oncogenic PIK3CA mutations, indicating a unique biochemical activation mechanism of action for the compounds of the invention.

At the cellular level, PI3Kα signalling induced by compounds of the present invention and insulin showed overall similar kinetics, including effective downregulation upon prolonged exposure, even in the continuous presence of ligand. This indicates that PI3Kα signalling driven by compounds of the present invention remains subject to the endogenous feedback mechanisms that operate within the PI3K pathway. Such transient PI3K activation may allow to temporarily and effectively boost endogenous protective and regenerative mechanisms. A consequence of this would also be that organismal administration of compounds of the present invention, in addition to being governed by the chemical characteristics and turnover of the compounds, will most likely result in transient PI3K pathway activation that differs from the sustained impact on signalling provided by constitutive oncogenic PIK3CA activation. Such mechanism of action could mitigate the concern that PI3Kα activators might induce or promote cancer. Moreover, studies have shown that mutant PIK3CA on its own is only a weak driver oncogene, with mice constitutively expressing the Pik3ca^{H1047R} hot-spot mutation not developing cancer within a year. Similarly, people with rare mosaic genetic activation of PIK3CA are not predisposed to cancer in adulthood. These data make it less likely that short-term and transient pharmacological PI3Kα activation would promote cancer.

In summary, the present inventors have now identified a novel class of PI3Kα-activating compounds, having application as biochemical probes. They have further provided proof-of-concept of the therapeutic potential of allosteric PI3Kα activation, including in tissue regeneration (e.g., in nerve regeneration) and tissue protection (e.g., in protection of the heart from ischaemia reperfusion injury), using the compounds of the present invention.

### SUMMARY OF THE INVENTION

The present invention provides compounds (‘compounds of the invention’). Such compounds, in particular, include compounds of formula (I), as defined herein, as well as tautomers, N-oxides, pharmaceutically acceptable salts, and solvates thereof.

The present invention also provides pharmaceutical compositions comprising a compound of the invention, in association with one or more pharmaceutically acceptable carriers.

The present invention also provides a compound of the invention, or a pharmaceutical composition of the invention, for use as a medicament, and, in particular, for use in a method for treating and/or preventing a disorder susceptible to treatment by PI3Kα activation.

The present invention also provides the use of a compound of the invention, or a pharmaceutical composition of the invention, for the manufacture of a medicament, in particular a medicament for use in a method for treating and/or preventing a disorder susceptible to treatment by PI3Kα activation.

The present invention also provides a method of treatment, in particular a method for treating and/or preventing a disorder susceptible to treatment by PI3Kα activation in a patient in need thereof, the method comprising administering a compound of the invention, or a pharmaceutical composition of the invention, to the patient.

### BRIEF DESCRIPTION OF THE DRAWINGS

FIG. 1|Biochemical mechanism of PI3Kα activation by UCL-TRO-1938. a, Structure of UCL-TRO-1938 (referred to in the text as 1938). b, Selectivity of 1938 for PI3Kα over PI3Kβ and PI3Kδ. c, Enzyme kinetics (calculated using kcat function in Prism 8) upon ATP titration on PI3Kα with or without 1938 and pY. d, Membrane binding of PI3Kα shown as FRET signal (I-I0). I, fluorescence intensity at 520 nm, I₀, fluorescence intensity at 520 nm in the absence of enzyme. e, Effect of 1938 on PI3Kα catalytic activity in the presence of a saturating dose of pY. f, Effect of 1938 on the catalytic activity of oncogenic mutants of PI3Kα. g, Effect of 1938 on membrane binding of oncogenic mutants of PI3Kα. h, Effect of the PI3Kα-selective inhibitor BYL719 on 1938-activated PI3Kα. i. Effect of 1938 on the IC₅₀ of BYL719 for PI3Kα. Data shown as mean±SEM, n=3 (b,c,e,i), n=2 (d,h), or n=4 (f,g) independent experiments. Statistical analysis performed with two way ANOVA, Tukey's multiple comparisons test (b,f,g) or Sidak's multiple comparisons test (h); one way ANOVA, Dunnett's multiple comparisons test (e).

FIG. 2|Structural mechanism of PI3Kα activation by 1938. a, Structural changes induced by 1938 in full-length p110α/p85α as assessed by HDX-MS, highlighted on the structure of p110α (gray)/niSH2-p85α (green) (pdb: 4ZOP). A surface model is shown in FIG. 10/extended data FIG. 2.

FIG. 3|1938 activates PI3Kα pathway signalling in cells. a, PIP₃ and PI(3,4)P₂ generation in cells. ai. Kinetic data from total internal fluorescence (TIRF) microscopy of PIK3CA-WT and PIK3CA-KO A549 cells expressing the PIP₃ reporter EGFP-PH-ARNO^{I303}x2 and treated with DMSO, 1938 (5 μM)+/−BYL719 (0.5 μM). Individual single cell traces are shown, with mean intensity values at each time point (F(t)) shown relative to the starting mean intensity (F₀). Thick lines specify the medians. The data are from a single experiment (1938/BYL719: WT (n=11), KO (n=8); DMSO/BYL719: WT (n=14), KO (n=4)). aii. Representative TIRF microscopy image of a PIK3CA-WT A549 cell stimulated with 1938, with subsequent neutralisation by BYL719. Images were from cells in the experiment in i. above, with addition of 1938 at t=27 min, followed by addition of BYL719 at t=87 min. Images were taken 1.3 min before addition of 1938; 2.3 min after addition of 1938 and 3.3 min after addition of BYL719. Individual pixel values were scaled by normalising to the mean intensity observed across all time points prior to stimulation (F_{baseline}). Scale bar: 11 μm. aiii. TIRF microscopy data from HeLa cells expressing the EGFP-tagged PIP₃ reporter PH-ARNO-I303Ex2 (ARNO) or the PI(3,4)P₂ reporter mCherry-cPH-TAPP1x3. Overlay plots (mean±SEM) were generated by scaling to minimum and maximum values of the normalised fluorescence intensity for each time point (Fn₍ₜ₎). PIP₃ reporter data are representative of 2 experiments, 29 (DMSO/1938) and 20 (BYL719/DMSO) single cells. PI(3,4)P₂ reporter data are representative of 4 experiments, 78 (DMSO/1938) and 33 (BYL719/DMSO) single cells. b, Dose-dependent 1938-induction of pAKT^{S473} (detected by automated Wes western blotting) in PI3Kα-WT and PI3Kα-null MEFs. BYL, BYL719; TGX, TGX-221 and IC, IC87144. All PI3K inhibitors were used at 5 μM. Representative blot shown (n=2 experiments). c, Kinetic analysis of 1938-induced pAKT^{S473} (detected by automated Wes western blotting) in A549 cells by a saturating concentration of 1938 (with or without BYL719) or a saturating concentration of insulin. Shown is a representative blot (n=3 experiments). d, Generation of pAKT^{S473} by 1938 compared with insulin in A549 cells (measured by ELISA). e, Left panel, Time course analysis of insulin- or 1938-induced PI3K/AKT/mTORC1 signalling in A549 cells (detected by ECL western blotting). Right panel, quantification of pAKT^{S473}/vinculin signal ratio, expressed as fold-change relative to control treatment with DMSO only. f, In vitro selectivity profile of 1938 on 133 protein kinases and 7 lipid kinases. g, Phosphoproteomic analysis PI3Kα-WT and PI3Kα-KO MEFs stimulated with 1938 or insulin (n=4 independent experiments). gi. Heat map: phosphosites significantly altered by stimulation relative to DMSO treatment. Green boxes, significantly upregulated phosphosites; magenta boxes, significantly downregulated phosphosites; white crosses: phosphosites not detected in a comparison. gii. Volcano plot of phosphosites differentially regulated by 1938 in PI3Kα-WT or PI3Kα-KO MEFs, relative to DMSO-treated cells of the same genotype. Venn diagrams: overlap of number of phosphosites identified and regulated by 1938 in PI3Kα-WT MEFs with sites that have been identified previously and are annotated in PhosphoSitePlus as regulated by insulin, IGF-1, LY294002 or MK2206. giii. Venn diagrams indicate the overlap of phosphosites regulated by 1938 and insulin in PI3Kα-WT MEFs.

FIG. 4|1938 activates PI3Kα-dependent cell biological responses in cells. PI3Kα-WT and PI3Kα-KO MEFs were stimulated with 1938 (with or without BYL719), insulin or FBS, followed by measuring the impact on a, cellular metabolic activity (assessed by measurement of cellular ATP content by CellTiter-Glo©). b, cell cycle progression (measured by EdU incorporation) or c, cell number (assessed by crystal violet staining). Data shown as mean±SEM (n=2 independent experiments) (a-c).

FIG. 5|Disease-relevant biological activities of 1938. a, Impact of 1938 on ischaemia reperfusion injury in isolated rat hearts. Hearts were subjected to 45 min global ischaemia, followed by 2 h reperfusion, with administration of DMSO (0.1%) or 1938 (5 μM) during the first 15 min of perfusion. Left panel, Sections of two representative tetrazolium-stained slices of hearts following ischaemia and reperfusion, with or without 1938 treatment. Red: live tissue; white: infarcted tissue. Cardiac electric activity (measured by ECG) in representative hearts treated with DMSO or 1938 is shown. b, Infarct size measured at the end of the 2 h reperfusion, in hearts administered either DMSO (n=6) or 1938 (n=6). c, pAKT^{S437} in hearts administered either DMSO (n=5), 1938 (n=6) or insulin (n=2). 1-way ANOVA with Tukey post-test. d, Impact of 1938 on neuronal regeneration in vitro and in vivo. DRG cultures were stimulated with 1938 in the presence or absence of BYL719 for 72 h, followed by measurement of neurite length. Shown are representative images of neurons stained with anti-β-III tubulin at the 72 h time point. Scale bar=1000 μm. Quantified data represent mean±SEM, n=3 independent experiments. e, Illustration of sciatic nerve crush injury (i), with arrowhead in (ii) showing the resulting lesion. Injury induction was followed by (iii) direct injection proximal to the injury, of a single dose of dH₂O or 1938 (2 μl of a 5 μM solution of 1938 in sterile dH₂O) and (iv) implantation of a minipump for continuous delivery of dH₂O or 1938 (100 μM solution of 1938 in dH₂O delivered continuously at 0.11 μl/h) for 21 days, followed by histological and functional analysis. f, Motor unit number estimation (MUNE) electrophysiological recordings from the tibialis anterior (TA) muscle. g, Compound muscle action potential (CMAP) recordings in the TA muscle following nerve stimulation proximal to the crush site. CMAP recovery is presented as a percentage of the contralateral side. h, Total number of choline acetyltransferase (ChAT)-positive motor axons in cross-sections of the distal common peroneal nerve that innervates the TA muscle. i, Representative immunohistochemistry image of a transverse section through the distal common peroneal nerve from a 1938-treated animal, showing ChAT- and neurofilament-positive motor axons with tissue architecture typical of normal tissue. Scale bar=50 μm. j, Proportion of neuromuscular junctions (NMJs) re-innervated by axons at the target TA muscle, as revealed by double staining with α-bungarotoxin (α-BTX) and neurofilament (NF). k, Representative immunohistochemistry image of TA muscle from a 1938-treated animal showing α-BTX-stained post-synaptic NMJ structure with associated neurofilament-positive neurons. Scale bar=20 μm. 1, Quantification of total axons (neurofilament) and motor axons (ChAT) in the sciatic nerve at 3 mm and 6 mm distal to the injury site after 21 days. n=5 per group, error bars are SD. CP=position of distal common peroneal nerve cross-sections. Two-tailed Student's t-tests, *=p<0.05, **=P<0.01. All data are from the 21 day endpoint.

FIG. 6|PI3K activator induced cell death in lung cancer cells. Shows the ability of PI3K activators to induce cell death in lung cancer cells in the presence and absence of a PI3Kα-selective inhibitor.

FIG. 7|PI3K activator induced cell death in lung cancer cells. Shows the ability of PI3K activators to induce cell death in lung cancer cells in the presence and absence of PI3K pathway inhibitors.

FIG. 8|Short term exposure to PI3Kα-activator induces cell death. Shows the ability of TRO-1938 to induce cell death after a short exposure time in a H460, H1975 and U87-MG cancer cell lines.

FIG. 9|Extended data FIG. 1. Shows the activation of class IA PI3K isoforms by a concentration range of pY, as described in more detail in Example 1.

FIG. 10|Extended data FIG. 2. Shows a surface model of full-length p110α/p85α, showing changes induced by 1938 as assessed by HDX-MS.

FIG. 11|Shows that 1938 provides significant cardioprotection in an in vivo model of IRI in mice (left panel), with a corresponding increase in pAKT^{S473} levels in the hearts of these mice (right panel).

FIG. 12|Extended data FIG. 4. TIRF microscopy data from HeLa cells expressing the EGFP-tagged PIP₃ reported PH-ARNO-1303Ex2 (ARNO) or the PI(3,4)P₂ reporter mCherry-cPH-TAPP1x3, as described in more detail in Example 4.

FIG. 13|Extended data FIG. 5. Time course analysis of 1938-induced pAKT^{S473} and pS6^{S240/44} in MCF10A cells by 1938 in the presence of absence of BYL719, as described in more detail in Example 4.

FIG. 14|Extended data FIG. 6. In vitro kinase inhibition profile of 1938 (1 μM) on 133 protein kinases and 7 lipid kinases, as described in more detail in Example 5.

FIG. 15|Extended data FIG. 7. Effect of 1938 on in vitro kinase activity of the PI3K-related kinases ATM and mTORC1 (mTOR/RAPTOR/LST8 complex), as described in more detail in Example 5.

FIG. 16|Extended data FIG. 8. a Experimental design and workflow of phosphoproteomics experiment, as described in more detail in Example 5. b Validation of phosphoproteomics conditions, as described in more detail in Example 5. c Plot showing how insulin stimulation induces phosphorylation of expected PI3K targets in PI3Kα-WT MEFs, as described in more detail in Example 5. d Plots showing the high experimental reproducibility of the phosphoproteomics experiment.

FIG. 17|Extended data FIG. 9. Plots showing the effect of stimulating PI3Kα-WT and PI3Kα-KO MEFs with 1938 for 24, 48 or 72 h, as described in more detail in Example 6.

FIG. 18| Extended data FIG. 10. Western blot showing pAKT^{S473} induction in primary adult rat cardiomyocytes, as described in more detail in Example 7.

FIG. 19|Extended data FIG. 11. Left Control experiment to test the biological activity of 1938 post-freezing. Right Blot showing the induction of pAKT^{S473} in exposed sciatic nerves, when injected with vehicle or 1938 or bathed in a solution of vehicle or 1938, as described in more detail in Example 7.

FIG. 20. a, Structural changes induced by 1938 in full-length p110α/p85α as assessed by HDX-MS, highlighted on the structure of p110α/ninterSH2-p85α (pdb: 4ZOP). b,i., ii. Crystal structure binding mode of 1938 to p110α. iii. Comparative view of 1938 binding in a pocket on p110α, the region analogous to the p110α pocket in p110β, and then analogous pocket in p110δ. iv. Comparative activation of WT PI3Kα and mutant PI3Kα with pY and 1938. The mutants incorporate mutations of p110α residues in the vicinity of the pocket that accommodates 1938.

FIG. 21. a, MEFs were stimulated for the indicated time points with 1938 (5 μM) or for 2 min with PDGF (20 ng/ml) or insulin (100 nM), followed by lipid extraction and PIP3 measurement by mass spectrometry. b, MEFs were stimulated for 2 min with increasing doses of 1938 or PDGF, followed by lipid extraction and PIP3 measurement by mass spectrometry. c, A549 cells were stimulated for 2 min with increasing doses of 1938 or insulin, or 10 ng/ml PDGF, followed by lipid extraction and PIP₃ measurement by mass spectrometry.

FIG. 22 (Updated version of FIG. 1)|Biochemical mechanism of PI3Kα activation by UCL-TRO-1938. a, Structure of UCL-TRO-1938 (referred to in the text as 1938). b, Selectivity of 1938 for PI3Kα over PI3Kβ and PI3Kδ. c, Enzyme kinetics (calculated using Michaelis-Menten kinetic analysis in Prism 8) upon ATP titration on PI3Kα with or without 1938 and pY. d, Membrane binding of PI3Kα shown as FRET signal (I-I0). I, fluorescence intensity at 520 nm, I₀, fluorescence intensity at 520 nm in the absence of enzyme. e, Effect of 1938 on PI3Kα catalytic activity in the presence of a saturating dose of pY. f, Effect of 1938 on the catalytic activity of oncogenic mutants of PI3Kα. g, Effect of 1938 on membrane binding of oncogenic mutants of PI3Kα. h, Effect of the PI3Kα-selective inhibitor BYL719 on 1938-activated PI3Kα. i. Effect of 1938 on the IC₅₀ of BYL719 for PI3Kα. Data shown as mean±SEM, n=3 (b,c,e,i), n=2 (d,h), or n=4 (f,g) independent experiments. Statistical analysis performed with two way ANOVA, Tukey's multiple comparisons test (b,f,g) or Sidak's multiple comparisons test (h); one way ANOVA, Dunnett's multiple comparisons test (e).

### DEFINITIONS

Compounds of the present invention may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. Geometric isomers of double bonds such as olefins bonds can also be present in the compounds described herein, and all such stable isomers are contemplated. Cis and trans geometric isomers of the compounds are described and may be isolated as a mixture of isomers or as separated isomeric forms. All chiral, diastereomeric, racemic forms and all geometric isomeric forms of a structure are intended, unless the specific stereochemistry or isomeric form is specifically indicated. Hence, the compounds of the present invention include all possible chiral, diastereomeric and racemic forms thereof and all possible geometric isomers thereof. When no specific mention is made of the configuration (cis, trans, R, S, etc.) of a compound (or of an asymmetric carbon), then any one of the isomers or a mixture of more than one isomer is intended. Limitation to a particular asymmetric form of a compound is only intended where expressly indicated.

The processes for preparation can use racemates, enantiomers, or diastereomers as starting materials. When enantiomeric or diastereomeric products are prepared, they can be separated by conventional methods, for example, by chromatographic or fractional crystallization. The prepared compounds may be in the free or hydrate form.

Compounds of the present invention encompass both compounds in which: (a) all contained atoms are in their natural isotopic form (“natural isotopic form of the compound”); and (b) compounds in which one or more contained atoms are in a non-natural isotopic form (“unnatural variant isotopic form of the compound”), for instance compounds comprising isotopic replacement, enrichment, or depletion. An unnatural variant isotopic form of the compound may thus contain one or more artificial or uncommon isotopes such as deuterium (2H or D), carbon-11 (¹¹C), carbon-13 (¹³C), carbon-14 (¹⁴C), nitrogen-13 (¹³N), nitrogen-15 (¹⁵N), oxygen-15 (¹⁵O), oxygen-17 (¹⁷O), oxygen-18 (¹⁸O), phosphorus-32 (³²P), sulphur-35 (³⁵S), chlorine-36 (³⁶Cl), chlorine-37 (³⁷Cl), fluorine-18 (¹⁸F) iodine-123 (¹²³I), iodine-125 (¹²⁵I) in one or more atoms or may contain an increased proportion of said isotopes as compared with the proportion that predominates in nature in one or more atoms.

Unnatural variant isotopic forms of the compound comprising radioisotopes may, for example, be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Unnatural variant isotopic forms which incorporate deuterium i.e. ²H or D may afford certain therapeutic advantages resulting from greater metabolic stability, for example, increased in vivo half-life or reduced dosage requirements, and hence may be preferred in some circumstances. Further, unnatural variant isotopic forms may be prepared which incorporate positron emitting isotopes, such as ¹¹C, ¹⁸F, ¹⁵O and ¹³N, and would be useful in Positron Emission Topography (PET) studies for examining substrate receptor occupancy.

The following are definitions of terms used in this specification. The initial definition provided for a group or term herein applies to that group or term throughout the present specification, individually or as part of another group, unless otherwise indicated. Preferably, the molecular weight of compounds of the present invention is less than about 500, 550, 600, 650, 700, 750, or 800 grams per mole.

Preferably, the molecular weight is less than about 800 grams per mole. More preferably, the molecular weight is less than about 750 grams per mole. Even more preferably, the molecular weight is less than about 700 grams per mole.

The term “substituted” as used herein, means that any one or more hydrogens on the designated atom is replaced with a selection from the indicated group, provided that the designated atom's normal valency is not exceeded, and that the substitution results in a stable compound (for instance, avoiding unstable acetals or similar groups).

When any variable (e.g., R⁴, R^{b}, etc.) occurs more than one time in any constituent or formula for a compound, its definition at each occurrence is independent of its definition at every other occurrence. Thus, for example, if a group is shown to be substituted with 0-3 R⁴, then said group may optionally be substituted with up to three R⁴ groups and R⁴ at each occurrence is selected independently from the definition of R⁴. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

As used herein, “alkyl” is intended to include both branched and straight-chain saturated aliphatic hydrocarbon groups having the specified number of carbon atoms. For example, “C₁-C₆ alkyl”, is intended to include C₁, C₂, C₃, C₄, C₅, and C₆ alkyl groups. Additionally, for example, “C₁-C₆ alkyl” denotes alkyl having 1 to 6 carbon atoms. Examples of alkyl include, but are not limited to, methyl, ethyl, n-propyl, i-propyl, n-butyl, i-butyl, sec-butyl, t-butyl, n-pentyl, n-hexyl, 2-methylbutyl, 2-methylpentyl, 2-ethylbutyl, 3-methylpentyl, and 4-methylpentyl.

The phrase “pharmaceutically acceptable” is employed herein to refer to those compounds, materials, compositions, and/or dosage forms which are, within the scope of sound medical judgment, suitable for use in contact with the tissues of human beings and animals, especially human beings, without excessive toxicity, irritation, allergic response, or other problem or complication, commensurate with a reasonable benefit/risk ratio.

The compounds of the present invention encompass pharmaceutically acceptable salts (in particular, pharmaceutically acceptable salts of compounds of formula (I), as well as solvates, N-oxides, and tautomers thereof). As used herein, “pharmaceutically acceptable salts” refer to derivatives of the disclosed compounds wherein the parent compound (e.g., of formula (I)) is modified by making pharmaceutically acceptable acid or base salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

The pharmaceutically acceptable salts of the present invention can be synthesized from the parent compound (e.g., of formula (I)) which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, PA, 1985, p. 1418, the disclosure of which is hereby incorporated by reference.

Non-pharmaceutically acceptable salt forms of the compounds of the invention may be of use during the preparation of non-salt or pharmaceutically acceptable salt forms. Consequently, the invention also encompasses a non-pharmaceutically acceptable salt of a compound of formula (I).

The compounds of the present invention encompass solvates (in particular, solvates of compounds of formula (I), as well as salts, N-oxides, and tautomers thereof). Solvates include, and preferably are, hydrates. Methods of solvation are generally known in the art.

The compounds of the present invention encompass tautomers (in particular, tautomers of compounds of formula (I), as well as salts and solvates thereof). Some compounds of the invention may exist in a plurality of tautomeric forms, in which hydrogen atoms are transposed to other parts of the molecules and the chemical bonds between the atoms of the molecules are consequently rearranged. It should be understood that all tautomeric forms, insofar as they may exist, are encompassed by the compounds of the present invention.

In cases wherein there are nitrogen atoms (e.g., amines) on compounds of the present invention (e.g. of formula (I)), these can be converted to N-oxides by treatment with an oxidizing agent (e.g., MCPBA and/or hydrogen peroxides) to afford other compounds of this invention. Thus, all shown nitrogen atoms are considered to cover both the shown nitrogen and its N-oxide (N→O) derivative. The compounds of the present invention therefore encompass N-oxides (in particular, N-oxides of compounds of formula (I), as well as salts, solvates and tautomers thereof).

For avoidance of doubt, many compounds of the invention can exist simultaneously in the form of two or more of a tautomer, N-oxide, pharmaceutically acceptable salt, and solvate of a particular parent compound (e.g. a compound of formula (I)), and all such possible compounds are encompassed within the definition of “compound of the invention”. Hence, a compound of the invention (to the extent chemically possible in view of the relevant formula (I) expressly includes any one of the following: (1) a compound of formula (I) (which may also be referred to herein as a “free base form” of the compound); (2) a tautomer of formula (I); (3) an N-oxide of formula (I); (4) a pharmaceutically acceptable salt of formula (I); (5) a solvate of formula (I); (6) an N-oxide of a tautomer of formula (I); (7) a pharmaceutically acceptable salt of a tautomer of formula (I); (8) a solvate of a tautomer of formula (I); (9) a pharmaceutically acceptable salt of an N-oxide of a tautomer of formula (I); (10) a solvate of an N-oxide of a tautomer of formula (I); (11) a solvate of a pharmaceutically acceptable salt of a tautomer of formula (I); (12) a solvate of a pharmaceutically acceptable salt of an N-oxide of a tautomer of formula (I); (13) a pharmaceutically acceptable salt of an N-oxide of formula (I); (14) a solvate of an N-oxide of formula (I); (15) a solvate of a pharmaceutically acceptable salt of an N-oxide of a tautomer of formula (I); and (16) a solvate of a pharmaceutically acceptable salt of formula (I).

Compounds of the present invention are, subsequent to their preparation, preferably isolated and purified to obtain a composition containing an amount by weight equal to or greater than 99% compound (“substantially pure”), which is then used or formulated as described herein. Such “substantially pure” compounds are also part of the present invention.

“Stable compound” and “stable structure” are meant to indicate a compound that is sufficiently robust to survive isolation to a useful degree of purity from a reaction mixture, and formulation into an efficacious therapeutic agent.

Purely for the avoidance of doubt, it should be noted that wherever a compound is disclosed as being either a particular structure (e.g. a particular general chemical formula, such as of formula (I)) or a tautomer, N-oxide, pharmaceutically acceptable salt, or solvate thereof, then, unless expressly indicated to the contrary, any disclosed further embodiment of that compound (e.g. “A compound . . . according to . . . ”), and describing additional limitations to the recited structure, continues to embrace any such tautomer, N-oxide, pharmaceutically acceptable salt, or solvate thereof.

As used herein, “treating and/or preventing” or “treatment and/or prevention” of a disease-state in a mammal, particularly a human, include: (a) preventing the disease-state from occurring in a mammal, in particular, when such mammal is predisposed to the disease-state but has not yet been diagnosed as having it; (b) inhibiting the disease-state, i.e., slowing or arresting its development; and/or (c) relieving the disease-state, i.e., causing regression of the disease state or a reduction in associated symptoms.

“Therapeutically effective amount” is intended to include an amount of a compound that is effective to achieve a desirable effect in treating and/or preventing a disease-state. A desirable effect is typically clinically significant and/or measurable, for instance in the context of (a) preventing the disease-state from occurring in a mammal, in particular, when such mammal is predisposed to the disease-state but has not yet been diagnosed as having it; (b) inhibiting the disease-state, i.e., slowing or arresting its development; and/or (c) relieving the disease-state, i.e., causing regression of the disease state or a reduction in associated symptoms. The therapeutically effective amount may be one that is sufficient to achieve the desirable effect either when the compound is administered alone, or alternatively when it is administered in combination with one or more further APIs, which either are further compounds of the invention or are different from the compounds of the invention. Furthermore, a therapeutically effective amount is typically an amount that is sufficient to activate PI3Kα, again when administered either alone or in combination with one or more further APIs (which may also activate PI3Kα, or alternatively may exert their pharmacological effects by a different mechanism). Thus, “therapeutically effective amount” is intended to include an amount of a combination of compounds that each are compounds of the invention that is effective to activate PI3Kα. The combination of compounds is preferably a synergistic combination.

Synergy, as described, for example, by Chou and Talalay, Adv. Enzyme Regul. 1984, 22: 27-55, occurs when the effect (in this case, activation of PI3Kα) of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, or some other beneficial effect of the combination compared with the individual components.

For avoidance of doubt, a “therapeutically effective amount” as recited herein can be achieved by any suitable dosage regimen, including but not limited to exemplary dosage regimens described elsewhere herein. Hence, for example, references herein to administering a therapeutically effective amount of a compound by a particular administration route including achieving the therapeutically effective amount via a single dose or by plural doses administered by the specified administration route. For instance, orally administering a therapeutically effective amount includes both orally administering a single dose and orally administering any plural number of doses, provided that a therapeutically effective amount is thereby achieved by oral administration.

The present invention further includes compositions comprising one or more compounds of the present invention and one or more pharmaceutically acceptable carriers.

A “pharmaceutically acceptable carrier” refers to media generally accepted in the art for the delivery of biologically active agents to animals, in particular, mammals. Pharmaceutically acceptable carriers are formulated according to a number of factors well within the purview of those of ordinary skill in the art. These include, without limitation: the type and nature of the active agent being formulated; the subject to which the agent-containing composition is to be administered; the intended route of administration of the composition; and, the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Remington's Pharmaceutical Sciences, 17th ed., 1985, which is incorporated herein by reference in its entirety.

### DETAILED DESCRIPTION OF THE INVENTION

### Compounds of the Present Invention

The present invention provides a compound that (a) is of formula (I):

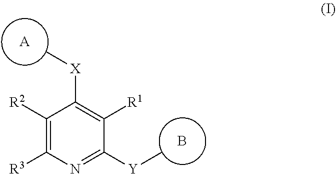

or (b) is a tautomer, N-oxide, pharmaceutically acceptable salt, or solvate thereof; wherein:
X is bond or NH;
Y is bond or NH;
with the proviso that at least one of X and Y is NH;
R¹ is H, F or CH₃;
R² is H, F, Cl, Br, —COR⁴, —SO₂R⁵, —SOR⁵, —CN, —NO, —NO₂ or —NR⁶₃⁺;
R³ is H, CH₃, C₂-C₆ alkyl substituted with 0 to 3 R⁷, —COR⁴, —SO₂R⁵, —SOR⁵, —CN, —NO, —NO₂ or —NR⁶₃⁺;
R⁴ is independently selected from H, C₁-C₆ alkyl substituted with 0 to 3 R⁷, —OH, —OR⁸, —NH₂, —NHR⁸ or —NR⁸₂;
R⁵ is independently selected from C₁-C₆ alkyl substituted with 0 to 3 R⁷, —OH, —OR⁸, —NH₂, —NHR⁸ or —NR⁸₂;
R⁶ is independently selected from C₁-C₃ alkyl;
R⁷ is independently selected from O—C₁-C₃ alkyl, F or Cl;
R⁸ is independently selected from C₁-C₆ alkyl substituted with 0 to 3 R⁷;
ring A is selected from a ring within group I, group II, group III, group IV and group V,
wherein * denotes attachment to X;
group I is group I-1, i.e. is:

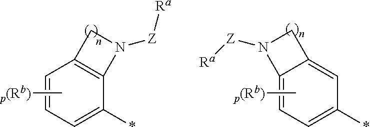

group II is group II-1, i.e. is:

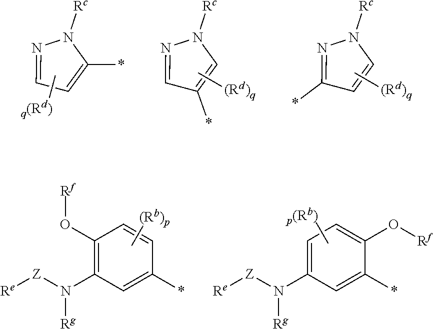

group III is group III-1, i.e. is:

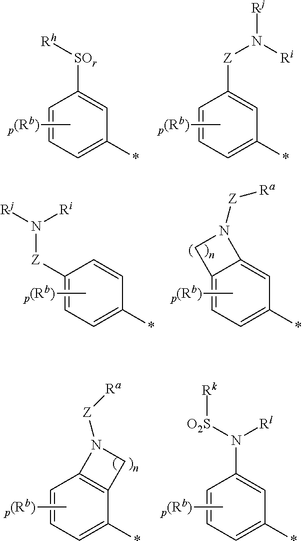

group IV is group IV-1, i.e. is:

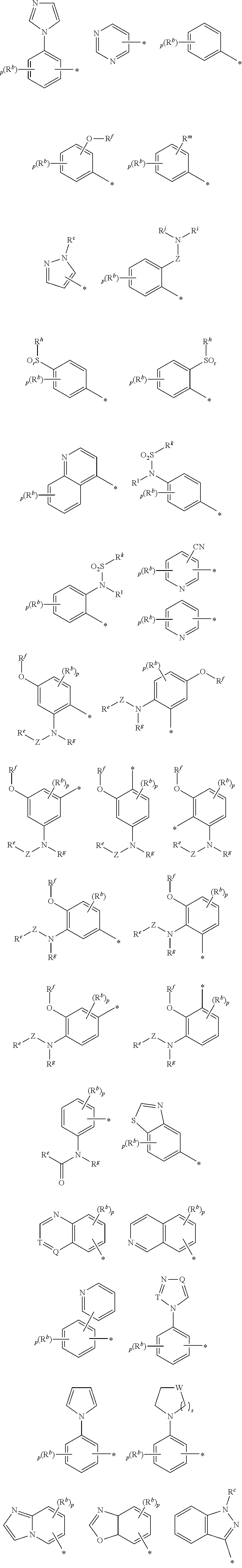

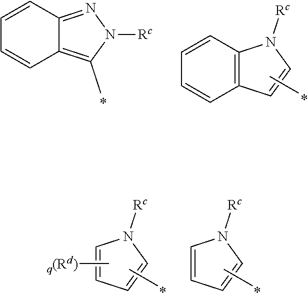

group V is group V-1, i.e. is:

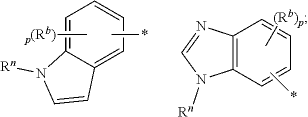

ring B is selected from a ring within group IA, group IIA, group IIA, group IVA, and group VA, wherein ^{$} denotes attachment to Y;
group IA is group IA-1, i.e. is:

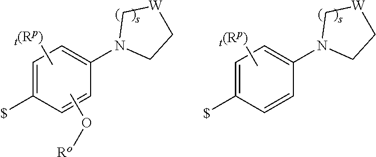

group IIA is group IIA-1, i.e. is:

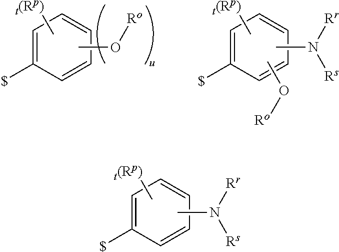

group IIIA is group IIIA-1, i.e. is:

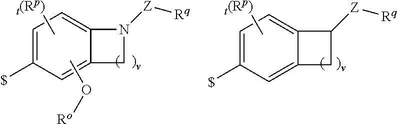

group IVA is group IVA-1, i.e. is:

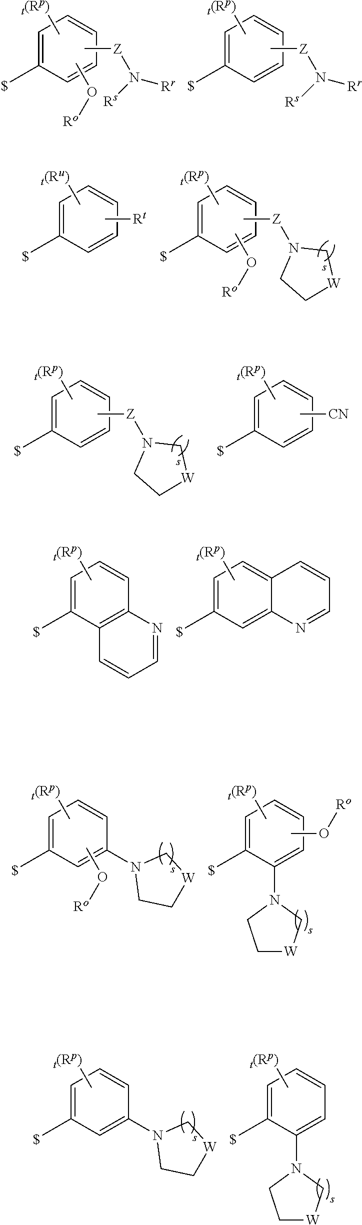

group VA is group VA-1, i.e. is:

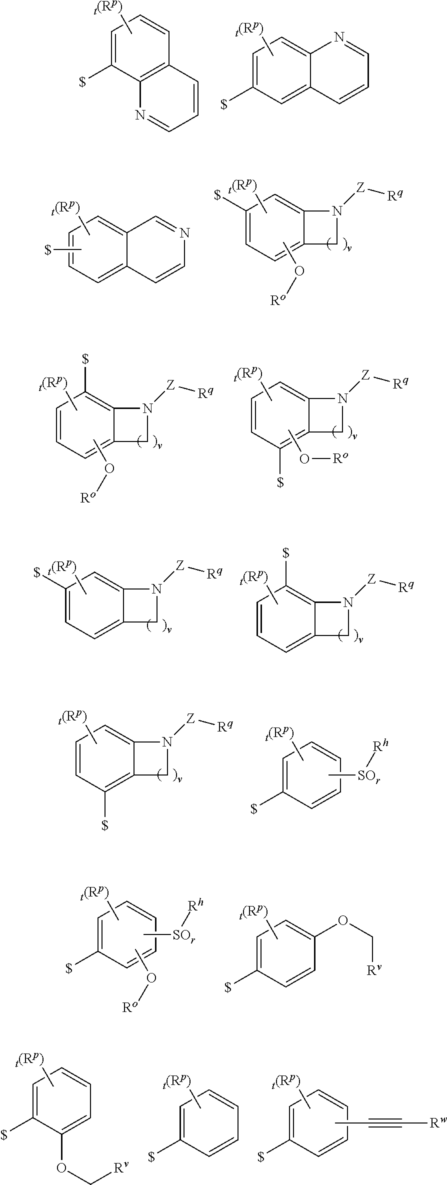

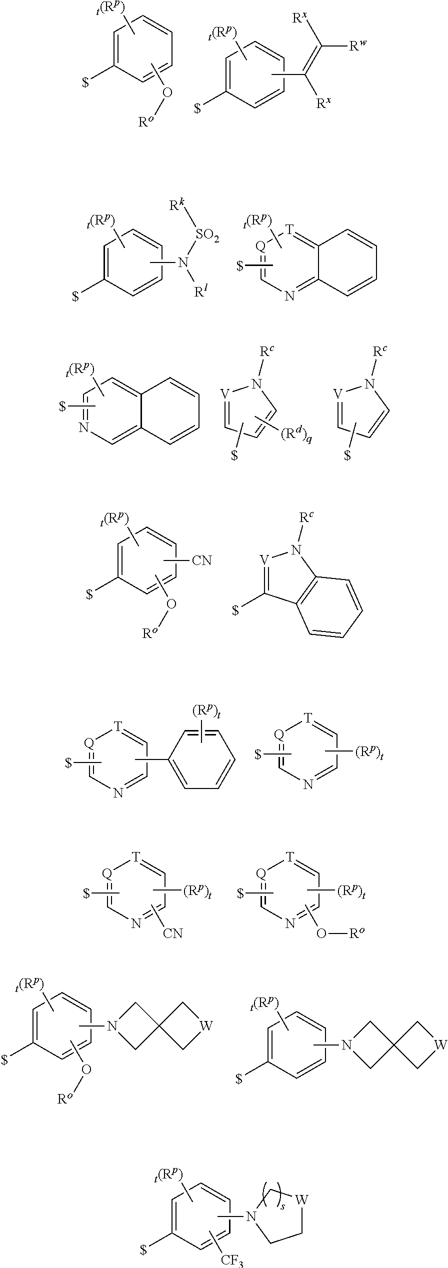

wherein:
Q and T are each selected from CH or N, with the proviso that at most one of Q and T may be N;
V is CH or N;
W is CH₂, O, NR, S, S(O) or S(O)₂;
Z is C(O), S(O) or S(O)₂;
R^{a} is C₁-C₆ alkyl substituted with 0 to 3 substituents independently selected from O—C₁-C₃ alkyl, F and Cl;
R^{b} is independently selected from C₁-C₆ alkyl, F and Cl;
R^{c} is C₁-C₆ alkyl substituted with 0 to 3 substituents independently selected from O—C₁-C₃ alkyl, F and Cl;
R^{d} is independently selected from C₁-C₆ alkyl substituted with 0 to 3 substituents independently selected from O—C₁-C₃ alkyl, F and Cl; or phenyl substituted with 0 to 3 substituents independently selected from O—C₁-C₃ alkyl, F and Cl;
R^{e} is C₁-C₆ alkyl substituted with 0 to 3 substituents independently selected from O—C₁-C₃ alkyl, F and Cl;
R^{f} is C₁-C₆ alkyl substituted with 0 to 3 substituents independently selected from O—C₁-C₃ alkyl, F and Cl;
R^{g} is H or C₁-C₃ alkyl;
R^{h} is C₁-C₆ alkyl substituted with 0 to 3 substituents independently selected from O—C₁-C₃ alkyl, F and Cl;
Rⁱ is C₁-C₆ alkyl substituted with 0 to 3 substituents independently selected from O—C₁-C₃ alkyl, F and Cl;
R^{j} is H or C₁-C₃ alkyl
R^{k} is C₁-C₆ alkyl substituted with 0 to 3 substituents independently selected from O—C₁-C₃ alkyl, F and Cl;
R^{l} is H or C₁-C₃ alkyl;
R^{m} is C₁-C₆ alkyl substituted with 0 to 3 substituents independently selected from O—C₁-C₃ alkyl, F and Cl;
Rⁿ is H or C₁-C₃ alkyl
R^{o} is independently selected from C₁-C₆ alkyl substituted with 0 to 3 substituents independently selected from O—C₁-C₃ alkyl, F and Cl; or phenyl substituted with 0 to 3 substituents independently selected from O—C₁-C₃ alkyl, F and Cl;
R^{p} is independently selected from C₁-C₆ alkyl, F, Cl and Br
R^{q} is C₁-C₆ alkyl substituted with 0 to 3 substituents independently selected from O—C₁-C₃ alkyl, F and Cl;
R^{r} is H or C₁-C₆ alkyl substituted with 0 to 3 substituents independently selected from O—C₁-C₃ alkyl, F and Cl
R^{s} is H or C₁-C₃ alkyl
R^{t} is C₁-C₆ alkyl substituted with 0 to 3 substituents independently selected from O—C₁-C₃ alkyl, F and Cl;
R^{u} is independently selected from C₁-C₆ alkyl, F and Cl;
R^{v} is phenyl substituted with 0-2 substituents selected from C₁-C₆ alkyl, F and Cl;
R^{w} is H or C₁-C₃ alkyl;
R^{x} is independently selected from H or C₁-C₃ alkyl;
R^{y} is H, C₁-C₆ alkyl substituted with 0 to 3 substituents independently selected from O—C₁-C₃ alkyl, F and Cl; benzyl substituted with 0 to 3 substituents independently selected from C₁-C₆ alkyl, F and Cl; or C₃-C₆ cycloalkyl substituted with 0 to 3 substituents independently selected from C₁-C₆ alkyl, O—C₁-C₃ alkyl, F and Cl;
n is 1 to 3;
p is 0 to 2;
q is 1 or 2;
r is 0 to 2;
s is 1 to 3;
t is 0 to 2;
u is 2 to 3;
v is 1 to 3;
and wherein:
   when X is NH; Y is NH; R² is H; and ring A is a ring within group I or group II; then: ring B is a ring within group IA, group IIA, group IIIA, group IVA or group VA;
   when X is NH; Y is NH; R² is H; and ring A is a ring within group III, then: ring B is a ring within group IA, group IIA or group IIIA;
   when X is NH; Y is NH R² is H; and ring A is a ring within group IV, then: ring B is a ring within group IA or group IIA;
   when X is NH; Y is NH; R² is H; and ring A is a ring within group V, then: ring B is a ring within group IA;
   when X is NH; Y is NH; and R² is F, Cl, Br, —COR⁴, —SO₂R⁵, —SOR⁵, —CN, —NO, —NO₂ or —NR⁶₃⁺; then: ring A is a ring within group I or group II and ring B is a ring within group IA;
   when X is bond and Y is NH, then: R² is H; ring A is a ring within group I, group II and group III; and ring B is a ring within group IA; and
   when X is NH and Y is bond, then: R² is H; ring A is a ring within group I, group II and group III; and ring B is a ring within group IA, group IIA, and group IIIA;
and with the proviso that the compound of formula (I) is not (a) a compound selected from the group:

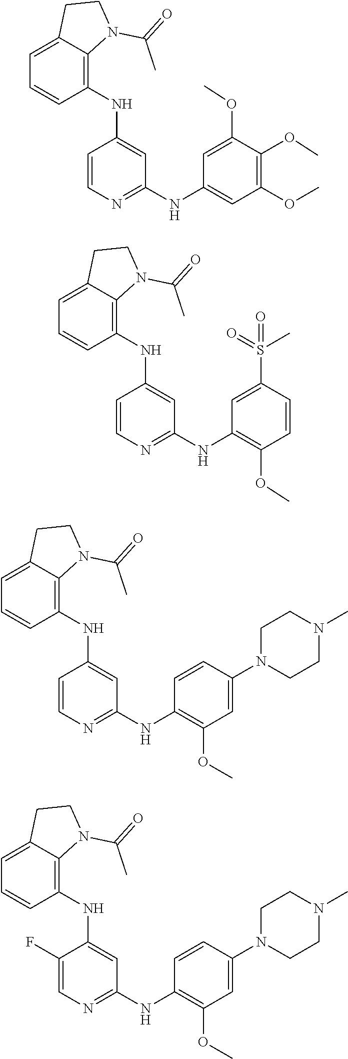

nor (b) a tautomer, N-oxide, pharmaceutically acceptable salt, or solvate thereof.

In a preferred embodiment, X is NH and Y is NH.

In another embodiment, X is bond and Y is NH. In a further alternative embodiment, X is NH and Y is bond.

Preferably, R¹ is H or F. For instance, R¹ is H. Alternatively, R¹ is F.

Preferably, R² is H, F, Cl, Br, —COR⁴, —SO₂R⁵, —CN, —NO₂ or —NR⁶₃⁺. Further preferably, R² is H, F, Cl, Br, —COR⁴, —SO₂R⁵, or —CN. Further preferably still, R² is H, F, Cl, or Br. Even more preferably, R² is H or F. Most preferably, R² is H.

Preferably, R³ is H, CH₃, C₂-C₆ alkyl substituted with 0 to 3 R⁷, —COR⁴, —SO₂R⁵, —CN, —NO₂ or —NR⁶₃⁺. Further preferably, R³ is H, CH₃, C₂-C₆ alkyl substituted with 0 to 3 R⁷, —COR⁴, —SO₂R⁵, or —CN. Further preferably still, R³ is H, CH₃ or C₂-C₆ alkyl substituted with 0 to 3 R⁷. Even more preferably, R³ is H, CH₃ or C₂-C₆ alkyl. Even more preferably still, R³ is H or CH₃. Most preferably, R³ is H.

Preferably, R⁴ is independently selected from —OR⁸, —NH₂, —NHR⁸ or —NR⁸₂.

Preferably, R⁵ is independently selected from —OR⁸, —NH₂, —NHR⁸ or —NR⁸₂.

Preferably, R⁶ is independently selected from C₁-C₂ alkyl. Most preferably, R⁶ is CH₃.

Where an alkyl group is substituted with 0 to 3 R⁷, then the number of R⁷ groups is 0 (i.e. the alkyl group is unsubstituted), 1, 2 or 3, preferably being 0, 1 or 2, more preferably 0 or 1 and most preferably 0 (i.e. the alkyl group is unsubstituted).

Preferably, R⁷ is independently selected from O—C₁-C₂ alkyl, F or Cl. Further preferably, R⁷ is independently selected from OCH₃, F or Cl.

Preferably, R⁸ is independently selected from C₁-C₃ alkyl substituted with 0 to 3 R⁷. Further preferably, R⁸ is independently selected from C₁-C₃ alkyl. Further preferably still, R⁸ is independently selected from C₁-C₂ alkyl. Most preferably, R⁸ is CH₃.

Thus, in a preferred combination (combination A1):
R² is H, F, Cl, Br, —COR⁴, —SO₂R⁵, or —CN;
R³ is H, CH₃, C₂-C₆ alkyl substituted with 0 to 3 R⁷, —COR⁴, —SO₂R⁵, or —CN;
R⁴ and R⁵ are each independently selected from —OR⁸, —NH₂, —NHR⁸ or —NR⁸₂;
R⁶ is independently selected from C₁-C₂ alkyl;
R⁷ is independently selected from OCH₃, F or Cl; and
R⁸ is independently selected from C₁-C₃ alkyl.

In a further preferred combination (combination A2):
R² is H, F, Cl, or Br; and
R³ is H, CH₃ or C₂-C₆ alkyl.

In a more preferred combination (combination A3):
R² is H or F; and
R³ is H or CH₃.

In a more preferred combination still (combination A4):
R² is H or F;
and R³ is H.

In the compound of formula (I), with respect to ring A, in a particularly preferred embodiment, group I is group I-2, i.e. is:

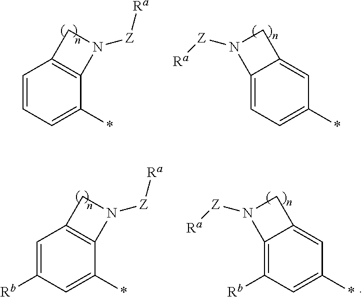

More preferably, group I is group I-3, i.e. is:

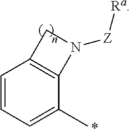

Most preferably, group I is group I-4, i.e. is:

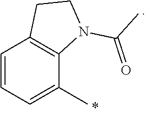

In an embodiment, group II is group II-2, i.e. is:

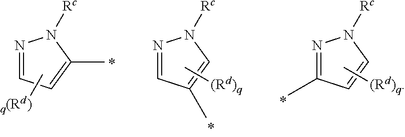

In an alternative embodiment, group II is group II-2′, i.e. is:

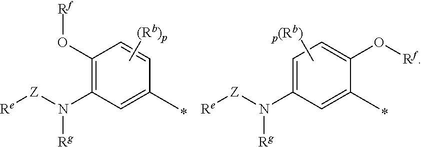

In a preferred embodiment, group II is group II-3, i.e. is:

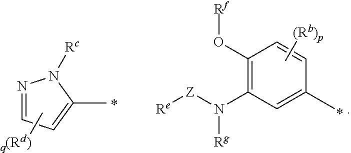

In a particularly preferred embodiment, group II is group II-4, i.e. is:

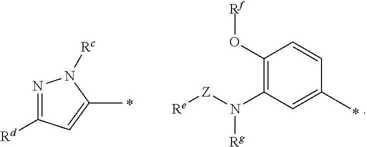

More preferably, group II is group II-5, i.e. is:

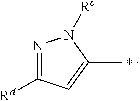

Most preferably, group II is group II-6, i.e. is:

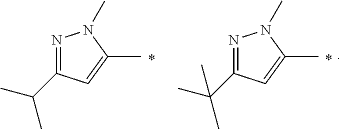

In an embodiment, group III is group III-2, i.e. is:

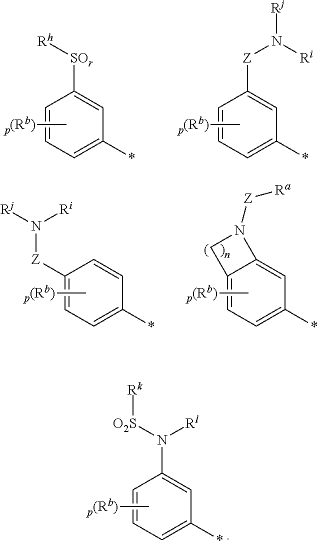

In another embodiment, group III is group III-3, i.e. is:

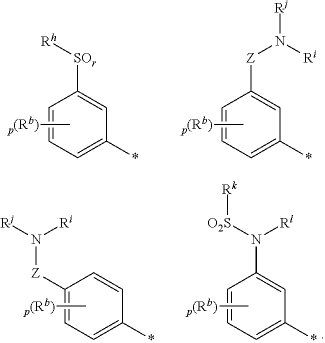

In a preferred embodiment, group III is group III-4, i.e. is:

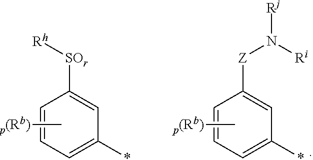

In a particularly preferred embodiment, group III is group III-5, i.e. is:

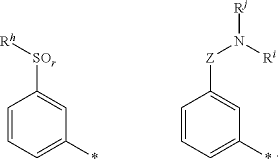

Most preferably, group III is group III-6, i.e. is:

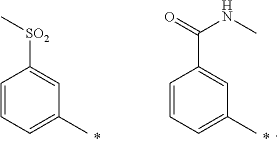

In an embodiment, group IV is group IV-2, i.e. is:

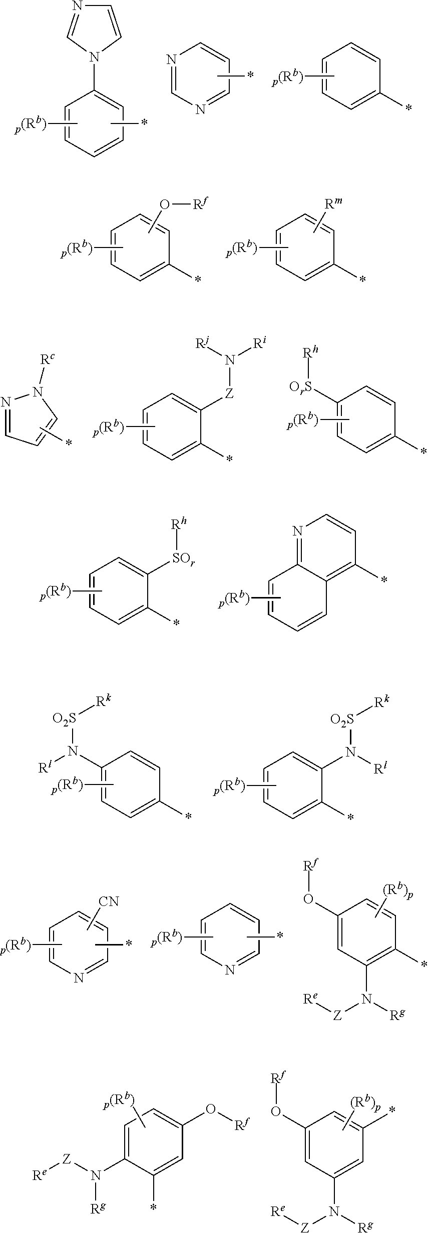

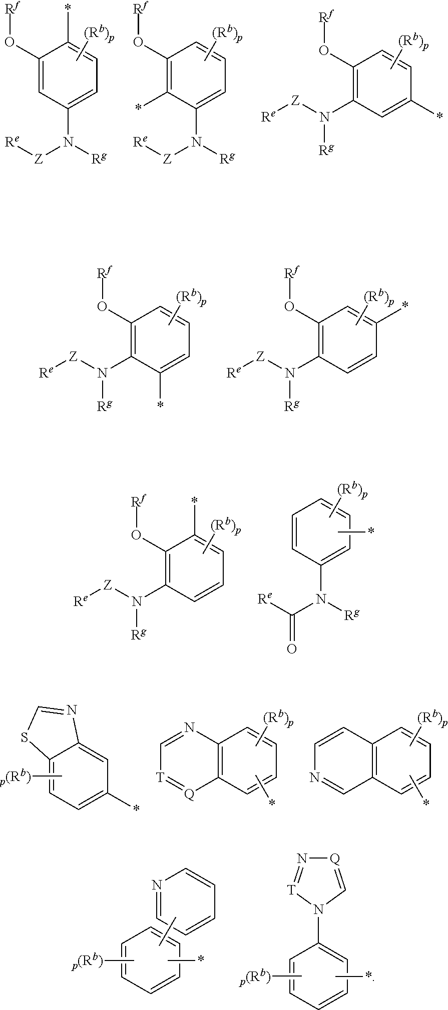

In a preferred embodiment, group IV is group IV-3, i.e. is:

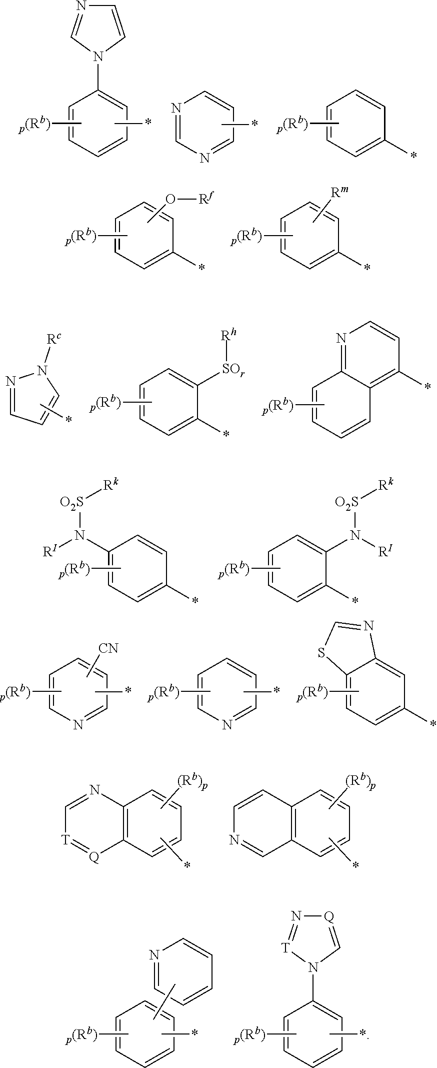

In a preferred embodiment, group IV is group IV-4, i.e. is:

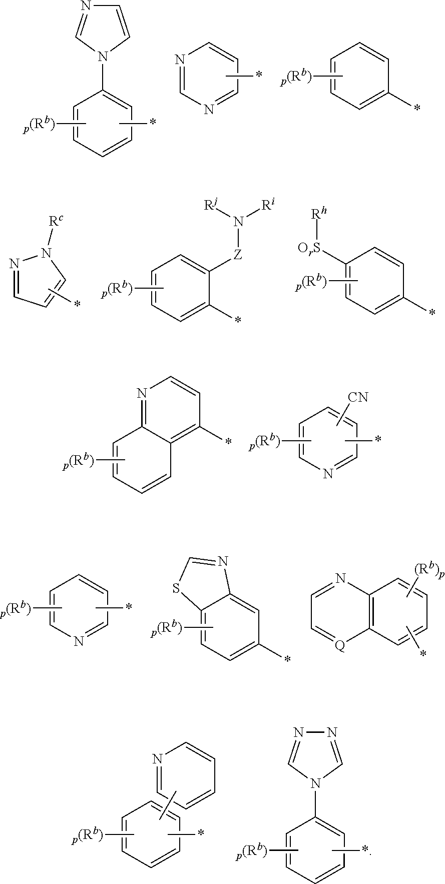

In a particularly preferred embodiment, group IV is group IV-5, i.e. is:

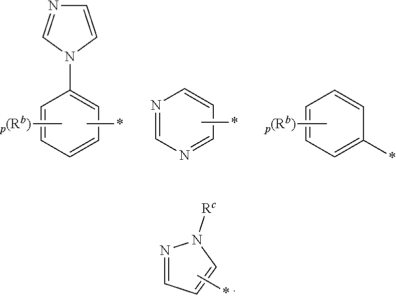

Most preferably, group IV is group IV-6, i.e. is:

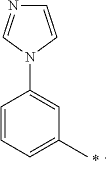

In a preferred embodiment, group V is group V-2, i.e. is:

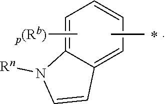

Most preferably, group V is group V-3, i.e. is:

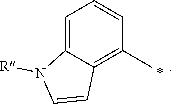

In the compound of formula (I), with respect to Ring B, in a particularly preferred embodiment, group IA is group IA-2, i.e. is:

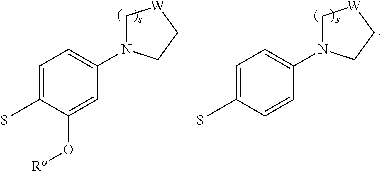

Most preferably, group IA is group IA-3, i.e. is:

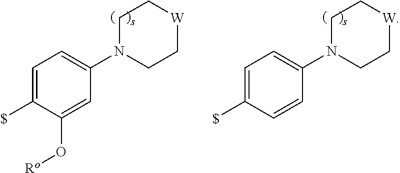

In a preferred embodiment, group IIA is group IIA-2, i.e. is:

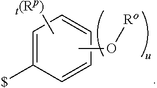

In a particularly preferred embodiment, group IIA is group IIA-3, i.e. is:

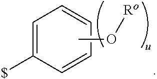

Most preferably, group IIA is group IIA-4, i.e. is:

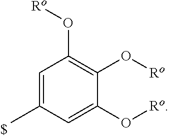

In an embodiment, group IIIA is group IIIA-2, i.e. is:

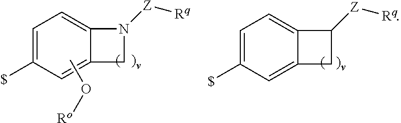

Most preferably, group IIIA is group IIIA-4, i.e. is:

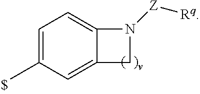

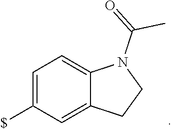

In an embodiment, group IVA is group IVA-2, i.e. is:

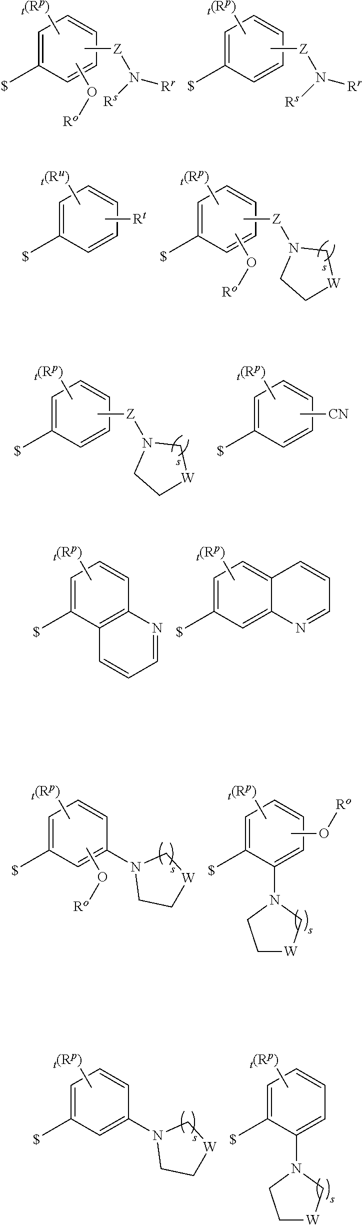

In a preferred embodiment, group IVA is group IVA-3, i.e. is:

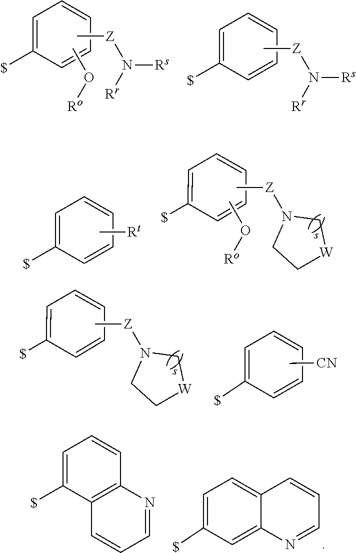

In a particularly preferred embodiment, group IVA is group IVA-4, i.e. is:

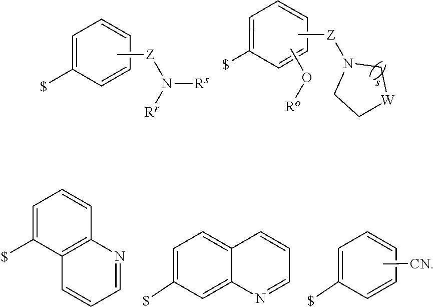

In a particularly preferred embodiment, group IVA is group IVA-5, i.e. is:

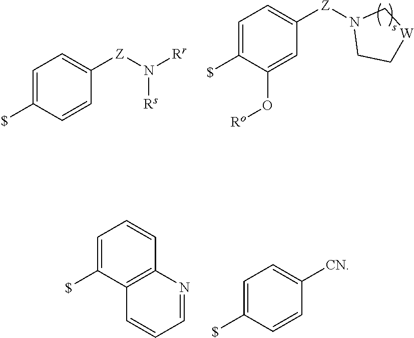

In a most preferred embodiment, group IVA is group IVA-6, i.e. is:

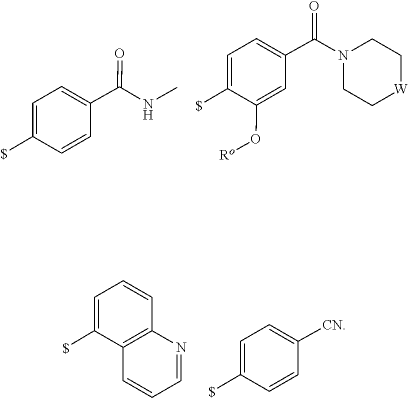

In an embodiment, group VA is group VA-2, i.e. is:

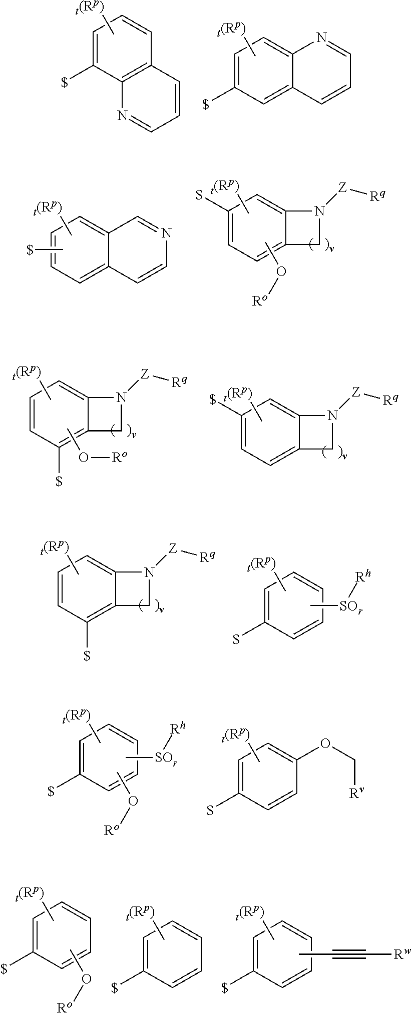

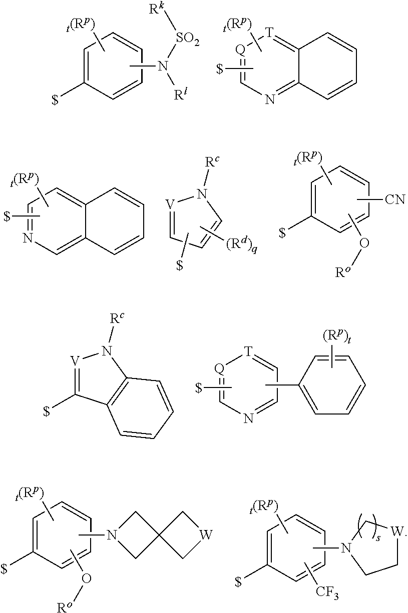

In a preferred embodiment, group VA is group VA-3, i.e. is:

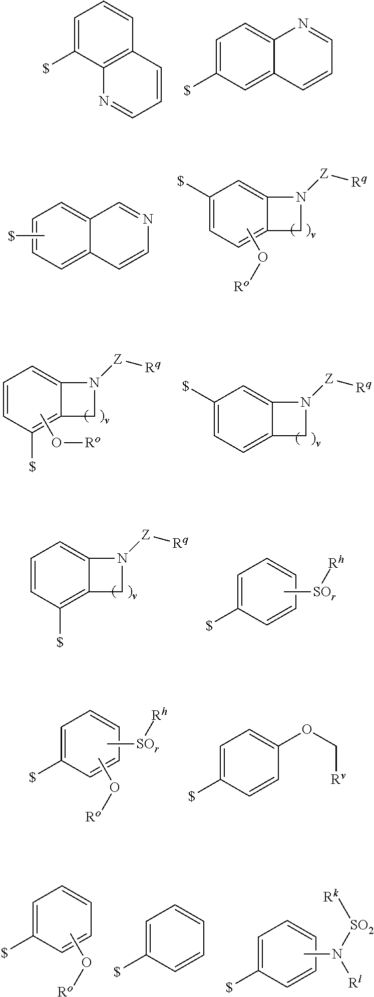

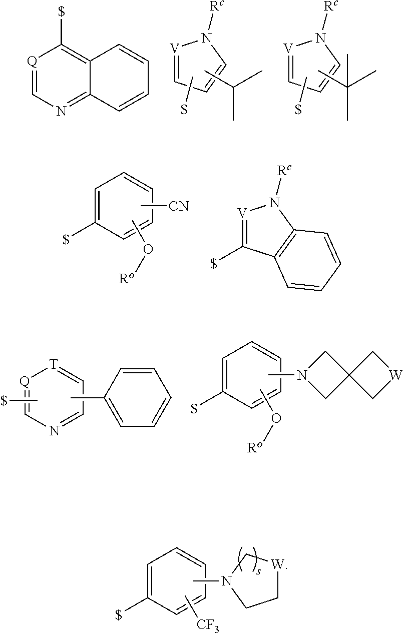

In a further preferred embodiment, group VA is group VA-4, i.e. is.

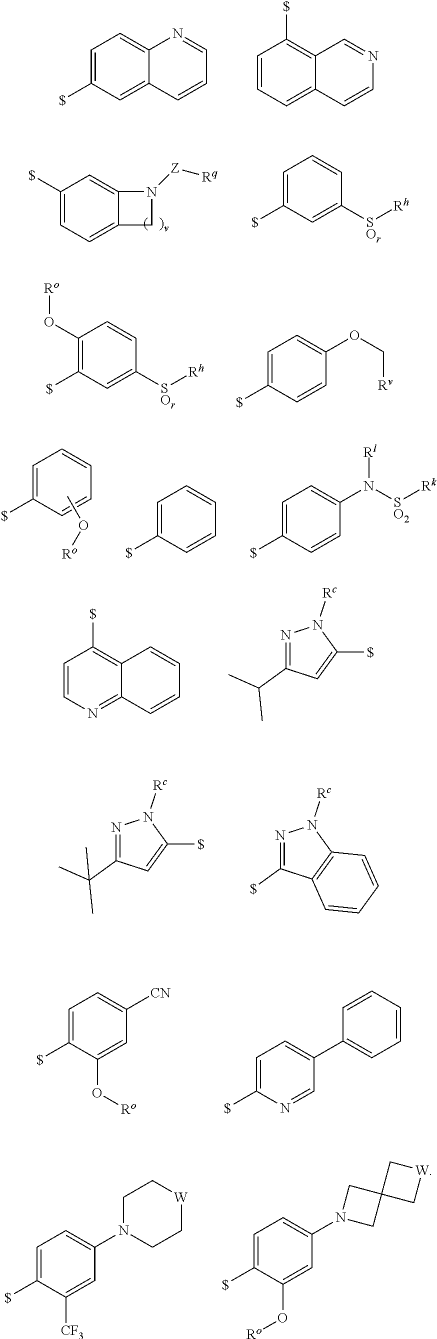

Most preferably, group VA is group VA-5, i.e. is:

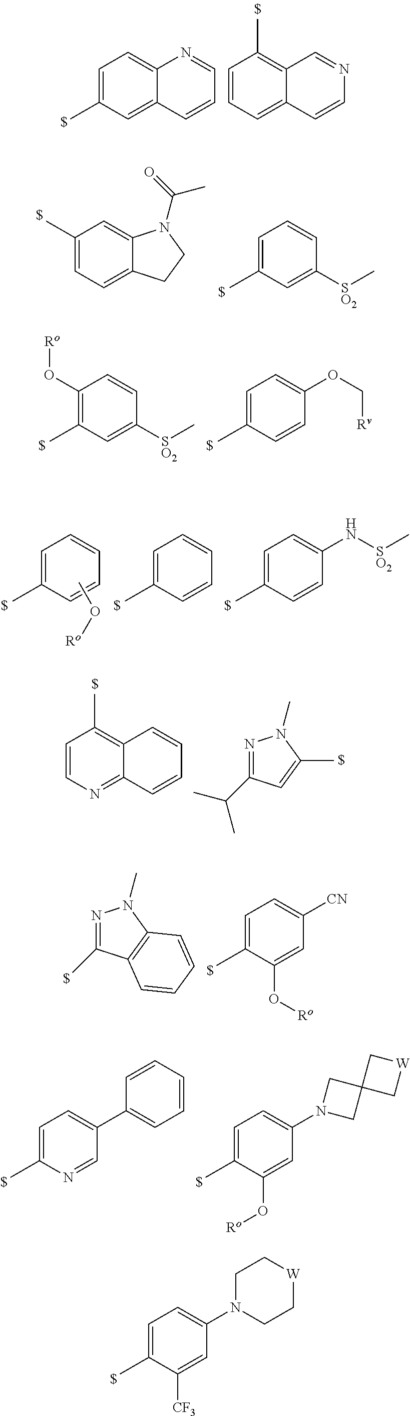

Thus, in a preferred combination (combination B1):
group I is group I-1;
group II is group II-1;
group III is group III-1;
group IV is group IV-2;
group V (if present) is group V-2;
group IA is group IA-1;
group IIA is group IIA-1;
group IIIA is group IIIA-1;
group IVA is group IVA-1; and
group VA (if present) is group VA-2.

In another preferred combination (combination B2):
group I is group I-1;
group II is group II-1;
group III is group III-1;
group IV is group IV-3;
group V (if present) is group V-2;
group IA is group IA-1;
group IIA is group IIA-1;
group IIIA is group IIIA-1;
group IVA is group IVA-1; and
group VA (if present) is group VA-3.

In another preferred combination (combination B3):
group I is group I-1;
group II is group II-1;
group III is group III-1;
group IV is group IV-4;
group V (if present) is group V-2;
group IA is group IA-1;
group IIA is group IIA-1;
group IIIA is group IIIA-1;
group IVA is group IVA-1; and
group VA (if present) is group VA-3.

In another preferred combination (combination B4):
group I is group I-1;
group II is group II-1;
group III is group III-1;
group IV is group IV-5;
group V (if present) is group V-2;
group IA is group IA-1;
group IIA is group IIA-1;
group IIIA is group IIIA-1;
group IVA is group IVA-1; and
group VA (if present) is group VA-3.

In another preferred combination (combination B5):
group I is group I-1;
group II is group II-2;
group III is group III-2;
group IV is group IV-5;
group V (if present) is group V-2;
group IA is group IA-1;
group IIA is group IIA-2;
group IIIA is group IIIA-2;
group IVA is group IVA-2; and
group VA (if present) is group VA-3.

In another preferred combination (combination B6):
group I is group I-1;
group II is group II-2′;
group III is group III-2;
group IV is group IV-5;
group V (if present) is group V-2;
group IA is group IA-1;
group IIA is group IIA-2;
group IIIA is group IIIA-2;
group IVA is group IVA-2; and
group VA (if present) is group VA-3.

In another preferred combination (combination B7):
group I is group I-1;
group II is group II-3;
group III is group III-3;
group IV is group IV-5;
group V (if present) is group V-2;
group IA is group IA-1;
group IIA is group IIA-3;
group IIIA is group IIIA-2;
group IVA is group IVA-3; and
group VA (if present) is group VA-3.

In another preferred combination (combination B8):
group I is group I-1;
group II is group II-3;
group III is group III-4;
group IV is group IV-5;
group V (if present) is group V-2;
group IA is group IA-1;
group IIA is group IIA-3;
group IIIA is group IIIA-2;
group IVA is group IVA-4; and
group VA (if present) is group VA-3.

In another preferred combination (combination B9):
group I is group I-1;
group II is group II-3;
group III is group III-4;
group IV is group IV-5;
group V (if present) is group V-2;
group IA is group IA-1;
group IIA is group IIA-3;
group IIIA is group IIIA-2;
group IVA is group IVA-4; and
group VA (if present) is group VA-3.

In another preferred combination (combination B10):
group I is group I-2;
group II is group II-4;
group III is group III-5;
group IV is group IV-6;
group V (if present) is group V-2;
group IA is group IA-2;
group IIA is group IIA-4;
group IIIA is group IIIA-3;
group IVA is group IVA-5; and
group VA (if present) is group VA-3.

In another preferred combination (combination B11):
group I is group I-3;
group II is group II-5;
group III is group III-5;
group IV is group IV-6;
group V (if present) is group V-3;
group IA is group IA-3;
group IIA is group IIA-4;
group IIIA is group IIIA-3;
group IVA is group IVA-5; and
group VA (if present) is group VA-4.

In another preferred combination (combination B12):
group I is group I-4;
group II is group II-6;
group III is group III-6;
group IV is group IV-6;
group V (if present) is group V-3;
group IA is group IA-3;
group IIA is group IIA-4;
group IIIA is group IIIA-4;
group IVA is group IVA-6; and
group VA (if present) is group VA-5.

In the compound of formula (I), with respect to Ring A and Ring B, each of the substituents thereon can be defined more narrowly as follows.

Preferably, Q is selected from CH or N.

Preferably, T is CH.

Preferably, W is CH₂, O, NR^{y}, or S(O)₂. Most preferably, W is CH₂, O or NR^{y}.

Preferably, Z is C(O) or S(O)₂. Most preferably, Z is C(O).

Preferably, R^{a} is C₁-C₃ alkyl substituted with 0 to 1 substituents selected from O—C₁-C₃ alkyl, F and Cl. Further preferably, R^{a} is C₁-C₃ alkyl substituted with 0 to 1 substituents selected from O—C₁-C₃ alkyl. Most preferably, R^{a} is CH₃, CH₂CH₃, or CH₂OCH₃.

Preferably, R^{b} is independently selected from C₁-C₃ alkyl, F and Cl. Further preferably, R^{b} is independently selected from C₁-C₃ alkyl. Most preferably, R^{b} CH₃.

Preferably, R^{o} is C₁-C₃ alkyl substituted with 0 to 3 substituents independently selected from O—C₁-C₃ alkyl, F and Cl. Further preferably, R^{c} is C₁-C₃ alkyl. Most preferably, R^{c} is CH₃.

Preferably, R^{d} is independently selected from C₁-C₄ alkyl substituted with 0 to 1 substituents independently selected from O—C₁-C₃ alkyl, F and Cl; or phenyl substituted with 0 to 1 substituents independently selected from O—C₁-C₃ alkyl, F and Cl. Further preferably, R^{d} is independently selected from C₁-C₄ alkyl or phenyl. Most preferably, R^{d} is independently selected from CH(CH₃)₂ or C(CH₃)₃.

Preferably, R^{e} is C₁-C₃ alkyl substituted with 0 to 1 substituents independently selected from O—C₁-C₃ alkyl, F and Cl. Further preferably, R^{e} is C₁-C₃ alkyl. Most preferably, R^{e} is CH₃.

Preferably, R^{f} is C₁-C₃ alkyl substituted with 0 to 1 substituents independently selected from O—C₁-C₃ alkyl, F and Cl. Further preferably, R^{f} is C₁-C₃ alkyl. Further preferably still, R^{f} is CH₃, CH₂CH₃, or CH(CH₃)₂.

Preferably, R^{g} is H or CH₃. Most preferably, R^{g} is H.

Preferably, R^{h} is C₁-C₃ alkyl substituted with 0 to 1 substituents independently selected from O—C₁-C₃ alkyl, F and Cl. Further preferably, R^{h} is C₁-C₃ alkyl. Most preferably, R^{h} is CH₃.

Preferably, Rⁱ is C₁-C₃ alkyl substituted with 0 to 1 substituents independently selected from O—C₁-C₃ alkyl, F and Cl. Further preferably, Rⁱ is C₁-C₃ alkyl. Most preferably, Rⁱ is CH₃.

Preferably, R^{j} is H or CH₃. Most preferably, R^{j} is H.

Preferably, R^{k} is C₁-C₃ alkyl with 0 to 1 substituents independently selected from O—C₁-C₃ alkyl, F and Cl. Further preferably, R^{k} is C₁-C₃ alkyl. Most preferably, R^{k} is CH₃.

Preferably, R^{l} is H or CH₃. Most preferably, R^{l} is H.

Preferably, R^{m} is C₁-C₄ alkyl substituted with 0 to 1 substituents independently selected from O—C₁-C₃ alkyl, F and Cl. Further preferably, R^{m} is C₁-C₄ alkyl. Further preferably still, R^{m} is C₁-C₃ alkyl. Most preferably, R^{m} is CH₃.

Preferably, Rⁿ is H or CH₃. Most preferably, Rⁿ is H.

Preferably, R^{o} is independently selected from C₁-C₃ alkyl substituted with 0 to 1 substituents independently selected from O—C₁-C₃ alkyl, F and Cl; or phenyl substituted with 0 to 1 substituents independently selected from O—C₁-C₃ alkyl, F and Cl. Further preferably, R^{o} is independently selected from C₁-C₃ alkyl substituted with 0 to 1 substituents independently selected from O—C₁-C₃ alkyl, F and Cl; or phenyl. Further preferably still, R^{o} is independently selected from C₁-C₃ alkyl substituted with 0 to 1 substituents independently selected from O—C₁-C₃ alkyl; or phenyl. Even more preferably, R^{o} is independently selected from C₁-C₃ alkyl or phenyl. Even more preferably still, R^{o} is independently selected from C₁-C₃ alkyl. Most preferably, R^{o} is independently selected from CH₃, CH₂CH₃, or CH(CH₃)₂.

Preferably, R^{p} is independently selected from C₁-C₃ alkyl, F, Cl and Br. Further preferably, R^{p} is independently selected from C₁-C₃ alkyl. Most preferably, R^{p} is CH₃.

Preferably, R^{q} is C₁-C₃ alkyl substituted with 0 to 1 substituents independently selected from O—C₁-C₃ alkyl, F and Cl. Further preferably, R^{q} is C₁-C₃ alkyl substituted with 0 to 1 substituents independently selected from O—C₁-C₃ alkyl. Most preferably, R^{q} is CH₃, CH₂CH₃, or CH₂OCH₃.

Preferably, R^{r} is H or C₁-C₃ alkyl substituted with 0 to 1 substituents independently selected from O—C₁-C₃ alkyl, F and Cl. Further preferably, R^{r} is H or C₁-C₃ alkyl. Further preferably still, R^{r} is C₁-C₃ alkyl. Most preferably, R^{r} is CH₃.

Preferably, R^{s} is H or CH₃. Most preferably, R^{s} is H.

Preferably, R^{t} is C₁-C₄ alkyl substituted with 0 to 1 substituents independently selected from O—C₁-C₃ alkyl, F and Cl. Further preferably, R^{t} is C₁-C₄ alkyl. Further preferably still, R^{t} is C₃-C₄ alkyl. Most preferably, R^{t} is C(CH₃)₃.

Preferably, R^{u} is independently selected from C₁-C₃ alkyl, F and Cl. Further preferably, R^{u} is independently selected from C₁-C₃ alkyl. Most preferably, R^{u} is CH₃.

Preferably, R^{v} is phenyl substituted with 0-1 substituents selected from C₁-C₃ alkyl, F and Cl. Further preferably, R^{v} is phenyl substituted with 0-1 substituents selected from C₁-C₃ alkyl. Most preferably, R^{v} is phenyl.

Preferably, R^{w} is H or CH₃. Most preferably, R^{w} is H.

Preferably, R^{x} is independently selected from H or CH₃. Most preferably, R^{x} is H.

Preferably, R^{y} is H, C₁-C₃ alkyl substituted with 0 to 1 substituent selected from O—C₁-C₃ alkyl, F and Cl; benzyl substituted with 0 to 1 substituent selected from C₁-C₃ alkyl, F and Cl; or C₃-C₅ cycloalkyl substituted with 0 to 1 substituent selected from C₁-C₃ alkyl, O—C₁-C₃ alkyl, F and Cl. Further preferably, R^{y} is H, C₁-C₃ alkyl substituted with 0 to 1 substituent selected from O—C₁-C₃ alkyl; benzyl substituted with 0 to 1 substituent selected from C₁-C₃ alkyl; or is C₃-C₅ cycloalkyl substituted with 0 to 1 substituent selected from C₁-C₃ alkyl. Further preferably still, R^{y} is C₁-C₃ alkyl substituted with 0 to 1 substituent selected from O—C₁-C₃ alkyl; benzyl substituted with 0 to 1 substituent selected from C₁-C₃ alkyl; or is C₃-C₅ cycloalkyl substituted with 0 to 1 substituent selected from C₁-C₃ alkyl. Even more preferably, R^{y} is C₁-C₃ alkyl substituted with 0 to 1 substituent selected from O—C₁-C₃ alkyl; benzyl; or is cyclopropyl. Even more preferably still, R^{y} is C₁-C₃ alkyl substituted with 0 to 1 substituent selected from O—C₁-C₃ alkyl; or is cyclopropyl. Even further preferably still, R^{y} is C₁-C₃ alkyl. Most preferably, R^{y} is CH₃, CH₂CH₃, or CH(CH₃)₂.

Preferably, n is 2 or 3. Most preferably, n is 2.

Preferably, p is 0 or 1. More preferably, p is 0.

Preferably, q is 1.

Preferably, r is 1 or 2. Most preferably, r is 2.

Preferably, s is 1 or 2. Most preferably, s is 2.

Preferably, t is 0 or 1. More preferably, t is 0.

Preferably, u is 3.

Preferably, v is 2 or 3. Most preferably, v is 2.

Thus, in a preferred combination (combination C1):
Q is selected from CH or N;
T is CH;
W is CH₂, O, NR^{y}, or S(O)₂;
Z is C(O) or S(O)₂;
R^{a} is C₁-C₃ alkyl substituted with 0 to 1 substituents selected from O—C₁-C₃ alkyl, F and Cl;
R^{b} is independently selected from C₁-C₃ alkyl, F and Cl;
R^{c} is C₁-C₃ alkyl substituted with 0 to 3 substituents independently selected from O—C₁-C₃ alkyl, F and Cl;
R^{d} is independently selected from C₁-C₄ alkyl substituted with 0 to 1 substituents independently selected from O—C₁-C₃ alkyl, F and Cl; or phenyl substituted with 0 to 1 substituents independently selected from O—C₁-C₃ alkyl, F and Cl;
R^{e} is C₁-C₃ alkyl substituted with 0 to 1 substituents independently selected from O—C₁-C₃ alkyl, F and Cl;
R^{f} is C₁-C₃ alkyl substituted with 0 to 1 substituents independently selected from O—C₁-C₃ alkyl, F and Cl;
R^{g} is H or CH₃;
R^{h} is C₁-C₃ alkyl substituted with 0 to 1 substituents independently selected from O—C₁-C₃ alkyl, F and Cl;
Rⁱ is C₁-C₃ alkyl substituted with 0 to 1 substituents independently selected from O—C₁-C₃ alkyl, F and Cl;
R^{j} is H or CH₃;
R^{k} is C₁-C₃ alkyl with 0 to 1 substituents independently selected from O—C₁-C₃ alkyl, F and Cl;
R^{l} is H or CH₃;
R^{m} is C₁-C₄ alkyl substituted with 0 to 1 substituents independently selected from O—C₁-C₃ alkyl, F and Cl;
Rⁿ is H or CH₃;
R^{o} is independently selected from C₁-C₃ alkyl substituted with 0 to 1 substituents independently selected from O—C₁-C₃ alkyl, F and Cl; or phenyl substituted with 0 to 1 substituents independently selected from O—C₁-C₃ alkyl, F and Cl;
R^{p} is independently selected from C₁-C₃ alkyl, F, Cl and Br;
R^{q} is C₁-C₃ alkyl substituted with 0 to 1 substituents independently selected from O—C₁-C₃ alkyl, F and Cl;
R^{r} is H or C₁-C₃ alkyl substituted with 0 to 1 substituents independently selected from O—C₁-C₃ alkyl, F and Cl;
R^{s} is H or CH₃;
R^{t} is C₁-C₄ alkyl substituted with 0 to 1 substituents independently selected from O—C₁-C₃ alkyl, F and Cl;
R^{u} is independently selected from C₁-C₃ alkyl, F and Cl;
R^{v} is phenyl substituted with 0-1 substituents selected from C₁-C₃ alkyl, F and Cl;
R^{w} is H or CH₃;
R^{x} is independently selected from H or CH₃;
R^{y} is H, C₁-C₃ alkyl substituted with 0 to 1 substituent selected from O—C₁-C₃ alkyl, F and Cl; or is benzyl substituted with 0 to 1 substituent selected from C₁-C₃ alkyl, F and Cl; or is C₃-C₅ cycloalkyl substituted with 0 to 1 substituent selected from C₁-C₃ alkyl, 0-C₁-C₃ alkyl, F and Cl;
n is 2 or 3;
p is 0 or 1;
q is 1;
r is 1 or 2;
s is 1 or 2;
t is 0 or 1;
u is 2 or 3; and
v is 2 or 3.

In a more preferred combination (combination C2):
Q and T are as defined in combination C1;
W is CH₂, O or NR^{y};
Z is C(O);
R^{a} is C₁-C₃ alkyl substituted with 0 to 1 substituent selected from O—C₁-C₃ alkyl;
R^{b} is independently selected from C₁-C₃ alkyl;
R^{c} is C₁-C₃ alkyl;
R^{d} is independently selected from C₁-C₄ alkyl or phenyl;
R^{e} is C₁-C₃ alkyl;
R^{f} is C₁-C₃ alkyl;
R^{g} is H;
R^{h} is C₁-C₃ alkyl;
Rⁱ is C₁-C₃ alkyl;
R^{j} is H;
R^{k} is C₁-C₃ alkyl;
R^{l} is H;
R^{m} is C₁-C₄ alkyl;
Rⁿ is H;
R^{o} is independently selected from C₁-C₃ alkyl substituted with 0 to 1 substituents independently selected from O—C₁-C₃ alkyl, F and Cl; or phenyl;
R^{p} is independently selected from C₁-C₃ alkyl;
R^{q} is C₁-C₃ alkyl substituted with 0 to 1 substituent selected from O—C₁-C₃ alkyl;
R^{r} is H or C₁-C₃ alkyl;
R^{s} is H or CH₃;
R^{t} is C₁-C₄ alkyl;
R^{u} is independently selected from C₁-C₃ alkyl;
R^{v} is phenyl substituted with 0-1 substituents selected from C₁-C₃ alkyl;
R^{w} is H;
R^{x} is H; and
R^{y} is H, C₁-C₃ alkyl substituted with 0 to 1 substituent selected from O—C₁-C₃ alkyl; or is benzyl substituted with 0 to 1 substituents selected from C₁-C₃ alkyl; or is C₃-C₅ cycloalkyl substituted with 0 to 1 substituent selected from C₁-C₃ alkyl;
n is 2;
p is 0 or 1;
q is 1;
r is 2;
s is 2;
t is 0 or 1;
u is 2 or 3; and
v is 2.

In a more preferred combination (combination C3):
Q and T are as defined in combination C1;
W, Z, R^{a}, R^{b}, R^{c}, R^{d}, R^{o}, R^{f}, R^{g}, R^{h}, Rⁱ, R^{j}, R^{k}, R^{l}, R^{m}, Rⁿ, R^{p}, R^{q}, R^{r}, R^{s}, R^{t}, R^{u}, R^{w}, R^{x}, n, p, q, r, s, t, u, and v are as defined in combination C2;
R^{o} is independently selected from C₁-C₃ alkyl substituted with 0 to 1 substituents independently selected from O—C₁-C₃ alkyl; or phenyl;
R^{v} is phenyl;
R^{y} is C₁-C₃ alkyl substituted with 0 to 1 substituent selected from O—C₁-C₃ alkyl; or is benzyl substituted with 0 to 1 substituent selected from C₁-C₃ alkyl; or is C₃-C₅ cycloalkyl substituted with 0 to 1 substituent selected from C₁-C₃ alkyl.

In a further preferred combination (combination C4):
Q and T are as defined in combination C1;
W, Z, R^{a}, R^{b}, R^{c}, R^{d}, R^{e}, R^{f}, R^{g}, R^{h}, Rⁱ, R^{j}, R^{k}, R^{l}, R^{m}, Rⁿ, R^{p}, R^{q}, R^{r}, R^{s}, R^{t}, R^{u}, R^{w}, R^{x}, n, p, q, r, s, t, u, and v are as defined in combination C2;
R^{o} and RV are as defined in combination C3; and
R^{y} is C₁-C₃ alkyl substituted with 0 to 1 substituent selected from O—C₁-C₃ alkyl; or is benzyl; or is cyclopropyl.

In an even further preferred combination (combination C5):
Q and T are as defined in combination C1;
W, Z, R^{a}, R^{b}, R^{c}, R^{d}, R^{e}, R^{f}, R^{g}, R^{h}, Rⁱ, R^{j}, R^{k}, R^{l}, R^{m}, Rⁿ, R^{p}, R^{q}, R^{r}, R^{s}, R^{t}, R^{u}, R^{w}, R^{x}, n, p, q, r, s, t, u, and v are as defined in combination C2;
R^{o} and RV are as defined in combination C3; and
R^{y} is C₁-C₃ alkyl substituted with 0 to 1 substituent selected from O—C₁-C₃ alkyl; or is cyclopropyl.

In an even further preferred combination (combination C6):
Q and T are as defined in combination C1;
W, Z, R^{b}, R^{c}, R^{e}, R^{f}, R^{g}, R^{h}, Rⁱ, R^{j}, R^{k}, R^{l}, Rⁿ, R^{p}, R^{u}, R^{w}, R^{x}, n, p, q, r, s, t, and v are as defined in combination C2;
R^{v} is as defined in combination C3;
R^{a} is CH₃, CH₂CH₃, or CH₂OCH₃;
R^{d} is CH(CH₃)₂ or C(CH₃)₃;
R^{m} is C₁-C₃ alkyl;
R^{o} is independently selected from C₁-C₃ alkyl or phenyl;
R^{q} is CH₃, CH₂CH₃, or CH₂OCH₃;
R^{r} is C₁-C₃ alkyl;
R^{s} is H;
R^{t} is C₃-C₄ alkyl;
R^{y} is C₁-C₃ alkyl; and
u is 3.

In an even further preferred combination (combination C7):
Q and T are as defined in combination C1;
W, Z, R^{b}, R^{c}, R^{e}, R^{f}, R^{g}, R^{h}, Rⁱ, R^{j}, R^{k}, R^{l}, Rⁿ, R^{p}, R^{u}, R^{w}, R^{x}, n, p, q, r, s, t, and v are as defined in combination C2;
R^{v} is as defined in combination C3;
R^{a}, R^{d}, R^{m}, R^{q}, R^{r}, R^{s}, R^{t}, R^{y}, and u are as defined in combination C6; and
R^{o} is independently selected from C₁-C₃ alkyl.

In an even further preferred combination (combination C8):
Q and T are as defined in combination C1;
W, Z, R^{b}, R^{c}, R^{e}, R^{f}, R^{g}, R^{h}, Rⁱ, R^{j}, R^{k}, R^{l}, Rⁿ, R^{p}, R^{u}, R^{w}, R^{x}, n, p, q, r, s, t, and v are as defined in combination C2;
R^{v} is as defined in combination C3;
R^{a}, R^{d}, R^{m}, R^{q}, R^{r}, R^{s}, R^{t}, and u are as defined in combination C6.
R^{o} is independently selected from CH₃, CH₂CH₃, or CH(CH₃)₂; and
R^{y} is CH₃, CH₂CH₃, or CH(CH₃)₂.

In an even further preferred still combination (combination C9):
Q and T are as defined in combination C1;
W, Z, R^{g}, R^{j}, R^{l}, Rⁿ, R^{w}, R^{x}, n, p, q, r, s, t, and v are as defined in combination C2;
R^{v} is as defined in combination C3;
R^{a}, R^{d}, R^{q}, R^{s}, and u are as defined in combination C6.
R^{o} and R^{y} are as defined in combination C9;
R^{b} is CH₃;
R^{c} is CH₃;
R^{e} is CH₃;
R^{f} is CH₃, CH₂CH₃, or CH(CH₃)₂;
R^{h} is CH₃;
Rⁱ is CH₃;
R^{k} is CH₃;
R^{m} is CH₃;
R^{p} is CH₃;
R^{r} is CH₃
R^{u} is CH₃; and
R^{t} is C(CH₃)₃.

In a most preferred combination (combination C10):
Q and T are as defined in combination C1;
W, Z, R^{g}, R^{j}, R^{l}, Rⁿ, R^{w}, R^{x}, n, q, r, s, and v are as defined in combination C2;
R^{v} is as defined in combination C3;
R^{a}, R^{d}, R^{q}, R^{s}, and u are as defined in combination C6.
R^{o} and R^{y} are as defined in combination C7;
R^{c}, R^{e}, R^{f}, Rⁱ, R^{k}, R^{m}, R^{p}, R^{r}, R^{u}, and R^{t} are as defined in combination C10; and
p is 0; and
t is 0.

In the above combination C10, it will be understood that definitions for R^{b} and R^{p} have been omitted because p and t are each 0.

In the present invention, preferred combinations of the above combinations are as follows:

the combination of combination A1, combination B1, and combination C1; the combination of combination A2, combination B1, and combination C1; the combination of combination A3, combination B1, and combination C1; the combination of combination A4, combination B1, and combination C1; the combination of combination A1, combination B2, and combination C2; the combination of combination A2, combination B2, and combination C2; the combination of combination A3, combination B2, and combination C2; the combination of combination A4, combination B2, and combination C2; the combination of combination A1, combination B3, and combination C3; the combination of combination A2, combination B3, and combination C3; the combination of combination A3, combination B3, and combination C3; the combination of combination A4, combination B3, and combination C3; the combination of combination A1, combination B4, and combination C4; the combination of combination A2, combination B4, and combination C4; the combination of combination A3, combination B4, and combination C4; the combination of combination A4, combination B4, and combination C4; the combination of combination A1, combination B5, and combination C5; the combination of combination A2, combination B5, and combination C5; the combination of combination A3, combination B5, and combination C5; the combination of combination A4, combination B5, and combination C5; the combination of combination A1, combination B6, and combination C6; the combination of combination A2, combination B6, and combination C6; the combination of combination A3, combination B6, and combination C6; the combination of combination A4, combination B6, and combination C6; the combination of combination A1, combination B7, and combination C7; the combination of combination A2, combination B7, and combination C7; the combination of combination A3, combination B7, and combination C7; the combination of combination A4, combination B7, and combination C7; the combination of combination A1, combination B8, and combination C8; the combination of combination A2, combination B8, and combination C8; the combination of combination A3, combination B8, and combination C8; the combination of combination A4, combination B8, and combination C8; the combination of combination A1, combination B9, and combination C9; the combination of combination A2, combination B9, and combination C9; the combination of combination A3, combination B9, and combination C9; the combination of combination A4, combination B9, and combination C9; the combination of combination A1, combination B10, and combination C10; the combination of combination A2, combination B10, and combination C10; the combination of combination A3, combination B10, and combination C10; the combination of combination A4, combination B10, and combination C10; the combination of combination A1, combination B11, and combination C10; the combination of combination A2, combination B11, and combination C10; the combination of combination A3, combination B11, and combination C10; the combination of combination A4, combination B11, and combination C10; the combination of combination A1, combination B12, and combination C10; the combination of combination A2, combination B12, and combination C10; the combination of combination A3, combination B12, and combination C10; and the combination of combination A4, combination B12, and combination C10.

In the above combinations, it will be understood that insofar as any variable in combination C₁-C₁₀ is not present in any of the combinations B1-B12, then that variable in combination C₁-C₁₀ is to be disregarded.

In a preferred embodiment, the compound of the invention is a compound that (a) is of formula (Ia):

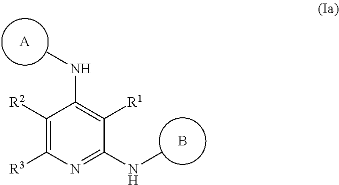

or (b) is a tautomer, N-oxide, pharmaceutically acceptable salt, or solvate thereof; wherein R¹, R², R³, ring A and ring B are as defined above (inclusive of any of the combinations defined above) and wherein each of * and ^{$} in the structures of ring A and ring B denotes attachment to the relevant NH group.

In a further preferred embodiment, the compound of the invention is a compound that (a) is of formula (Ib):

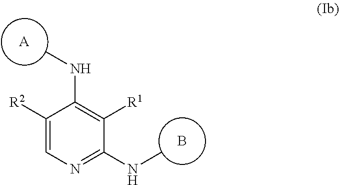

or (b) is a tautomer, N-oxide, pharmaceutically acceptable salt, or solvate thereof; wherein R¹, R², ring A and ring B are as defined above (inclusive of any of the combinations defined above) and wherein each of * and ^{$} in the structures of ring A and ring B denotes attachment to the relevant NH group.

In the present invention, it will be understood that it is important to balance the nature of the ring A and the ring B in order to attain good results. In the present invention, the groups for ring A and ring B have been listed in order of preference, i.e. group I is the most preferred group for ring A whilst group V is the least preferred, and group IA is the most preferred group for ring B whilst group VA is the least preferred. As defined by the present invention, it is permissible to combine together more preferred A ring group with less preferred B ring group and vice versa. It is also possible to combine together the more preferred A ring groups with the more preferred B ring groups. However, it is not permissible to combine together least preferred A ring groups with the least preferred B ring groups.

It is noted that, generally, it is preferred for each of ring A and ring B (particularly ring B) to be relatively electron rich. Where a ring A or ring B has an electron withdrawing substituent, it is generally preferred for it to be further substituted with an electron donating substituent.

Where ring A is selected from a ring within group I, group II, group III, group IV and group V as defined above; and ring B is selected from a ring within group IA, group IIA, group IIA, group IVA, and group VA as defined above; then:
it is preferred that:
when X is NH; Y is NH; R² is H; and ring A is a ring within group I, then: ring B is a ring within group IA, group IIA, group IIIA, group IVA or group VA; and
when X is NH; Y is NH; R² is H; and ring A is a ring within group II; then: ring B is a ring within group IA, group IIA, group IIIA or group IVA.
   It is also preferred (independently, in or combination with the preferred bullet points above) that:
when X is NH; Y is NH; R² is H; and ring A is a ring within group III; then: ring B is a ring within group IA or group IIA.

In a preferred embodiment, ring A is a ring within group I, group II, group III and group IV as defined above; and ring B is a ring within group IA, group IIA, group IIIA and group IVA as defined above; and
when X is NH; Y is NH; R² is H; and ring A is a ring within group I or group II; then: ring B is a ring within group IA, group IIA, group IIIA or group IVA;
when X is NH; Y is NH; R² is H; and ring A is a ring within group III, then: ring B is a ring within group IA, group IIA or group IIIA;
when X is NH; Y is NH; R² is H; and ring A is a ring within group IV, then: ring B is a ring within group IA.

In this embodiment, it is further preferred that:
when X is NH; Y is NH; R² is H; and ring A is a ring within group I; then: ring B is a ring within group IA, group IIA, group IIIA or group IVA; and
when X is NH; Y is NH; R² is H; and ring A is a ring within group II, then: ring B is a ring within group IA, group IIA or group IIIA.

In this embodiment, it is also further preferred (independently, or in combination with the further preferred bullet points above) that:
when X is NH; Y is NH; R² is H; and ring A is a ring within group III, then: ring B is a ring within group IA or group IIA.

In this embodiment, it is even further preferred that:
when X is NH; Y is NH; R² is H; and ring A is a ring within group II, then: ring B is a ring within group IA or group IIA; and
when X is NH; Y is NH; R² is H; and ring A is a ring within group III, then: ring B is a ring within group IA or group IIA.

In another preferred embodiment, ring A is a ring within group I, group II and group III as defined above; and ring B is a ring within group IA, group IIA, and group IIIA as defined above; and
when X is NH; Y is NH; R² is H; and ring A is a ring within group I; then: ring B is a ring within group IA, group IIA or group IIIA;
when X is NH; Y is NH; R² is H; and ring A is a ring within group II, then: ring B is a ring within group IA or group IIA;
when X is NH; Y is NH; R² is H; and ring A is a ring within group III, then: ring B is a ring within group IA.

In another preferred embodiment, ring A is a ring within group I or group II as defined above; and ring B is a ring within group IA or group IIA as defined above; and
when X is NH; Y is NH; R² is H; and ring A is a ring within group I; then: ring B is a ring within group IA or group IIA;
when X is NH; Y is NH; R² is H; and ring A is a ring within group II, then: ring B is a ring within group IA.

In another preferred embodiment, ring A is a ring within group I as defined above; and ring B is a ring within group IA as defined above.

It is also generally preferred that:
when X is NH; Y is NH; and R² is F, Cl, Br, —COR⁴, —SO₂R⁵, —SOR⁵, —CN, —NO, —NO₂ or —NR⁶₃⁺; then: ring A is a ring within group I and ring B is a ring within group IA.

It is also generally preferred that:
when X is bond and Y is NH, then: R² is H; ring A is a ring within group I or group II; and ring B is a ring within group IA.

It is also generally preferred that:
when X is NH and Y is bond, then: R² is H; ring A is a ring within group I or group II; and ring B is a ring within group IA or group IIA.

It is further generally preferred that:
when X is NH and Y is bond, then: R² is H; ring A is a ring within group I or group II; and ring B is a ring within group IA or group IIA.

It will be understood that each of the preferred embodiments above can be combined with each of the specific combinations (i.e. combination A1, combination B1, combination C1, and the combinations thereof) defined herein. Insofar as any combination as defined herein contains groups (i.e. groups V or VA) or substituents (i.e. Rⁿ, R^{w}, R^{x}) that are not included within the embodiments above, those groups or substituents are to be disregarded.

For the avoidance of doubt, it will be understood that each and every bullet point described herein as defining the combinations of ring A and ring B is considered to be a separate (i.e. separable) embodiment of the invention.

In a particularly preferred embodiment, the present invention provides a compound that (a) is of formula (Ia):

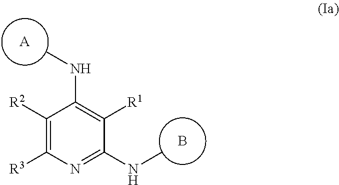

or (b) is a tautomer, N-oxide, pharmaceutically acceptable salt, or solvate thereof; wherein:
R¹ is H or F;
R² is H or F;
R³ is H or CH₃;
ring A is selected from a ring within group I, group II, group III and group IV, wherein * denotes attachment to the NH;
group I is:

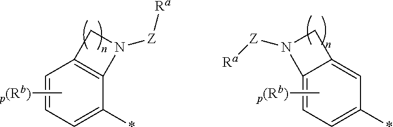

group II is:

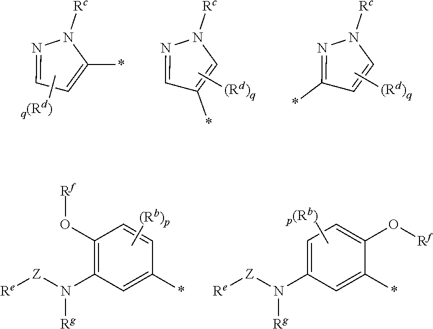

group III is:

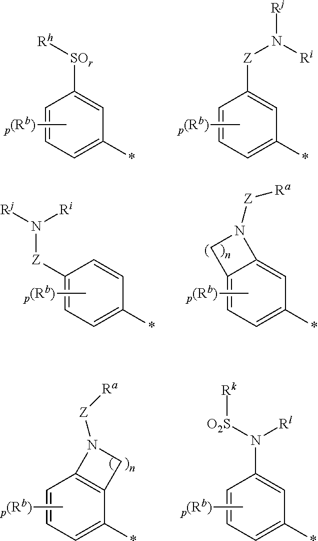

group IV is:

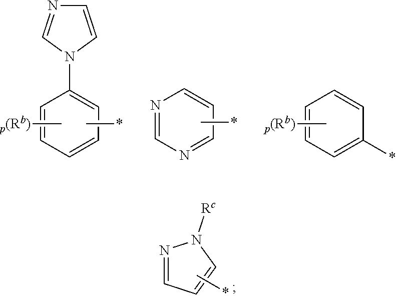

ring B is selected from a ring within group IA, group IIA, group IIA and group IVA, wherein ^{$} denotes attachment to the NH;
group IA is:

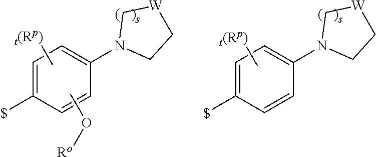

group IIA is:

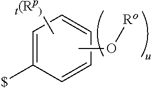

group IIIA is:

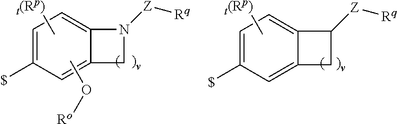

group IIIVA is:

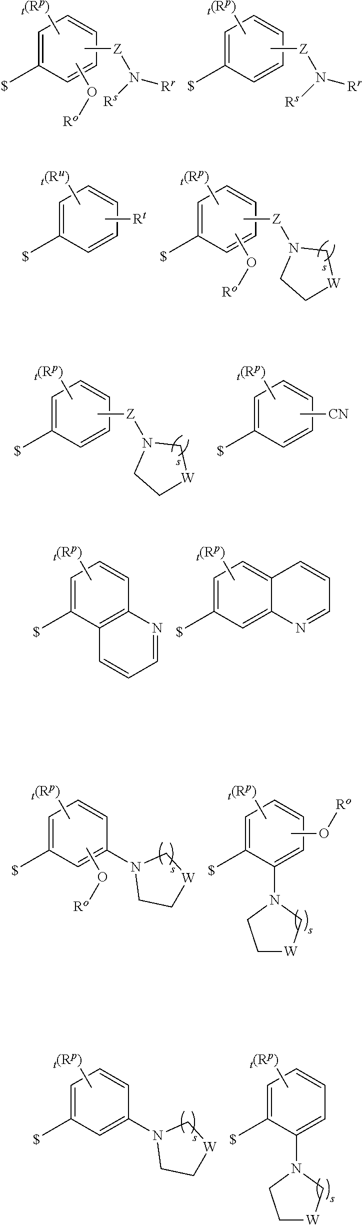

wherein:
W is CH₂, O, or NR^{y};
Z is C(O);
R^{a} is C₁-C₃ alkyl substituted with 0 to 1 substituent selected from O—C₁-C₃ alkyl;
R^{b} is independently selected from C₁-C₃ alkyl;
R^{c} is C₁-C₃ alkyl;
R^{d} is independently selected from C₁-C₄ alkyl or is phenyl;
R^{e} is C₁-C₃ alkyl;
R^{f} is C₁-C₃ alkyl;
R^{g} is H;
R^{h} is C₁-C₃ alkyl;
Rⁱ is C₁-C₃ alkyl;
R^{j} is H;
R^{k} is C₁-C₃ alkyl;
R^{l} is H;
R^{o} is independently selected from C₁-C₃ alkyl substituted with 0 to 1 substituent selected from O—C₁-C₃ alkyl; or is phenyl;
R^{p} is independently selected from C₁-C₃ alkyl
R^{q} is C₁-C₃ alkyl substituted with 0 to 1 substituent selected from O—C₁-C₃ alkyl;
R^{r} is H or C₁-C₃ alkyl;
R^{s} is H or C₁-C₃ alkyl;
R^{t} is C₁-C₄ alkyl;
R^{u} is independently selected from C₁-C₃ alkyl;
R^{y} is C₁-C₃ alkyl substituted with 0 to 1 substituent selected from O—C₁-C₃ alkyl; or is cyclopropyl;
n is 2;
p is 0 or 1;
q is 1;
r is 2;
s is 2;
t is 0 or 1;
u is 2 to 3;
v is 2;
and wherein:
   when R² is H; and ring A is a ring within group I; then: ring B is a ring within group IA, group IIA, group IIIA or group IVA;
   when R² is H; and ring A is a ring within group II, then: ring B is a ring within group IA, group IIA or group IIIA;
   when R² is H; and ring A is a ring within group III, then: ring B is a ring within group IA, group IIA or group IIIA;
   when R² is H; and ring A is a ring within group IV, then: ring B is a ring within group IA;
   when R² is F then: ring A is a ring within group I or group II and ring B is a ring within group IA;
and with the proviso that the compound of formula (Ia) is not (a) a compound selected from the group:

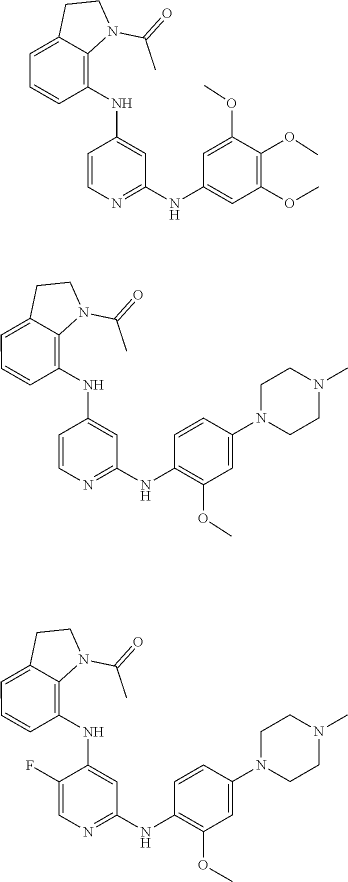 [SMILES: COc1cc(Nc2cc(Nc3cccc4c3N(C(C)=O)CC4)ccn2)cc(OC)c1OC ; COc1cc(N2CCN(C)CC2)ccc1Nc1cc(Nc2cccc3c2N(C(C)=O)CC3)ccn1 ; COc1cc(N2CCN(C)CC2)ccc1Nc1cc(Nc2cccc3c2N(C(C)=O)CC3)c(F)cn1]

nor (b) a tautomer, N-oxide, pharmaceutically acceptable salt, or solvate thereof.

In a further particularly preferred embodiment, the present invention provides a compound that (a) is of formula (Ib):

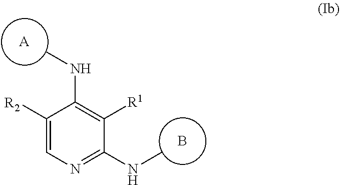

or (b) is a tautomer, N-oxide, pharmaceutically acceptable salt, or solvate t ereof; wherein:
R¹ is H or F;
R² is H or F;
ring A is selected from a ring within group I, group II, group III and group IV, wherein * denotes attachment to the NH;
group I is:

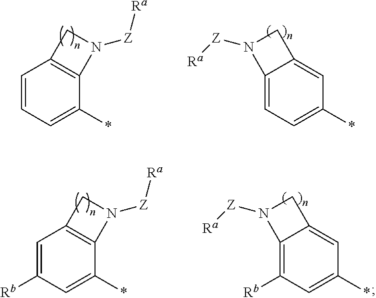

group II is:

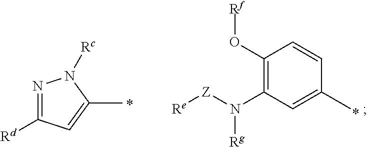

group III is:

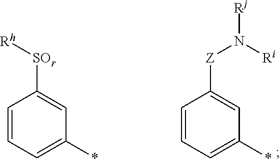

group IV is:

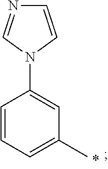

ring B is selected from a ring within group IA, group IIA, group IIA and group IVA, wherein ^{$} denotes attachment to the NH;
group IA is:

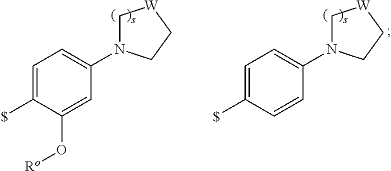

group IIA is:

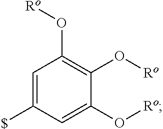

group IIIA is:

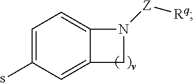

group IIIVA is:

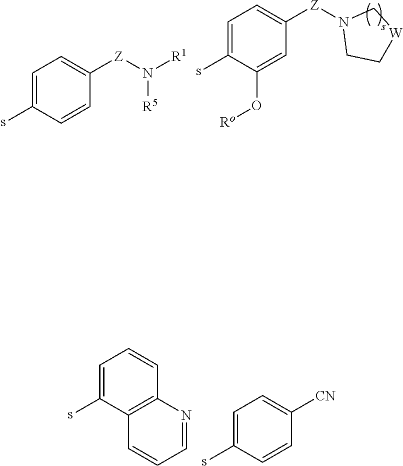

wherein:
W is CH₂, O, or NR^{y};
Z is C(O);
R^{a} is CH₃, CH₂CH₃, or CH₂OCH₃;
R^{b} is C₁-C₃ alkyl;
R^{c} is C₁-C₃ alkyl;
R^{d} is CH(CH₃)₂ or C(CH₃)₃;
R^{e} is C₁-C₃ alkyl;
R^{f} is C₁-C₃ alkyl;
R^{g} is H;
R^{h} is C₁-C₃ alkyl;
Rⁱ is C₁-C₃ alkyl;
R^{j} is H;
R^{o} is independently selected from CH₃, CH₂CH₃, or CH(CH₃)₂;
R^{q} is CH₃, CH₂CH₃, or CH₂OCH₃;
R^{r} is C₁-C₃ alkyl;
R^{s} is H;
R^{y} is CH₃, CH₂CH₃, or CH(CH₃)₂;
n is 2;
r is 2;
s is 2;
v is 2;
and wherein:
   when R² is H; and ring A is a ring within group I; then: ring B is a ring within group IA, group IIA, group IIIA or group IVA;
   when R² is H; and ring A is a ring within group II, then: ring B is a ring within group IA or group IIA; and
   when R² is H; and ring A is a ring within group III, then: ring B is a ring within group IA or group IIA.
   when R² is H; and ring A is a ring within group IV, then: ring B is a ring within group IA;
   when and R² is F; then: ring A is a ring within group I or group II and ring B is a ring within group IA;
and with the proviso that the compound of formula (Ib) is not (a) a compound selected from the group:

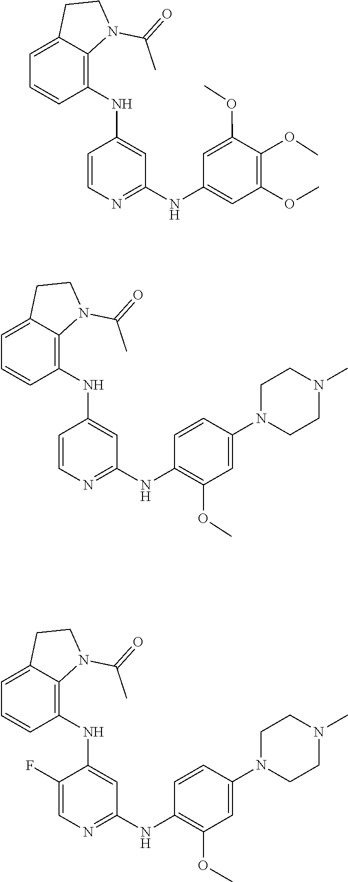 [SMILES: COc1cc(Nc2cc(Nc3cccc4c3N(C(C)=O)CC4)ccn2)cc(OC)c1OC ; COc1cc(N2CCN(C)CC2)ccc1Nc1cc(Nc2cccc3c2N(C(C)=O)CC3)ccn1 ; COc1cc(N2CCN(C)CC2)ccc1Nc1cc(Nc2cccc3c2N(C(C)=O)CC3)c(F)cn1]

nor (b) a tautomer, N-oxide, pharmaceutically acceptable salt, or solvate thereof.

Preferably, the compound of the invention is a compound selected from:

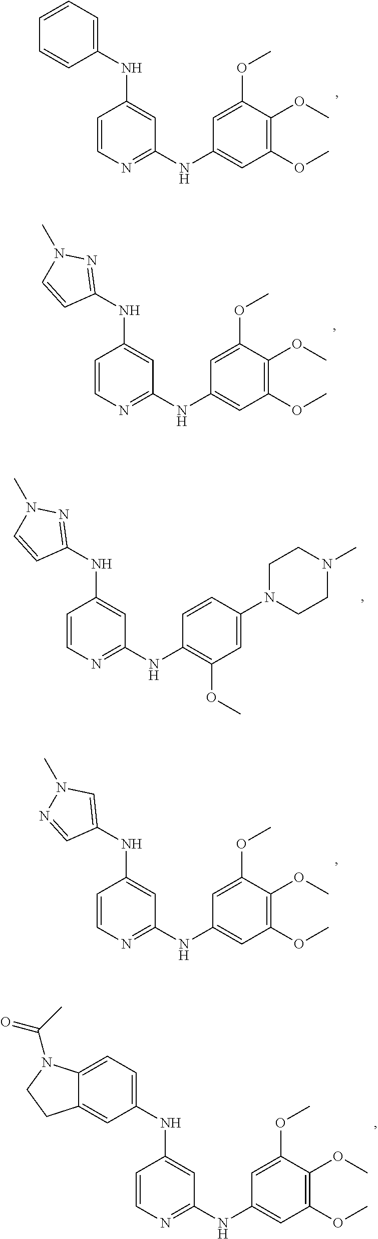 [SMILES: COc1cc(Nc2cc(Nc3ccccc3)ccn2)cc(OC)c1OC ; COc1cc(Nc2cc(Nc3ccn(C)n3)ccn2)cc(OC)c1OC ; COc1cc(N2CCN(C)CC2)ccc1Nc1cc(Nc2ccn(C)n2)ccn1 ; COc1cc(Nc2cc(Nc3cnn(C)c3)ccn2)cc(OC)c1OC ; COc1cc(Nc2cc(Nc3ccc4c(c3)CCN4C(C)=O)ccn2)cc(OC)c1OC]

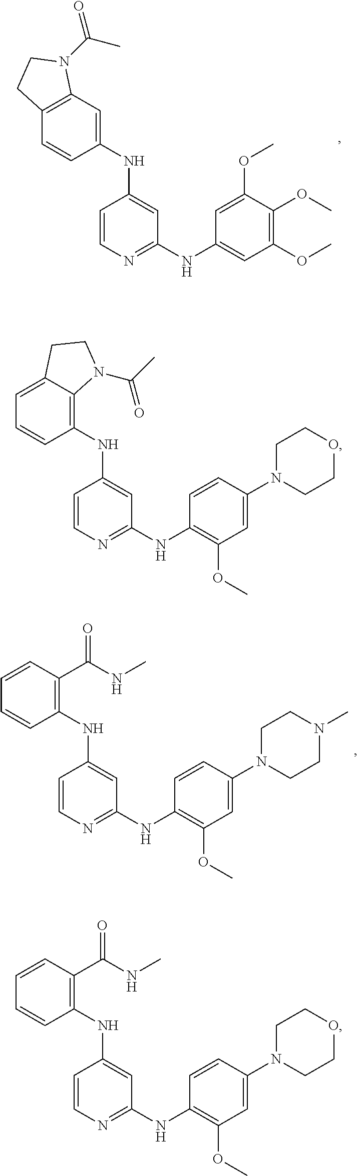 [SMILES: COc1cc(Nc2cc(Nc3ccc4c(c3)N(C(C)=O)CC4)ccn2)cc(OC)c1OC ; COc1cc(N2CCOCC2)ccc1Nc1cc(Nc2cccc3c2N(C(C)=O)CC3)ccn1 ; CNC(=O)c1ccccc1Nc1ccnc(Nc2ccc(N3CCN(C)CC3)cc2OC)c1 ; CNC(=O)c1ccccc1Nc1ccnc(Nc2ccc(N3CCOCC3)cc2OC)c1]

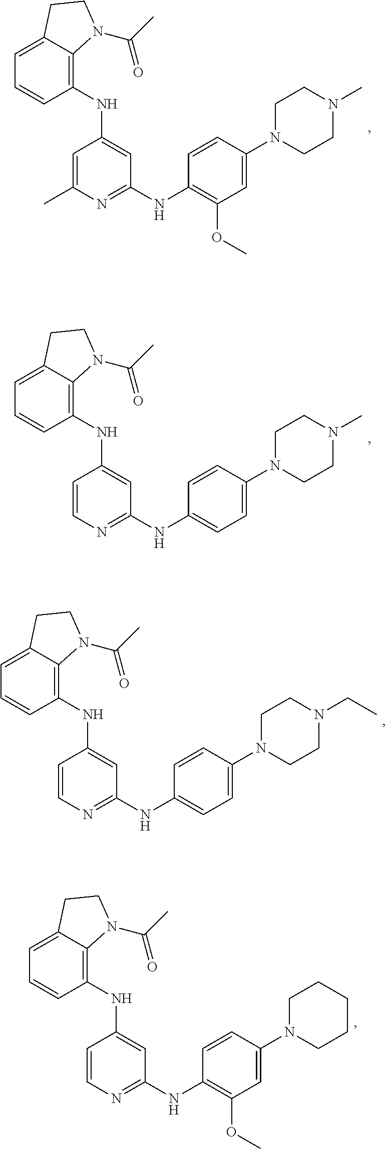 [SMILES: COc1cc(N2CCN(C)CC2)ccc1Nc1cc(Nc2cccc3c2N(C(C)=O)CC3)cc(C)n1 ; CC(=O)N1CCc2cccc(Nc3ccnc(Nc4ccc(N5CCN(C)CC5)cc4)c3)c21 ; CCN1CCN(c2ccc(Nc3cc(Nc4cccc5c4N(C(C)=O)CC5)ccn3)cc2)CC1 ; COc1cc(N2CCCCC2)ccc1Nc1cc(Nc2cccc3c2N(C(C)=O)CC3)ccn1]

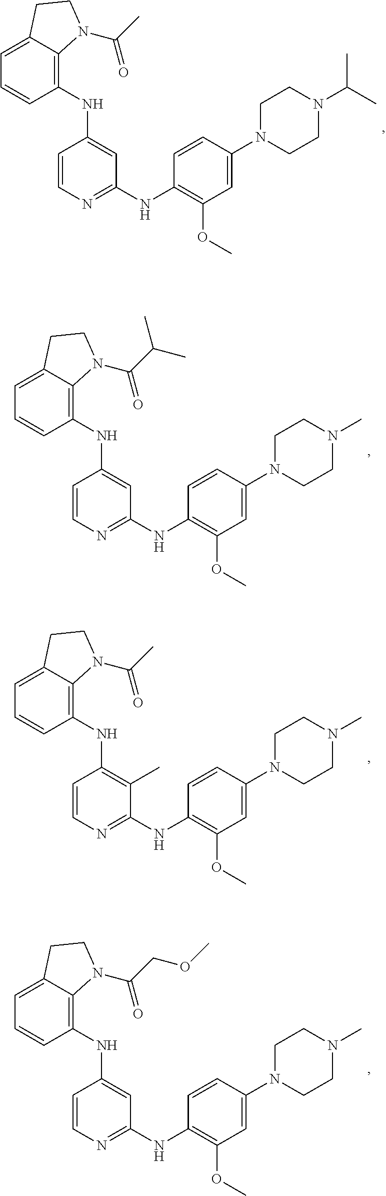 [SMILES: COc1cc(N2CCN(C(C)C)CC2)ccc1Nc1cc(Nc2cccc3c2N(C(C)=O)CC3)ccn1 ; COc1cc(N2CCN(C)CC2)ccc1Nc1cc(Nc2cccc3c2N(C(=O)C(C)C)CC3)ccn1 ; COc1cc(N2CCN(C)CC2)ccc1Nc1nccc(Nc2cccc3c2N(C(C)=O)CC3)c1C ; COCC(=O)N1CCc2cccc(Nc3ccnc(Nc4ccc(N5CCN(C)CC5)cc4OC)c3)c21]

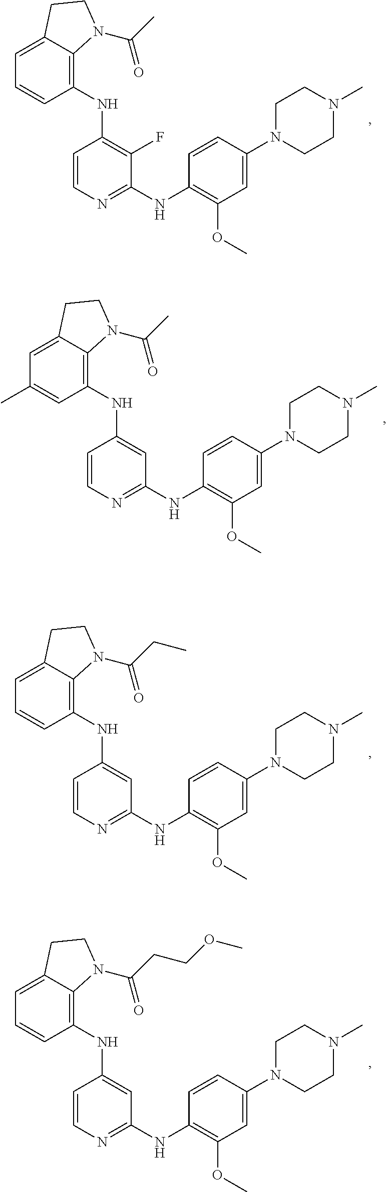

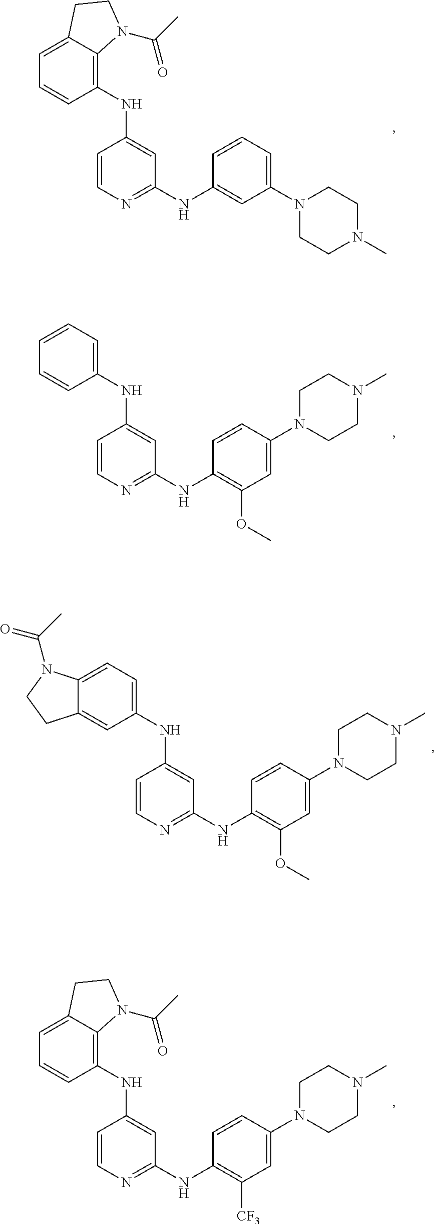 [SMILES: CC(=O)N1CCc2cccc(Nc3ccnc(Nc4cccc(N5CCN(C)CC5)c4)c3)c21 ; COc1cc(N2CCN(C)CC2)ccc1Nc1cc(Nc2ccccc2)ccn1 ; COc1cc(N2CCN(C)CC2)ccc1Nc1cc(Nc2ccc3c(c2)CCN3C(C)=O)ccn1 ; CC(=O)N1CCc2cccc(Nc3ccnc(Nc4ccc(N5CCN(C)CC5)cc4C(F)(F)F)c3)c21]

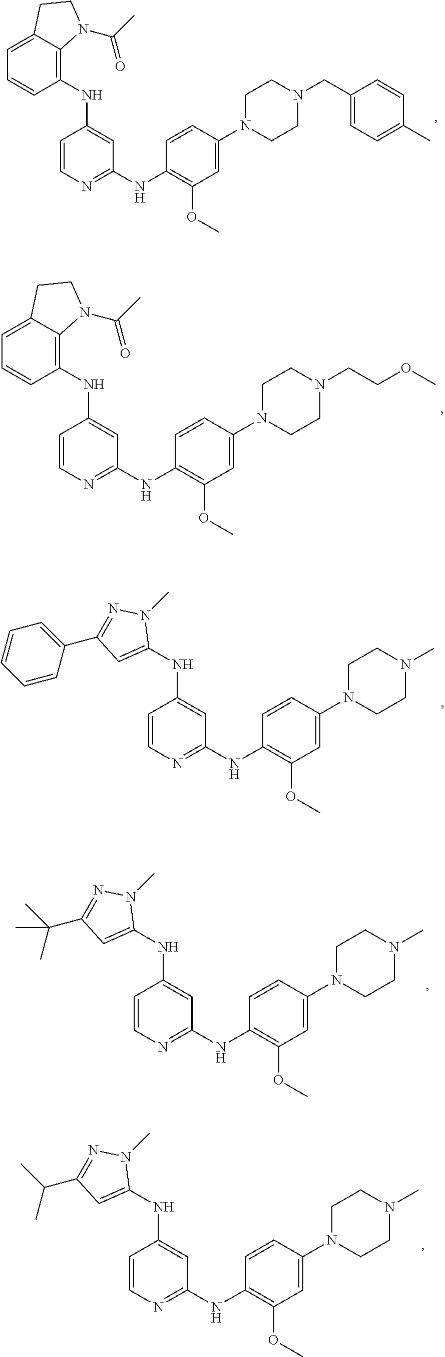

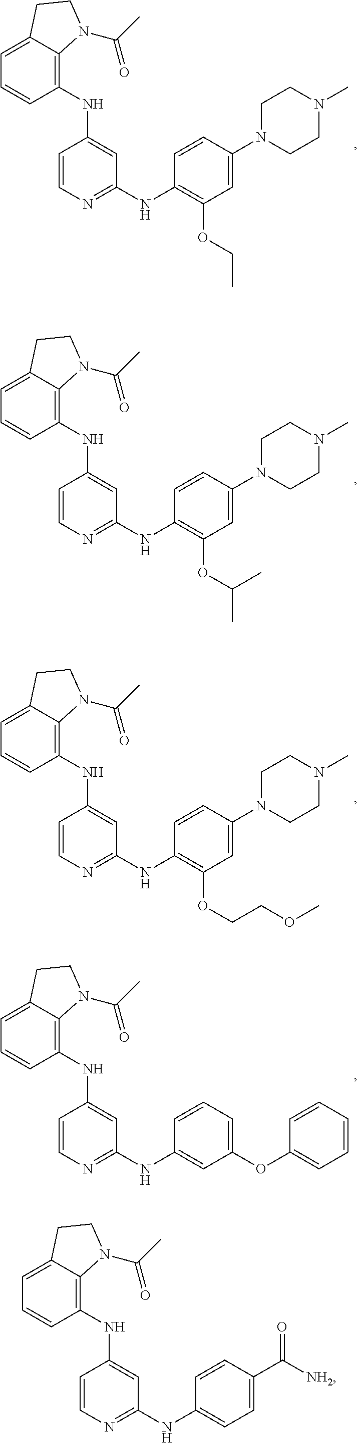 [SMILES: CCOc1cc(N2CCN(C)CC2)ccc1Nc1cc(Nc2cccc3c2N(C(C)=O)CC3)ccn1 ; CC(=O)N1CCc2cccc(Nc3ccnc(Nc4ccc(N5CCN(C)CC5)cc4OC(C)C)c3)c21 ; COCCOc1cc(N2CCN(C)CC2)ccc1Nc1cc(Nc2cccc3c2N(C(C)=O)CC3)ccn1 ; CC(=O)N1CCc2cccc(Nc3ccnc(Nc4cccc(Oc5ccccc5)c4)c3)c21 ; CC(=O)N1CCc2cccc(Nc3ccnc(Nc4ccc(C(N)=O)cc4)c3)c21]

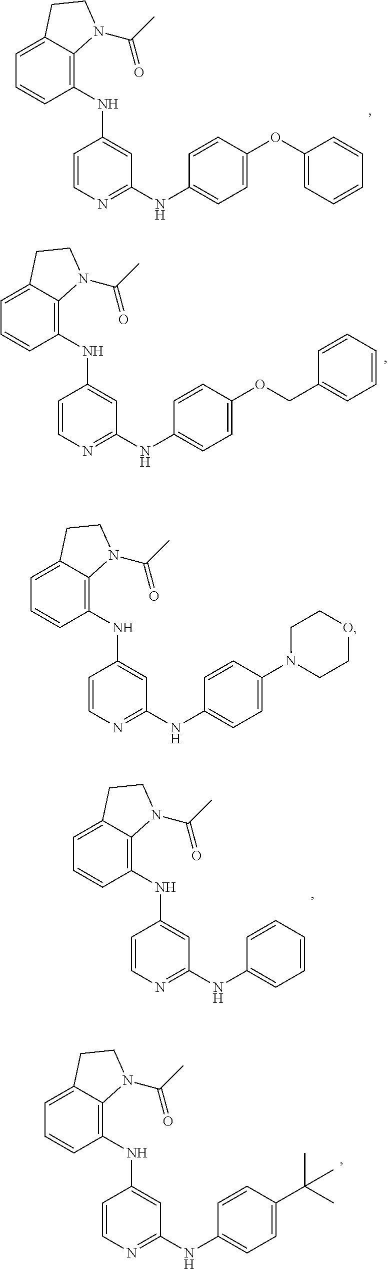 [SMILES: CC(=O)N1CCc2cccc(Nc3ccnc(Nc4ccc(Oc5ccccc5)cc4)c3)c21 ; CC(=O)N1CCc2cccc(Nc3ccnc(Nc4ccc(OCc5ccccc5)cc4)c3)c21 ; CC(=O)N1CCc2cccc(Nc3ccnc(Nc4ccc(N5CCOCC5)cc4)c3)c21 ; CC(=O)N1CCc2cccc(Nc3ccnc(Nc4ccccc4)c3)c21 ; CC(=O)N1CCc2cccc(Nc3ccnc(Nc4ccc(C(C)(C)C)cc4)c3)c21]

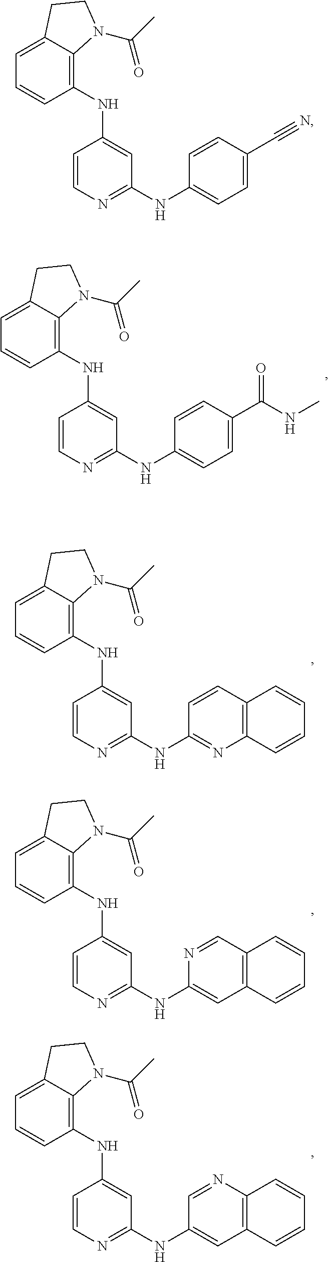

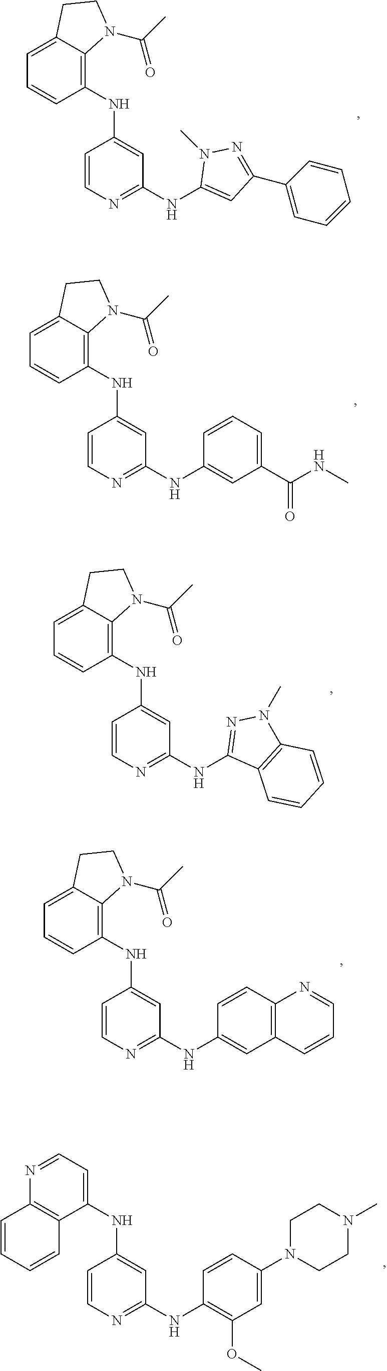 [SMILES: CC(=O)N1CCc2cccc(Nc3ccnc(Nc4cc(-c5ccccc5)nn4C)c3)c21 ; CNC(=O)c1cccc(Nc2cc(Nc3cccc4c3N(C(C)=O)CC4)ccn2)c1 ; CC(=O)N1CCc2cccc(Nc3ccnc(Nc4nn(C)c5ccccc45)c3)c21 ; CC(=O)N1CCc2cccc(Nc3ccnc(Nc4ccc5ncccc5c4)c3)c21 ; COc1cc(N2CCN(C)CC2)ccc1Nc1cc(Nc2ccnc3ccccc23)ccn1]

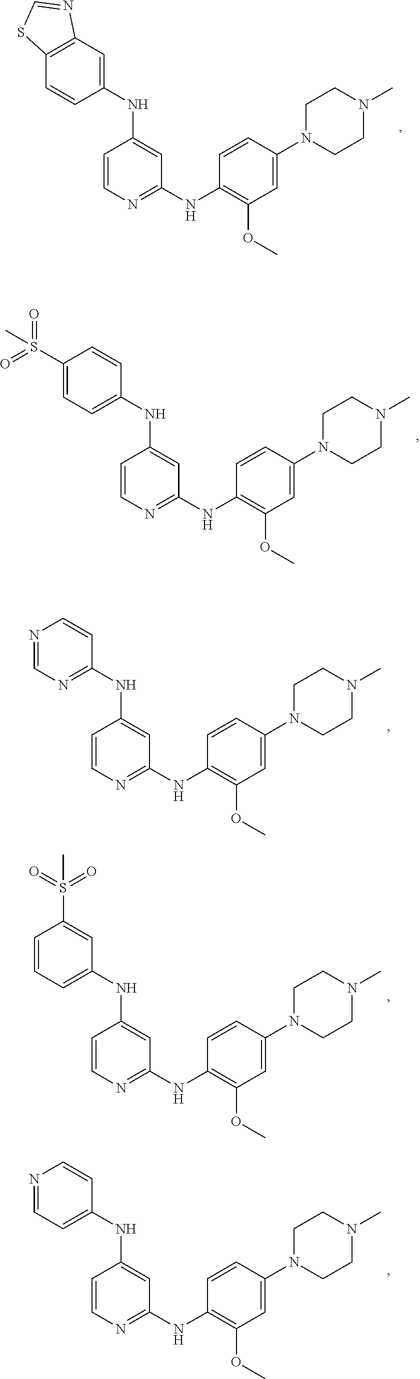 [SMILES: COc1cc(N2CCN(C)CC2)ccc1Nc1cc(Nc2ccc3scnc3c2)ccn1 ; COc1cc(N2CCN(C)CC2)ccc1Nc1cc(Nc2ccc(S(C)(=O)=O)cc2)ccn1 ; COc1cc(N2CCN(C)CC2)ccc1Nc1cc(Nc2ccncn2)ccn1 ; COc1cc(N2CCN(C)CC2)ccc1Nc1cc(Nc2cccc(S(C)(=O)=O)c2)ccn1 ; COc1cc(N2CCN(C)CC2)ccc1Nc1cc(Nc2ccncc2)ccn1]

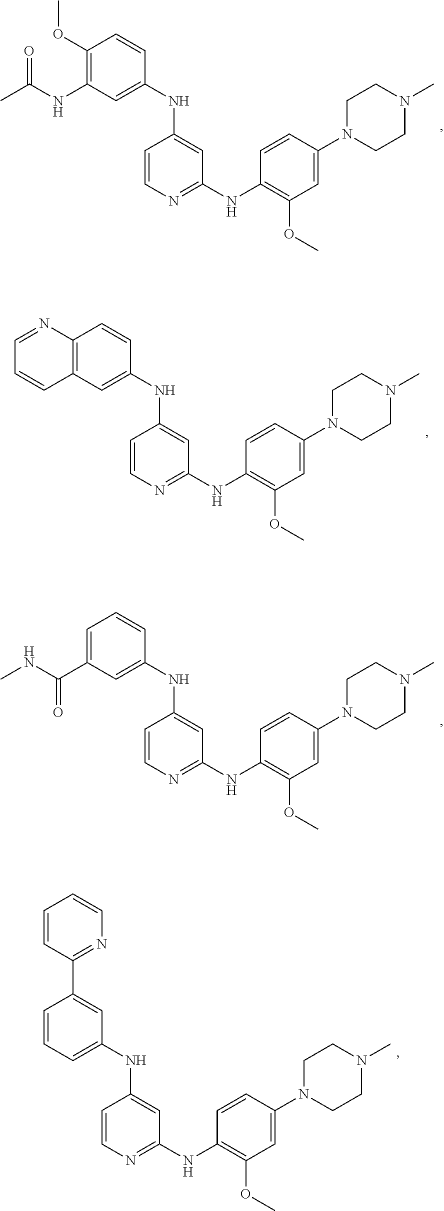

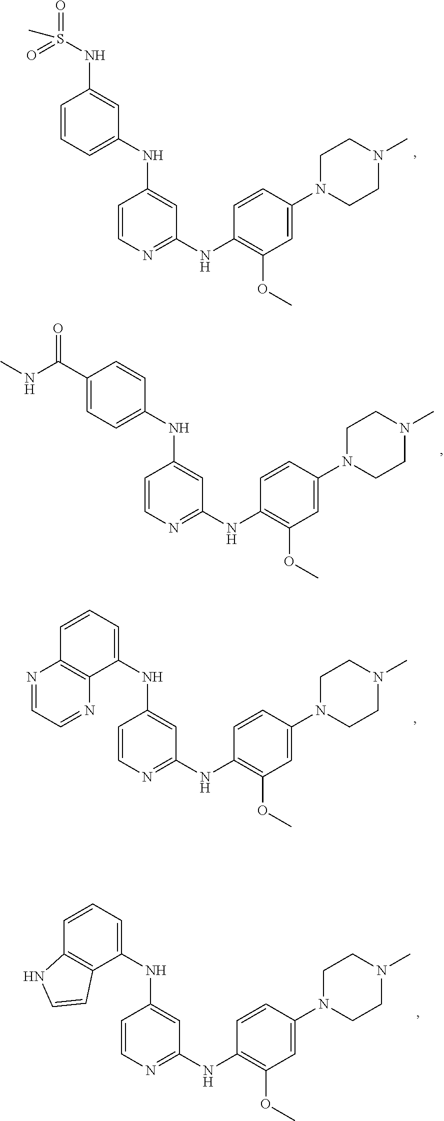

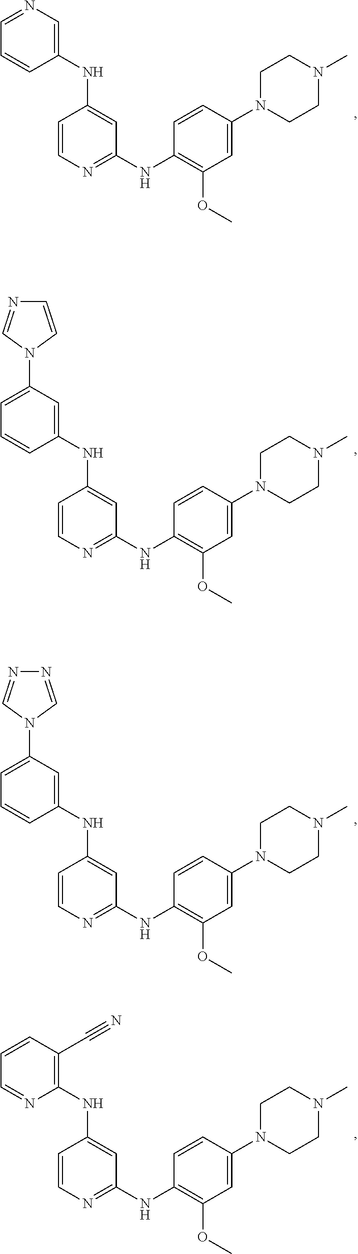 [SMILES: COc1cc(N2CCN(C)CC2)ccc1Nc1cc(Nc2cccnc2)ccn1 ; COc1cc(N2CCN(C)CC2)ccc1Nc1cc(Nc2cccc(-n3ccnc3)c2)ccn1 ; COc1cc(N2CCN(C)CC2)ccc1Nc1cc(Nc2cccc(-n3cnnc3)c2)ccn1 ; COc1cc(N2CCN(C)CC2)ccc1Nc1cc(Nc2ncccc2C#N)ccn1]

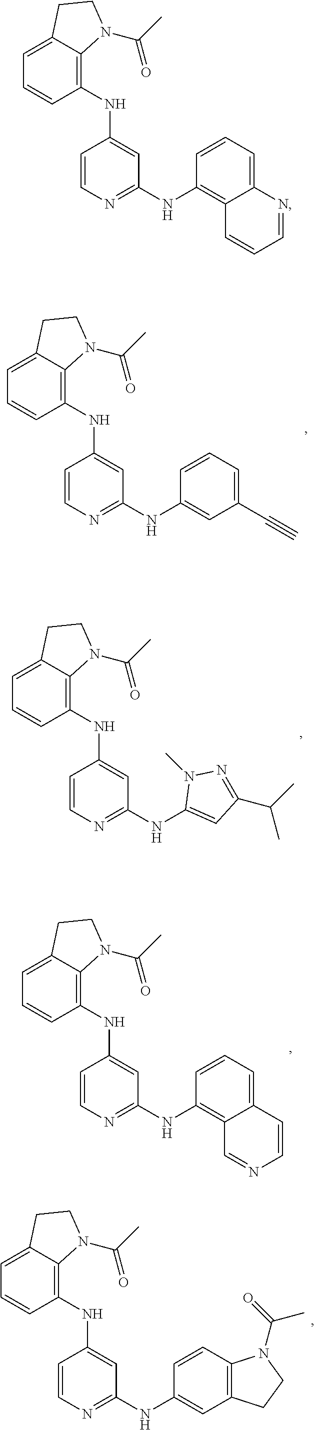 [SMILES: CC(=O)N1CCc2cccc(Nc3ccnc(Nc4cccc5ncccc45)c3)c21 ; C#Cc1cccc(Nc2cc(Nc3cccc4c3N(C(C)=O)CC4)ccn2)c1 ; CC(=O)N1CCc2cccc(Nc3ccnc(Nc4cc(C(C)C)nn4C)c3)c21 ; CC(=O)N1CCc2cccc(Nc3ccnc(Nc4cccc5ccncc45)c3)c21 ; CC(=O)N1CCc2cc(Nc3cc(Nc4cccc5c4N(C(C)=O)CC5)ccn3)ccc21]

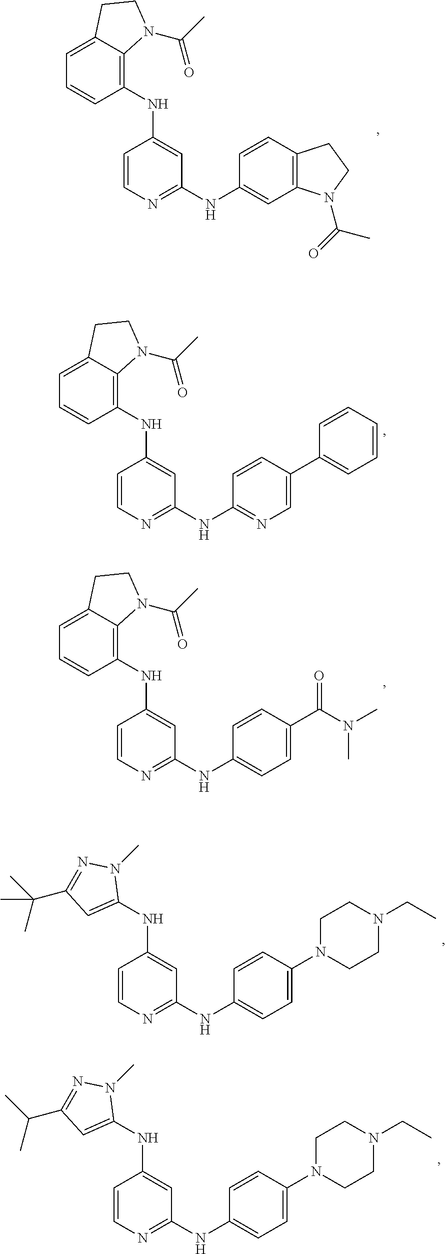

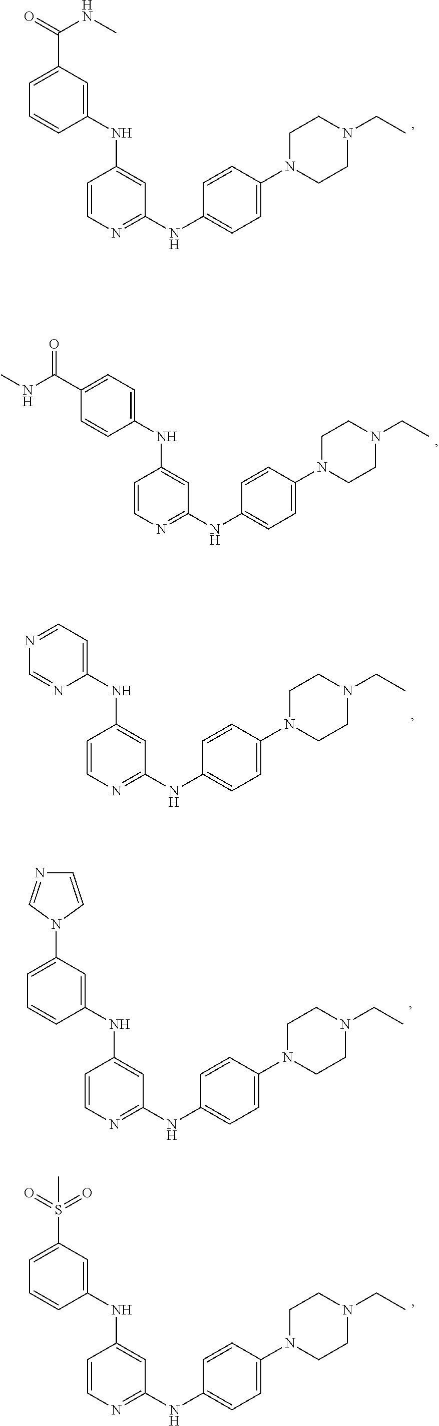

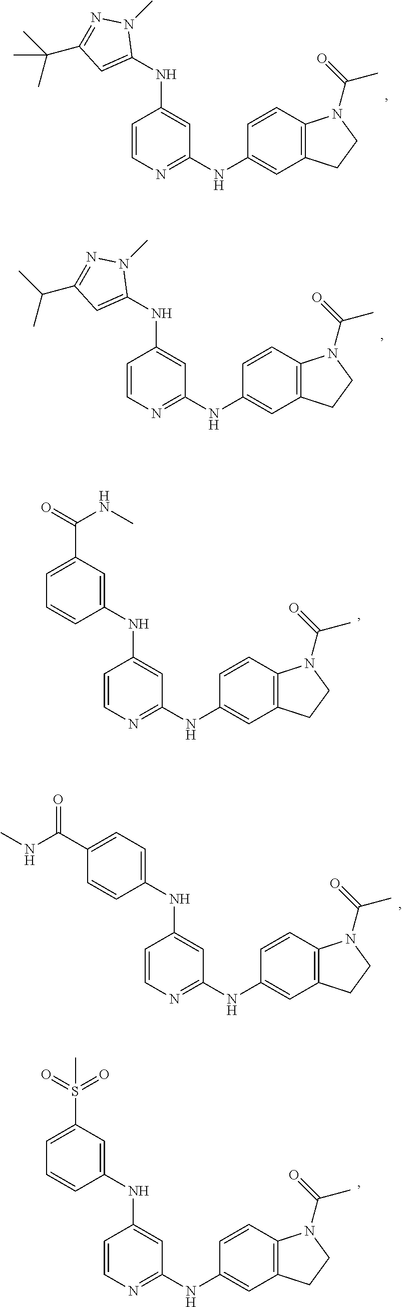

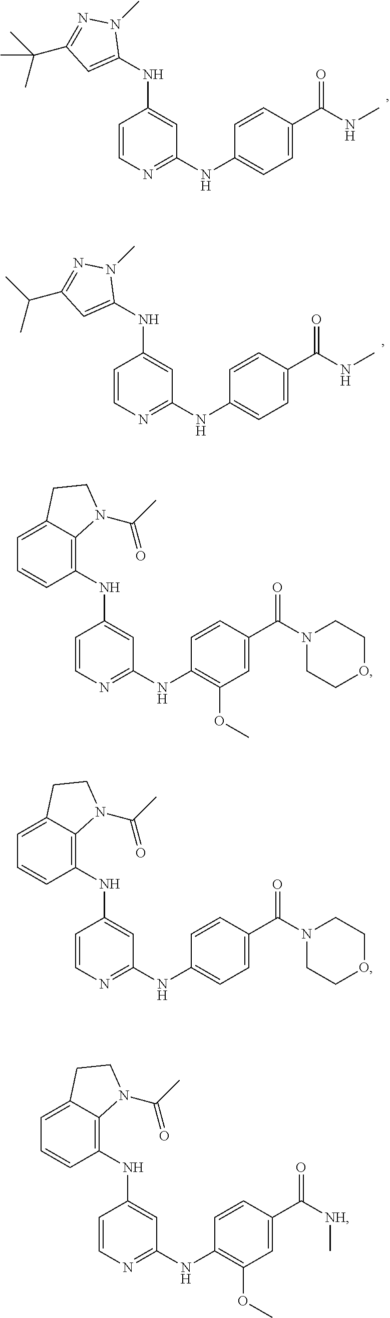

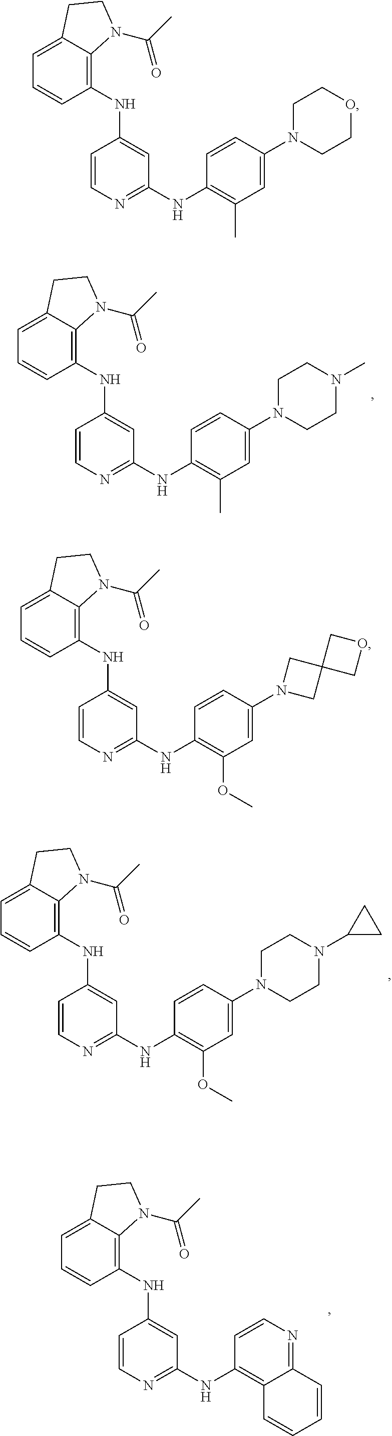 [SMILES: CC(=O)N1CCc2cccc(Nc3ccnc(Nc4ccc(N5CCOCC5)cc4C)c3)c21 ; CC(=O)N1CCc2cccc(Nc3ccnc(Nc4ccc(N5CCN(C)CC5)cc4C)c3)c21 ; COc1cc(N2CC3(COC3)C2)ccc1Nc1cc(Nc2cccc3c2N(C(C)=O)CC3)ccn1 ; COc1cc(N2CCN(C3CC3)CC2)ccc1Nc1cc(Nc2cccc3c2N(C(C)=O)CC3)ccn1 ; CC(=O)N1CCc2cccc(Nc3ccnc(Nc4ccnc5ccccc45)c3)c21]

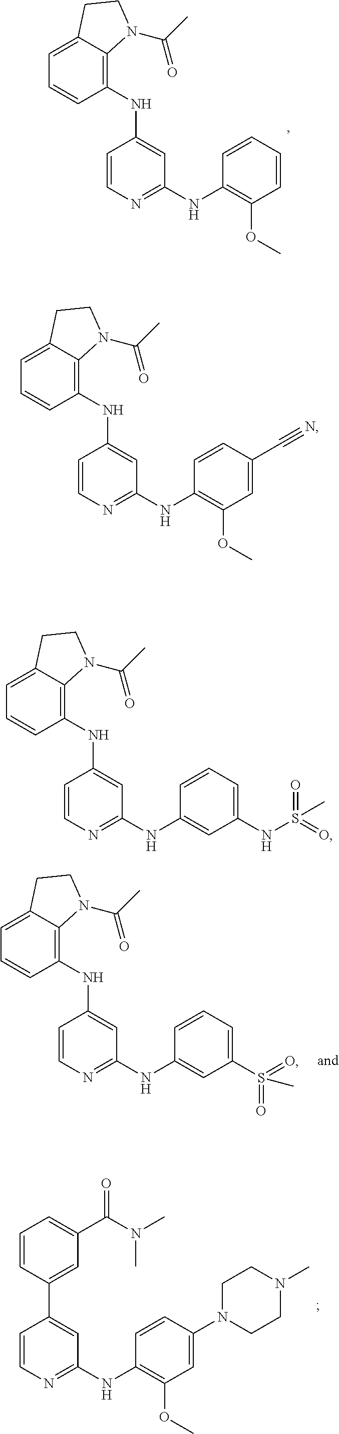

or a tautomer, N-oxide, pharmaceutically acceptable salt, or solvate thereof.

Preferably, the compound of the invention is a compound selected from:

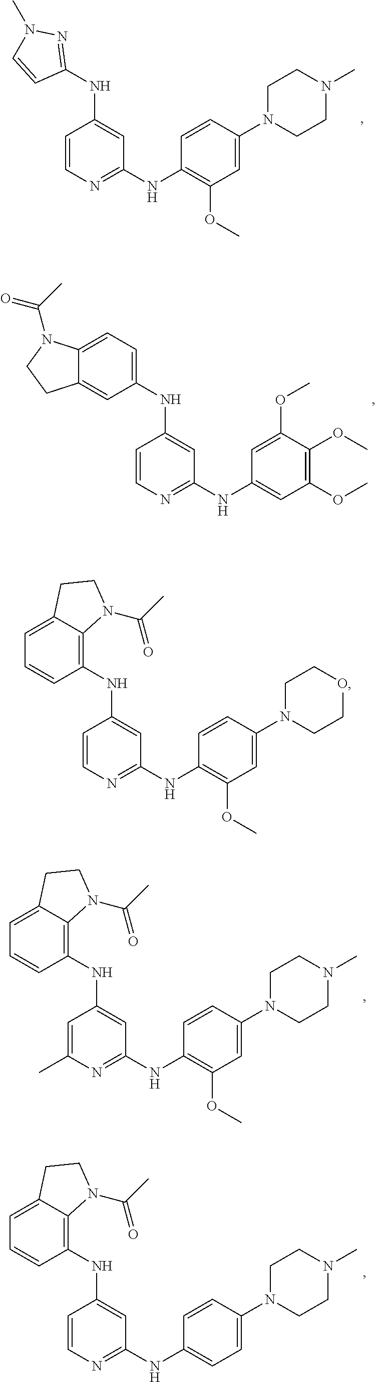 [SMILES: COc1cc(N2CCN(C)CC2)ccc1Nc1cc(Nc2ccn(C)n2)ccn1 ; COc1cc(Nc2cc(Nc3ccc4c(c3)CCN4C(C)=O)ccn2)cc(OC)c1OC ; COc1cc(N2CCOCC2)ccc1Nc1cc(Nc2cccc3c2N(C(C)=O)CC3)ccn1 ; COc1cc(N2CCN(C)CC2)ccc1Nc1cc(Nc2cccc3c2N(C(C)=O)CC3)cc(C)n1 ; CC(=O)N1CCc2cccc(Nc3ccnc(Nc4ccc(N5CCN(C)CC5)cc4)c3)c21]

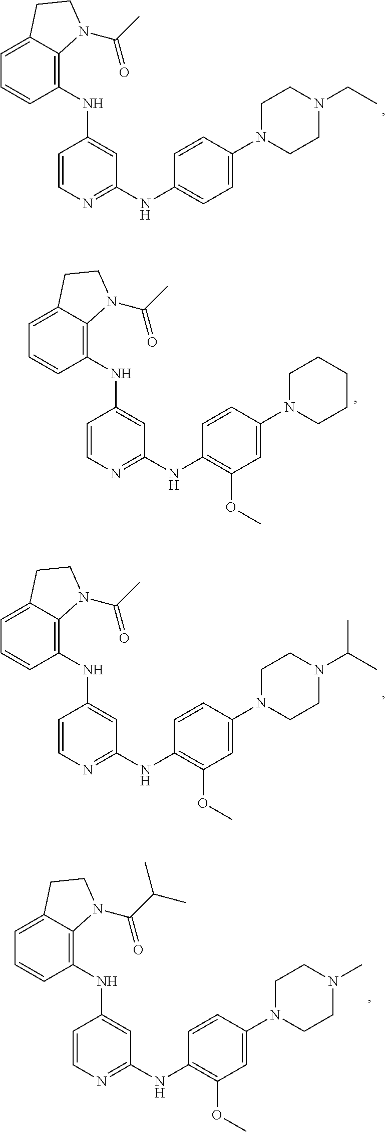 [SMILES: CCN1CCN(c2ccc(Nc3cc(Nc4cccc5c4N(C(C)=O)CC5)ccn3)cc2)CC1 ; COc1cc(N2CCCCC2)ccc1Nc1cc(Nc2cccc3c2N(C(C)=O)CC3)ccn1 ; COc1cc(N2CCN(C(C)C)CC2)ccc1Nc1cc(Nc2cccc3c2N(C(C)=O)CC3)ccn1 ; COc1cc(N2CCN(C)CC2)ccc1Nc1cc(Nc2cccc3c2N(C(=O)C(C)C)CC3)ccn1]

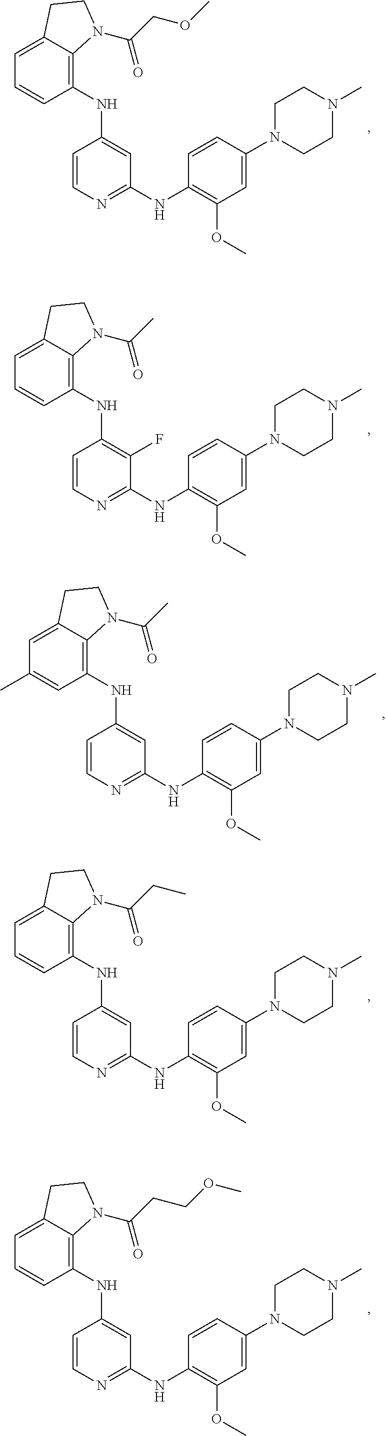

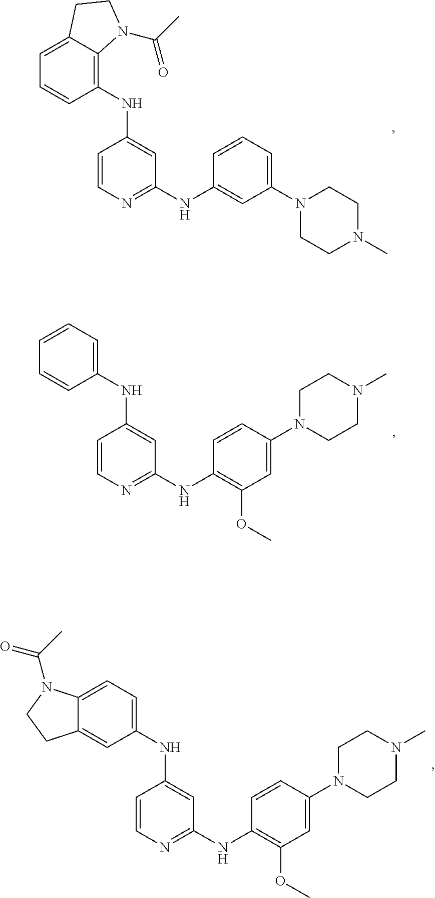 [SMILES: CC(=O)N1CCc2cccc(Nc3ccnc(Nc4cccc(N5CCN(C)CC5)c4)c3)c21 ; COc1cc(N2CCN(C)CC2)ccc1Nc1cc(Nc2ccccc2)ccn1 ; COc1cc(N2CCN(C)CC2)ccc1Nc1cc(Nc2ccc3c(c2)CCN3C(C)=O)ccn1]

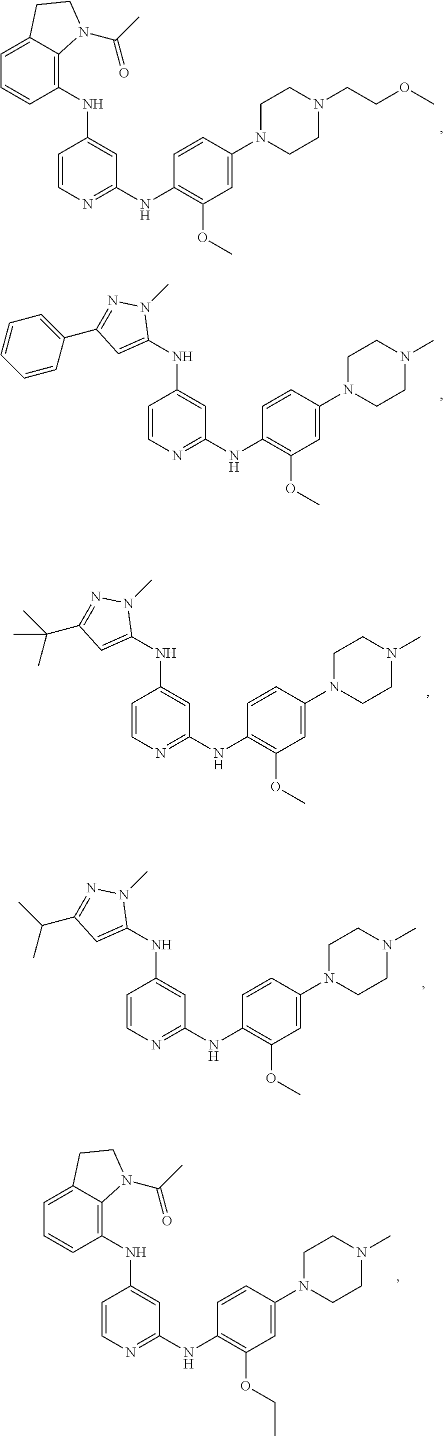

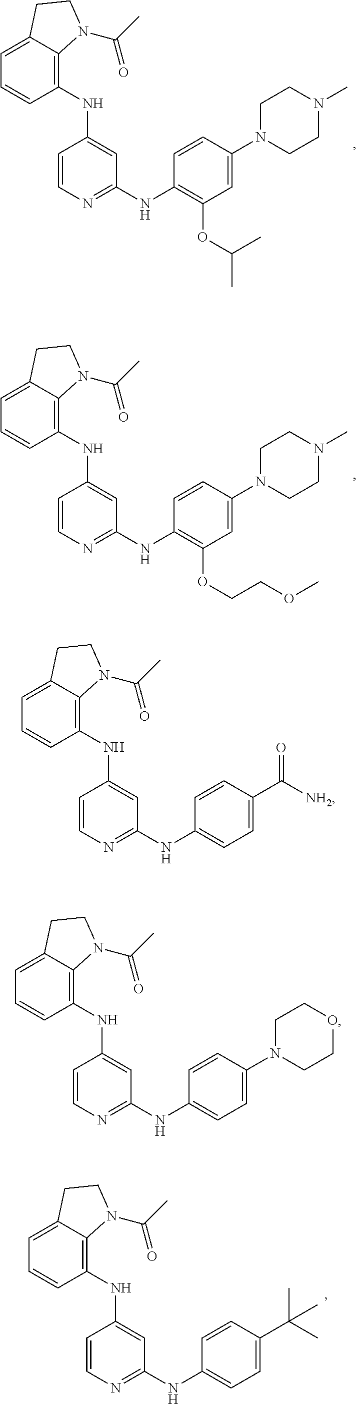 [SMILES: CC(=O)N1CCc2cccc(Nc3ccnc(Nc4ccc(N5CCN(C)CC5)cc4OC(C)C)c3)c21 ; COCCOc1cc(N2CCN(C)CC2)ccc1Nc1cc(Nc2cccc3c2N(C(C)=O)CC3)ccn1 ; CC(=O)N1CCc2cccc(Nc3ccnc(Nc4ccc(C(N)=O)cc4)c3)c21 ; CC(=O)N1CCc2cccc(Nc3ccnc(Nc4ccc(N5CCOCC5)cc4)c3)c21 ; CC(=O)N1CCc2cccc(Nc3ccnc(Nc4ccc(C(C)(C)C)cc4)c3)c21]

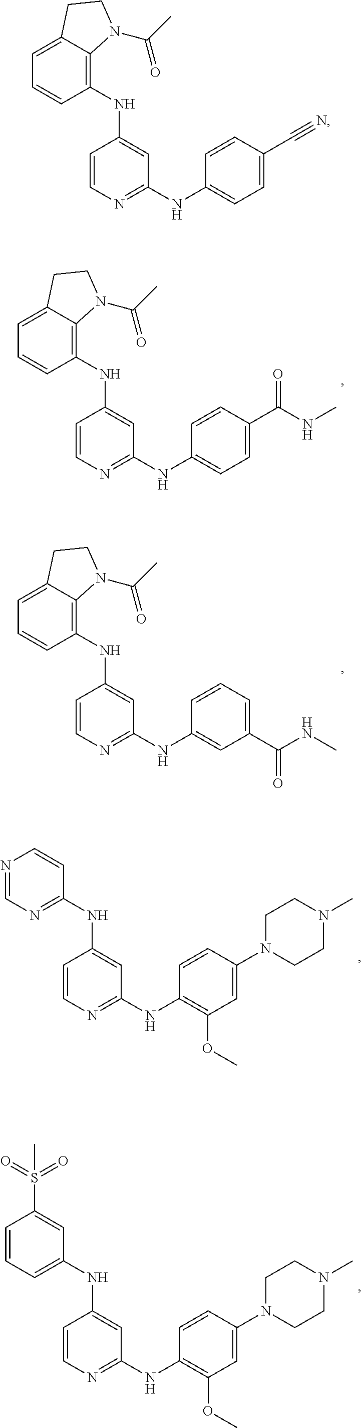 [SMILES: CC(=O)N1CCc2cccc(Nc3ccnc(Nc4ccc(C#N)cc4)c3)c21 ; CNC(=O)c1ccc(Nc2cc(Nc3cccc4c3N(C(C)=O)CC4)ccn2)cc1 ; CNC(=O)c1cccc(Nc2cc(Nc3cccc4c3N(C(C)=O)CC4)ccn2)c1 ; COc1cc(N2CCN(C)CC2)ccc1Nc1cc(Nc2ccncn2)ccn1 ; COc1cc(N2CCN(C)CC2)ccc1Nc1cc(Nc2cccc(S(C)(=O)=O)c2)ccn1]

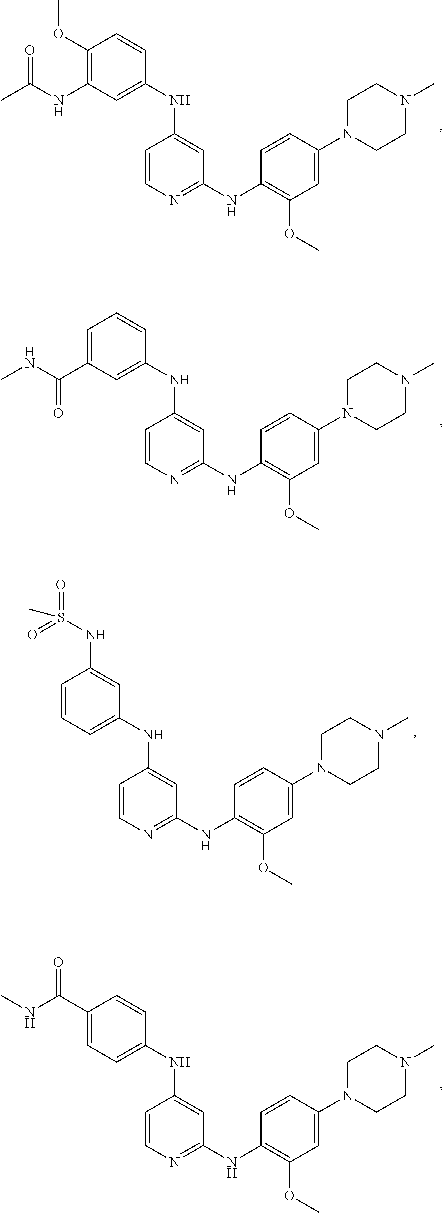

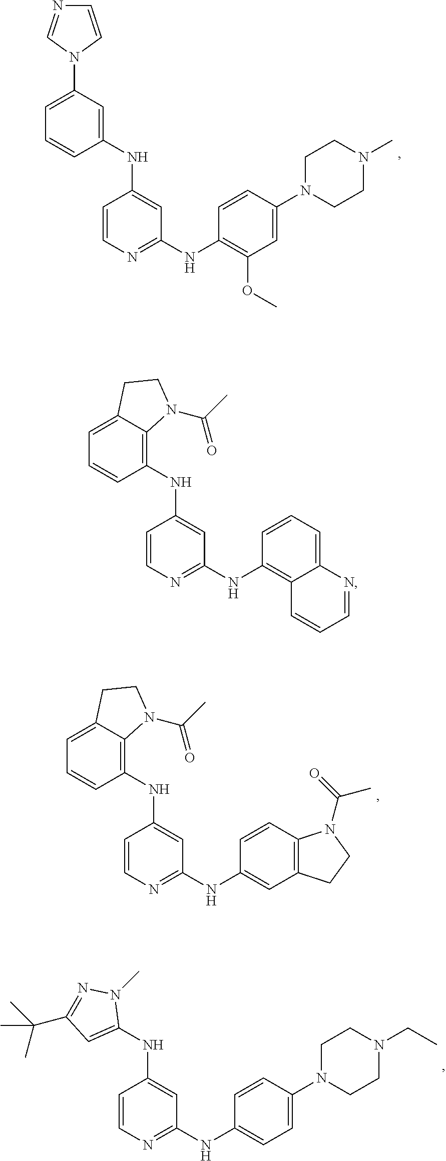

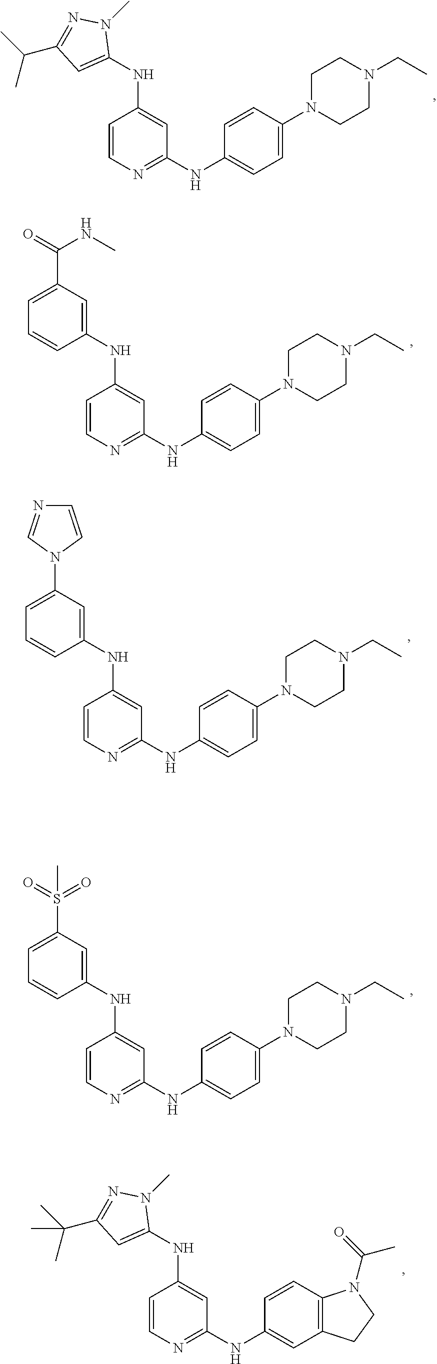

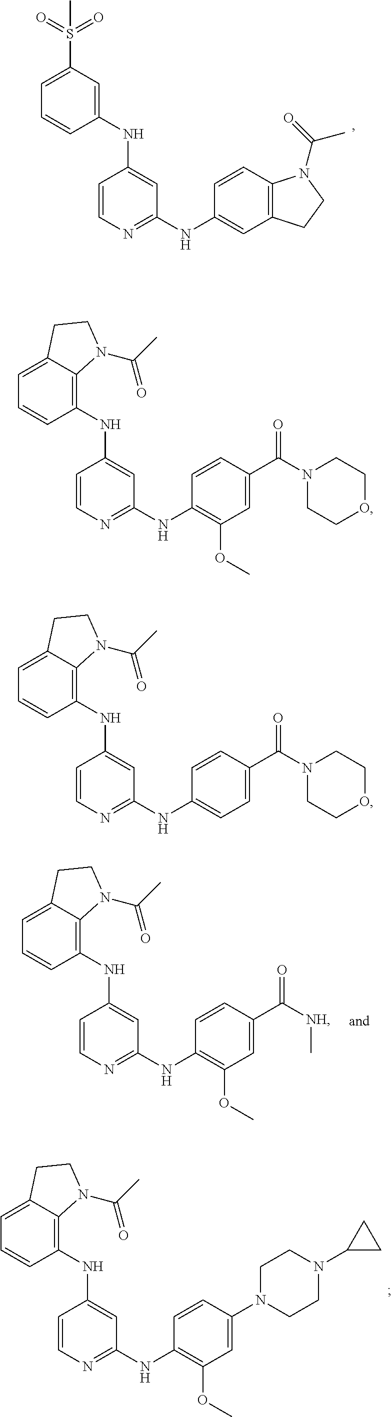 [SMILES: CC(=O)N1CCc2cc(Nc3cc(Nc4cccc(S(C)(=O)=O)c4)ccn3)ccc21 ; COc1cc(C(=O)N2CCOCC2)ccc1Nc1cc(Nc2cccc3c2N(C(C)=O)CC3)ccn1 ; CC(=O)N1CCc2cccc(Nc3ccnc(Nc4ccc(C(=O)N5CCOCC5)cc4)c3)c21 ; CNC(=O)c1ccc(Nc2cc(Nc3cccc4c3N(C(C)=O)CC4)ccn2)c(OC)c1 ; COc1cc(N2CCN(C3CC3)CC2)ccc1Nc1cc(Nc2cccc3c2N(C(C)=O)CC3)ccn1]

or a tautomer, N-oxide, pharmaceutically acceptable salt, or solvate thereof.

Further preferably, the compound of the invention is a compound selected from:

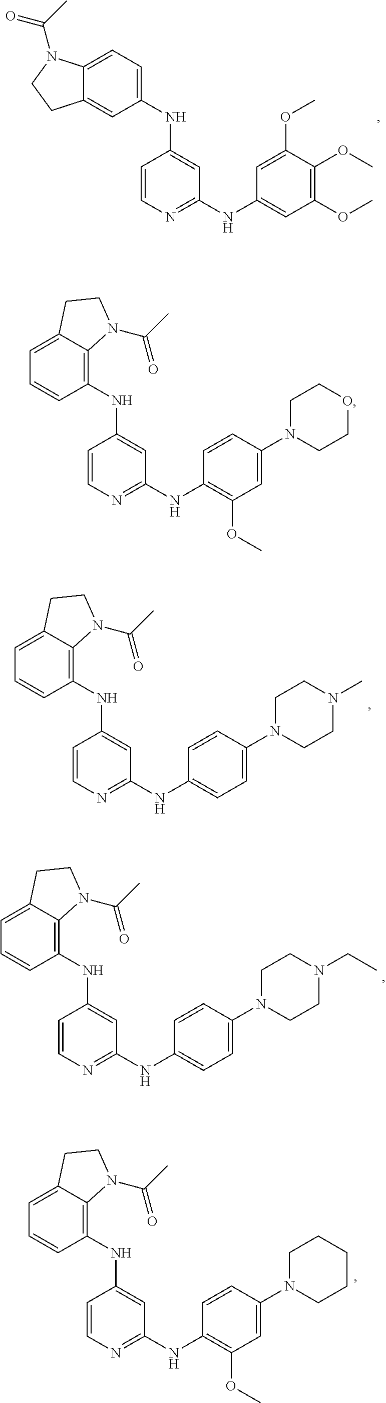 [SMILES: COc1cc(Nc2cc(Nc3ccc4c(c3)CCN4C(C)=O)ccn2)cc(OC)c1OC ; COc1cc(N2CCOCC2)ccc1Nc1cc(Nc2cccc3c2N(C(C)=O)CC3)ccn1 ; CC(=O)N1CCc2cccc(Nc3ccnc(Nc4ccc(N5CCN(C)CC5)cc4)c3)c21 ; CCN1CCN(c2ccc(Nc3cc(Nc4cccc5c4N(C(C)=O)CC5)ccn3)cc2)CC1 ; COc1cc(N2CCCCC2)ccc1Nc1cc(Nc2cccc3c2N(C(C)=O)CC3)ccn1]

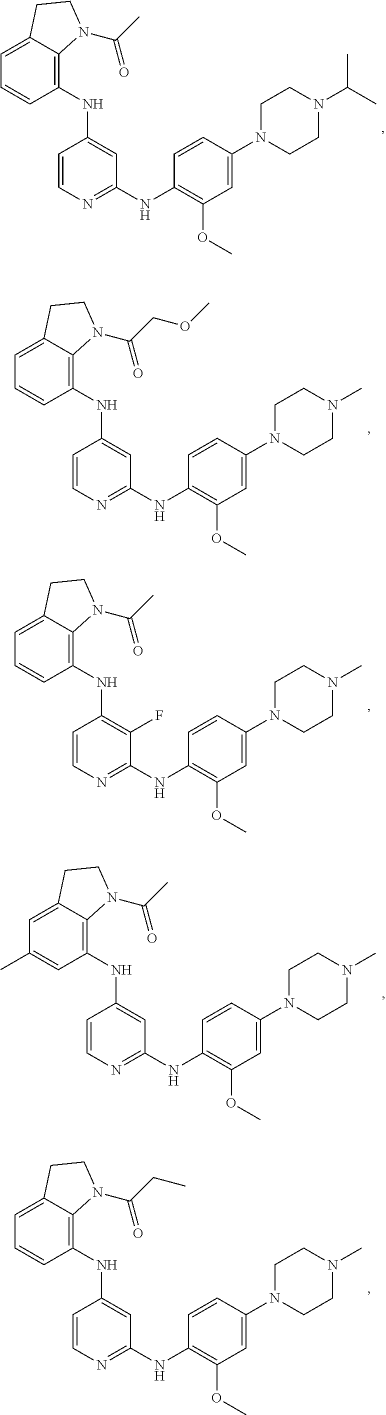

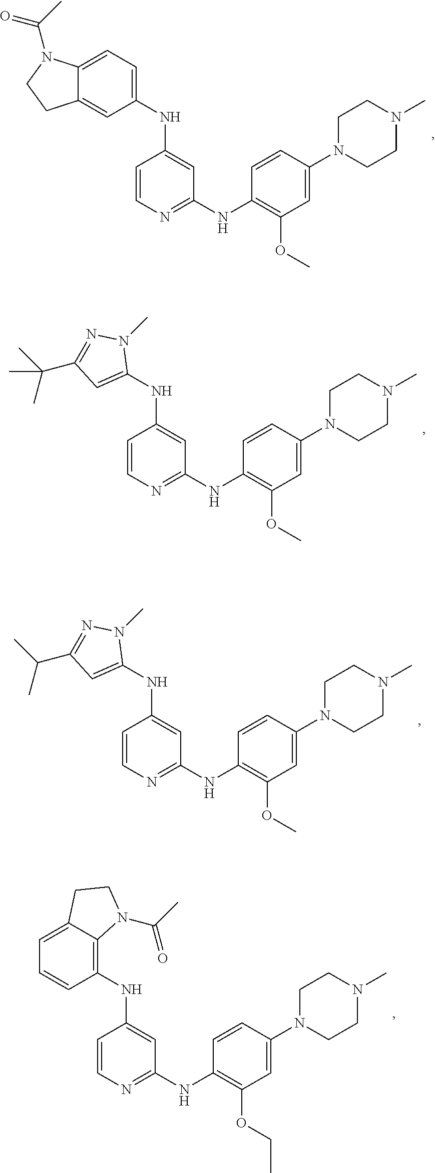

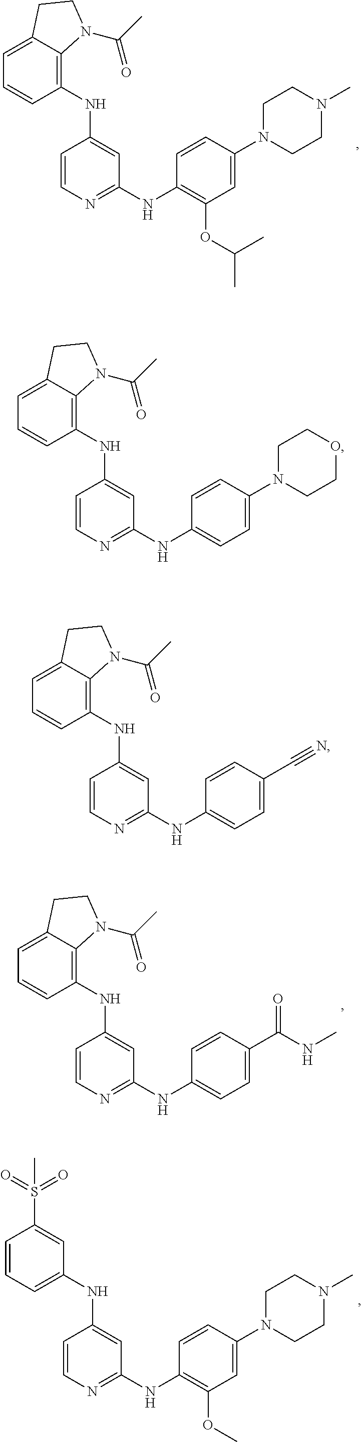 [SMILES: CC(=O)N1CCc2cccc(Nc3ccnc(Nc4ccc(N5CCN(C)CC5)cc4OC(C)C)c3)c21 ; CC(=O)N1CCc2cccc(Nc3ccnc(Nc4ccc(N5CCOCC5)cc4)c3)c21 ; CC(=O)N1CCc2cccc(Nc3ccnc(Nc4ccc(C#N)cc4)c3)c21 ; CNC(=O)c1ccc(Nc2cc(Nc3cccc4c3N(C(C)=O)CC4)ccn2)cc1 ; COc1cc(N2CCN(C)CC2)ccc1Nc1cc(Nc2cccc(S(C)(=O)=O)c2)ccn1]

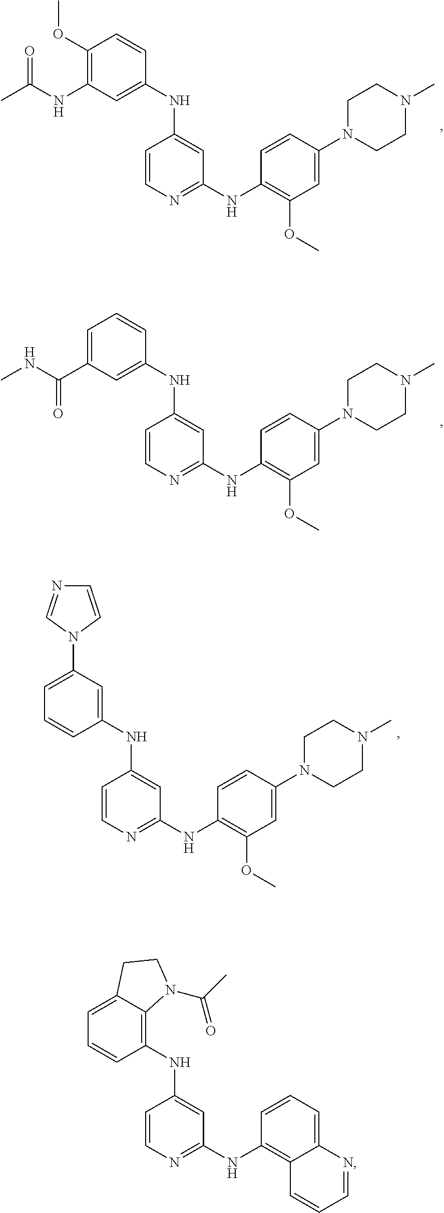

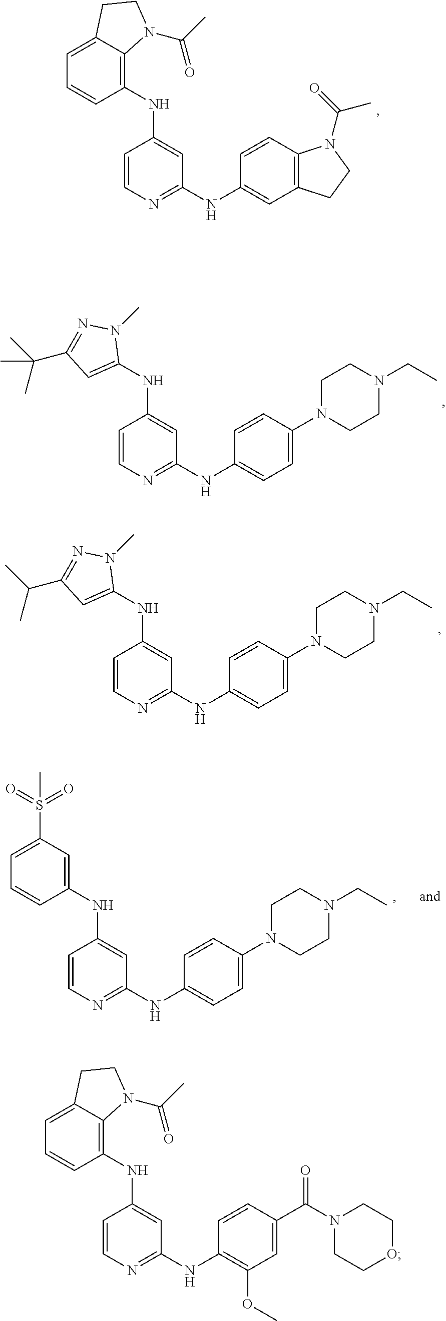

or a tautomer, N-oxide, pharmaceutically acceptable salt, or solvate thereof.

Most preferably, the compound of the invention is:

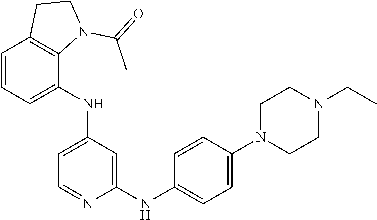 [SMILES: CCN1CCN(c2ccc(Nc3cc(Nc4cccc5c4N(C(C)=O)CC5)ccn3)cc2)CC1]

or a tautomer, N-oxide, pharmaceutically acceptable salt, or solvate thereof.

### Dosage, Formulation and Delivery

The compounds of the invention, when used in a method of treatment, can be administered alone, but generally will be administered with a pharmaceutical carrier selected on the basis of the chosen route of administration and standard pharmaceutical practice. Thus, preferably, the compounds of the present invention can be included in a pharmaceutical composition.

The compounds of the invention may be administered in intravenous (bolus or infusion), intraperitoneal, subcutaneous, or intramuscular form, all using dosage forms well known to those of ordinary skill in the pharmaceutical arts.

Alternatively, the compounds of the present invention may be included in a pharmaceutical composition that is suitable for oral administration. One exemplary such pharmaceutical composition is a solid dosage form suitable for oral administration (e.g., a tablet, capsule (each of which includes a sustained release or timed release formulation), pill, powder, or granule). Another exemplary such pharmaceutical composition is a liquid dosage form suitable for oral administration (e.g., an elixir, tincture, suspension, syrup, solution or emulsion).

Alternatively, the compounds of the invention may be administered in a pharmaceutical composition that is suitable for topical administration. Exemplary pharmaceutical compositions suitable for topical administration include creams, gels or lotions.

Alternatively, the compounds of the invention may be administered in a pharmaceutical composition that is suitable for buccal administration.

Alternatively, the compounds of the invention may be administered in a pharmaceutical composition that is suitable for nasal administration.

Alternatively, the compounds of the invention may be administered in a pharmaceutical composition that is suitable for opthalmic administration.

Alternatively, the compounds of the invention may be administered in a pharmaceutical composition that is suitable for rectal administration.

Alternatively, the compounds of the invention may be administered in a pharmaceutical composition that is suitable for vaginal administration.

Alternatively, the compounds of the invention may be administered in a form that is suitable for inhalation or insufflation.

In a specific embodiment, the compounds of the present invention may be administered topically. For example, the compounds of the present invention may be administered topically in a form that is suitable for wound healing. In another example, the compounds of the present invention may be administered topically in a form that is suitable for treating peripheral nerve injury.

In another specific embodiment, the compounds of the present invention may be administered in a form suitable for direct application to an exposed nerve, for instance in the treatment of peripheral nerve injury.

In another specific embodiment, the compounds of the present invention may be administered in a form suitable for percutaneous administration of the compound to an area adjacent to a nerve, for instance in the treatment of peripheral nerve injury.

In another specific embodiment, the compounds of the present invention may be administered by injection into a nerve, for instance in the treatment of peripheral nerve injury.

In another specific embodiment, the compounds of the present invention may be administered by controlled local delivery to an area adjacent to a nerve, for instance in the treatment of peripheral nerve injury. Suitable means for providing controlled local delivery include biomaterials for drug delivery and the use of minipumps.

In another specific embodiment, the compounds of the present invention may be administered by injection into the brain or spinal cord, for instance in the treatment of CNS injury.

In another specific embodiment, the compounds of the present invention may be administered by intrathecal delivery, for instance in the treatment of CNS injury.

In another specific embodiment, the compounds of the present invention may be administered by controlled local delivery to the nervous system, for instance in the treatment of CNS injury. Suitable means for providing controlled local delivery include biomaterials for drug delivery and the use of minipumps.

In another specific embodiment, the compounds of the present invention may be administered intravenously together with a stent. For example, the compounds of the present invention may be administered intravenously together with a stent in treatment after a stroke or a heart attack.

The dosage regimen for the compounds of the invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to prevent, counter, or arrest the progress of the thromboembolic disorder.

By way of general guidance, the daily oral dosage of each active ingredient, when used for the indicated effects, will range between about 0.001 to 1000 mg/kg of body weight, preferably between about 0.01 to 100 mg/kg of body weight per day, and most preferably between about 1.0 to 20 mg/kg/day.

Intravenously, the most preferred doses will range from about 1 to about 10 mg/kg/minute during a constant rate infusion. Compounds of the invention may be administered in a single daily dose, or the total daily dosage may be administered in divided doses of two, three, or four times daily.

Compounds of the invention can be administered in intranasal form via topical use of suitable intranasal vehicles, or via transdermal routes, using transdermal skin patches. When administered in the form of a transdermal delivery system, the dosage administration will, of course, be continuous rather than intermittent throughout the dosage regimen.

The compounds are typically administered in admixture with suitable pharmaceutical diluents, excipients, or carriers (collectively referred to herein as pharmaceutical carriers) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Compounds of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxide-polylysine substituted with palmitoyl residues. Furthermore, the compounds of the present invention may be coupled to a class of biodegradable polymers useful in achieving controlled release of a drug, for example, polylacetic acid, polyglycolic acid, copolymers of polylacetic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Dosage forms (pharmaceutical compositions) suitable for administration may contain from about 1 milligram to about 100 milligrams of active ingredient per dosage unit. In these pharmaceutical compositions the active ingredient will ordinarily be present in an amount of about 0.5-95% by weight based on the total weight of the composition.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance.

In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

Suitable pharmaceutical carriers are described in Remington's Pharmaceutical Sciences, Mack Publishing Company, a standard reference text in this field.

### Compounds/Composition for Use as a Medicament

In an embodiment, the compound or pharmaceutical composition of the present invention is for use as a medicament.

Compounds/Compositions for Use in a Method of Treating and/or Preventing a Disorder Susceptible to Treatment by PI3Kα Activation

In another embodiment, the compound or pharmaceutical composition of the present invention is for use in a method for treating and/or preventing a disorder susceptible to treatment by PI3Kα activation.

Thus, in one embodiment, the compound or pharmaceutical composition of the present invention is for use in a method for protecting tissues from ischaemia reperfusion injury, i.e. toxicity induced by the reoxygenation following the blockade of blood supply to a tissue. Specific examples of this embodiment may include application for use in a method for protecting tissues from ischaemia reperfusion injury in the case of stroke patients receiving tPA/mechanical thrombectomy (Koh, S. H. & Lo, E. H., J Cin Neurol 11, 297-304 (2015); Khan, H. et al. Brain research, 147399 (2021)) patients with myocardial infarction (heart attack) who receive percutaneous coronary intervention (angioplasty with stent) (Koh, S. H. & Lo, E. H., J Clin Neurol 11, 297-304 (2015); Rossello, X. et al. Basic Res Cardiol 112, 66 (2017); Wang, G. et al. The EMBO journal 37 (2018)) or re-oxygenation of other organs, such as kidney (Zhang, G. et al., Kidney Blood Press Res 43, 904-913 (2018); Xiang, H. et al. Clinical and experimental pharmacology & physiology 47, 1030-1040 (2020)).

In another embodiment, the compound or pharmaceutical composition of the present invention is for use in a method for protecting the human or animal body from ionising radiation (Chauhan, A. et al. Scientific reports 11, 1720 (2021)).

In another embodiment, the compound or pharmaceutical composition of the present invention is for use in a method for enhancing tissue regeneration, including in wound healing (including from skin-related injuries including burns (Gan, D. et al. Front Pharmacol 12, 631102 (2021); Sugita, H. et al. Am J Physiol Endocrinol Metab 288, E585-591 (2005); Park, K. K. et al. Science (New York, N.Y 322, 963-966 (2008)) and diabetic foot ulcers), regeneration of airway/lung epithelium in childhood wheeze and asthma (Iosifidis, T. et al. JCI insight 5 (2020)) and endothelial diseases in the eye/cornea, reducing the need for corneal transplantations to repair visual loss which is most commonly due to endothelial dysfunction (Sabater, A. L. et al. Invest Ophthalmol Vis Sci 58, 745-754 (2017)).

In another embodiment, the compound or pharmaceutical composition of the present invention is for use in a method for metabolic sensitization by overcoming insulin resistance in obesity and type 2 diabetes (Foukas, L. C. et al. Nature 441, 366-370 (2006); Knight, Z. A. et al. Cell 125, 733-747 (2006); Frevert, E. U. & Kahn, B. B. Molecular and cellular biology 17, 190-198 (1997); Cichy, S. B. et al. J Biol Chem 273, 6482-6487 (1998); Prakoso, D. et al. Am J Physiol Heart Circ Physiol 318, H840-H852 (2020)).

In another embodiment, the compound or pharmaceutical composition of the present invention is for use in a method for cancer treatment, by inducing cancer cell death through overactivation of the PI3K pathway, especially in cells with over-active PI3K (Klippel, A. et al. Molecular and cellular biology 18, 5699-5711 (1998); Chen, Z. et al. Nature 521, 357-361 (2015); Shojaee, S. et al. Nat Med 22, 379-387 (2016); Muschen, M. Nat Rev Cancer 18, 103-116 (2018)), a condition prevalent in therapy-resistant cancer (Jacobsen, K. et al. Nature communications 8, 410 (2017); Huang, W. C. & Hung, M. C. J Formos Med Assoc 108, 180-194 (2009)).

In another embodiment, the compound or pharmaceutical composition of the present invention is for use in a method for neuro-protection/regeneration (Ames, M. et al. Mol Biol Cell 31, 244-260 (2020); Cuesto, G. et al. J Neurosci 31, 2721-2733 (2011); Asua, D. et al. Neuroscience 370, 81-87 (2018)), including to protect from neuronal damage upon cancer treatment (Matsuda, S. et al. International journal of oncology 49, 1785-1790 (2016)). Genetic PI3Kα activation mediates axonal regeneration in neurons (Nieuwenhuis, B. et al. EMBO Mol Med 12, e11674 (2020)).

In another embodiment, the compound or pharmaceutical composition of the present invention is for use in a method for treating traumatic optic neuropathy e.g. retinal ganglion cell survival and axon regeneration (Morgan-Warren, P. J. et al. Invest Ophthalmol Vis Sci 54, 6903-6916 (2013).

In another embodiment, the compound or pharmaceutical composition of the present invention is for use in a method for neuro-protection/regeneration following CNS injury, including traumatic brain injury (Minnich, J. E. et al. Restor Neurol Neurosci 28, 293-309 (2010)) and spinal cord injury (Zhu, S. et al. Cell Prolif 53, e12860 (2020)). Also hypoxia/ischaemia/stroke (Houlton, J. et al. Front Neurosci 13, 790 (2019); Larpthaveesarp, A. et al. Brain Sci 5, 165-177 (2015)) and nerve root injury (Wang, R. et al. Nat Neurosci 11, 488-496 (2008)).

In another embodiment, the compound or pharmaceutical composition of the present invention is for use in a method for treating neurodegenerative diseases (Allen, S. J. et al. Pharmacol Ther 138, 155-175 (2013); Rai, S. N. et al. Neurotox Res 35, 775-795 (2019); Nagahara, A. H. & Tuszynski, M. H. Nat Rev Drug Discov 10, 209-219 (2011)) including Parkinson's disease (Yang, L., Wang, H., Liu, L. & Xie, A. Front Neurosci 12, 73 (2018); Jha, S. K et al. Int J Mol Cell Med 4, 67-86 (2015)), Alzheimer's disease (Nagahara, A. H. et al. Nat Med 15, 331-337 (2009)), Huntington's disease (Simmons, D. A. et al. Proc Natl Acad Sci USA 106, 4906-4911 (2009)), ALS (Yin, X. et al. Mol Cell Neurosci 68, 303-313 (2015)) and Rett syndrome (Castro, J., Mellios, N. & Sur, M. Curr Opin Neurol 26, 154-159 (2013)).

In another embodiment, the compound or pharmaceutical composition of the present invention is for use in a method for protecting/regenerating enteric neurons in the treatment of gastrointestinal mobility disorders e.g. as a result of diabetes (Anitha, M. et al. J Clin Invest 116, 344-356 (2006)).

Thus, non-limiting examples of disorders susceptible to treatment by PI3Kα activation, and which can be treated and/or prevented using the compound or pharmaceutical composition of the present invention, include ischaemia reperfusion injury; ionisation radiation damage; tissue damage (e.g. to promote tissue regeneration); childhood wheeze; asthma; endothelial diseases in the eye/cornea; obesity; type 2 diabetes; cancer (e.g. cancers exhibiting overactive PI3K, in particular in therapy-resistant cancer); neuronal damage; traumatic optic neuropathy; CNS injury (e.g. traumatic brain injury, spinal cord injury, hypoxia, ischaemia, stroke); neurodegenerative diseases (e.g. Parkinson's disease, Alzheimer's disease, Huntington's disease, ALS, and Rett syndrome); gastrointestinal mobility disorders (e.g. as a result of diabetes).

In a particularly preferred embodiment of the invention, the disorder susceptible to treatment by PI3Kα activation is peripheral nerve injury.

### Use for Manufacture of a Medicament

The present invention provides the use of a compound or pharmaceutical composition according to the invention for the manufacture of a medicament, preferably for use in a method for treating and/or preventing a disorder susceptible to treatment by PI3Kα activation.

It will be understood that all preferred disclosure provided above in connection with the compound or pharmaceutical composition according the invention for use according to the invention is equally contemplated as preferred in the context of the use of a compound or pharmaceutical composition according to the invention, for the manufacture of a medicament according to the present invention.

### Method of Treatment

The present invention also provides a method of treatment, in particular a method for treating and/or preventing a disorder susceptible to treatment by PI3Kα activation in a patient in need thereof, the method comprising administering a compound of the invention, or a pharmaceutical composition of the invention, to the patient.

It will be understood that all preferred disclosure provided above in connection with the compound or pharmaceutical composition according the invention for use according to the invention is equally contemplated as preferred in the context of the method of treatment according to the present invention.

### Use of Compounds

The present invention provides the use of the compounds of the invention as biochemical probes.

PI3K activation has also been shown to improve the success rate of in vitro fertilization by ex vivo activation of dormant follicles from cryopreserved ovarian tissue (Terren, C. & Munaut, C. Reprod Sci (2020)) or in primary ovarian insufficiency (Devenutto, L., Quintana, R. & Quintana, T. Hum Reprod Open 2020, hoaa046 (2020)). Consequently, the present invention further provides the use of the compounds of the invention in in vitro fertilisation techniques.

PI3K activation is also believed to be useful in tissue preservation for organ transplantation such as in kidney and liver transplants, by extending the life of the donor organ, improving transplantation outcomes. Consequently, the present invention further provides the use of the compounds of the invention in in vitro tissue preservation for organ transplantation.

### EXAMPLES

### Example 1

PI3Kα activation data for examples of the invention.

### Measurement of EC50 Values

EC50 values were determined by quantifying phosphorylation on S473 residues of Akt in A549 human adenocarcinoma cells by ELISA (enzyme linked immunoadsorbent assay, R&D Systems, DYC887BE). A549 cells were seeded into 96-well plates (50,000 cells/well) in DMEM supplemented with 10% foetal bovine serum and 1% penicillin-streptomyocin. Following 24 h in culture cells were starved for 24 h in serum-free DMEM before 15 minute treatment with a concentration response curve of compounds. Experimental compounds were diluted in DMSO into 10-point 1:3 concentration response curves in polypropylene V-bottom plates (SLS MIC9050). Concentration response curves were diluted to a 3× stock in serum-free DMEM. Standard 8-point concentration response curves were performed at 50, 16.7, 5.6, 1.9, 0.6, 0.2, 0.07, 0.02 μM and serum-free DMEM containing 0.5% DMSO was used as a negative control. Compound induced Akt phosphorylation was corrected to the DMSO negative controls and then normalised to an Eₘₐₓ of insulin induced Akt phosphorylation following 15 min treatment (1 μM insulin).

### Results

Each of the compounds disclosed herein demonstrates PI3Kα activation. The following table (Table 1) provides the average EC50 for each compound together with an indication of whether the compound achieves a greater than 50% response at 10 μM (preferred), or whether a less than 50% response at 10 μM is observed. Error! Not a valid link.

TABLE 1
PI3Kα activation data for compounds disclosed herein

| | | <50% | |
|---|---|---|---|
| Example | Av | response | |
| (Comp | EC50 | at | |
| ID) | (μM) | 10 μM | Structure |
| 51 (2031) | 0.31 | | 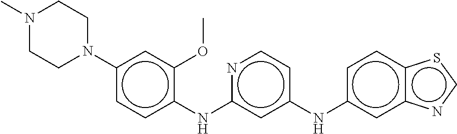 |
| 5 (1906) | 0.44 | X | 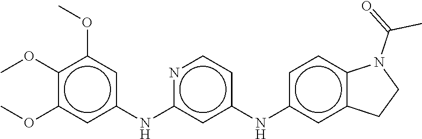 |
| 29 (1998) | 0.89 | | 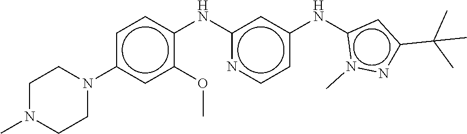 |
| 97 (2354) | 0.97 | | 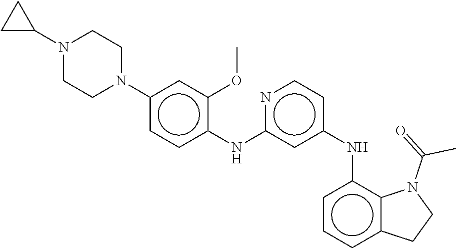 |
| 19 (1958) | 1.01 | | 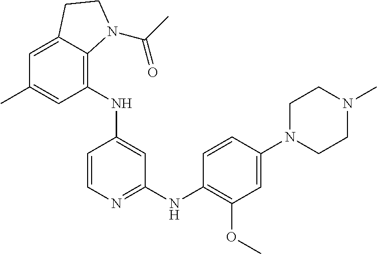 |
| 72 (2072) | 1.08 | | 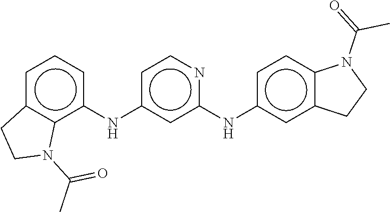 |
| 54 (2043) | 1.22 | | 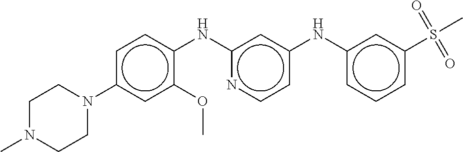 |
| 24 (1989) | 1.25 | | 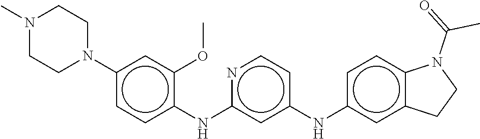 |
| 56 (2046) | 1.28 | | 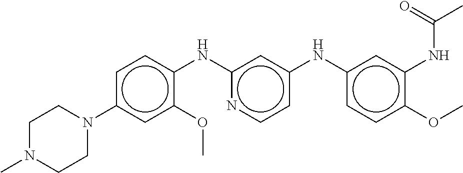 |
| 27 (1994) | 1.30 | | 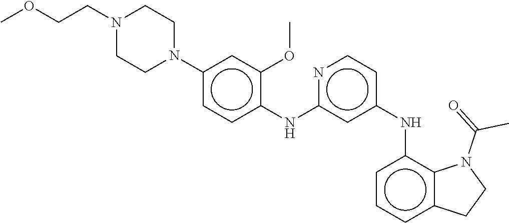 |
| 38 (2015) | 1.32 | | 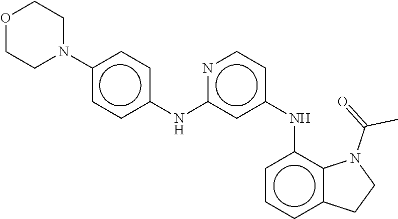 |
| 58 (2049) | 1.35 | | 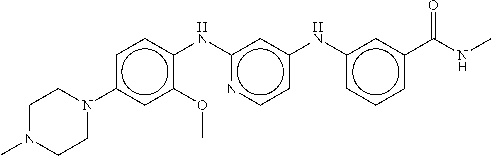 |
| 50 (2029) | 1.36 | | 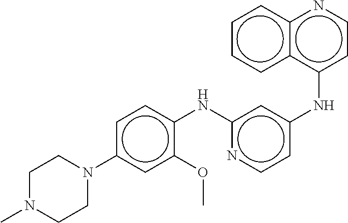 |
| 42 (2019) | 1.37 | | 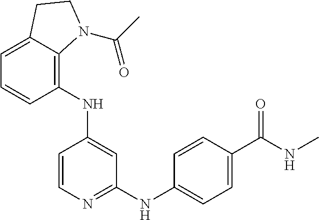 [SMILES: CNC(=O)c1ccc(Nc2cc(Nc3cccc4c3N(C(C)=O)CC4)ccn2)cc1] |
| 32 (2008) | 1.46 | | 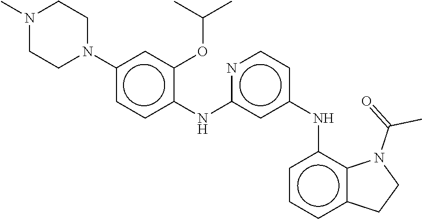 |
| 82 (2108) | 1.52 | | 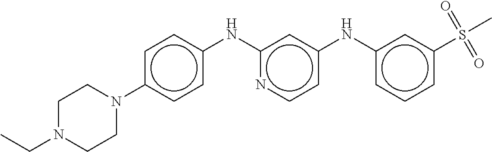 |
| 12 (1938) | 1.53 | | 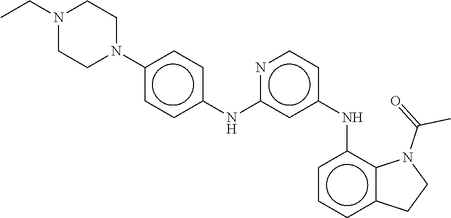 |
| 65 (2063) | 1.53 | | 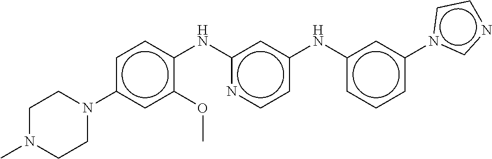 |
| 31 (2007) | 1.55 | | 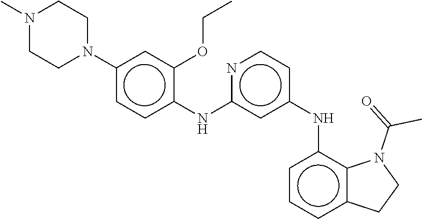 |
| R1 (1888) | 1.55 | | 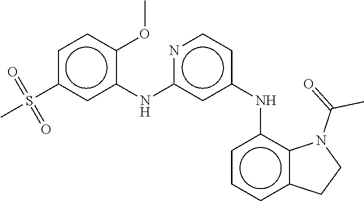 |
| 64 (2061) | 1.58 | | 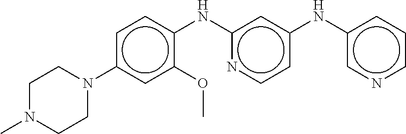 |
| R2 (1890) | 1.62 | | 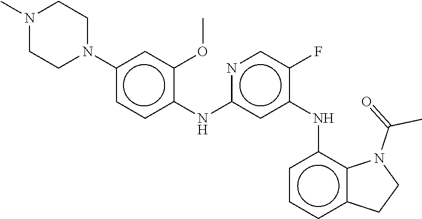 |
| R3 (1887) | 1.96 | | 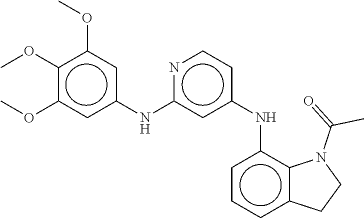 |
| 78 (2104) | 1.98 | | 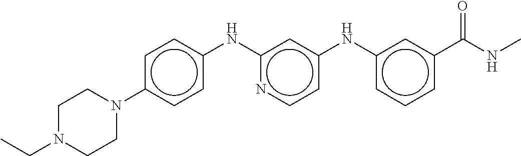 |
| 77 (2103) | 2.09 | | 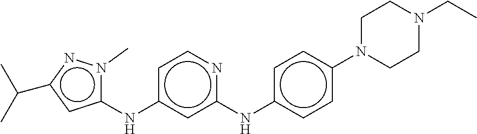 |
| 76 (2102) | 2.20 | | 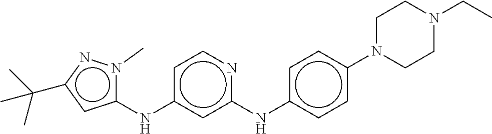 |
| 14 (1942) | 2.27 | | 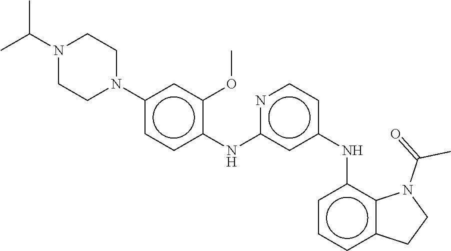 |
| 20 (1959) | 2.28 | | 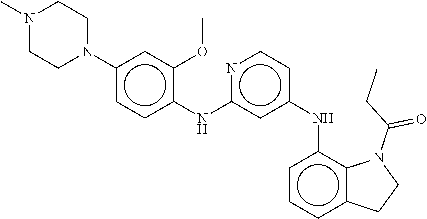 |
| 91 (2347) | 2.29 | | 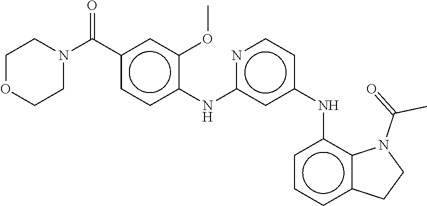 |
| 15 (1943) | 2.39 | | 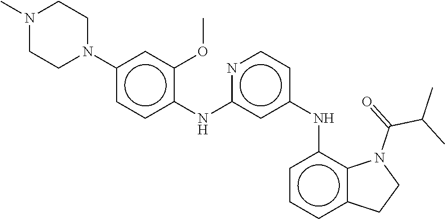 |
| 60 (2054) | 2.51 | | 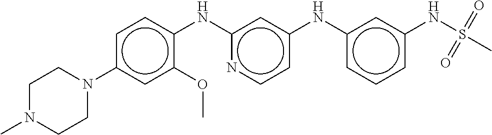 |
| R4 (1889) | 2.55 | | 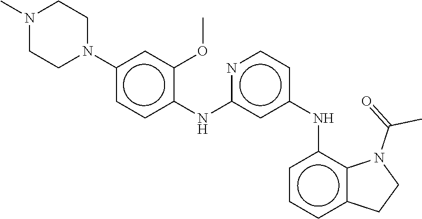 |
| 61 (2055) | 2.61 | | 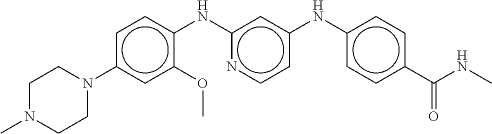 |
| 11 (1937) | 2.64 | | 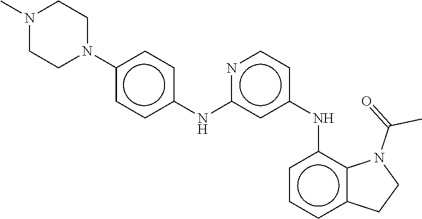 |
| 18 (1951) | 3.08 | | 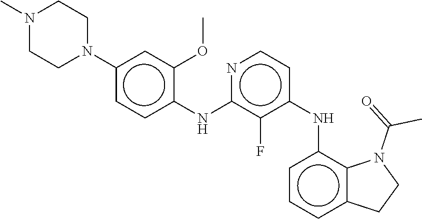 |
| 52 (2038) | 3.81 | | 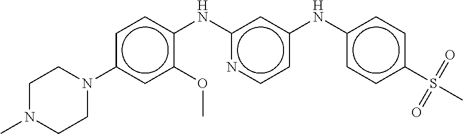 |
| 68 (2067) | 4.00 | | 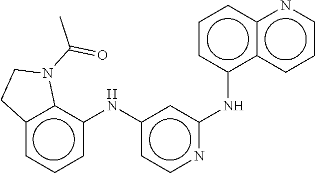 |
| 13 (1941) | 4.38 | | 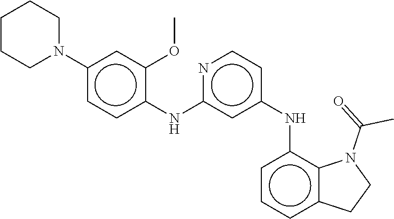 |
| 23 (1971) | 4.53 | | 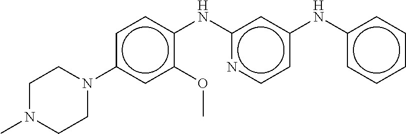 |
| 94 (2350) | 4.56 | | 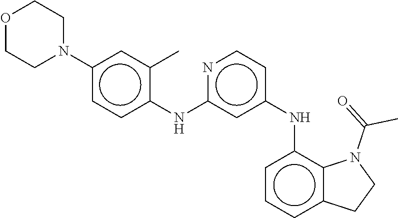 |
| 17 (1950) | 4.78 | | 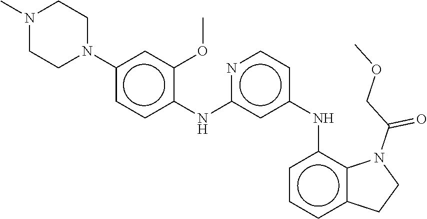 |
| 30 (2000) | 4.89 | | 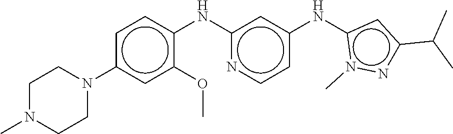 |
| 35 (2011) | 4.97 | | 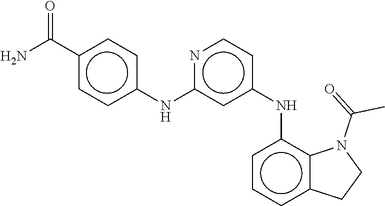 |
| 93 (2349) | 5.18 | | 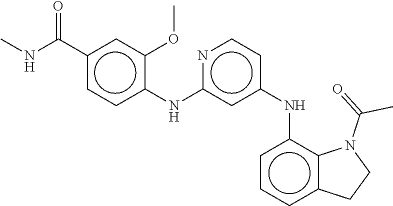 |
| 33 (2009) | 5.23 | | 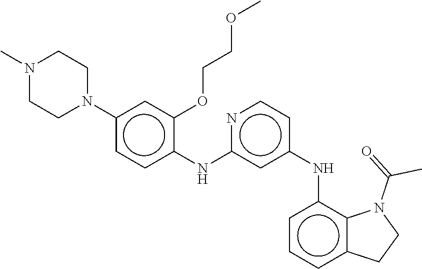 |
| 81 (2107) | 5.33 | | 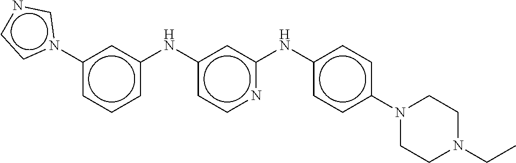 |
| 21 (1961) | 6.68 | | 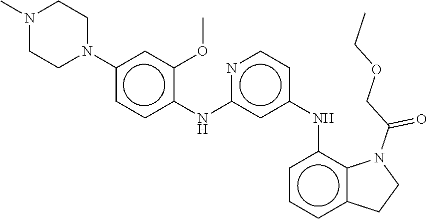 |
| 53 (2039) | 7.85 | | 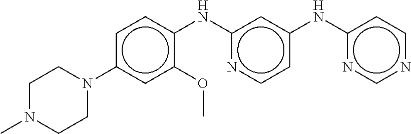 |
| 22 (1968) | 7.85 | | 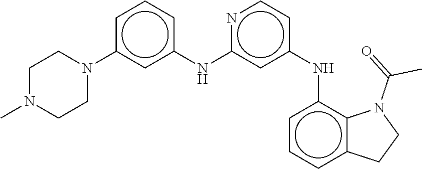 |
| 79 (2105) | 8.31 | | 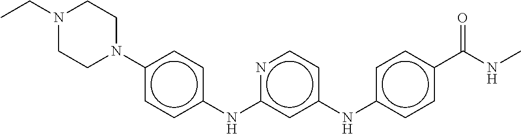 |
| 36 (2013) | 8.81 | | 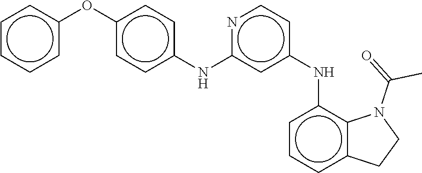 |
| 3 (1904) | 9.71 | | 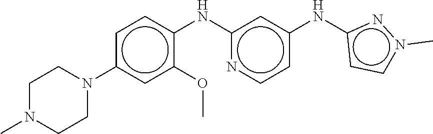 |
| 95 (2351) | 14.72 | | 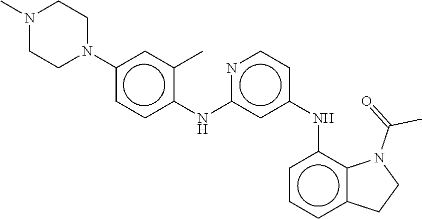 |
| 1 (1902) | 50.00 | | 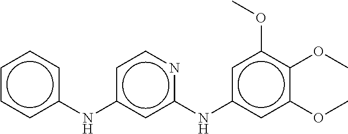 |
| 2 (1903) | 50.00 | | 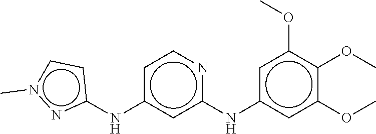 |
| 80 (2106) | 50.00 | | 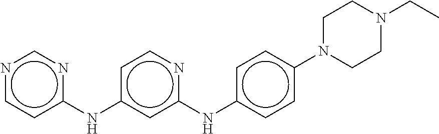 |
| 92 (2348) | >10.4 | | 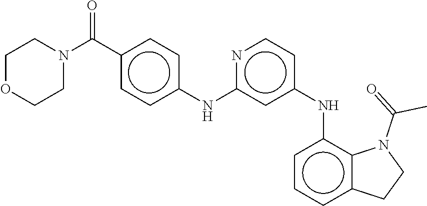 |
| 25 (1992) | >50 | | 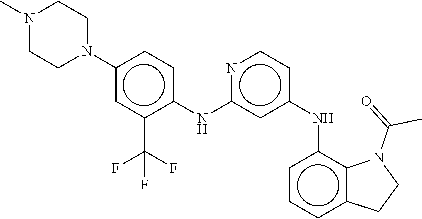 |
| 26 (1993) | >50 | | 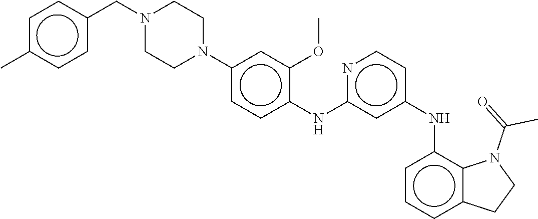 |
| 34 (2010) | >50 | | 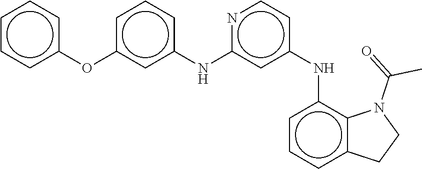 |
| 37 (2014) | >50 | | 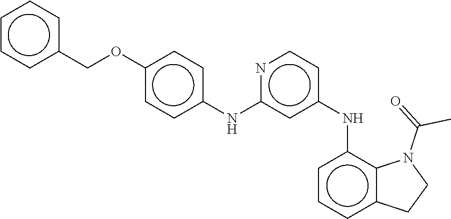 |
| 39 (2016) | >50 | | 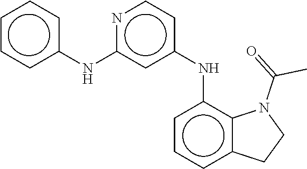 |
| 43 (2020) | >50 | | 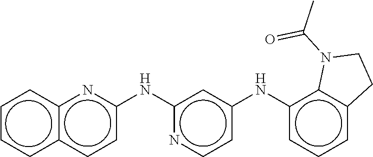 |
| 44 (2021) | >50 | | 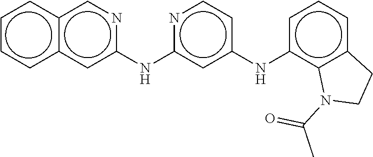 |
| 45 (2022) | >50 | | 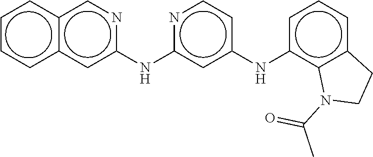 |
| 46 (2023) | >50 | | 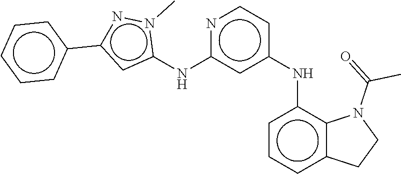 |
| 57 (2047) | >50 | | 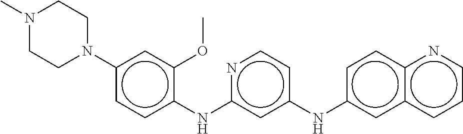 |
| 69 (2069) | >50 | | 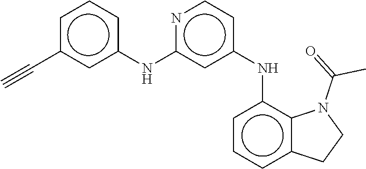 |
| 74 (2079) | >50 | | 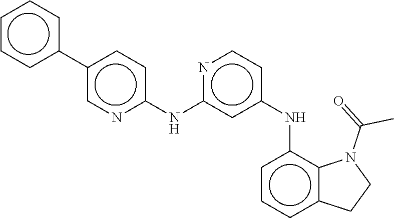 |
| 98 (2355) | >50 | | 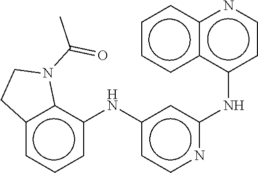 |
| 100 (2357) | >50 | | 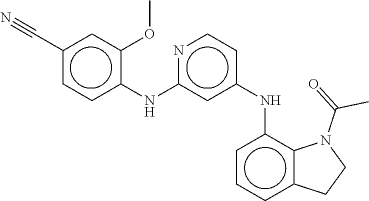 |
| 101 (2358) | >50 | | 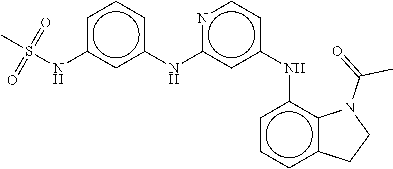 |
| 102 (2359) | >50 | | 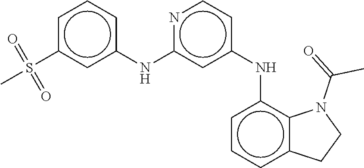 |
| 55 (2045) | >6.125 | | 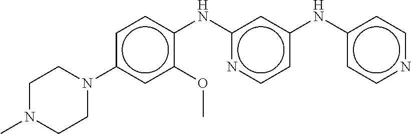 |
| 70 (2070) | 0.39 | X | 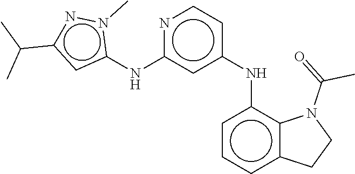 |
| 49 (2028) | 0.44 | X | 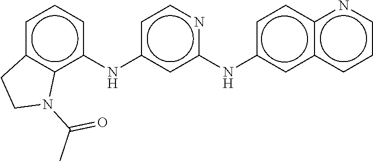 |
| 73 (2073) | 0.50 | X | 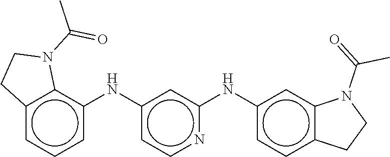 |
| 7 (1912) | 0.54 | X | 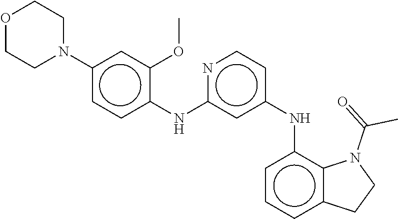 |
| 59 (2052) | 0.55 | X | 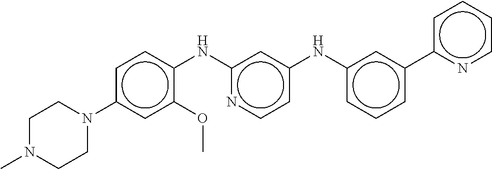 |
| R5 (1962) | 0.59 | X | 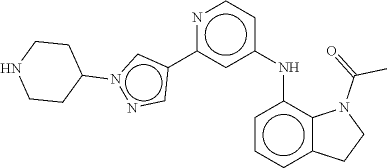 |
| 6 (1907) | 0.84 | X | 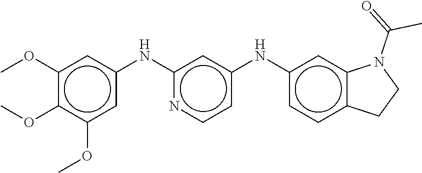 |
| 62 (2056) | 1.29 | X | 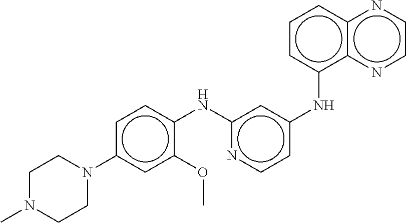 |
| 28 (1995) | 1.41 | X | 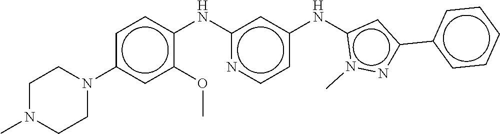 |
| 48 (2027) | 1.48 | X | 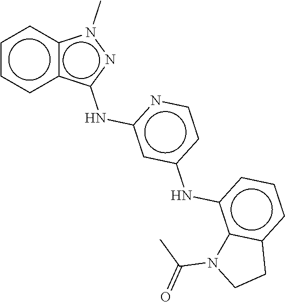 |
| 9 (1935) | 1.58 | X | 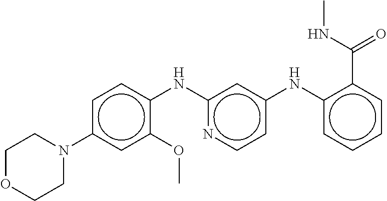 |
| 41 (2018) | 1.74 | X | 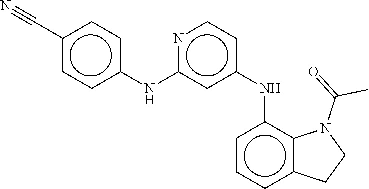 |
| 47 (2026) | 1.76 | X | 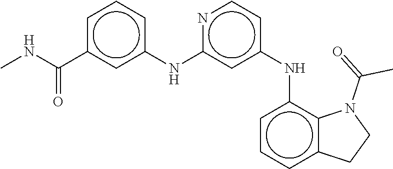 |
| 4 (1905) | 2.08 | X | 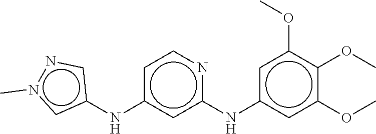 |
| 83 (2109) | 2.78 | X | 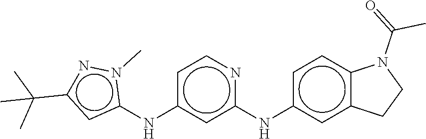 |
| 99 (2356) | 4.78 | X | 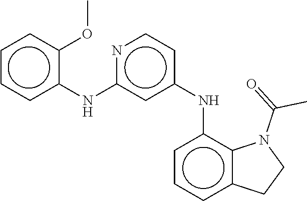 |
| 10 (1936) | 5.36 | X | 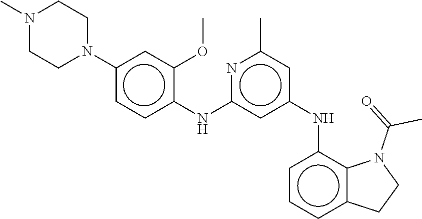 |
| 16 (1944) | 5.53 | X | 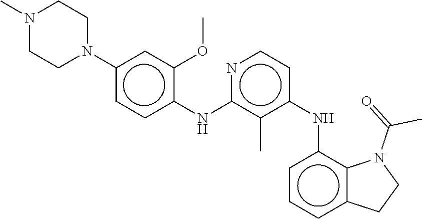 |
| 71 (2071) | 6.54 | X | 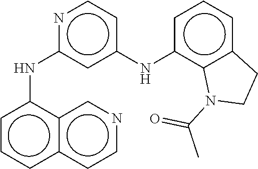 |
| 40 (2017) | 7.40 | X | 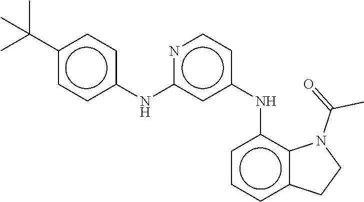 |
| 66 (2064) | 17.29 | x | 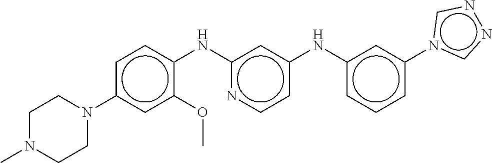 |
| 96 (2353) | >0.505 | X | 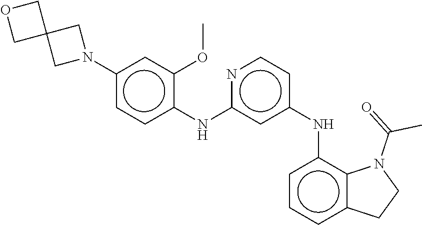 |
| 75 (2101) | >19.01 | X | 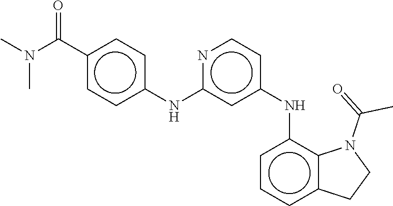 |
| 87 (2112) | >27 | X | 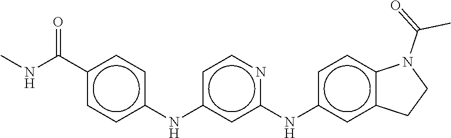 |
| 85 (2110) | >27.2 | X | 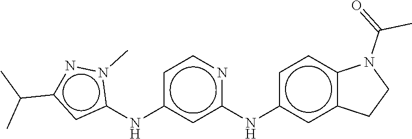 |
| 86 (2111) | >29 | X | 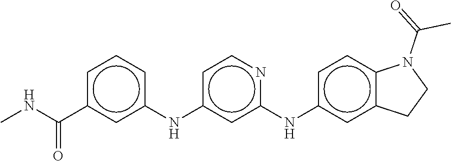 |
| 8 (1934) | >50 | X | 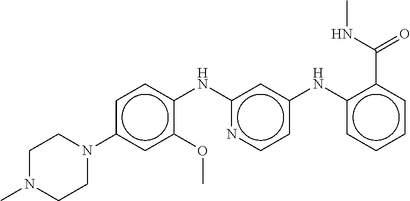 |
| 103 (1940) | >50 | X | 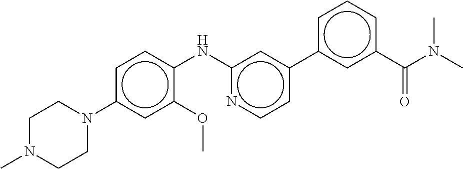 |
| 63 (2057) | >50 | X | 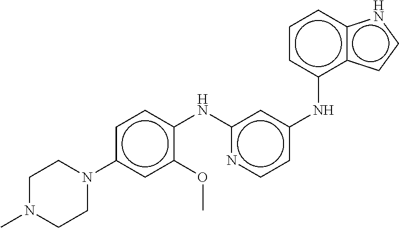 |
| 67 (2066) | >50 | X | 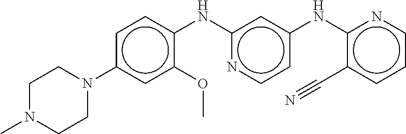 |
| 88 (2115) | >50 | X | 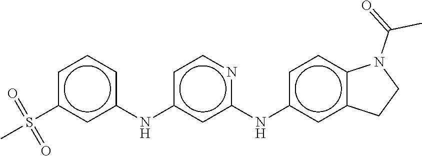 |
| 89 (2116) | >50 | X | 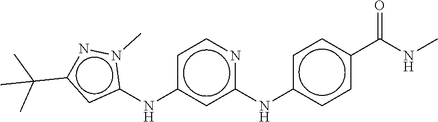 |
| 90 (2117) | >50 | X | 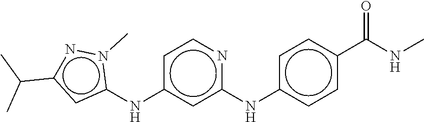 |
| 99 (2346) | >50 | X | 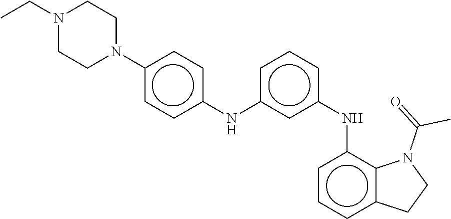 |

### Example 2

In vitro characterization of the activity of 1938, as a selected example compound of the invention.

### 1938 is an Allosteric, Non-ATP-Competitive Isoform-Selective Activator of PI3Kα

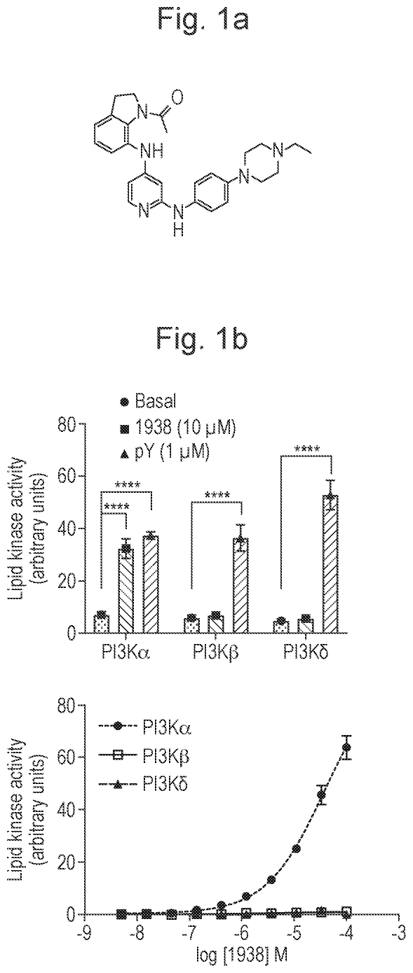

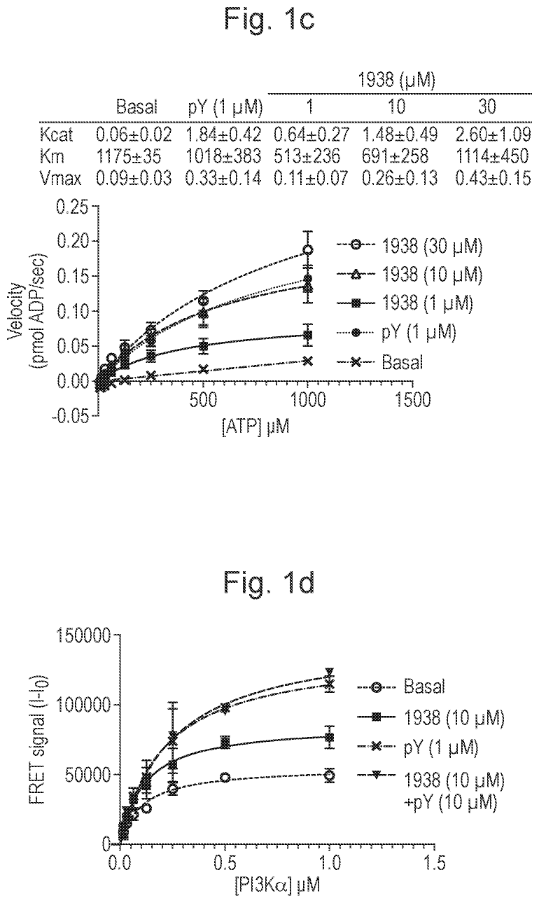

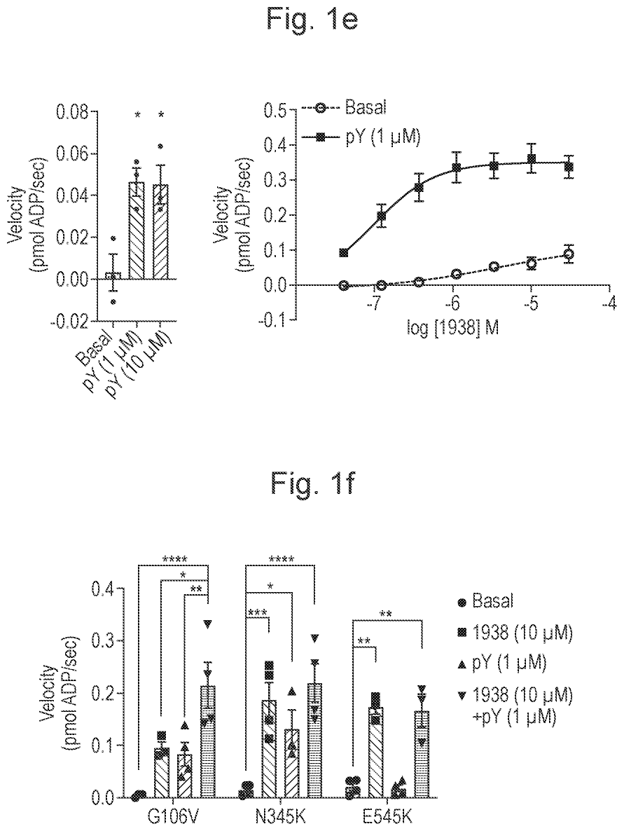

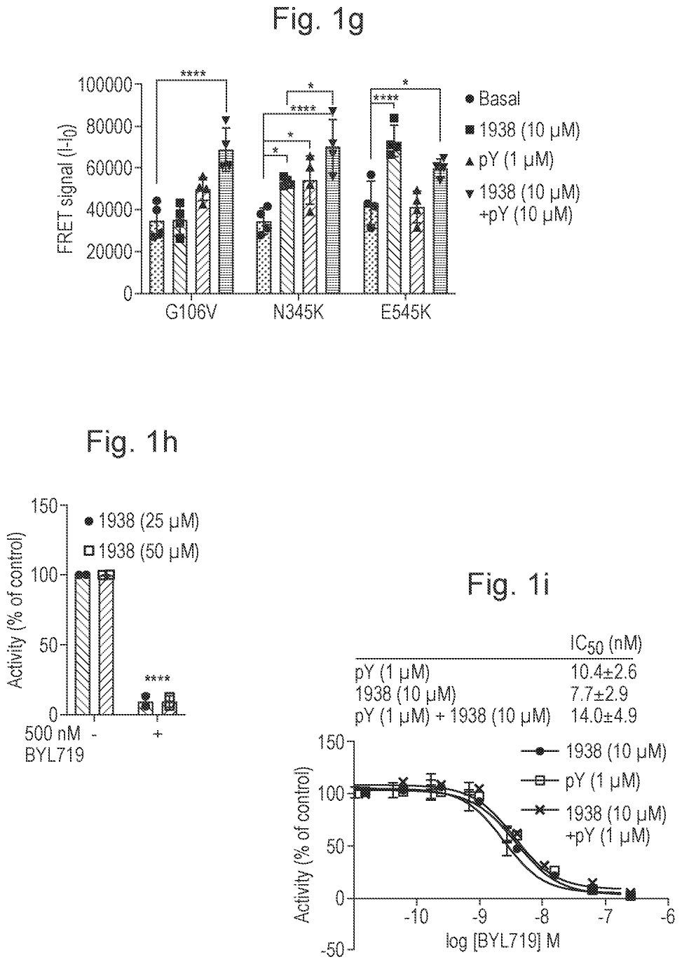

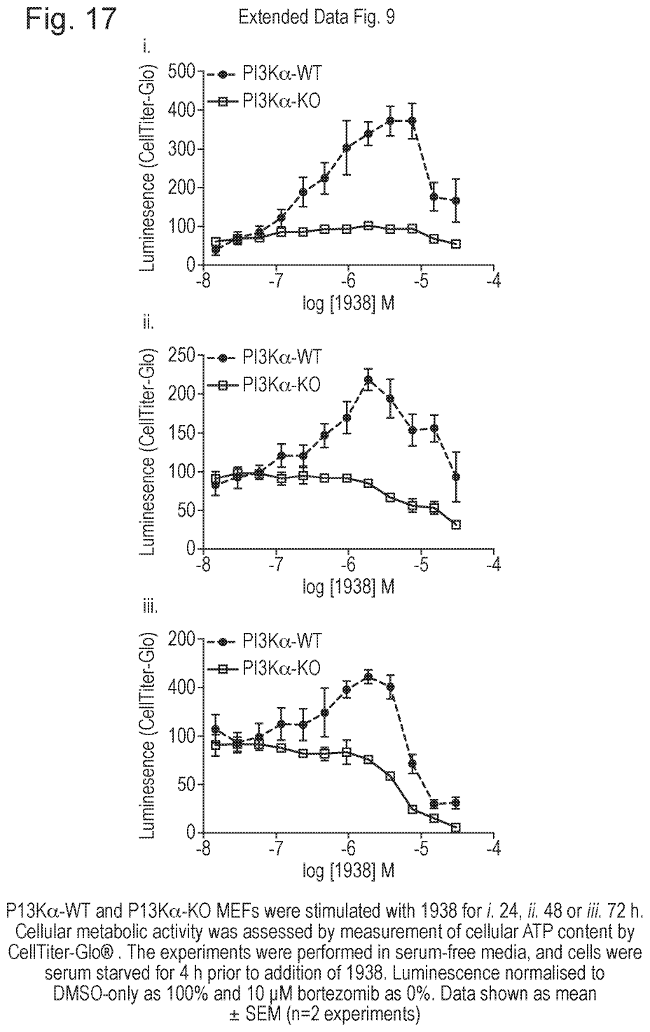

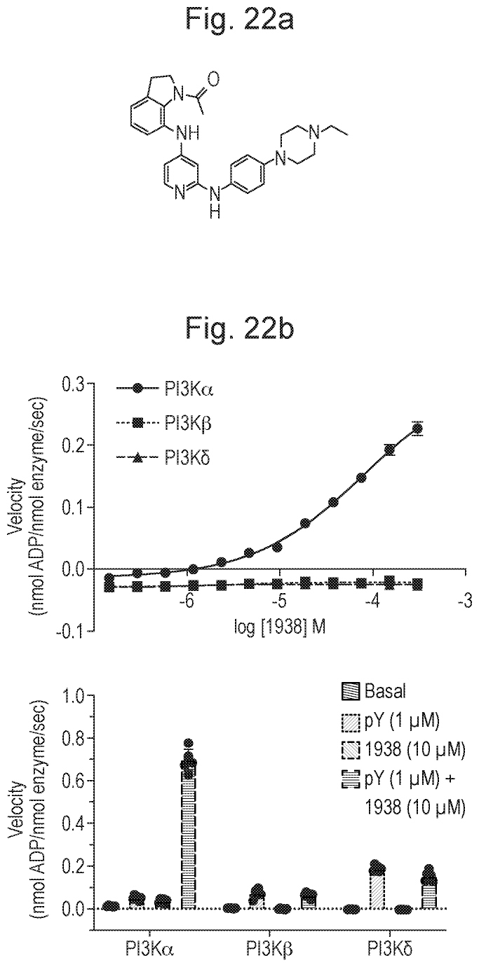

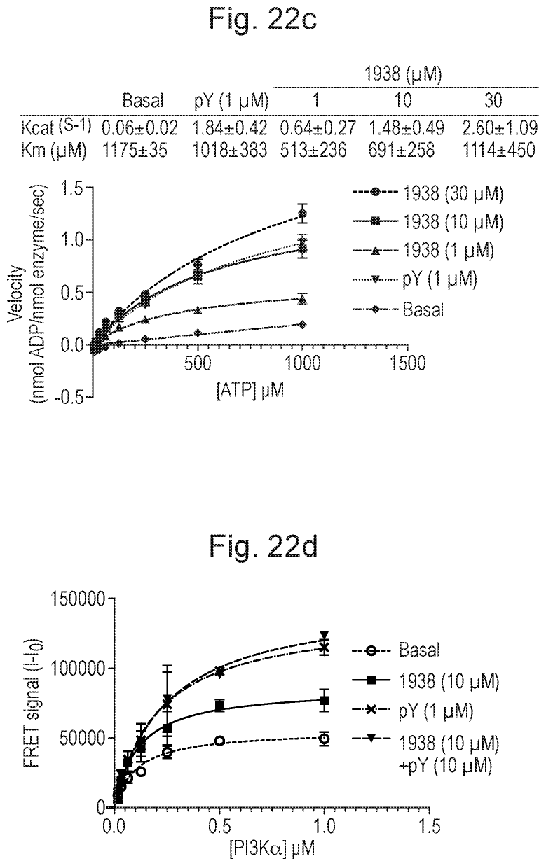

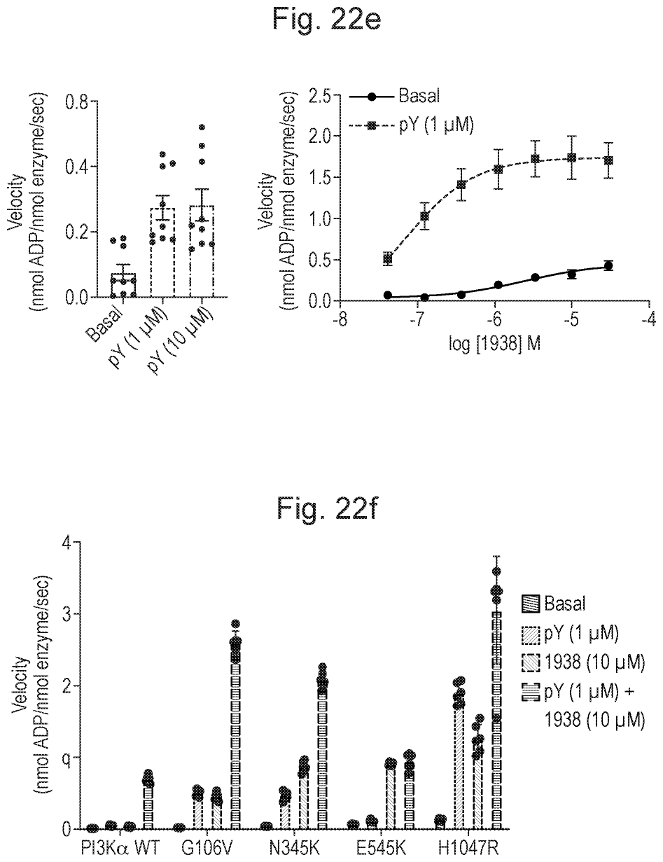

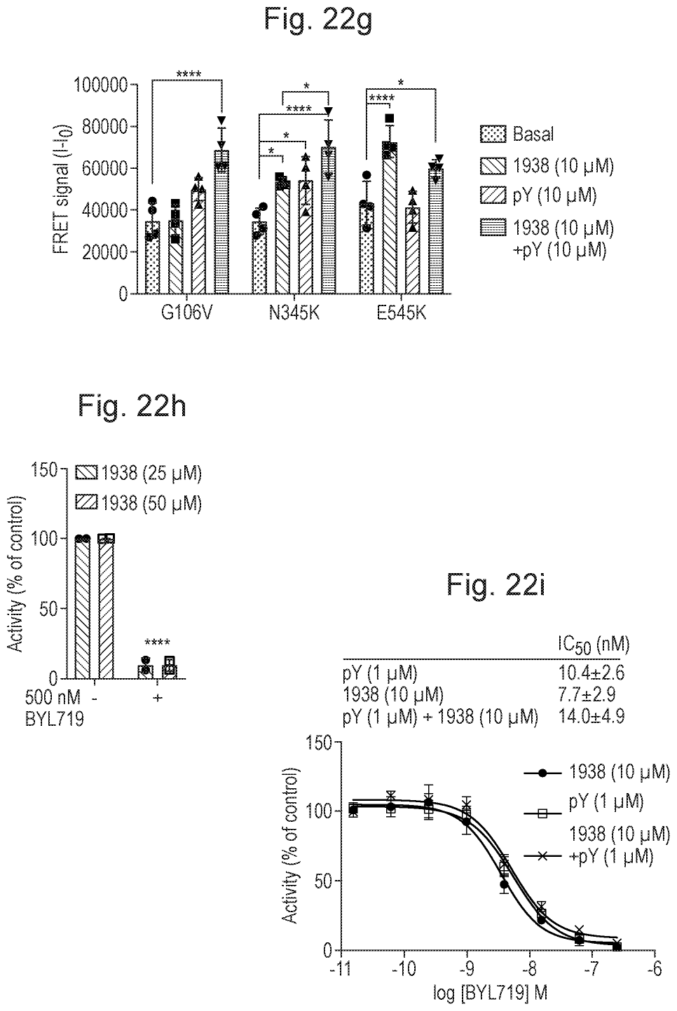

The effect of 1938 on the in vitro lipid kinase activity of p85α in complex with p110α, p110β or p110δ (further referred to as PI3Kα, PI3Kβ and PI3Kδ) was tested. As a positive control, a bis-phosphorylated phosphopeptide (a PDGF-receptor-derived peptide phosphorylated on Tyr-740 and Tyr-751, hereafter referred to as pY peptide) was used to mimic engagement of the p85α SH2 domains with tyrosine-phosphorylated peptides in membrane-bound receptors and adaptor proteins, known to activate the PI3Kα complex. Unlike pY, which activates all class IA PI3K isoforms (FIG. 9/Extended Data FIG. 1), 1938 was found, in a concentration-dependent manner, to activate PI3Kα but not PI3Kβ or PI3Kδ (FIG. 1b/FIG. 22b). Enzyme kinetic assays demonstrated that 1938, like pY, increased the turnover number (Kcat) and maximum rate of reaction (Vmax) of PI3Kα (FIG. 1c/FIG. 22c). Whereas pY did not affect the Km of PI3Kα for ATP, 1938 induced a modest lowering in this parameter of PI3Kα at 1 and 10 μM, but not at 30 μM 1938 (FIG. 1c). 1938 also induced increased binding of PI3Kα to lipid membranes, to a maximum level of about half of that induced by pY (FIG. 1d/FIG. 22d). Combination of a saturating concentration of pY (FIG. 1e, left panel/FIG. 22e, left panel) with 1938, led to synergistic PI3Kα activation (FIG. 1e, right panel/FIG. 22e, right panel), indicating that 1938 activates PI3Kα via a different mechanism or enhances activatory events beyond those induced by pY. This synergy is unlikely to involve changes in membrane binding, given that the combination of 1938 with pY did not further increase PI3Kα membrane association beyond that induced by pY (FIG. 1d/FIG. 22d).

The inventors next tested the effect of 1938 on different oncogenic mutants of p110α, each of which has been shown to activate the p85α/p110α complex through a different mechanism (Burke, J. E. et al. Proc Natl Acad Sci USA 109, 15259-15264 (2012)) (summarized in Table 2). 1938 activated the G106V and N345K mutants to levels comparable with stimulation with pY. Although the E545K mutant was insensitive to pY stimulation, as previously shown, it could be further activated by 1938 (FIG. 1f/FIG. 22f). Co-stimulation of 1938 with pY led to an additive activatory effect for G106V, but not for the N345K and E545K mutants (FIG. 1f/FIG. 22f). Whereas 1938 did not increase the membrane binding of G106V, it did so for N345K (to similar levels as that induced by pY), and E545K (whose membrane binding was not increased by pY, as previously reported) (FIG. 1g/FIG. 22g). Taken together, these data suggest that 1938 enhances multiple steps of the PI3Kα catalytic cycle and does not specifically mimic the mechanism of activation afforded by any single one of the oncogenic p110α mutations tested (Table 2).

TABLE 2
Impact of 1938 and selected hot-spot PI3KCA
mutations on p110α/p85α

| | | | | | Impact of 1938 |
|---|---|---|---|---|---|
| | Impact of PIK3CA mutation on | | | | on wild-type |
| | p110α/p85α | | | | p110α/p85α |
| Impact | G106V | N345K | E545K | H1047R | (this study) |
| Disruption of the | − | − | + | − | − |
| nSH2-helical | | | | | |
| interface | | | | | |
| Disruption of the | ++ | +++ | + | − | ++ |
| iSH2-C2 interface | | | | | |
| Movement of the | +++ | ++ | + | − | −/? |
| adapter- | | | | | |
| binding domain | | | | | |
| Deprotection of | − | − | − | +++ | − |
| membrane- | | | | | |
| binding regions | | | | | |
| Deprotection of | − | − | − | − | ++ |
| the hinge between | | | | | |
| N- & C-lobes | | | | | |
| Enhancement of | + | + | + | ++ | + |
| basal kinase | | | | | |
| activity | | | | | |
| Enhancement of | − | + | + | + | + |
| basal membrane | | | | | |
| binding affinity | | | | | |
| Enhancement of | + | + | − | +++ | +++ |
| pY-activated | | | | | |
| kinase activity | | | | | |
| Enhancement of | + | + | − | ++ | − |
| pY-activated | | | | | |
| membrane | | | | | |
| binding | | | | | |

The inventors next investigated the effect of the ATP-competitive PI3Kα-selective inhibitor BYL719 on 1938-activated PI3Kα. In vitro PI3Kα activity stimulated by 25-50 μM of 1938 was fully inhibited by 500 nM BYL719 (FIG. 1h/FIG. 22h), with an IC₅₀ value of 10-20 nM BYL719 for PI3Kα inhibition in the presence of 1938 (10 μM) and ATP (200 μM) that is similar to its previously-reported IC₅₀ for PI3Kα in the absence of 1938 (FIG. 1i/FIG. 22i). These data indicate that 1938 does not compete for ATP binding on PI3Kα, and that 1938-activated PI3Kα can still be fully inhibited by BYL719.

### Example 3

HDX-MS studies on 1938, as a selected example compound of the invention.

1938 Activates PI3K a by Disrupting Inhibitory Contacts Between p85α and p110α

The inventors used hydrogen-deuterium exchange mass spectrometry (HDX-MS) which allows the investigation of compound binding sites and conformational changes using soluble, native proteins, and which has been key to unravel the molecular mechanisms of physiological activation of class IA PI3Ks. Class IA PI3K activation occurs through de-inhibition of the auto-inhibited p85/p110 complex, via the release of inhibitory interactions between p85 and p110, upon binding of the complex to bis-phosphorylated motifs in membrane-resident receptors or cytosolic adaptors. These events include the release of the inhibition of p85α-nSH2 domain on the p110α-helical domain, disruption of the p85α-iSH2/p110α-C2 domain inhibitory interface, movement of the p85 adaptor domain in p110α relative to the rest of the catalytic subunit and interaction of the p110α kinase domain with the lipid membrane.

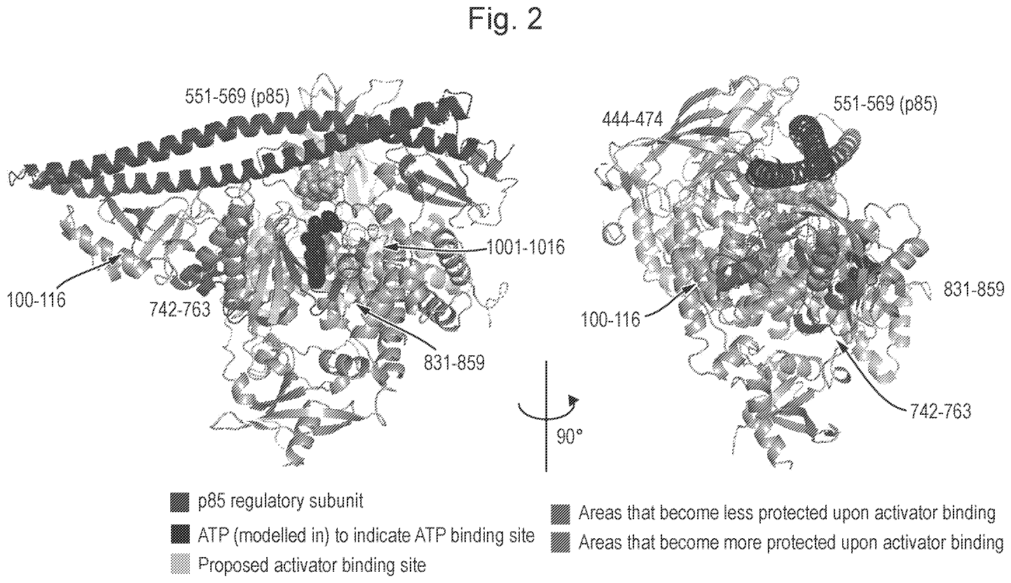

HDX-MS of 1938 incubated with PI3Kα demonstrated that a small loop consisting of amino acids (AA) 1001-1016 of p110α was more protected upon 1938 binding, implicating this region as the potential 1938 binding site on p110α (FIG. 2a; a surface model is shown in Extended Data FIG. 2). These observations also indicate that 1938 binds at the interface between the p85α-iSH2 domain and the p110α-C2 and -kinase domains, outside of the ATP-binding site (FIG. 2a). There was also an increase in solvent exchange rate in several additional regions of p85α/p110α, namely the p85α-iSH2 domain (AA 550-570) and multiple regions in p110α, namely (from N- to C-terminus): AA 444-474 (interface between p85α-iSH2 and p110α-C2 domain), and AA 848-859 (ATP-binding site). These observed structural changes suggest that 1938 likely activates PI3Kα by disrupting the inhibitory contacts between the p85α-iSH2 domain and the p110α-C2 domain, causing movements in the p110α ABD/RBD linker and kinase domain, resulting in enhanced catalytic activity. In addition to this, we conducted HDX-MS experiments with BYL719 and a mixture of both BYL719 and 1938. BYL719 produced a characteristic ATP-competitive footprint on PI3Kα, with strong protections on the 848-858 kinase domain linker region and the 767-781 region, as reported previously (see PMID: 28381646). Upon combination of the two compounds however, an amagalmated footprint of PI3Kα was observed, with the protections in the kinase linker region, and protections in the 1002-1016 pocket, along with exposures in the p85α-iSH2 interface—suggesting that PI3Kα is capable of accommodating both ligands simultaneously.

### Example 4

Cell-based characterization of the activity of 1938, as a selected example compound of the invention.

### 1938 Induces PI3Kα Pathway Activation in Cells

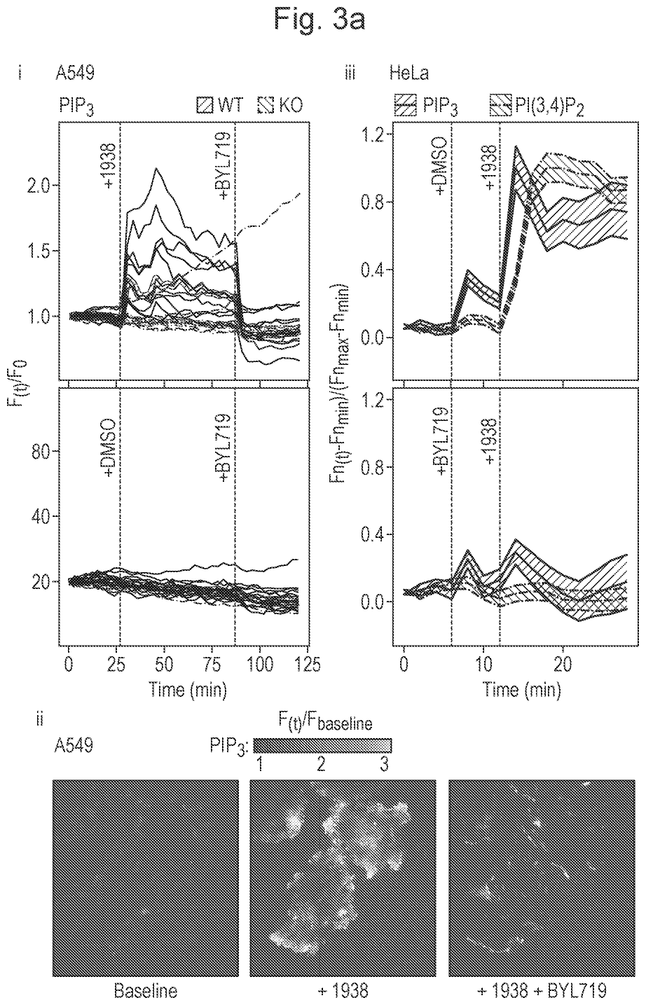

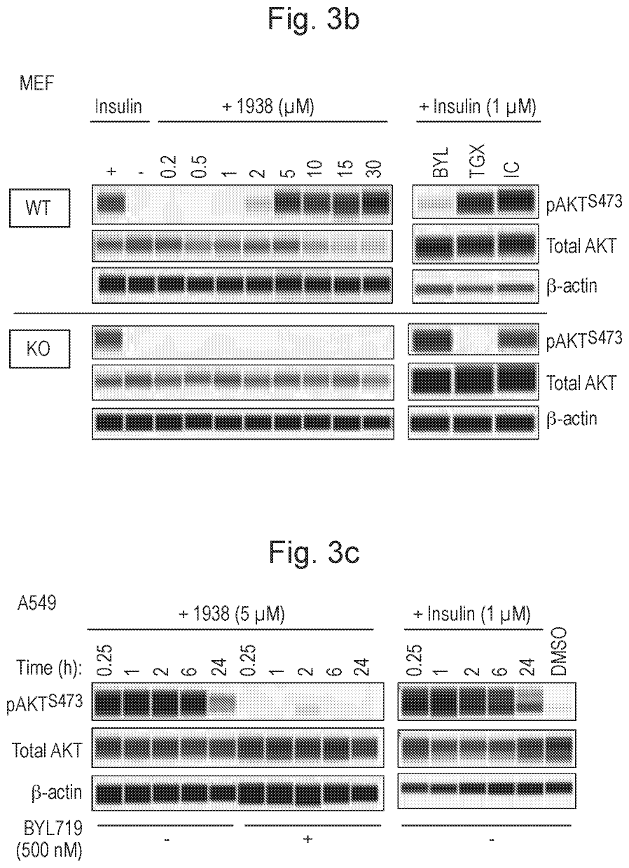

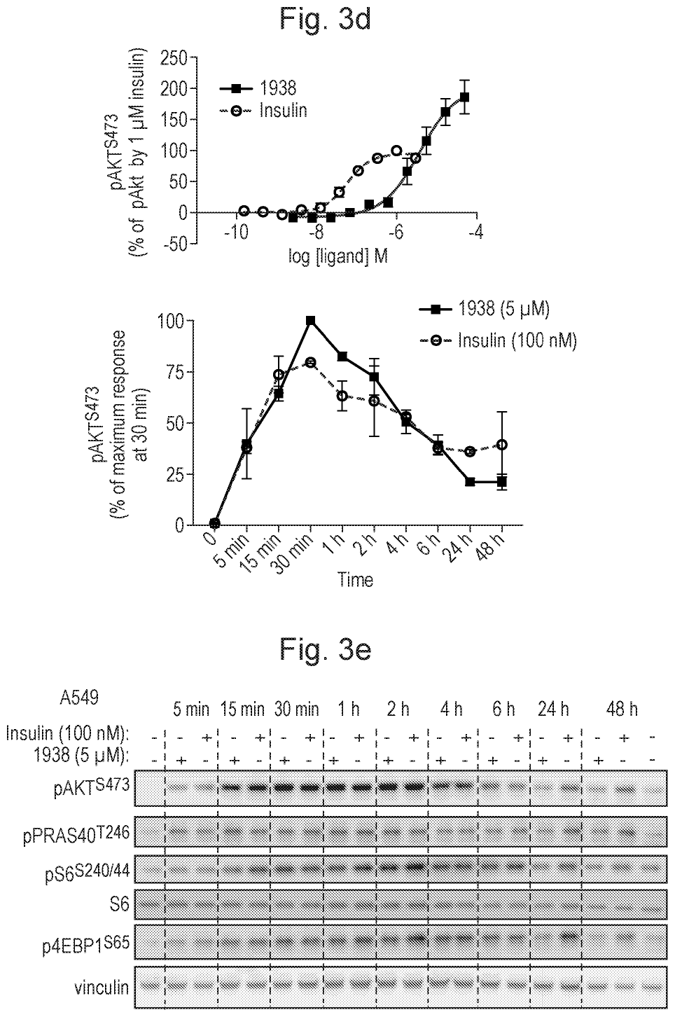

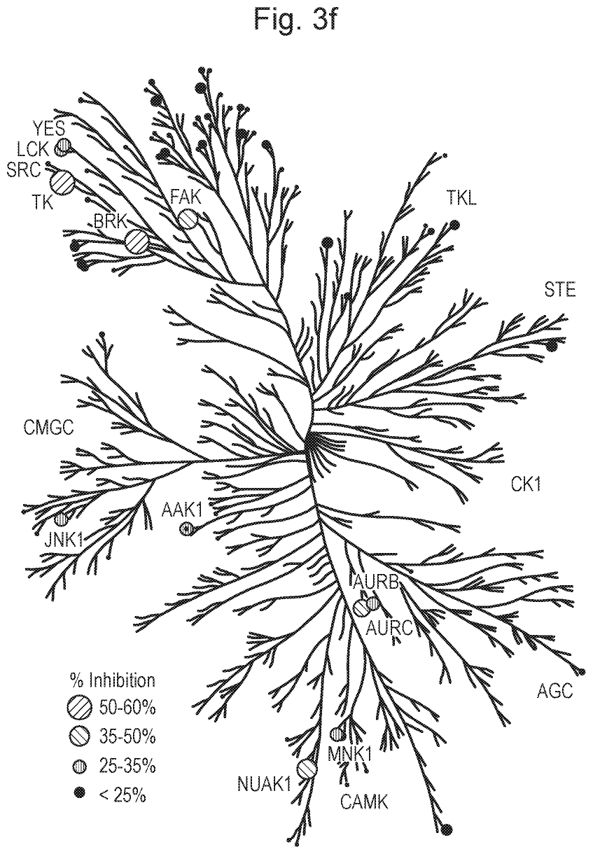

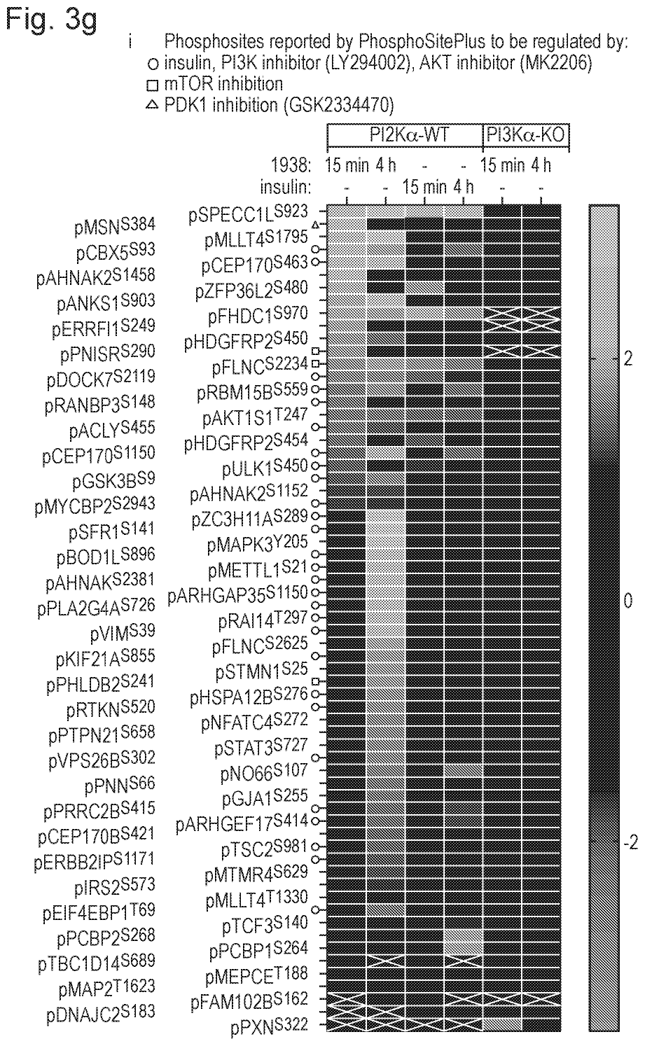

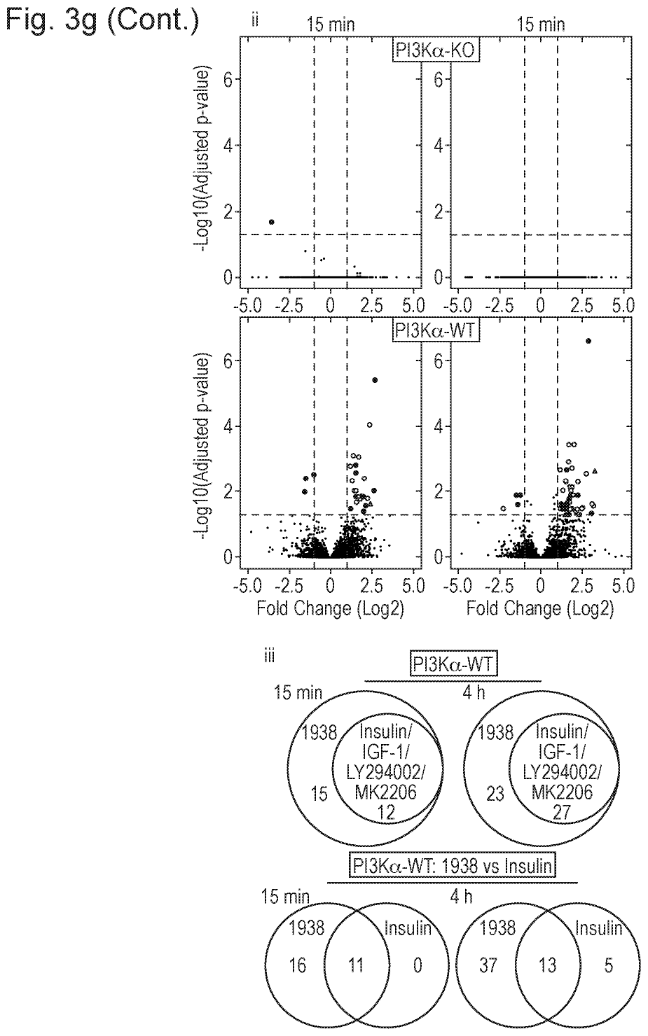

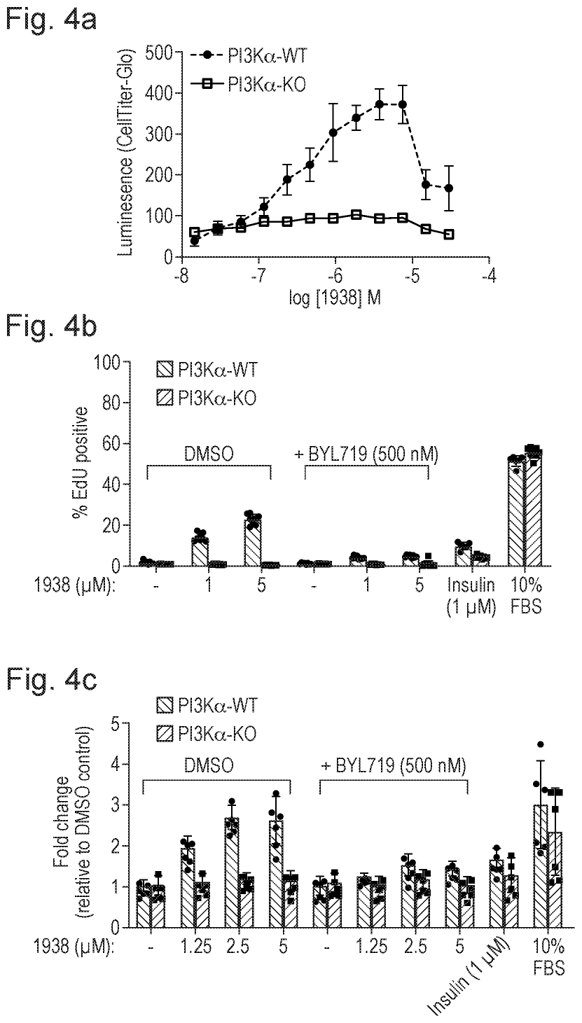

Class I PI3Ks convert the PtdIns(4,5)P₂ lipid in the plasma membrane to PtdIns(3,4,5)P₃ (or PIP₃) which is converted to PtdIns(3,4)P₂ by the action of 5-phosphatases. The inventors therefore tested whether stimulation of cells with 1938 led to the generation of PIP₃ and PI(3,4)P₂ using live imaging in cells expressing fluorescent biosensors that selectively bind these lipids. Within less than 3 min of treatment of serum-starved A549 cells, 1938 induced plasma membrane-associated PIP₃ production which could be fully and acutely neutralized by the addition of BYL719 (FIG. 3ai,ii). 1938 did not increase the PIP₃ signal in PIK3CA-null A549 cells (FIG. 3ai). In serum-starved HeLa cells, 1938 also induced an acute but, compared to A549 cells, a more transient BYL719-dependent burst of PIP₃ production, followed by a return to intermediate levels (FIG. 3aiii). This PIP₃ peak was followed by the generation of membrane-associated PI(3,4)P₂ (FIG. 3aiii), with a timing in line with the known mechanism of cellular generation of PI(3,4)P₂ from PIP₃ by the action of 5-phosphatases. Similar kinetics of PIP₃ and PI(3,4)P₂ production have previously been reported in HeLa cells stimulated with insulin. The small increases in signal following addition of BYL719 and 1938 in HeLa cells (FIG. 3aiii, lower panel) represent a non-specific response to medium addition seen only in these cells (FIG. 12/Extended Data FIG. 4).

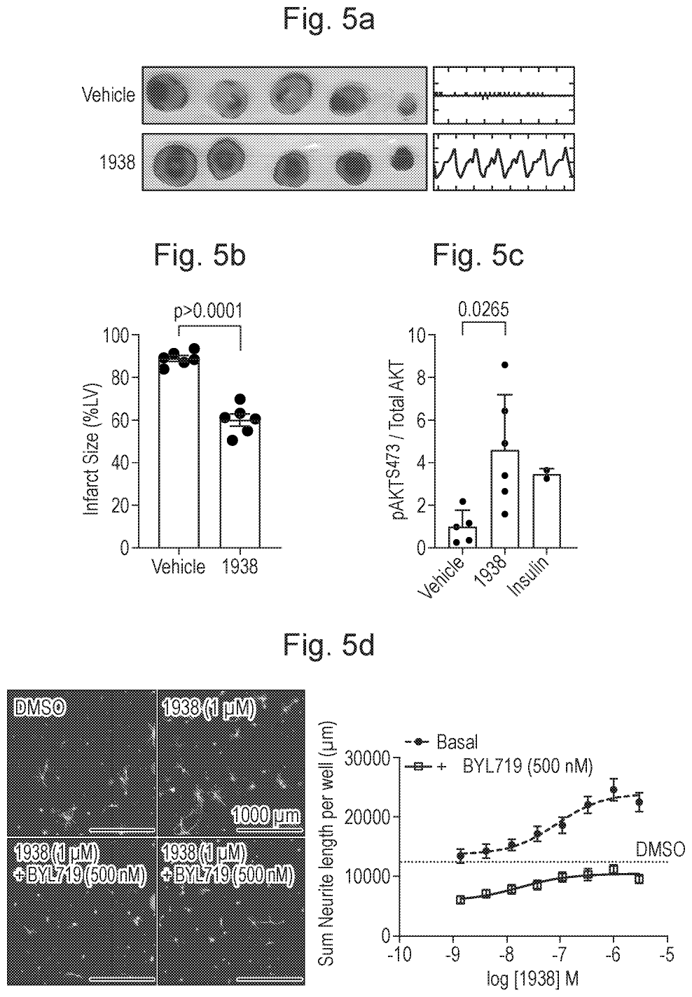

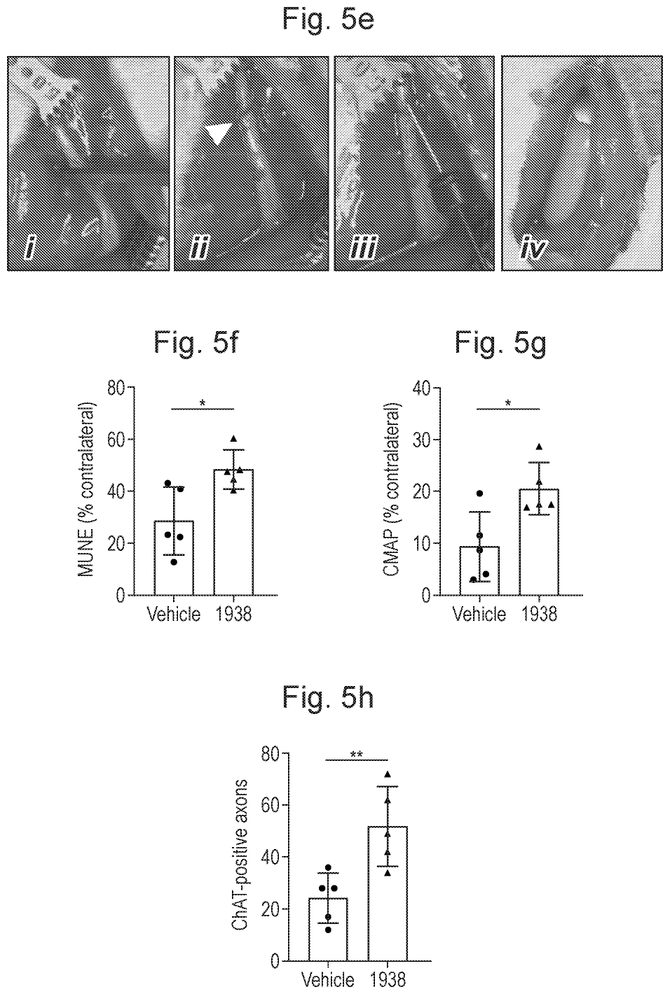

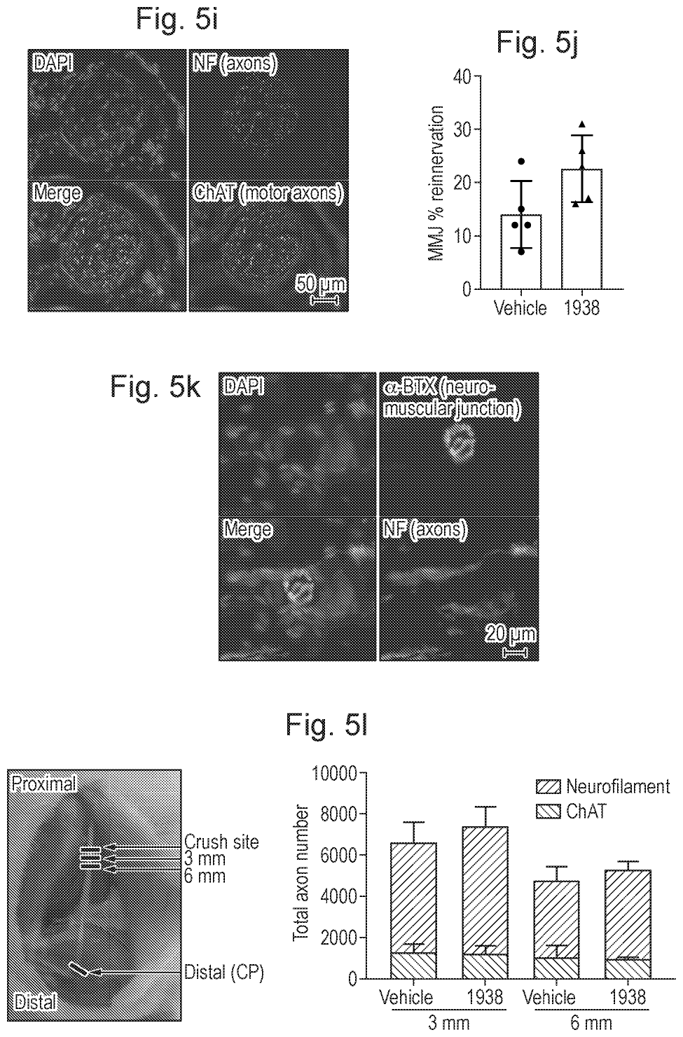

The inventors next monitored the activation of AKT, the best-known PI3K effector stimulated by PIP₃/PI(3,4)P₂ production in cells. Fifteen min treatment with 1938 increased the levels of pAKT^{S473} in a concentration-dependent manner (FIG. 3b) in mouse embryonic fibroblasts (MEFs), (FIG. 3b) while no pAKT^{S473} phosphorylation was observed in PI3Kα-null MEFs. As previously reported, PI3Kα-null MEFs still respond to insulin, but now in a PI3Kβ-dependent manner, as shown by sensitivity of insulin-stimulated pAKT^{S473} to the PI3Kβ-selective inhibitor TGX-221 (FIG. 3b). Co-treatment with BYL719 fully blocked AKT phosphorylation induced by 1938 in PI3Kα-wild-type MEFs (FIG. 3b), A549 cells (FIG. 3c) and MCF10A cells (FIG. 13/Extended Data FIG. 5). Upon 15 min treatment with 1938, the EC₅₀ for induction of pAKT was ˜2-4 μM in both mouse (MEFs; FIG. 3b) and human (A549) cells (FIG. 3d). A dose titration of 1938 and insulin in A549 cells revealed that in these cells 1938 can overactivate the PI3K pathway, as measured by AKT phosphorylation, beyond saturating doses of insulin, namely ˜200% of Eₘₐₓ of 1 μM insulin at doses of 5-10 μM 1938 (FIG. 3d). The induction of pAKT^{S473} in MEFs and A549 by 1938 (5 μM) was rapid (5 min; FIG. 3c, e; FIG. 13/Extended Data FIG. 5), reaching peak activation at 30 min and persisting for few hours before returning to levels slightly above baseline after 24 h or 48 h of stimulation (FIG. 3e). Similar observations were made for mTORC1 pathway activation, as measured by S6 (Ser240/44) and 4EBP1 (Ser65) phosphorylation (FIG. 3e). Interestingly, the pattern and kinetics of Akt/mTORC1 pathway activation was overall similar to that induced by insulin (FIG. 3c,e), suggesting that 1938-mediated PI3K pathway activation is subjected to the endogenous cellular feedback mechanisms that are known to operate in the PI3K signalling pathway. In summary, 1938 activates both proximal and distal signalling in a dose- and PI3Kα-dependent manner in rodent and human cells, demonstrating its ability to directly activate PI3Kα signalling in cells.

### Example 5

Unbiased assessment of signalling induced by 1938, as a selected example compound of the invention.

### Unbiased Assessment of Signalling Induced by 1938

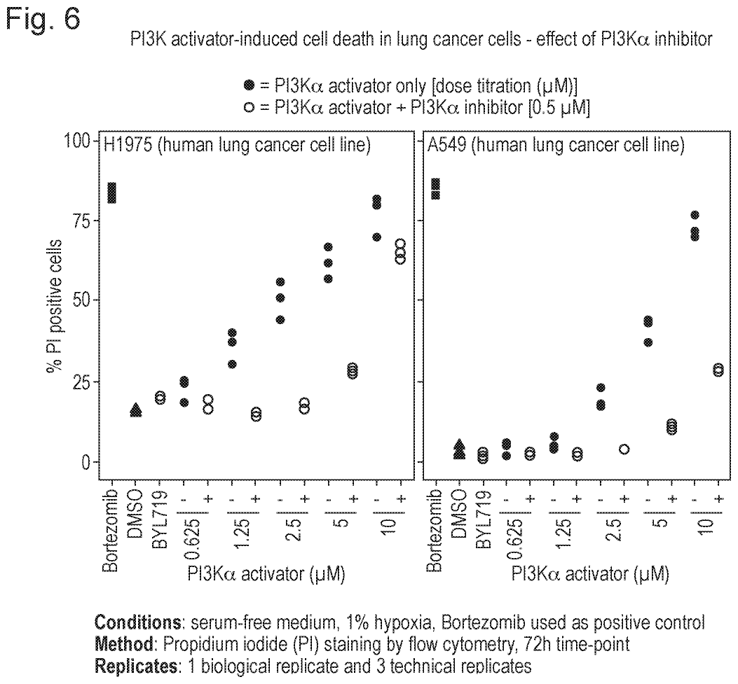

Given that the structure of 1938 contains a pyridine core, known to be a scaffold of multiple kinase inhibitors, the inventors next tested the impact of 1938 on the in vitro activity of a panel of 133 protein kinases and 7 lipid kinases (data represented as a KinMap (Eid, S. et al. BMC Bioinformatics 18, 16 (2017)) (FIG. 3f) or a waterfall plot (FIG. 14/Extended Data FIG. 6). At a concentration of 1 μM of 1938, 13 protein kinases were inhibited between 25-50%, with only two protein kinases, LCK and BRK, inhibited by more than 50% (58% and 56%, respectively). It is important to note that LCK and BRK were tested in vitro in the presence of 50 and 75 μM ATP, respectively: if 1938 acts as an ATP-competive inhibitor for these kinases, the % inhibition by 1938 is expected to be significantly lower in cells where the ATP concentration is known to be 1-10 mM.

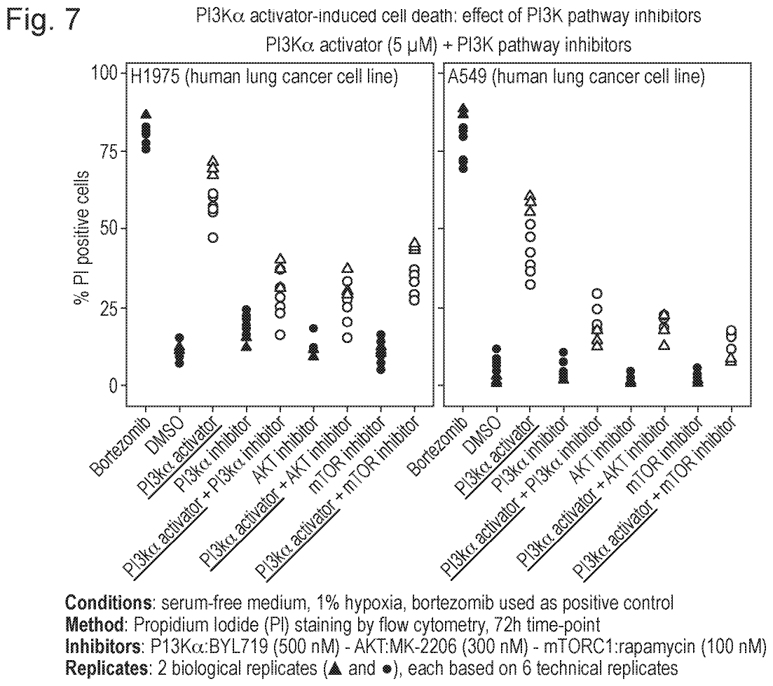

1938 did not affect the activity of other PI3K isoforms in the panel [PI3Kβ (PIK3CB), PI3Kγ (PIK3CG), PI3Kδ (PIK3CD), PI3K-C2α (PIK3C2A) and Vps34 (PIK3C3)] or the PI3K-related kinases PI4Kβ, mTOR and DNA-PK (FIG. 3f). In separate in vitro assays, 1938 did not affect the activity of the PI3K-related kinases ATM (FIG. 15/Extended Data FIG. 7) and mTORC1 (FIG. 14/Extended Data FIG. 7; tested as the mTOR/RAPTOR/LST8 complex; note that mTOR activity in the Thermofisher screen (FIG. 3f, FIG. 14/Extended Data FIG. 6) was tested using an mTOR monomer without any of its binding partners).

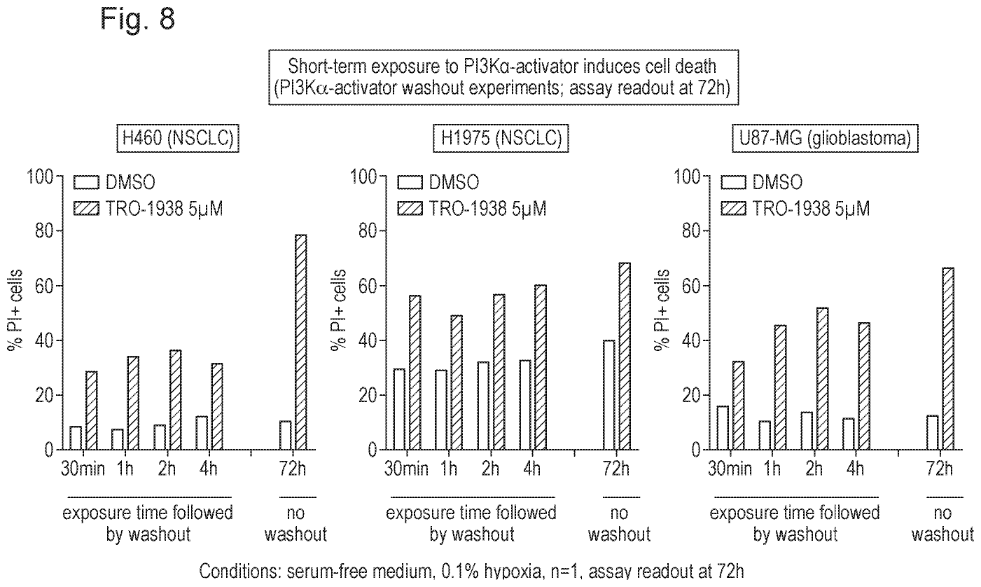

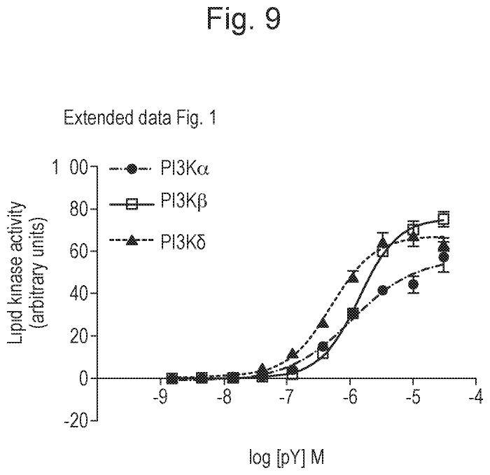

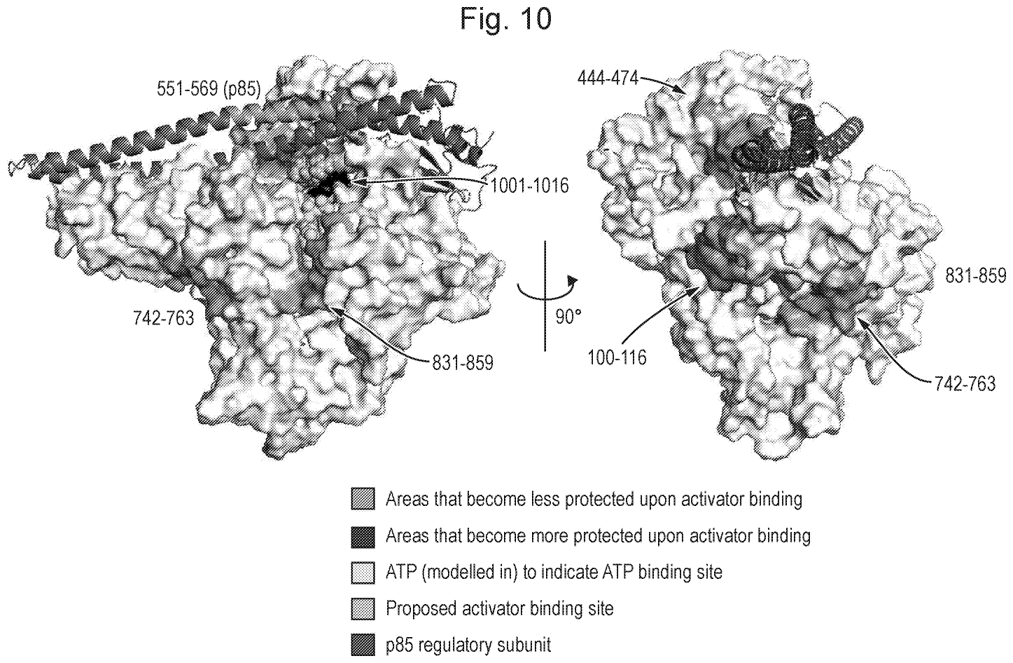

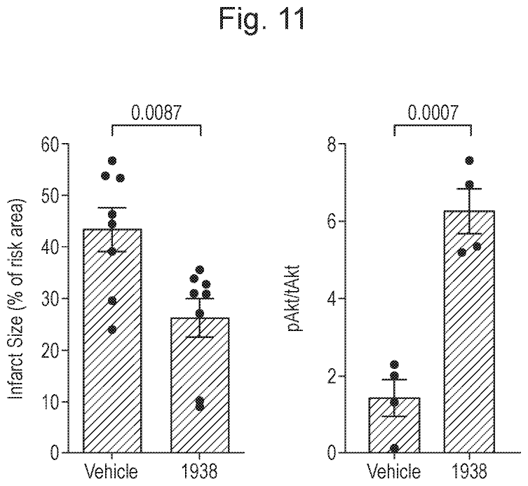

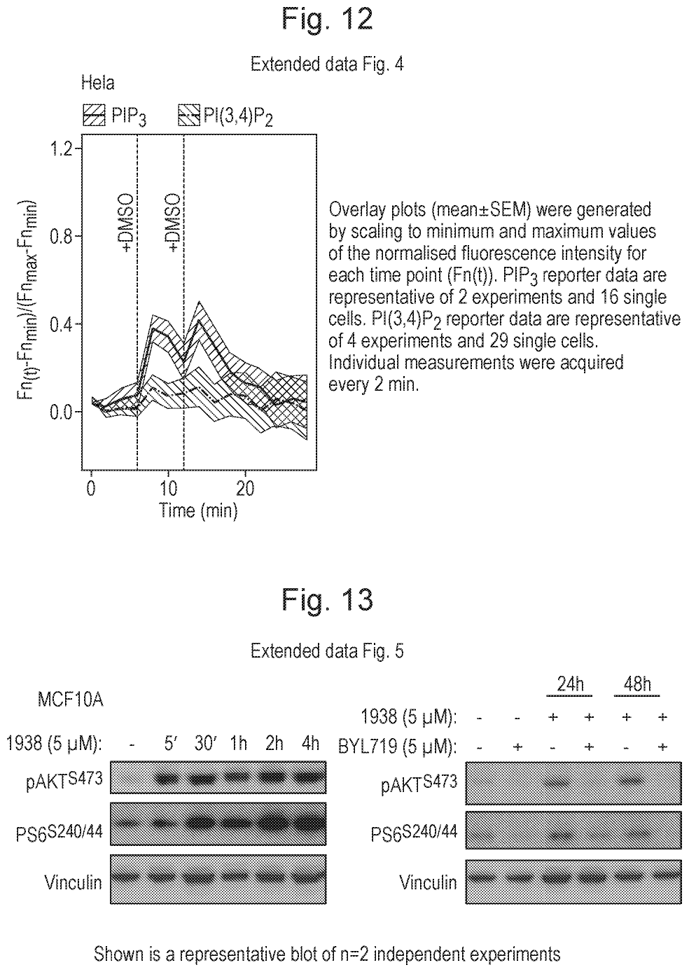

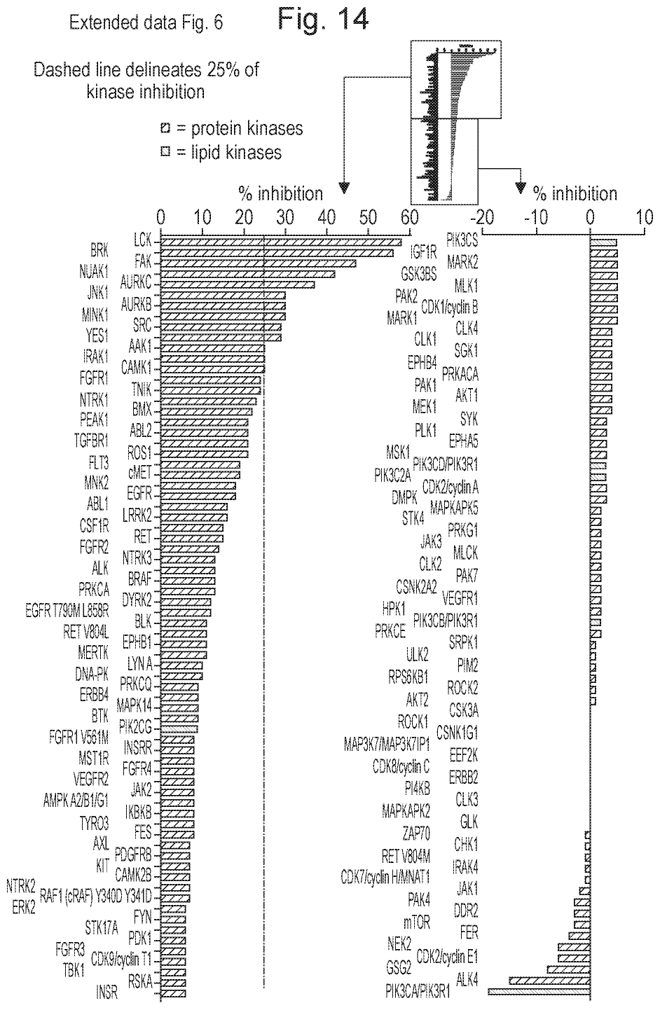

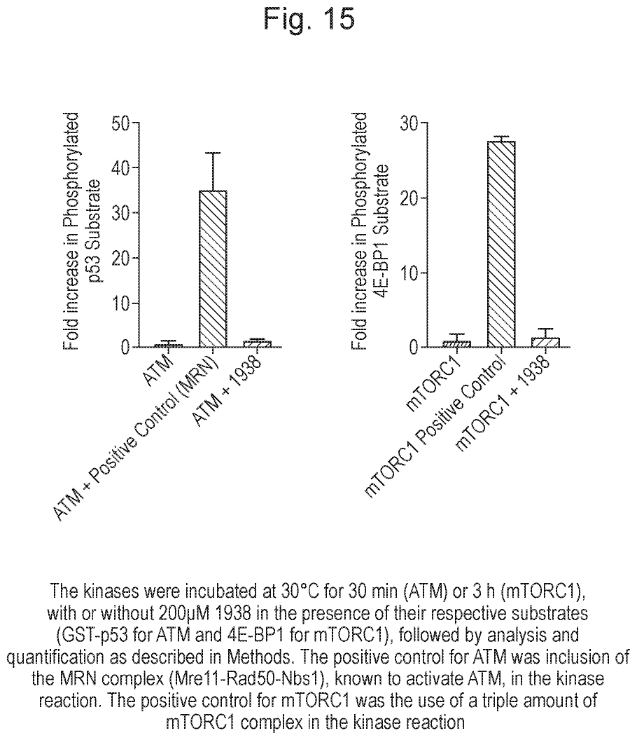

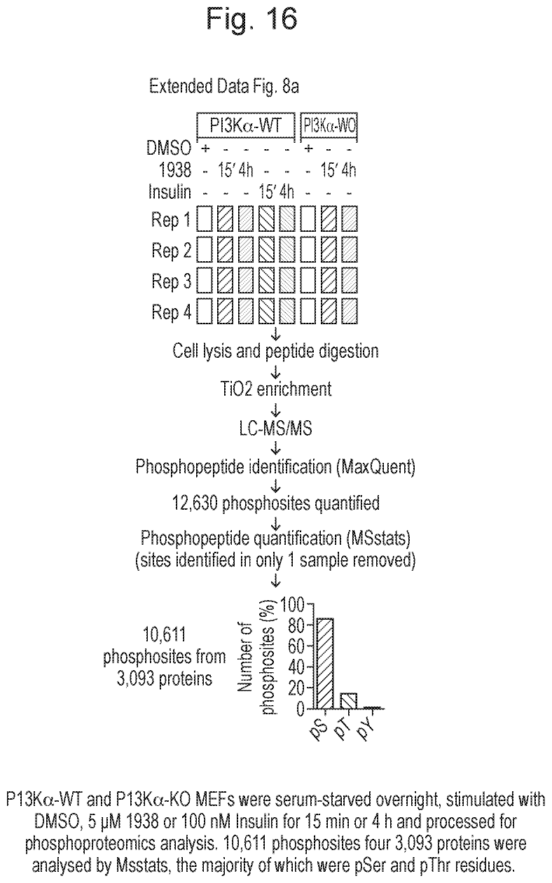

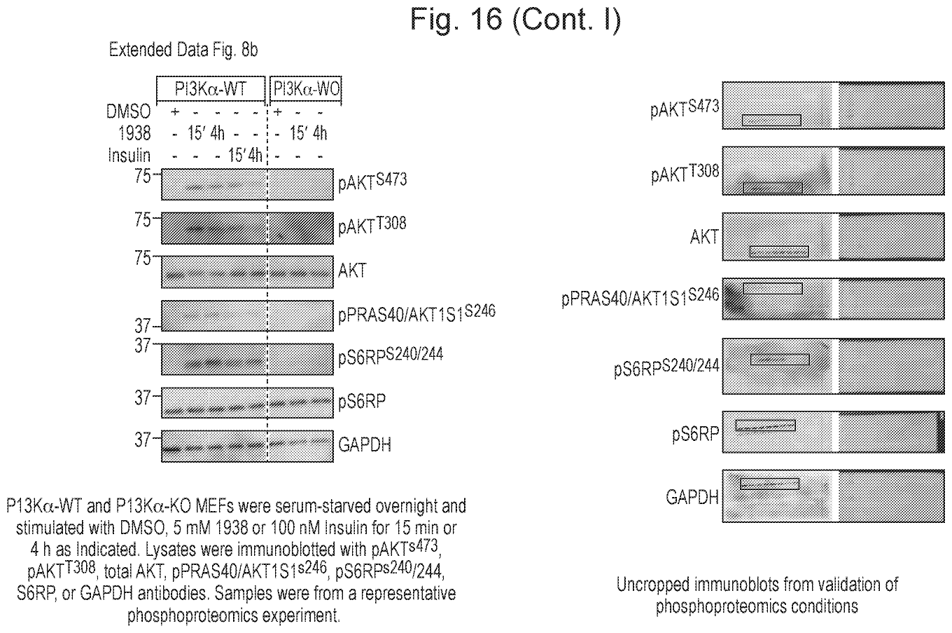

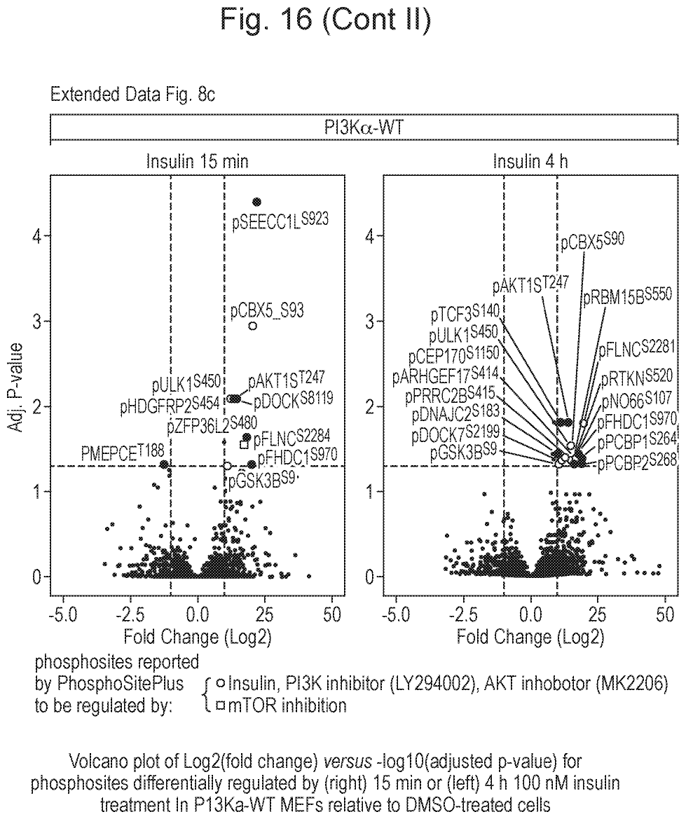

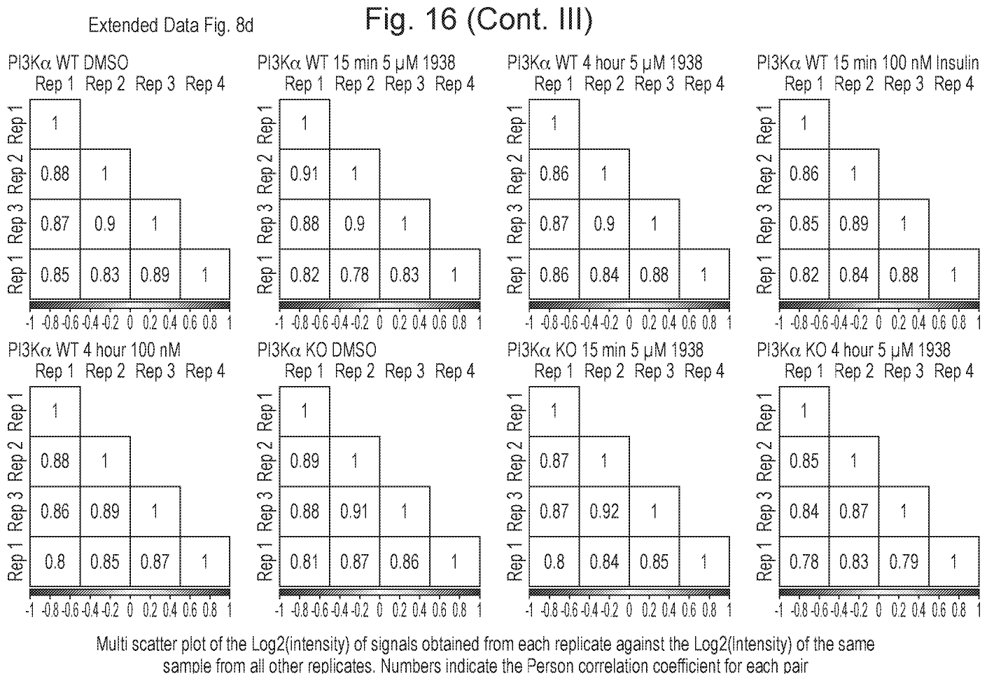

The inventors next investigated the impact of 1938 on cell signalling in an unbiased manner using phosphoproteomics. PI3Kα-WT and PI3Kα-KO MEFs were treated with 1938 or insulin for 15 min or 4 h (FIG. 16/Extended Data FIG. 8a,b), with phosphosites exhibiting >2-fold change relative to DMSO and adjusted p-value <0.05 defined as significantly regulated. The inventors quantified 10,611 phosphosites from 3,093 proteins of which 9100, 1420 and 91 were pSer, pThr and pTyr residues, respectively (FIG. 16/Extended Data FIG. 8a). In line with the data shown in FIG. 3a,b, 1938 had little signalling impact in PI3Kα-KO MEFs (FIG. 3gi,ii; FIG. 16/Extended Data FIG. 8b), with Paxillin (pPXN^{S322}) being the only phosphosite altered (downregulated upon 15 min 1938 treatment but not affected upon 4 h stimulation; FIG. 3gi,ii). In PI3Kα-WT MEFs, 1938 induced differential phosphorylation of 27 and 50 peptides at 15 min and 4 h treatment, respectively, the majority of which were upregulated (FIG. 3gi,ii). Upregulated phosphosites included the well-established PI3K pathway components pAKT1S1^{T247} (also known as PRAS40) and pGSK3B^{S9} (FIG. 3gi). Compared to vehicle-treated cells, insulin treatment of PI3Kα-WT MEFs induced differential phosphorylation of 11 and 18 sites at 15 min and 4 h, respectively (FIG. 16/Extended Data FIG. 8c). At both time points, substantial overlap was observed in the phosphosites regulated by 1938 and insulin in PI3Kα-WT MEFs (FIG. 3giii). The majority of phosphosites upregulated by 1938 and insulin at 4 h were similar to the sites upregulated by 15 min 1938 treatment (FIG. 3gi). Furthermore, approximately half of the 1938-controlled phosphosites have been previously reported (in PhosphoSitePlus) to be regulated by insulin, IGF-1, PI3K inhibition or AKT inhibition, with some linked to regulation by mTOR or PDK1 (FIG. 3gi,ii). Notably, some phosphosites upregulated by 1938 in PI3Kα-WT but not PI3Kα-KO MEFs, including top hits such as pSPCC1L^{S923}, pMSN^{S384} and pMAPK3^{Y205}, have not been previously linked to PI3K signalling as per PhosphoSitePlus (FIG. 3gi), highlighting the utility of 1938 as a tool compound to uncover novel signalling pathways downstream of PI3Kα.

### Example 6

In vitro assessment of PI3Kα-dependent cell biological responses induced by 1938, as a selected example compound of the invention.

### 1938 Induces PI3Kα-Dependent Cell Biological Responses

A role for PI3Kα in activation of anabolic metabolism, cell cycle progression and cell proliferation is well-established. In PI3Kα-WT MEFs, but not in PI3Kα-KO MEFs, 1938 dose-dependently increased metabolic activity, as measured by ATP content using the CellTiterGlo assay, with an EC₅₀ of ˜0.5 μM of 1938 (FIG. 4a). After 24 h incubation, concentrations of 1938 above ˜7.5 μM significantly decreased ATP levels in both PI3Kα-WT and PI3Kα-KO MEFs, indicative of PI3Kα-independent effects of 1938 at these concentrations (FIG. 4a). Upon longer incubation (48 h and 72 h), these non-PI3Kα-dependent effects of 1938 were observed at ≥2 μM (FIG. 17/Extended data FIG. 9).

In addition to increased metabolic activity, 1938 treatment of PI3Kα-WT MEFs also induced cell cycle progression (as measured by EdU incorporation; FIG. 4b) and an increase in cell number (as measured by crystal violet staining; FIG. 4c). These biological effects were not observed in PI3Kα-KO MEFs and could be fully neutralised by co-treatment with BYL719 in PI3Kα-WT MEFs (FIG. 4b,c). It is of interest to note that, unlike 72 h treatment with 1938, incubation with insulin under the same conditions did not lead to an increase in cell number (FIG. 4c), providing further for differential cellular activities of these agents, as suggested by our proteomics data (FIG. 3f).

### Example 7

Ex vivo and in vivo assessment of disease-relevant biological responses induced by 1938, as a selected example compound of the invention.

### Therapeutic Potential of Pharmacological PI3Kα Activation

Myocardial infarction is responsible for significant morbidity and mortality in patients with coronary artery disease. Despite the development of new anti-platelet and anti-thrombotic agents, timely reperfusion by percutaneous coronary intervention via catheterisation remains fundamental to heart tissue salvage. Paradoxically, such reperfusion also causes ischaemia reperfusion injury (IRI), tissue damage that occurs following the restoration of blood supply after a period without, and is also observed in intra-arterial device-based treatment of stroke. Finding ways to reduce IRI is vital to improving the long-term outcome of patients with myocardial infarction. Ischaemic preconditioning, an experimental method of protecting the heart from IRI, leads to the activation of kinases such MEK/ERK1/2 and PI3K/AKT as part of the so-called Reperfusion Injury Salvage Kinase (RISK) pathway, a cardioprotective pathway induced by the majority of cardioprotective agents, including insulin, the canonical activator of the PI3K/AKT pathway. Using PI3Kα inhibitors, we have previously shown that activation of PI3Kα is both necessary and sufficient for cardioprotection.

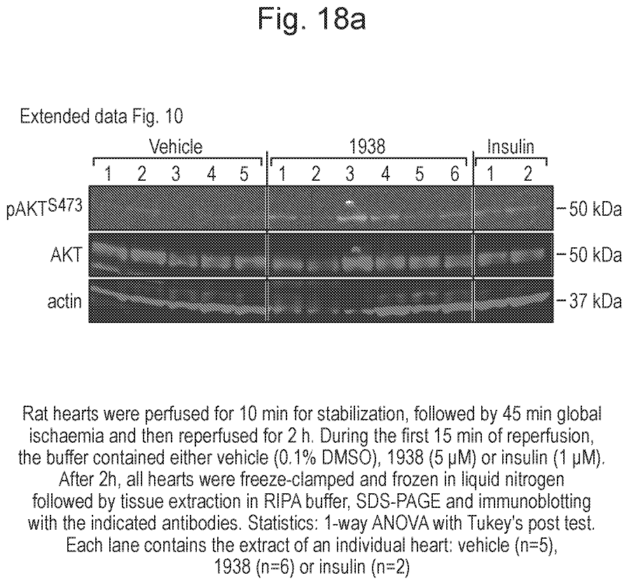

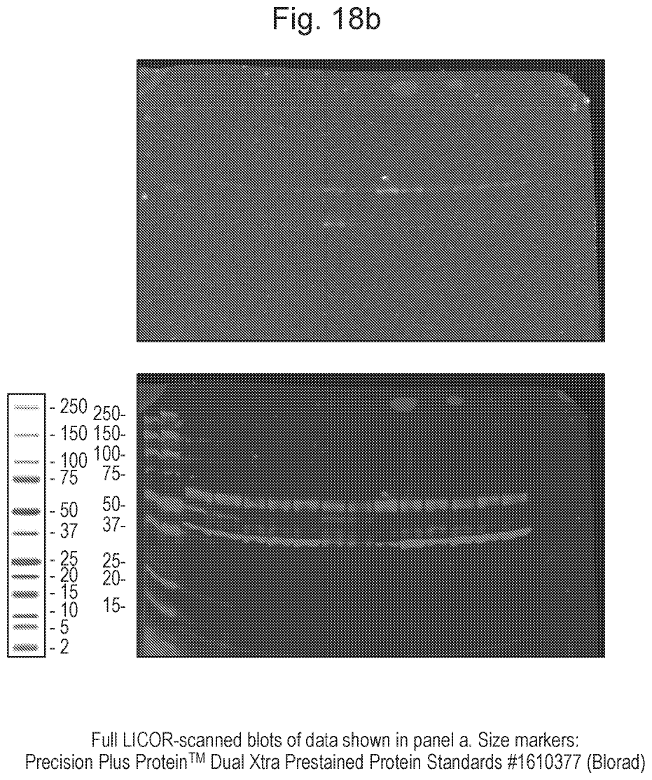

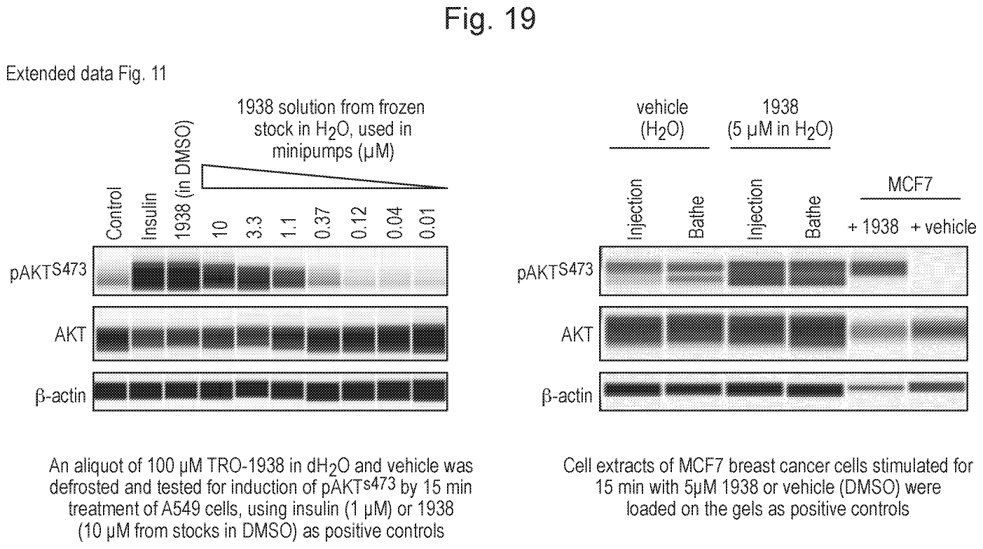

In the Langendorff ex vivo perfused rat heart, a well-established experimental model of IRI, 1938 was found to be a fast-acting agonist which, upon administration during the first 15 min of reperfusion, provided substantial tissue protection from IRI. This was evidenced by increased tissue survival and reduced infarct size (representative images shown in FIG. 5a; quantitated in FIG. 5b) and increased functionality (assessed by ECG; FIG. 5a), both associated with an increase in generation of pAKTS473 (FIG. 5c; full data shown in FIG. 18/Extended data FIG. 10). 1938 also provided significant cardioprotection in an in vivo model of IRI in mice (left panel in FIG. 11), with a corresponding increase in pAKT^{S473} levels in the hearts of these mice (right panel in FIG. 11). Given the observed rapid activation of PI3Kα, it could be envisaged that therapeutic application of a direct PI3Kα activator to a patient undergoing emergency coronary revascularization following myocardial infarction could be cardioprotective, and feasible in the clinical setting.

PI3K pathway activation has been extensively linked to neuroprotection and neuroregeneration, with a positive role for PI3Kα recently demonstrated in axonal regeneration using genetic approaches. Given that there are currently no small molecule clinical-stage treatments routinely used to stimulate neuronal regeneration such as for injury to peripheral nerves, the spinal cord or optic nerves, we explored the potential of PI3Kα activation in this context. In a dose-dependent manner, 1938 significantly increased neurite outgrowth in dissociated adult rat dorsal root ganglion (DRG) cultures, an in vitro model for neuroregeneration, with higher 1938 concentrations doubling the total length of neurites measured at 72 h (FIG. 5d). This increase in neurite outgrowth associated with 1938 was abrogated in the presence of the PI3Kα inhibitor BYL719 (FIG. 5d, right panel).

Stimulated by these observations, we next tested 1938 in the sciatic nerve crush model in the rat, an in vivo model of peripheral nerve injury and regeneration. Exploratory experiments showed induction of pAKT upon direct injection of 1938 or upon bathing of exposed sciatic nerves in a 1938 solution (FIG. 19/Extended Data FIG. 11), indicating that 1938 leads to PI3K pathway activation in this tissue when delivered locally. Immediately after the nerve crush (FIG. 5e, i-ii), 1938 was delivered via a single intraneural injection into the proximal crush site (FIG. 5e, iii) and via a minipump implanted adjacent to the nerve (FIG. 5e, iv), loaded with 1938 solution, for continuous delivery for the duration of the experiment. Analyses were conducted 3 weeks after injury.

Electrophysiological recordings from the tibialis anterior muscle during nerve stimulation proximal to the injury site showed a greater electrophysiological recovery upon 1938-treatment, as indicated by an increased motor unit number estimation (MUNE) (FIG. 5f) and greater compound muscle action potential (CMAP) recovery (FIG. 5g). This correlated with histological analyses which showed (1) an increase in 1938-treated animals in the number of choline acetyltransferase (ChAT)-positive motor axons [FIG. 5h; assessed in distal nerve sections from the common peroneal branch of the sciatic nerve, close to the point of re-innervation of the tibialis anterior muscle], with neurites grouped within normal fascicular nerve architecture (FIG. 5i); and (2) innervation of a proportion of neuromuscular junctions in the tibialis anterior muscles (FIG. 5j), with α-bungarotoxin (α-BTX) staining showing the characteristic distribution of post-synaptic acetylcholine receptors and neurofilament immunoreactivity detecting the neurons (representative example shown in FIG. 5k).

Analysis after 21 days is an early time point in terms of regeneration, with low level initial re-innervation of muscle expected in untreated animals. The histological detection of motor axons in the distal nerve and neuromuscular Junctions (NMJs) corresponds with improved electrophysiological reinnervation of the tibialis anterior muscle. Histological analysis of nerve sections closer to the point of injury (3 mm and 6 mm distal to the crush site) showed equivalent numbers of neurofilament- and ChAT-positive axons in treatment and control groups (FIG. 5l). This indicates that the improved functional muscle re-innervation associated with 1938 treatment is due to an acceleration of natural neuronal regeneration rather than a change in the overall number of regenerating neurites.

Taken together, the data above provide preclinical proof-of-principle of the therapeutic potential of short-term topical pharmacological PI3Kα activation in tissue protection and regeneration.

### Example 8

Assessment of the suitability of PI3Kα activation in cancer treatment.

### Therapeutic Potential of PI3Kα Activation in Cancer Treatment

The PI3Kα activators developed by the inventors can induce cell death of multiple cancer cell lines of diverse tissue origin in conditions of nutrient and O₂ deprivation, with no cytotoxic effect on primary rat neurons, human endothelial cells (HUVECs) and the immortalised but non-transformed MCF-10A breast epithelial cell line [Table 3; FIG. 6,7). This cell death is assessed by propidium iodide staining/FACS and can be partially neutralized by the PI3Kα inhibitor BYL719 (FIG. 6,7) or PI3K pathway inhibitors (FIG. 6,7). PI3Kα-induced overactivation cell death is observed 72 h after continuous exposure, with exposure for as little as 0.5-1 h drug exposure able to observe cytotoxic responses 72 h later (FIG. 8), indicating the possibility of pulsatile drug dosing in vivo.

TABLE 3
in vitro sensitivity of different cell
types to PI3Kα-induced cell death

| | | | PI3K-OCD |
|---|---|---|---|
| Tissue Origin | Cells | Cancer type/histology | response |
| Primary/non- | HUVECs | human umbilical vein | no |
| transformed | | endothelial cells | |
| | DRG neurons | primary rat dorsal | no |
| | | root ganglia neurons | |
| | MCF10A | non-tumourigenic | no |
| | | immortalised breast | |
| | | epithelial cell line | |
| LUNG | H1299 | NSCLC (non small | no |
| CANCER | | cell carcinoma) | |
| | H661 | NSCLC (large cell | partial |
| | | carcinoma) | |
| | A549 | NSCLC (non small | yes |
| | | cell carcinoma) | |
| | H460 | NSCLC (large cell | yes |
| | | carcinoma) | |
| | H1975 | NSCLC (non small | yes |
| | | cell carcinoma) | |
| | LLC (mouse) | NSCLC | yes |
| BREAST | MDA-MB-231 | Basal-like | partial |
| CANCER | BT549 | Basal-like | yes |
| | MCF7 | Luminal | yes |
| | T47D | Luminal | yes |
| GBM | U87-MG | glioblastoma | yes |
| COLORECTAL | HCT116 | colorectal carcinoma | yes |
| CANCER | COLO230 | colorectal carcinoma | yes |
| | HSR | | |

### Example 9

Supplementary crystallographic studies on 1938, as a selected example compound of the invention.

1938 Activates PI3Kα by Disrupting Inhibitory Contacts Between p85α and p110α

In order to understand how 1938 interacts with PI3Kα, the inventors attempted to crystallize PI3Kα in the presence of 1938. They first used a construct containing full length p110α and a truncated niSH2 p85α (p110α M232K L223K/p85α 307-593). Despite obtaining PI3Kα crystals that diffracted up to 2.2 Å resolution, no compound was visible, either upon co-crystallisation or upon compound soaking in preformed crystals (PDB: 7PG5). Co-crystallisation of PI3Kα with both 1938 and BYL719 resulted in crystals in which only density for BYL719 was visible (2.5 Å resolution, PDB: 7PG6). The inventors then used a construct containing only the p110α catalytic subunit, with the adaptor binding domain and lipid binding surface in the kinase domain deleted (p110α 105-1048). Co-crystallisation of p110α with 1938 did not yield crystals, however, the inventors were able to soak 1938 with preformed crystals and observed density for 1938. They obtained crystals that diffracted up to 2.4 Å for apo p110α 105-1048, and 2.5 Å for p110α 105-1048 soaked with 1938.

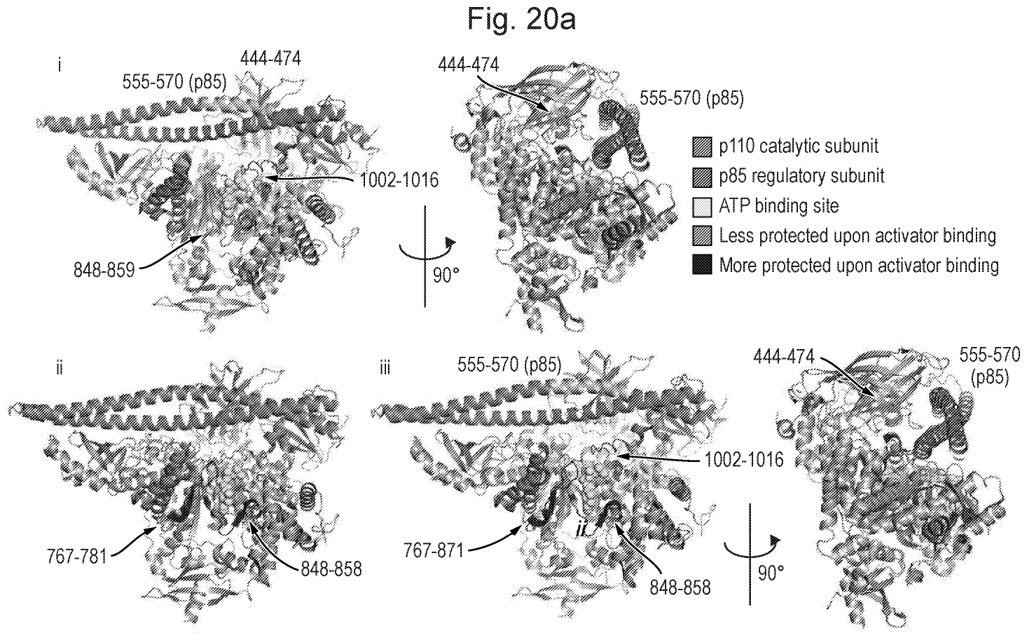

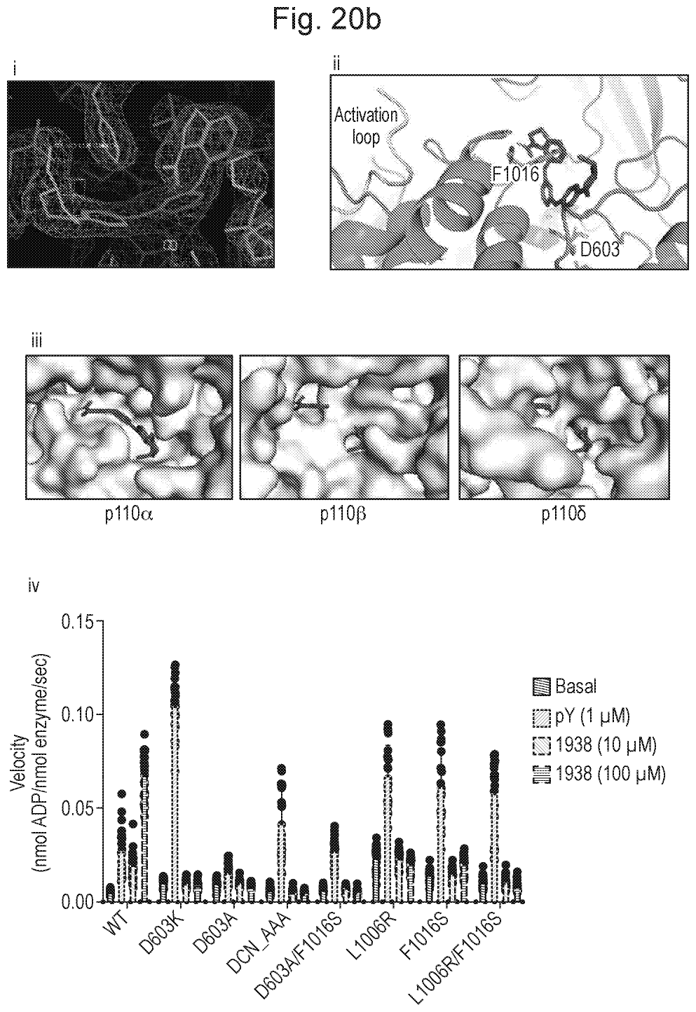

Agreeing with the HDX-MS results, the crystal structure shows that 1938 binds in a pocket surrounded by residues E365, I459, L540, D603, C604, N605, Y641, S1003, L1006, G1007 and F1016 (FIG. 20bi,ii). The core pyridine nitrogen in 1938 is sufficiently basic to be predominantly protonated at physiological pH and this NH⁺ makes key interactions with the sidechain of D603. It is worth noting that the protonated state of the core pyridine may explain the lack of protein kinase inhibition observed with 1938: in this protonated state, the molecule cannot form the donor-acceptor motif characteristic of standard protein kinase inhibitors. The acetylated indoline of 1938 sits in a pocket comprised of L1006, F1016 and I459, and makes face to edge interactions with F1016. Binding of 1938 induces F1016 to move away from the pocket in order to accommodate the ligand. The piperazine is surrounded by E365 and L540, and points out towards solvent.

Global conformational shifts are observed upon compound binding. The C2 domain and helical domain both moves away from the kinase domain. The loop (1002-1016) identified as more protected upon compound binding by HDX-MS moves away from the activator binding site in the p110α-1938 structure compared to apo. In addition, the alpha helix 1016-1026 also shifts away upon compound binding. The 1938 binding site is in proximity to the E542 and E545 hotspots (approx. 10 Å). This region is important for inhibition of p110α by the nSH2 domain of p85α, therefore it is possible that 1938 weakens the inhibitory effects of p85α on p110α, contributing to enzyme activation.

Interestingly, the p110α-1938 structure highlights a potential reason for lack of activity against both PI3Kβ and PI3Kδ isoforms. Comparing the structure of the 1938-binding pocket in p110α with the analogous regions in p110β (PDB: 2Y3A) and p110δ (PDB: 6PYU) showed that p110β and p110δ do not have pockets that could accommodate 1938 (FIG. 20biii). Key components of the compound binding mode are confirmed by preliminary structure-activity relationship analysis. Replacement of the core pyridine for a 2,4-pyrimidine (Compound 2152) results in >95% reduction in activity, consistent with the proposal that the equivalent nitrogen is no longer protonated at physiological pH and unable to form the key interaction with D603. Compounds 1887 and 1889 have activities comparable with 1938, indicating that modification or replacement of the piperazine can be tolerated (Table 4). However, complete removal of the piperazine (Compound 2016) reduces activity by more than 90%. Compound 2016 is also less soluble than 1938. The crystal structure shows that the piperazine points out towards solvent, suggesting that presence of the piperazine or tri-O-methyl substituted phenyl may be important in displacing water molecules and maintaining hydrophobic interactions with L1006 and F1016. The indoline is required for edge to face and hydrophobic interactions with F1016 and L1006R. The carbonyl group makes an internal hydrogen bond with the NH group linking the indoline and pyridine, holding the indoline in an orientation suitable for interacting with F1016. Replacement of the acetylated indoline with a pyrimidine (Compound 2106) reduces activity by more than 95%, potentially due to less favourable edge to face interactions with F1016, and increased flexibility of the pyridine.

The inventors then performed mutagenesis in attempt to generate 1938-resistant mutants. Based on the crystal structure and SAR data, we made the following mutants: D603K, D603A, 603DCN_AAA605 triple mutant, D603A/F1016S double mutant, L1006R, F1016S, and the L1006R/F1016S double mutant. Basal activities of D603K, D603A, DCN_AAA triple mutant, D603A/F1016S and L1006R/F1016S double mutants are comparable to that of the WT; L1006R (p<0.0001) and F1016S (p=0.0056) had basal activities significantly higher than WT. All the mutants could be further activated upon stimulation by pY, and yet all the mutants were resistant to activation by 1938 (FIG. 20biv).

TABLE 4
activation properties of selected compounds

| | | | Max. activity | Max. activity |
|---|---|---|---|---|
| | | | relative to pΥ | relative to |
| Cpd | Structure | EC₅₀ (μM) | ctrl (%) | 1938 (%) |
| 1938 | 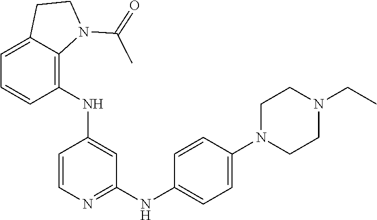 [SMILES: CCN1CCN(c2ccc(Nc3cc(Nc4cccc5c4N(C(C)=O)CC5)ccn3)cc2)CC1] | 58 ± 28 | 397 ± 60 | 100 |
| 1887 | 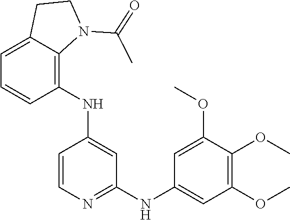 [SMILES: COc1cc(Nc2cc(Nc3cccc4c3N(C(C)=O)CC4)ccn2)cc(OC)c1OC] | 36 ± 5 | 318 ± 68 | 80 |
| 1889 | 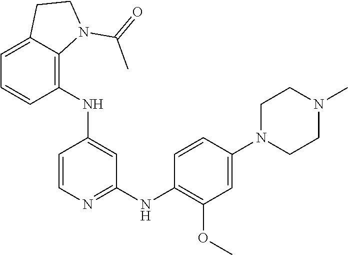 [SMILES: COc1cc(N2CCN(C)CC2)ccc1Nc1cc(Nc2cccc3c2N(C(C)=O)CC3)ccn1] | 56 ± 24 | 408 ± 110 | 102 |
| 2016 | 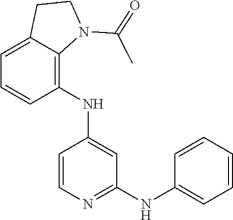 [SMILES: CC(=O)N1CCc2cccc(Nc3ccnc(Nc4ccccc4)c3)c21] | NA | 36 ± 8 | 9 |
| 2106 | 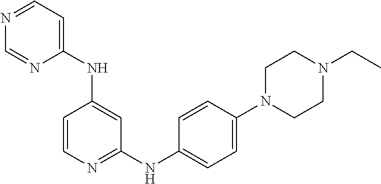 [SMILES: CCN1CCN(c2ccc(Nc3cc(Nc4ccncn4)ccn3)cc2)CC1] | NA | 14 ± 13 | 4 |
| 2152 | 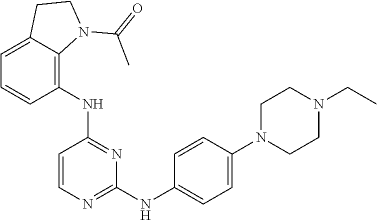 [SMILES: CCN1CCN(c2ccc(Nc3nccc(Nc4cccc5c4N(C(C)=O)CC5)n3)cc2)CC1] | NA | 16 ± 8 | 4 |

### Example 10

Supplementary cell-based characterization of the activity of 1938, as a selected example compound of the invention.

### 1938 Induces PI3Kα Pathway Activation in Cells

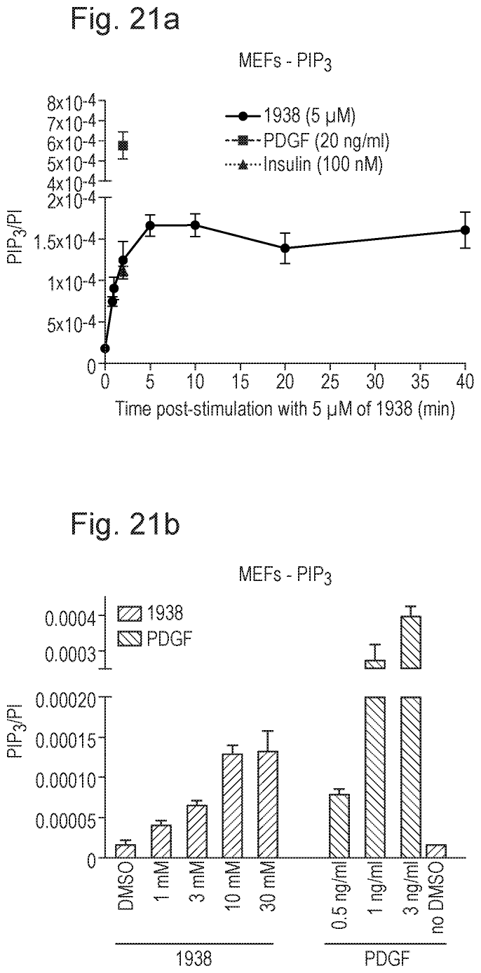

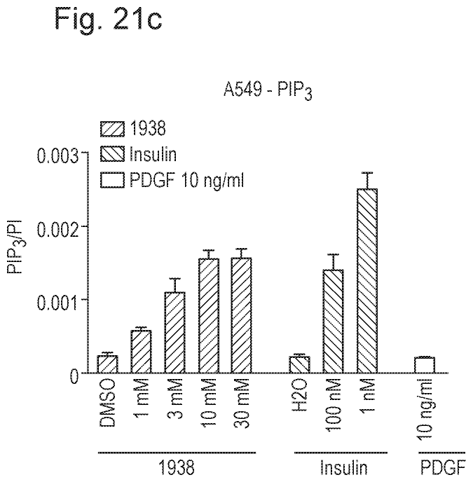

Class I PI3Ks phosphorylate the PtdIns(4,5)P₂ lipid in the plasma membrane to generate PtdIns(3,4,5)P₃ (or PIP₃), which can be converted to PtdIns(3,4)P₂ by the action of 5-phosphatases. Treatment of MEFs (Mouse Embryonic Fibroblasts) with 1938 very rapidly (within 30 sec) led to increased PIP₃ levels, as assessed by mass spectrometry, maxing at 5 min and maintained at this maximum level for up to 40 min (FIG. 21a). At the 2 min time point, the levels of PIP₃ induced by 1938 were comparable to those induced by insulin, but lower than those induced by PDGF. The observation of different levels of PIP₃ induced by these growth factors is in line with the notion that PI3Kα is the sole mediator of PIP₃ production downstream of insulin. This contrasts with PDGF, which activates both PI3Kα and PI3Kβ, with the latter PI3K isoform contributing substantially to acute PDGF-stimulated PIP₃ generation in MEFs, correlating with the higher PIP₃ levels induced by this agonist compared to insulin and 1938 (FIG. 21a). In the same experiment as in FIG. 21a, clear PI(3,4)P₂ signal was detected in MEFs upon PDGF stimulation but not with 1938 (at 5 μM). This is consistent with a higher threshold for detection of PI(3,4)P₂ compared to PIP₃ by mass spectrometry (due primarily to background contamination, as discussed by Malek et al.), together with the relatively lower PI3K activation by 1938 compared to a high dose of PDGF, as is also illustrated by the experiments shown below.

When tested at different doses at a fixed 2 min time point, PIP₃ induction by 1938 in MEFs was found to have an EC₅₀ of ˜5 μM, plateauing around 10 μM, at a substantially lower level of PIP₃ to that induced by PDGF at 1 ng/ml or 3 ng/ml (FIG. 21b). These maximal 1938-induced PIP₃ levels are below those required to give rise to sufficient PI(3,4)P₂ to be detectable by mass spectrometry, a conclusion also supported by the observation that substantial levels of PIP₃ induced by lower doses of PDGF (e.g. 0.5 ng/ml) were also not sufficient to give rise to levels of PI(3,4)P₂ detectable by mass. Similar to what was observed for MEFs, stimulation of A549 cells for 2 min with a dose range of 1938 revealed that the PIP₃ response to 1938 also maxed out at 10 μM (FIG. 21c). A strong PIP₃ response was also observed with insulin in these cells, with no PIP₃ induced by PDGF, in line with the absence of this receptor in epithelial cells, including in A549.

### Methods

### PI3K Protein Expression and Purification

Full-length p110α was expressed in a complex with full-length p85α (for biochemistry and HDX-MS) or with a truncated p85-niSH2 protein (for crystallography). A p110α construct lacking the adaptor binding domain and lipid binding surface (105-1048) was also used for crystallography (Chen et al. (2014) Protein Sci 23: 1332-1340).

Expression and purification p110α (LMB-MRC plasmid OP831) in complex with full-length p85α (LMB-MRC plasmid OP809) was performed as described (Burke, J. E. et al. Proc Natl Acad Sci USA 109, 15259-15264 (2012).). The oncogenic mutants G106V (LMB-MRC plasmid JB35), N345K (LMB-MRC plasmid OP661) and E545K (LMB-MRC plasmid OP663) were also purified using this protocol. Briefly, 10 litres of Spodoptera frugiperda (Sf9) cell culture at a density of 1.0×10⁶ cells/ml were co-infected with a p85α-encoding virus [LMB-MRC plasmid LOP809]. and a virus encoding p110α with an N-terminal 6×His tag followed by a tobacco etch virus (TEV) protease site [LMB-MRC plasmid OP831]. After a 48 h infection at 27° C., cells were harvested and washed with PBS. Cell pellets were then resuspended in Lysis Buffer (20 mM Tris pH 8.0, 300 mM NaCl, 5% glycerol, 10 mM Imidazole pH 8.0, 2 mM β-mercaptoethanol, 1 EDTA-free protease inhibitor tablet (Roche) per 50 ml of buffer) and sonicated at 4° C. for 7 min in 15 sec intervals followed by a 15 sec wait. Cell lysate was then centrifuged at 45,000 g for 45 min at 4° C. Supernant was then filtered using a 0.45 μM filter before being passed over 2×5 ml HisTrap FF (Cytiva) Columns (equilibrated in NiNTA Buffer [20 mM Tris pH 8.0, 300 mM NaCl, 5% glycerol, 10 mM imidazole (pH 8.0), 2 mM β-mercaptoethanol]) at a 3 ml/min flow rate. Columns were then washed using a 20 mM imidazole wash, and protein was eluted in a gradient to NiNTA B Buffer (20 mM Tris pH 8.0, 300 mM NaCl, 5% glycerol, 200 mM imidazole (pH 8.0), 2 mM β-mercaptoethanol). PI3Kα containing fractions were then pooled and diluted 1:2 with Salt Dilution Buffer (20 mM Tris pH 8.0, 1 mM DTT) to reduce NaCl concertation to 100 mM. This solution was then passed over a HiTrap Heparin (Cytiva) Column (equilibrated in Hep A Buffer (20 mM Tris pH 8.0, 100 mM NaCl, 2 mM β-mercaptoethanol)) at a rate of 3 ml/min. PI3Kα was eluted using a gradient to HEP B Buffer (20 mM Tris pH 8.0, 1 M NaCl, 2 mM β-mercaptoethanol). Protein containing fractions were then pooled and concentrated to 8 mg/ml, before being loaded onto a Superdex 200 16/60 column, equilibrated in Gel Filtration Buffer (20 mM HEPES pH 7.4, 100 mM NaCl, 2 mM TCEP), run at 1 ml/min at 4° C. PI3Kα-containing fractions were pooled and concentrated to 2.5 mg/ml before being flash-frozen in liquid nitrogen and stored at −80° C.

Expression and purification p110α in complex with p85α-niSH2 was performed as follows. Sf9 insect cells were cultured in Insect-XPRESS with L-Glutamine medium (Lonza BE12-730Q) at 27° C. and infected with baculovirus encoding both p110α and p85α-niSH2 [LMB-MRC plasmid GM129] at a density of 1.6-1.8×10⁶ cells/ml. The culture was incubated for 48 h after infection, and cells were collected and washed with PBS, flash-frozen in liquid N₂ and stored at −80° C. For purification, cell pellets were resuspended in 100 ml of lysis buffer (20 mM Tris, 150 mM NaCl, 5% glycerol, 2 mM β-mercaptoethanol, 0.02% CHAPS, pH 8.0) containing EDTA-free Protease inhibitor tablets (Roche, 1 tablet per 50 ml of solution) and 500 μl DNAse I. The suspension was sonicated for 10 min on ice, with 10 sec on and 10 sec off. The lysate was then centrifuged at 35,000 rpm for 45 min using a Ti45 rotor at 4° C. The samples were loaded onto a StrepTrap (Cytiva) column in S300 buffer (20 mM Tris, 300 mM NaCl, 5% glycerol, 2 mM TCEP, pH 8.0). Once the protein was loaded, the column as washed with buffer A (20 mM Tris, 100 mM NaCl, 5% glycerol, 1 mM TCEP, pH 8.0). The column was eluted using a gradient from 1-100% buffer B (buffer A containing 5 mM d-Desthiobiotin). Fractions of the p110α/p85α-niSH2 peak were pooled and TEV protease (0.8 mg/ml) was added at the ratio of 1:10 and left at 4° C. to cleave overnight. Protein was loaded onto a 5 ml HiTrap Heparin HP column (Cytiva) washed with buffer A, and eluted with a gradient of 1-100% buffer C (20 mM Tris, 1 M NaCl, 1 mM TCEP, pH 8.0). The fractions were collected, concentrated and loaded on a Superdex 200 26/60 HiLoad gel filtration column (Cytiva) and eluted in 20 mM Tris, 200 mM NaCl, 2 mM TCEP, 1% betaine, 1% ethylene glycol and 0.02% CHAPS, pH 7.2. The peak fractions were pooled and concentrated to 10-13 mg/ml using Amicon Ultra-15 Centrifugal filters 100K (Millipore), as measured by a NanoDrop at 280 nm. The protein was then flash-frozen in liquid nitrogen and stored at −80° C. Purity of protein was checked using SDS-PAGE.

Expression and purification of truncated human p110α (105-1048) were performed as follows. Sf9 insect cells (9 L) were cultured in Insect-XPRESS with L-Glutamine medium (Lonza BE12-730Q) at 27° C. and infected with baculovirus encoding the p110α subunit [LMB-MRC plasmid OP798] at a density of 1.6×10⁶ cells/ml. The culture was incubated for 48 h after infection, cells were collected, flash-frozen in liquid N₂ and stored at −80° C. For purification, cell pellets were resuspended in 360 ml of lysis buffer (20 mM Tris, 150 mM NaCl, 5% glycerol, 1 mM TCEP, pH 8.0) containing EDTA-free Protease inhibitor tablets (1 tablet per 50 ml of solution), 0.5 mM PEFA and 36 μl of Piece® Universal Nuclease For Cell Lysis. The suspension was sonicated for 5 min on ice, with 10 sec on and 10 sec off. The lysate was then centrifuged at 35,000 rpm for 35 min using a Ti45 rotor at 4° C. The samples were filtered through a 5 m filter and loaded onto a StrepTrap (Cytiva) column equilibrated in lysis buffer. Once the sample was loaded, the column was washed with 20 mM Tris, 300 mM NaCl, 5% glycerol, 1 mM TCEP, pH 8.0, and then with 20 mM Tris, 150 mM NaCl, 5% glycerol, 1 mM TCEP, pH 8.0. Then 5 ml TEV solution at 0.14 mg/ml was added onto the column and left at 4° C. to cleave overnight. Protein was loaded onto a 5 ml HiTrap Heparin HP column (Cytiva) equilibrated in 20 mM Tris, 150 mM NaCl, 5% glycerol, 1 mM TCEP, pH 8.0, and eluted with a gradient of 1-100% of 20 mM Tris, 1 M NaCl, 1 mM TCEP, pH 8.0. The fractions were collected, concentrated and loaded on a Superdex 200 16/60 HiLoad gel filtration column (Cytiva) and eluted in 50 mM Tris, 100 mM NaCl, 2% ethylene glycol, and 1 mM TCEP, pH 8.0. The peak fractions were pooled and concentrated to 5.83 mg/ml using Amicon Ultra-15 Centrifugal filters 50K (Millipore), as measured by a NanoDrop at 280 nm. The protein was then flash-frozen in liquid nitrogen and stored at −80° C. Purity of protein was checked using SDS-PAGE.

Full-length p110β/p85α and p110δ/p85α were cloned and expressed in a similar manner but using a streptavidin-tag instead of a his-tag. Briefly, 5 litres of Spodoptera frugiperda (Sf9) cell culture at a density of 1.0×10⁶ cells/ml were co-infected with both a p85α-encoding virus and a virus encoding p110β/δ with an N-terminal Strep-tag followed by a tobacco etch virus (TEV) protease site (plasmid OP832 for p110p, plasmid OP833 for p110δ and plasmid of OP809 for p85α). After a 48 h infection at 27° C., cells were harvested and washed with PBS. Cell pellets were then resuspended in Lysis Buffer (20 mM Tris pH 8.0, 150 mM NaCl, 5% glycerol, 2 mM β-mercaptoethanol, 1 EDTA-free protease inhibitor tablet (Roche) per 50 ml of buffer) and sonicated at 4° C. for 7 min in 15 sec intervals followed by a 15 sec wait. Cell lysate was then centrifuged at 45,000 g for 45 min at 4° C. Supernant was then filtered using a 0.45 μM filter before being passed over 1×5 ml StrepTap No 1 (GE Healthcare) Columns (equilibrated in 100S Buffer [20 mM Tris pH 8.0, 100 mM NaCl, 5% glycerol, 1 mM TCEP]) at a 3 ml/min flow rate. Column was then washed using 70 ml 100S Buffer, followed by 80 ml S300 Buffer (20 mM Tris pH 8.0, 300 mM NaCl, 5% glycerol, 1 mM TCEP) followed by 50 ml S100 Buffer. 5 ml of 0.1 mg/ml His6TEV protease (p30) in S100 Buffer was injected onto the column and was incubated at 4° C. for 4 h. The column was then attached to a Heparin column, and the purification protocol proceeded as for PI3Kα.

### Fluorescence Polarization Assay

PIP₃ production was measured using a fluorescence polarization assay (#K-1100; Echelon Biosciences, Salt Lake City, UT, USA) and carried out in 384-well microtitre plates. PI3Kα, liposomes and ATP were all diluted in the reaction buffer (20 mM HEPES, 50 mM NaCl, 50 mM KCl, 3 mM MgCl₂, 1 mM EGTA, 1 mM TCEP, pH 7.4) and added to the microtitre plate at a final reaction concentration of 10 nM PI3Kα, 75 μg/ml liposomes and 10 μM ATP. The reaction was carried out for 45 min at room temperature and quenched with the PIP₃ detector and TAMRA probe, before being read in a Hidex Sense platereader using λ544±20 and λ590±20 polarizing filters. Data was normalised to the TAMRA probe alone and TAMRA plus detector for minimum and maximum PIP₃ production, respectively.

### Microscale Thermophoresis

MST experiments were performed using an automated Monolith NT.115 (NanoTemper Technologies, Munich, Germany). Fluorescence labelling of PI3Kα with the NT647 dye was performed in accordance with manufacturer protocol using the RED-NHS protein labelling Kit (NanoTemper Technologies, Munich, Germany). PI3Kα was diluted to a final concentration of 2.5 nM in reaction buffer (20 mM HEPES, 100 mM NaCl, 0.1% Tween-20 and 2 mM TCEP, pH 7.4). Compounds were serially diluted in neat DMSO and added to the enzyme to a final concentration of 3% DMSO. Premium treated capillaries, IR laser powers of 80% and LED intensity of 10% were used. Data was analysed with the NanoTemper Analysis software with ΔFₙₒᵣₘ values (ΔFₙₒᵣₘ=Fₕₒₜ/F_{cold}) used to define compound binding.

### ADP-Glo™ Kinase Assay

Kinase reactions were performed with ADP-Glo kinase assay kit (Promega Corporation). The enzyme, substrate and compounds were diluted in reaction buffer (20 mM HEPES, 50 mM NaCl, 50 mM KCl, 3 mM MgCl₂, 1 mM EGTA, 1 mM TCEP, pH 7.4). Final concentrations of PI3Kα and PI3Kδ used were 25 nM and 50 nM for PI3Kβ. Liposomes (5% brain PI(4,5)P₂, 20% brain phosphatidylserine, 45% brain phosphatidylethanolamine, 15% brain phosphatidylcholine, 10% cholesterol, 5% sphingomyelin (Avanti Polar Lipids)) were used at a final concentration of 1 mg/ml. The pY sequence is ESDGG(pY)MDMSKDESID(pY)VPMLDMKGDIKYADIE.

For compound profiling, the reaction mixture contained 2 μl PI3K enzyme, 2 μl compound and/or pY and 2 μl of liposome substrate mixed with ATP. ATP was used at a final concentration of 500 μM for PI3Kα and PI3Kβ and at 200 μM for PI3Kδ, unless otherwise stated. The final DMSO concentration in the assay was 1%. The experiments were performed at room temperature for 3 h using 384 white-polystyrene plates (Corning #3824) before addition of 6 μl of ADP-Glo R1 to terminate the reaction. The plate was incubated for 45 min, followed by addition of 12 μl of ADP-Glo R2 and incubated further for 60 min in the dark. Luminescence was read using a Sense (Hidex) plate reader. Compound data were corrected to the no enzyme DMSO negative control and expressed as a percentage of the internal positive control (1 μM pY), equivalent to maximal activation (Eₘₐₓ). All analyses were performed using GraphPad Prism 7.

For characterisation of the effects of 1938 on in vitro PI3K enzymology, all reactions were performed at room temperature with 384 white-polystyrene plates (Corning #3574). The final DMSO concentration in the assay was between 0.5%-1.8%. The reaction mixture contained 2 μl PI3K enzyme, 2 μl compound and/or pY and 2 μl of liposome substrate mixed with ATP. ATP was used at a final concentration of 200 μM, unless otherwise stated. The enzyme and compounds were pre-incubated for 10 min prior to addition of substrate. The reaction was allowed to proceed for 45 min at room temperature, before addition of 6 μl of ADP-Glo R1 to terminate the reaction. The plate was incubated for 60 min, followed by addition of 12 μl of ADP-Glo R2 and incubated further for 60 min in the dark. For enzyme kinetic calculations, data was expressed as velocity (pmol of ADP generated/sec). ADP-ATP standard curves were performed according to the manufacturer's instructions, and all analyses were performed using GraphPad Prism 8.

### FRET Membrane Binding Assay

Membrane binding assays were performed as previously published (Burke, J. E. et al. Proc Natl Acad Sci USA 109, 15259-15264 (2012)). Briefly, liposomes were prepared with 5% (w/v) brain PtdIns(4,5)P₂, 20% brain phosphatidylserine, 35% brain phosphatidylethanolamine, 15% brain phosphatidylcholine, 10% cholesterol, 5% sphingomyelin, and 10% dansyl-phosphatidylserine (Avanti Polar Lipids). PI3Kα was used at a final concentration of 0.5 μM. Protein solutions were preincubated with 10 μM pY or compounds for 10 min before addition of liposomes. Liposomes were used at a final concentration of 50 μg/ml. The reaction mixture contained 5 μl enzyme, 2 μl compound and 3 μl liposomes, all diluted in 30 mM HEPES, 50 mM NaCl, pH 7.4. The reaction was allowed to proceed for 10 min at room temperature in 384 black-polystyrene plates (Corning #3544) on an orbital shaker at 200 rpm. FRET signals were measured using PHERAStar (BMG) with a 280 nm excitation filter with 350 nm and 520 nm emission filters to measure Dansyl-PS FRET emissions, respectively. FRET signal shown as I-I0, where I is the intensity at 520 nm, and 10 is the intensity at 520 nm for the solution in the absence of protein.

### HDX-MS

Sample preparation: HDX-MS experiments were carried out as described previously (Anandapadamanaban, M. et al. Science (New York, N.Y 366, 203-210 (2019)). Briefly, 5 μM PI3Kα was incubated either in the absence of compound, with 300 μM 1938 in a 1% DMSO-containing Protein Dilution Buffer (50 mM Tris pH 7.5, 150 mM NaCl, 2 mM TCEP), with 100 μM BYL719 or with both 1938 and BYL719. 5 μl PI3Kα either with or without compound was then incubated with 45 μl D₂O Buffer (50 mM Tris pH 7.5, 150 mM NaCl, 2 mM TCEP, 1% DMSO with or without 50 μM 1938, 90.6% D₂O) for 5 timepoints (0.3 sec/3 sec/30 sec/300 sec/3000 sec, with the 0.3 sec timepoint being a 3 sec timepoint conducted at 0° C.) before being quenched with 20 μl ice-cold Quench Solution (2 M Guanidinium Chloride, 2.4% Formic Acid), and being rapidly snap-frozen in liquid nitrogen prior to storage at −80° C.). In total, three biological replicates, i.e., three separate protein preparations, each with exchange experiments were carried out in triplicate were conducted. Results illustrated are from a single, representative biological replicate. Data acquisition and analysis were as follows: Each sample was thawed and injected onto an M-Class Acquity UPLC with HDX Technology (Waters) kept at 0.1° C. Proteins were digested in-line using an Enzymate Pepsin Column (Waters, 186007233) at 15° C. for 2 min. Peptic peptides were then eluted onto an Acquity UPLC BEH C18 Column (Waters, 186002346) equilibrated in Pepsin-A buffer (0.1% formic acid) and separated using a 3-43% gradient of Pepsin-B buffer (0.1% formic acid, 99% acetonitrile) over 16 min. Data were collected on a Waters Cyclic IMS, with an electrospray ionisation source, from 50-2000 m/z. Data were collected in the HDMSe mode. A single pass of the cyclic IMS was conducted. A “blank” sample of protein dilution buffer with quench was run between samples, and carry-over of peptides was routinely monitored. Five replicates were used to identify non-deuterated peptides. Criteria used to include peptides in the HDX-MS dataset were: minimum intensity 5000, minimum sequence length 5, maximum sequence length 25, a minimum of 3 fragment ions, a minimum of 0.1 products per amino acid, a minimum score of 6.62, a maximum MH+ Error of 10 ppm, identification in at least two datasets with a retention time RSD of less than 10%. Data was analysed using Protein Lynx Global Server (Waters) and DynamX (Waters). All peptides were manually inspected for EX1 kinetics and sufficient quality of the peptide envelope. Data quality, experiment design, and reporting of data meets the criteria as determined by the HDX-MS community (Masson, G. R. et al. Nat Methods 16, 595-602 (2019)). Uptake files were created using Baryonyx.

Kinase Profiling, mTORC1 and ATM Kinase Assays

133 protein kinases and 7 lipid kinases were counterscreened, with 1988 used at 1 μM, using the Adapta, Lantha and Z-LYTE assays (SelectScreen Kinase Profiling Service; Thermofisher—experimental details of these assays can be found here: https://www.thermofisher.com/uk/en/home/industrial/pharma-biopharma/drug-discovery-development/target-and-lead-identification-and-validation/kinasebiology/kinase-activity-assays.html. The tree representation in KinMap generated courtesy of Cell Signaling Technology, Inc. (www.cellsignal.com). mTORC1 (mTOR/RAPTOR/LST8) protein complex and ATM kinase and substrates were produced as previously described (Anandapadamanaban, M. et al. Science (New York, NY 366, 203-210 (2019); Baretic, D. et al. Sci Adv 3, e1700933 (2017). Screening of 1938 was conducted using SuperSep Phos-Tag 50 μmol/l 100×100×6.6 mm 17-well (192-18001/199-18011) gels. For ATM assays, 100 nM ATM was incubated for 30 min at 30° C. with 5 μM GST-p53 and 1 mM ATP, in the absence or presence of 200 μM 1938 in ATM Kinase Buffer (50 mM HEPES pH 7.5, 100 mM NaCl, 10% glycerol, 2 mM Trichloroethylene, 5 mM MgCl₂). As a positive control for ATM activation, the same reaction was carried out with 100 nM ATM/5 μM GST-p53/1 mM ATP in the presence of 100 nM Mre11-Rad50-Nbs1 (MRN) complex, a known activator of ATM. For mTORC1 assays, 50 nM mTORC1 complex (mTOR/LST8/RAPTOR) was incubated for 3 h at 30° C. with 15 μM 4E-BP1, 10 mM MgCl₂ and 250 μM ATP, in the absence or presence of 200 μM 1938. As a ‘positive’ control, 150 nM mTORC1 complex (mTOR/LST8/RAPTOR) was incubated for 3 h at 30° C. with 15 μM 4E-BP1, 10 mM MgCl₂ and 250 μM ATP. Kinase reactions were quenched by addition of SDS-PAGE Loading Buffer (as per manufacturer's instructions) and freezing at −20° C. before being run on the Phos-tag gels at 150 V for 90 min. Gels were then stained using InstantBlue™ Coomassie stain, and then quantified using BioRad Image Lab Software. Kinase assays were carried out in triplicate.

Co-Crystallisation of p110α/p85α niSH2-Compound Complexes

An initial screen of approximately 2000 conditions was performed using the LMB robotic crystallization setup (Stock, D. et al. Prog Biophys Mol Biol 88, 311-327 (2005)). p110α/p85α niSH2 was either pre-incubated with 100 μM of BYL719 for 1 h, or pre-incubated with 100 μM BYL719 for 1 h followed by incubation with 500 μM 1938 for 1 h. Sitting drops were set up by mixing 100 nl of reservoir with 100 nl of protein solution (10 mg/ml) in 96-well MRC-plates. Initial crystals were obtained in 0.2 M KSCN, 0.1 M sodium cacodylate, and between 8-30% of PEG 2K, PEG 4K, PEG 5K and PEG 6K (w/v), or in 80 mM KSCN, 30% PEG 1K (w/v), 150 mM MES, pH 6.0. For optimisation, the crystallisation was set in a sparse matrix layout by varying the concentrations PEG and KSCN in hanging drops by mixing 1 μl of 5.5 mg/ml protein with 1 μl of reservoir, and the best diffracting crystals were obtained in 16% PEG 1K (w/v), 150 nM KSCN, 150 mM MES pH 6.0; 9% PEG 4K (w/v), 180 mM KSCN, 100 mM sodium cacodylate; 10% PEG 5K MME (w/v), 160 nM KSCN, 100 mM sodium cacodylate. Crystals were also soaked between 1-20 h in 10 mM 1938.

Crystallisation of p110α-Compound Complexes

All crystallisation experiments were performed at 20° C. An initial screen of approximately 2300 conditions was performed using the LMB robotic crystallization setup⁹. p110α was either pre-incubated with 500 μM of 1938 or 1% DMSO for 1 h. Sitting drops were set up by mixing 100 nl of reservoir with 100 nl of protein solution (5.8 mg/ml) in 96-well MRC-plates. Crystals for apo were obtained from the Morpheus II screen, in 12.5% (w/v) PEG 4K, 20% (v/v) 1,2,6-hexanetriol, 40 mM Polyamines, 0.1 M MOPSO/bis-tris pH 6.5; and in 12.5% (w/v) PEG 4K, 20% (v/v) 1,2,6-hexanetriol, 90 mM LiNaK, 0.1 M MOPSO/bis-tris pH 6.5. For optimisation with 1938, crystallisation was set up in 96-well MRC-plates by varying the concentrations of PEG, 1,2,6-hexanetriol and polyamine or LiNaK in sitting drops by mixing either 200 nl of 5.8 mg/ml protein with 200 nl of reservoir, or 500 nl of 5.8 mg/ml protein with 500 nl of reservoir. Crystals only formed under apo conditions. These apo crystals were then soaked for 1.5-2 h in 20 mM 1938 (20% DMSO). For data collection, crystals for apo were obtained in conditions containing 12.5% (w/v) PEG 4K, 20% (v/v) 1,2,6-hexanetriol, 90 mM LiNaK, 0.1 M MOPSO/bis-tris pH 6.5 and crystals soaked with 1938 were obtained in conditions containing 12.5% (w/v) PEG 4K, 20% (v/v) 1,2,6-hexanetriol, 50 mM Polyamines, 0.1 M MOPSO/bis-tris pH 6.5. Harvested crystals were cryo-cooled in liquid nitrogen prior to data collection.

Xray Crystal Structure Determination for p110α 105-1048

X-ray diffraction for single crystals of p110α 105-1048 alone and soaked with 1938 were collected using a synchrotron X-ray source. Images were processed using automated image processing with Xia. Initial phases were obtained with molecular replacement, using Phaser in the CCP4 suite, with an initial model from PDB entry 4TUU. Models were manually adjusted to the densities, using COOT, and the structures were refined with PHENIX. The structure in the presence of 1938 showed density in a pocket with walls made up of atoms from residues E365, I459, L540, D603, C604, N605, Y641, S1003, L1006, G1007 and F1016. This pocket was not previously occupied in any ligand in any structure for p110α. The mode of binding was consistent with prior HDX-MS results. A 3D model was built for 1938 from its chemical structure, using PHENIX ELBOW, and this model agreed well with the density in the 1938-soaked crystal. This pocket was empty in a structure obtained from a crystal that was not soaked with 1938. The protein/ligand complex was manually adjusted and refined using COOT and PHENIX. Representations of the complex were prepared using PyMOL and Chimera.

### Detection of Protein Phosphorylation Using Wes™

Experiments with A549 cells and MEFs were performed separately using slightly different protocols. Briefly, A549 cells were seeded at 200,000 cells per well in 24-well plates in DMEM (10% FBS+1% P/S) and allowed to adhere overnight. The next day, cells were washed once with PBS before addition of serum-free DMEM for 24 h. On the day of treatment, cells were incubated in fresh serum-free DMEM prior to treatment. 15 min pre-treatment with either PI3Kα inhibitor (BYL719, 500 nM) or 0.1% DMSO was performed prior to compound addition for 15 min at 37° C., 5% CO₂. Cells were washed with cold RIPA buffer (Thermo, supplemented with protease and phosphatase inhibitors (Roche). MEFs were seeded at 500,000 cells/well in a 12-well plate and allowed to adhere overnight. The next day they were serum-starved for 4 h prior to treatment with 1 M insulin or 1938 (0.2 to 30 μM, final DMSO concentration of 0.5%) for 1 h at 37° C., 5% CO₂. The cells were then washed with cold PBS and lysed in in 50 mM Tris.HCl pH 7.4, 1% Triton-X100, 100 mM NaCl, 50 mM NaF, 5 mM EDTA, 2 mM EGTA, 10 mM Na₄P₂O₇ and Protease/Phosphatase inhibitor cocktail from Merck. The lysate was collected and centrifuged at 15,000 rpm for 15 min at 4° C., supernatant collected and stored at −80° C. Western blotting was performed by Wes™ (ProteinSimple) according to the manufacturer's instructions. Antibodies for pAKT-S473 (CST #4060), total AKT (CST #9272) were used at 1:50; β-actin (CST #4970) was used at 1:100.

### Detection of AKT Phosphorylation by ELISA

A549 cells were seeded at 50,000 cells per well in 96-well plates in DMEM (10% FBS+1% P/S). The next day cells were washed once with PBS before addition of serum-free DMEM for 24 h. On the day of treatment cells, were incubated in fresh serum-free DMEM prior to treatment. Compounds solubilised to 10 mM in DMSO were diluted 1:3 in an 8-point concentration response curve in DMSO. Concentration response curves were diluted in serum-free DMEM by transfer into intermediate plates using a BRAVO liquid handler (Agilent). Intermediate plates were then used to treat cell plates using the BRAVO liquid handler. Compound concentration response curves had atop concentration of 50 μM and a final well concentration of 0.5% DMSO. Cell plates were treated for 15 min at 37° C., 5% CO₂ before being washed with ice-cold PBS and lysed in lysis buffer 6 (R&D Systems #895561) and freezing at −80° C. Levels of pAKT-S473 were determined using the phospho-AKT (S473) pan-specific Duoset IC ELISA (R&D Systems #DYC887BE) in 96-well white high-binding plates (Corning #3922) according to manufacturer's instructions. Endpoint luminescence was measured using a Sense (Hidex) platereader. Compound data were corrected to the negative DMSO control and expressed as a percentage the internal insulin control (1 μM), equivalent to the maximal activation (Eₘₐₓ) induced by insulin. Data were transformed and EC₅₀ data were determined by variable slope (4 parameters) non-linear regression using Prism 7 (Graphpad).

### Cell Culture

Immortalised PI3Kα-WT and PI3Kα-KO MEFs were generated and described previously (Foukas, L. C. et al. Proc Natl Acad Sci USA 107, 11381-11386 (2010)). MEFs were cultured in DMEM containing 10% FBS and 1% penicillin-streptomycin and starved in serum-free DMEM with 1% penicillin-streptomycin at 37° C. and 5% CO₂. A549 cells were cultured either in DMEM Glutamax (Gibco #31966021) supplemented with 10% FBS and 1% penicillin-streptomycin, or in RPMI 1640 medium supplemented with 10% FBS, 1 mM sodium pyruvate and 1% penicillin-streptomycin. For starvation experiments, A549 cells were incubated in serum-free RPMI containing 1 mM sodium pyruvate and 1% penicillin-streptomycin. All cell cultures were regularly tested to be negative for Mycoplasma.

### Generation of PIK3CA-Null A549 Cells by CRISPR/Cas9 Gene Targeting

Generation of pooled PIK3CA-null A549 cells was outsourced to Synthego Corporation. Briefly, the PIK3CA gene was targeted with synthetic ribonucleoprotein (RNP) complexes including the following single guide RNA (sgRNA) sequence: 5′-CUCUACUAUGAGGUGAAUUG-3′ (located within PIK3CA exon 3). In parallel, control cultures were exposed to the Cas9 protein without sgRNA, henceforth referred to as “WT cultures”. Single-cell clones were established from both WT and targeted cultures by limiting dilution, thereby ensuring seeding of maximum 1 cell per well of a 96-well plate. To promote recovery, subcloned cells in 96-wells were cultured in a 1:1 mixture of standard A549 complete medium and conditioned medium. Conditioned medium was prepared from WT cultures 2 days post-passaging by centrifuging the medium at 1000 g for 10 min, followed by 0.22 μm PES filtration and storage at 4° C. (−80° C. for storage exceeding 2 weeks). The medium was replenished every 2-3 days, as gently as possible to prevent cells from dislodging. Once cells reached sub-confluence, they were expanded to 24-well plates and 25 cm² flasks, followed by genotyping and cell banking.

For genotyping, genomic DNA was extracted from replicas of the cells cultured in 24-well plates using 50 μl QuickExtract solution (Cambridge Bioscience #QE0905T) and the following thermocycling conditions: 68° C. for 15 min, 95° C. for 10 min, 4° C. HOLD. The edited locus was amplified by standard PCR using GoTAQ G2 MasterMix (2×) (Promega #M7822) with 2 μl QuickExtract-processed genomic DNA and the following primers: F 5′-TCTACAGAGTTCCCTGTTTGC-3′; R 5′-AGCACTCAACTATATCTTGTCAGT-3′. Annealing and extension were performed at 55° C. for 30 sec and 72° C. for 30 sec, respectively. The PCR reactions were cleaned up with ExoSAP-IT Express (Thermo Fisher Scientific #75001.1.ML) according to the manufacturer's instructions, at 37° C. for 30 min followed by 80° C. for 1 min. The cleaned-up reactions were submitted for Sanger sequencing (Eurofins Genomics). Subsequent analyses of the Sanger sequencing traces were performed using Synthego's open-source ICE tool. Next, all predicted knock-out (KO) clones were validated by Western blotting for the PIK3CA protein using two complementary antibodies (CST #4249 and #4255; each used at 1:1000 dilution in 1×TBS/T with 3% BSA). Clones exhibiting complete loss of expression were kept for further experimental studies.

### Mass Spectrometry-Based Phosphoproteomics

PI3Kα-WT and PI3Kα-KO MEFs, grown in 15 cm dishes, were serum-starved overnight in DMEM with 1% penicillin-streptomycin and stimulated by the addition of 0.05% DMSO, 5 mM 1938 in final 0.05% DMSO or 100 nM insulin (Sigma, 15016) for 15 min or 4 h. Cells were lysed in 500 dl urea lysis buffer [50 mM triethylammonium bicarbonate, 8 M urea, cOmplete™, EDTA-free protease inhibitor cocktail (1:50 dilution) (Roche, 11873580001), 1 PhosSTOP tablet (Roche, 4906845001), 1 mM sodium orthovanadate] and lysates sonicated until clear for ˜10 min with cooling breaks on ice. Protein concentration was measured using a BCA protein assay (Pierce #23227). 300 μg of protein was reduced with 5 mM Tris(2-carboxyethyl)phosphine hydrochloride (Sigma, C4706) at 37° C. for 20 min and alkylated using 10 mM 2-chloroacetamide (Sigma, 22790) for 20 min at room temperature in the dark. Proteins were digested with LysC for 3.5 h at 30° C. Samples were then diluted with 50 mM triethylammonium bicarbonate (Sigma, T7408) to reduce the urea concentration to 1.5 M, followed by an overnight peptide digestion with trypsin at 37° C. Digest reactions were quenched by the addition of 10% trifluoroacetic acid (EMD Millipore 302031-M) to a final pH of 2.0. Sample desalting was performed using 35-350 μg C18 columns (HMM S18V; The Nest Group, Inc., Southborough, MA, USA) according to the manufacturer' specifications. TiO₂ (Hichrome Titansphere TiO₂, 10 μm capacity, 100 mg, GL Sciences #5020-75010) was used for phosphoenrichment. Following peptide loading onto TiO₂, the beads were sequentially washed with 1 M glycolic acid (Sigma #124737)/80% acetonitrile/5% trifluoroacetic acid, followed by 80% acetonitrile/0.2% trifluoroacetic acid and 20% acetonitrile before elution with 5% ammonium hydroxide. Enriched samples were desalted using 7-70 μg C18 columns (HUM S18V; The Nest Group, Inc., Southborough, MA, USA) according to the manufacturer's specifications. Dried phosphopeptide samples were stored at −80° C. and resuspended in 10% formic acid immediately prior to analysis. nLC-MS/MS was performed on a Q-Exactive Orbitrap Plus interfaced to a NANOSPRAY FLEX ion source and coupled to an Easy-nLC 1000 (Thermo Scientific). Fifty percent of each sample was analysed as 10 μl injections. Peptides were separated on a 27 cm fused silica emitter, 75 μm diameter, packed in-house with Reprosil-Pur 200 C18-AQ, 2.4 μm resin (Dr. Maisch, Ammerbuch-Entringen, Germany) using a linear gradient from 5% to 30% acetonitrile/0.1% formic acid over 180 min, at a flow rate of 250 nl/min. Peptides were ionised by electrospray ionisation using 1.9 kV applied immediately prior to the analytical column via a microtee built into the nanospray source with the ion transfer tube heated to 320° C. and the S-lens set to 60%. Precursor ions were measured in a data-dependent mode in the orbitrap analyser at a resolution of 70,000 and a target value of 3e6 ions. The ten most intense ions from each MS1 scan were isolated, fragmented in the HCD cell, and measured in the Orbitrap at a resolution of 17,500.

### Peptide Identification, Quantification and Statistical Analysis of Phosphoproteomics Data

Raw data were analysed with MaxQuant⁸⁴ (version 1.5.5.1) where they were searched against the mouse UniProt database (http://www.uniprot.org/, downloaded Apr. 12, 2018) using default settings. Carbamidomethylation of cysteines was set as fixed modification, and oxidation of methionines, acetylation at protein N-termini, phosphorylation (STY) were set as variable modifications. Enzyme specificity was set to trypsin with maximally 2 missed cleavages allowed. To ensure high confidence identifications, peptide-spectral matches, peptides, and proteins were filtered at a less than 1% false discovery rate (FDR). Label-free quantification in MaxQuant was used with a LFQ minimum ratio count of 2, Fast LFQ selected and the ‘skip normalisation’ option selected. The ‘match between runs’ feature was selected with a match time window of 0.7 min and an alignment time window of 20 min. The ‘phospho(STY)Sites.txt’ MaxQuant output file was processed with an in-house R script to obtain an ‘Annotated PhosphoSite.txt’ by merging each protein accession number with its corresponding phosphosite. This file, together with the ‘evidence.txt’ MaxQuant output file and an experimental design ‘annotation.csv’ file, was further processed by removing contaminants and reversed sequences, log 2 data transformation, and the removal of phosphosites with 0 or 1 valid values across all runs. High experimental reproducibility was observed, as evidenced by an average Pearson Correlation Coefficient of r=0.862 for biological replicates (FIG. 16/Extended Data FIG. 8d). Quantified phosphopeptides were analysed within the model-based statistical framework MSstats (version 3.20.0, run through RStudio (version 1.2.5042, R version 4.0.0)). Data were log 2 transformed, quantile normalised, and a linear mixed-effects model was fitted to the data. The group comparison function was employed to test for differential abundance between conditions. P-values were adjusted to control the FDR using the Benjamini-Hochberg procedure (Benjamini, Y. & Hochberg, J R Stat Soc B 57, 289-300 (1995)).

The mass spectrometry proteomics data have been deposited to the ProteomeXchange Consortium via the PRIDE24 partner repository with the dataset identifier PXD027993. Reviewer account details: Username: reviewer_pxd027993@ebi.ac.uk; Password: FSaiKH6M).

### Total Internal Fluorescence (TIRF) Microscopy of Phosphoinositide Reporters

TIRF microscopy which allows selective imaging of the small cell volume, including the plasma membrane, directly adjacent to the coverslip onto which cells have been seeded. HeLa or A549 cells were seeded in Matrigel-coated (Corning #354230; diluted in Opti-MEM at 1:50) 8-well chamber slides (glass bottom, 1.55 refractive index; Thermo Fisher Scientific #155409) at a density of 5,000 cells/well. The following day, cells were transfected with 50 ng (A549) or 10 ng (HeLa) PIP₃ reporter plasmid (GFP-PH-ARNO^{I303E}x2) (Goulden, B. D. et al. J Cell Biol 218, 1066-1079 (2019) using FuGENE® HD Transfection Reagent (Promega #E2311), at a 3:1 Fugene:DNA ratio according to the manufacturer's instructions. To ensure low yet uniform expression of the reporters in HeLa cells, and to aid in the identification of the critical TIRF angle for imaging, these cells were also co-transfected with 200 ng iRFP-tagged Paxillin plasmid (generated by conventional restriction enzyme-based subcloning from an mCherry-Paxillin plasmid, Addgene #50526). In separate experiments, HeLa cells were also transfected with 10 ng or 50 ng of the PI(3,4)P₂ reporter mCherry-cPH-TAPP1x3 (Goulden, B. D. et al. J Cell Biol 218, 1066-1079 (2019)); the use of 50 ng of this reporter enabled easier visualisation in the TIRF field, however the kinetics of the response remained unchanged and results from both experiments were pooled.

Following another 24 h post-transfection, cells were switched to 150 μl serum-free Fluorobrite™ DMEM (Thermo Fisher Scientific #A1896701; supplemented with L-glutamine (2 mM) and 1% penicillin-streptomycin for 3 h prior to time-lapse imaging on a 3i Spinning Disk Confocal microscope fitted with a sCMOS Prime95B (Photometric) sensor for TIRF, with full temperature (37° C.) and CO₂ (5%) control throughout the acquisitions. A 100×1.45 NA plan-apochromatic oil-immersion TIRF objective was used to deliver the laser illumination beam (40-50% power) at the critical angle for TIRF and for acquisition of the images by epifluorescence (300-500 msec exposure) using single bandpass filters (445/20 nm and 525/30 nm). Acquisition was performed in sequential mode, without binning, using Slidebook 6.0 and an acquisition rate of 2 or 3 min as indicated. Individual treatments were added at the specified times at 2× to 5× concentration in the same imaging medium, ensuring correct final concentration and sufficient mixing with the existing medium solution. BYL719 (Advanced ChemBlocks Inc #R16000) was used at at a high concentration of 0.5 μM (to achieve pan-class I PI3K inhibition).

Image analyses of total reporter intensities were performed with the Fiji open source image analysis package. The region of interest (ROI) corresponding to the footprint of the individual cell across time points were defined using minimal intensity projection to select only pixels present across all time points, following prior subtraction of camera noise (rolling ball method, radius=500 pixels) and xy drift correction, intensity levels over time were measured. These analyses were performed with a custom-written FIJI/ImageJ macro. A second macro was used to generate scaled images, with normalisation of all pixels to pre-treatment average intensity (Fₜ/F_{baseline}). All other quantifications were performed using the open source software R/RStudio. All macros and analysis scripts are provided via the Open Science Framework (https://osf.io/gzxfm/?view_onlv=8de666831f5b444087a0ab7c6cf3a636).

### CellTiter-Glo® Cell Assay

MEFs were seeded at 5000 cells per well in 96-well plates in DMEM supplemented with 10% FBS and 1% P/S, and allowed to attach overnight. The next day, cells were serum starved for 4 h prior to compound treatment in fresh serum-free DMEM. Compounds solubilised in DMSO were diluted 1:2 in a 12-point concentration response curve in DMSO. Intermediate plates were prepared by transferring 4 μl of compounds in DMSO into 96 μl of serum-free DMEM media. This was then used to treat cell plates by transferring 12.5 μl of solution from the intermediate plate into 87.5 μl of serum-free DMEM in the cell plates. Compound concentration response curves had a top concentration of 30 μM and a final well concentration of 0.5% DMSO. Cell plates were incubated for 24 h, 48 h or 72 h at 37° C., 5% CO₂, followed by determination of cell survival using the CellTiter-Glo® reagent according to manufacturer's instructions (Promega #G7571). Endpoint luminescence was measured using CLARIOstar (BMG). Compound data were analyzed using GraphPad Prism 8.

### Measurement of Cell Proliferation by Crystal Violet Staining

MEFs were seeded at 5000 cells per well in 96-well plates in DMEM supplemented with 10% FBS and 1% P/S, and allowed to attach overnight. The next day, cells were serum-starved for 5 h prior to compound addition in fresh serum-free DMEM. After different time points, cells were rapidly washed with distilled H₂O before fixed and stained in a solution of 0.5% crystal violet (Sigma-Aldrich cat #C0775) in 20% methanol (v:v) as described (Feoktistova, M. et al. Cold Spring Harb Protoc 2016, pdb prot087379 (2016). Briefly, after 20 min incubation at room temperature on a rocking platform, fixed and stained cells were washed 3 times with distilled H₂O and plates air-dried overnight. 200 μl methanol was next added to each well and the plates were incubated at room temperature for 20 min on a bench rocker, followed by measurement of optical density at 570 with a plate reader.

### Measurement of Cell Cycle Progression by Edu Staining

Click-IT EdU strategy was used according to manufacturer instructions (Sigma-Aldrich #BCK-FC488-50). Briefly, MEFs were seeded at 50,000 cells per well in 6-well plates in DMEM supplemented with 10% FBS and 1% P/S, and allowed to attach overnight. The next day, cells were serum-starved for 5 h prior to compound addition in fresh serum-free DMEM. At different time points, cells were pulsed for 3 h with 10 μM EdU, followed by collection by trypsinization and fixation with 3.7% FA in PBS for 15 min in the dark, washed in 3% BSA and permeabilized in 1× saponin-based permeabilization buffer for 20 min in the dark. EdU was then detected using the FAM-azide assay cocktail for 30 min in the dark. Cells were washed twice in 1× saponin-based permeabilization buffer followed by analysed with flow cytometer (Novocyte Advanteon flow cytometer, Agilent).

### Animals

Adult (>200 g) Wistar rats (Charles River, UK) were housed in groups of 4-5 per cage and maintained on a 14:10-h light/dark cycle with ad lib access to food and water. All experiments were conducted in accordance with the UK Animals (Scientific Procedures) Act (1986) and the European Communities Council Directives (86/609/EEC), with approval from the University College London Animal Welfare and Ethical Review Board.

### In Vivo Model of Ischaemia Reperfusion Injury in Mice

Male C57/BL6 mice weighing 25-30 g were used throughout. Animals received humane care in accordance with the United Kingdom Home Office Guide on the Operation of Animal (Scientific Procedures) Act 1986, Project Licence PPL70/15358.

Animals were anaesthetised with intraperitoneal (i.p.) sodium pentobarbital at a dose of 100 mg/kg. The mice were intubated by tracheotomy and ventilated with room air using a small animal ventilator (MinVent, Type 845, Hugo Sachs Elektronik, Harvard Apparatus). The mice were then placed on a heating pad and the rectal temperature monitored and maintained at ˜37° C. using a temperature controller. During the experiments, both ECG and heart rate were continuously recorded using a PowerLab (Adinstrument, USA). The chest was opened in the intercostal space between the 3^{rd} and 4^{th} ribs to expose the heart, and a suture was placed around the left anterior descending (LAD) coronary artery followed by a snare to allow the occlusion and opening of the LAD. The left external jugular vein was cannulated for drug administration.

By tightening the suture snare to occlude the LAD coronary artery, the heart were subjected to 40 min ischaemia, which was confirmed by both ST-segment elevation on the ECG and a change in heart colour. After 40 min, the snare was loosened and the heart allowed to reperfuse for the next 120 min. 15 min prior to reperfusion, 50 μl of DMSO vehicle or 10 mg/kg 1938 compound in DMSO, was slowly injected via the jugular vein. The person carrying out the experiment was blinded to the treatment groups.

After 120 min reperfusion, the chest was re-opened, the heart was removed and cannulated via the thoracic aorta, and blood within the heart was washed out with saline. The LAD coronary artery was then re-occluded with the suture that had been left loosely in place following ischaemia, and the hearts were injected with 2% Evans blue to delineate the area at risk. These hearts were then frozen at −80° C. for ˜10 min and subsequently cut into 5-6 slices of ˜0.5 mm thickness. The heart slices were incubated in triphenyltetrazolium chloride (10 mg/ml) solution at 37° C., pH 7.4 for ˜15 min to delineate viable (stained red) from the necrotic tissue (white regions). Slices were then transferred to 10% formalin solution and fixed overnight. The heart slices without right ventricular wall were then scanned using a Cannon digital scanner. The total area of myocardium, the non-ischaemic area (which is stained with Evans blue), and the infarct area (i.e. the white area) of each slice were measured using Image-J software. The “area at risk” was calculated by subtraction of the non-ischaemic area (blue area) from the whole slice area and expressed as “percentage of the left ventricle”, and “infarct size” calculated as infarct area as a percentage of the area at risk. 4 mice died during the experiment, before reperfusion (3 in DMSO group, 1 in 1938 group) and were excluded from analysis.

Analysis of tissue samples by Western blotting was performed as follows. 50 μl of DMSO vehicle or 10 mg/kg 1938 compound in DMSO, was injected via the jugular vein of anaesthetized and intubated mice as described above. After 15 min, the chest was opened and the heart removed and freeze-clamped in liquid nitrogen. Hearts were then homogenized in lysis buffer [100 mM Tris.HCl, 300 mM NaCl, 1% IGEPAL, pH 7.4 supplemented with protease inhibitors (78438; Thermo Fisher Scientific) and phosphatase inhibitors (78427; Thermo Fisher Scientific)], by disruption using a pestle and mortar and sonicated on ice 5 times for 3 sec. The supernatant was then collected and after the addition of NuPAGE™ LDS Sample Buffer (4×) (Thermo Fisher Scientific), samples were boiled and stored at −80° C. until SDS-polyacrylamide gel electrophoresis (SDS-PAGE) was performed. 20 μg of protein per well was loaded on a 10% NuPAGE Bis-Tris gel (Invitrogen), resolved by SDS-PAGE, and transferred to PVDF membranes (Millipore) for Western blot analysis. Membranes were incubated with primary antibodies in 5% BSA/TBS-0.1% Tween-20 overnight at 4° C., washed three times for 10 min with TBS-0.1% Tween then incubated with secondary antibodies in 5% BSA/TBS-0.1% Tween for 1 h, followed by washing three times for 10 min with TBS-0.1% Tween. Antibodies used were mouse monoclonal antibody to $-actin (Santa Cruz; sc-47778; used at 1:2000), mouse monoclonal antibody to total Akt (Cell Signaling Technology; CST2920; used at 1:1000) and rabbit antibodies from Cell Signaling Technology to phospho-Akt Thr308 (CST2965; used at 1:1000) or phospho-Akt Ser473 (CST9271; used at 1:1000). Secondary antibodies used were IRDye 680LT goat anti-mouse and IRDye 800CW goat anti-rabbit (LI-COR Biosciences). Proteins were visualized and quantified using the Odyssey Imaging System (LI-COR Biosciences).

### Quantification of Neurite Outgrowth

Dorsal root ganglion (DRG) neurons were isolated from adult male (>250 g) Wistar rats as described, with DRGs from each rat cultured separately (Rayner, M. L. D. et al. Anatomical record 301, 1628-1637 (2018)). Following culling via schedule 1 (rising concentration of CO₂), the spinal column was removed and stored in PBS on ice. Cord tissue was removed to expose the DRG and roots in the intervertebral foramen and the DRGS removed with forceps and scalpel under a dissecting microscope (Olympus SZ40). DRGs were manually cleaned by removal of roots, capsule and capillaries with forceps and then placed in DMEM supplemented with P/S. DRGs were treated with 0.125% collagenase type IV solution at 37° C. for 90 min, and then mechanically dissociated by trituration using a 1 ml pipette. The collagenase solution was removed by 2 rounds of centrifugation in complete DMEM (DMEM with 1% P/S and 10% FBS) at 400×g for 5 min, followed by resuspension of the DRG cell pellet in complete DMEM supplemented with 0.01 mM cytosine arabinoside. DRGs were plated in 75-cm² flasks coated with 0.1 mg/ml poly-D-lysine and incubated at 37° C., 5% CO₂. 24 h later, DRGs were resuspended by trypsinisation and the trypsin was removed by centrifugation at 190×g for 4 min. The resultant cell pellet was resuspended by mechanical trituration in Neurobasal-A medium (Gibco #10888022) supplemented with B-27 (Gibco #17504044), 2 mM L-Glutamine (Merck #G7513) and 1% penicillin/streptomycin. DRGs were plated onto 0.1 mg/ml poly-D-lysine-coated clear bottom black-walled 384-well plates (Greiner 781090) at a density of 1,000 cells/well. Cells were incubated at 37° C., 5% CO₂ for 24 h. Prior to treatment, cells were washed with supplemented Neurobasal-A medium using a BRAVO liquid handler (Agilent) to a uniform volume. 1938 solubilised at 3 mM in DMSO was diluted 1:3 in an 8-point concentration response curve in DMSO. Drugs in concentration response curves were diluted in supplemented Neurobasal-A medium by transfer into intermediate plates using a BRAVO liquid handler. Intermediate plates were then used to treat cell plates using the BRAVO liquid handler (final concentration of 0.1% DMSO in the DRG cultures). The PI3Kα inhibitor BYL-719 (final concentration of 500 nM in the DRG cultures) or vehicle (0.005% DMSO in supplemented Neurobasal-A medium; was added 15 min prior to the addition of the 1938 concentration response curve (total concentration of 0.105% DMSO in the DRG cultures). After incubation for 72 h at 37° C. and 5% CO₂, cells were fixed by addition of 4% paraformaldehyde for 20 min. Wells were washed 3 times in PBS with 0.05% Tween-20 (PBST) before permeabilisation in PBS with 0.1% Triton X-100. Wells were washed 3 more times with PBST before blocking with fish skin gelatin/PBST for 1 h at room temperature. The wells were then incubated overnight at 4° C. with primary antibody against the B—III tubulin neuronal marker; abcam #ab18207; 1:1000). The following day, cells were washed 3 times in PBST using the BRAVO liquid handler before incubation with anti-rabbit Alexafluor-488 (1:2000, A-11008) for 1 h at room temperature. Cells were washed 3 times with PBST using the BRAVO liquid handler before staining with Hoechst 33342 nucleic acid stain (Thermo Scientific #62249; 1:2000) for 20 min protected from light. Cells were washed another 3 times with PBST and 3 times with PBS and cell plates stored at 4° C. protected from light before imaging. Image acquisition was performed using Opera (PerkinElmer) high-content screening system using the 20× water objective. Images of cell nuclei and β-III tubulin-positive cells were captured using excitation/emission wavelengths λ380/455 and λ490/518, respectively. 9 fields per well were captured and analysed using the CSIRO Neurite Analysis 2 logarithm in Columbus analysis software (Perkin Elmer). Neurites were defined using the following parameters: Smoothing window 0 pixels (px), Linear window 15 px, Contrast >1.5, Diameter ≥3 px, Gap closure distance ≤17 px, Gap closure quality 0, Debarb length ≤40 px, Body thickening 1 px, Tree length ≤0 px. Within each experiment treatments were performed in quadruplicate and data are represented at the average of biological repeats (n=3) ±standard error of the mean. Variable slope nonlinear regression (4 parameters) was performed in Prism 7. Whole well representative images were captured using Cytation 3 (Biotek) imaging plate reader using a 10× objective. A montage of images were captured before stitching and deconvolution in Gen 5 software (Biotek). Images of cell nuclei and β-III tubulin-positive cells were captured using excitation/emission wavelengths λ380/455 and λ490/518, respectively.

### Control Experiments for Nerve Crush Assays

Experiments to test the stability of 1938 in aqueous solution and the biological activity of 1938 on exposed rat sciatic nerves were performed as follows.

Lyophilised 1938 was solubilised in autoclaved dH₂O to 100 μM. Solubilisation required sonication at 30° C. for 25 min before passing through a 0.22 μm filter. Aliquots of 1938 (at 5 μM and 100 μM) or vehicle were frozen at −20° C. in aliquots for later use on separate experimental days. An aliquot of 100 μM TRO-1938 and vehicle was defrosted and tested on A549 cells to test activity (FIG. 17/Extended Data FIG. 9, left panel). Cells were seeded in 24-well plates at 200,000 cells/well in DMEM+Glutamax supplemented with 10% FBS and 1% Pen/Strep. Prior to treatment, cells were washed and incubated with serum-free DMEM+Glutamax. Cells were treated with an 8 point 1:3 dose response of 1938 diluted in serum-free DMEM+Glutamax starting from 10 μM for 15 min at 37° C. Cells were then washed in ice-cold PBS and lysed in RIPA buffer supplemented with protease and phosphatase inhibitors. Lysates were analysed by automated Western blot (Wes) (data shown in FIG. 17/Extended Data FIG. 9; left panel).

Test experiment to assess if 1938 could induce pAkt generation in exposed rat sciatic nerves, adult male Sprague Dawley rats (>250 g; n=2) were anaesthetised using isoflurane, the left leg sciatic nerve was exposed and injected with 2 μl vehicle (sterile dH₂O) or 1938 (5 μM in sterile dH₂O). Meanwhile the sciatic nerve of the right leg was exposed and bathed in 250 μl of vehicle (sterile dH₂O) or 1938 (5 μM in sterile dH₂O). Each animal received one vehicle and one compound treatment. The treatments were left on for 30 min prior to washing the bathed nerves with sterile PBS and culling via sodium pentobarbital injection according to local regulations, the animal by schedule 1. Nerves were then harvested, washed in fresh 4° C. PBS and stored in a fresh vial before snap freezing in liquid nitrogen. Frozen sciatic nerves were homogenised in RIPA buffer supplemented with protease and phosphatase inhibitors using a mortar and pestle homogeniser. The subsequent crude lysates were centrifuged at 10,000×g for 10 min at 4° C., the supernatant harvested and stored at −80° C. prior to automated western blot (Wes) analysis for pAkt and controls (FIG. 17/Extended Data FIG. 9, right panel).

### Rat Sciatic Nerve Crush Injury and 1938 Treatment

Adult female Sprague Dawley rats (230-280 g, n=10, Charles River, UK) were anaesthetised by isoflurane inhalation in an induction chamber (5% isoflurane in O₂, 0.8 l/min). Anaesthesia was maintained with 1.5-2.5% isoflurane inhalation, and the left sciatic nerve exposed at mid-thigh level.

The nerve was crushed by application of constant pressure using fully closed sterile type 4 tweezers (TAAB) for 15 sec. This was repeated two more times at the same point, with 45° rotation between each crush. The injury site was marked with a 10/0 epineurial non-absorbant suture (Ethicon). Following injury, frozen aliquots of 1938 solution and vehicle were defrosted. A single 2 μl injection of 1938 solution (5 μM in sterile H₂O) or vehicle (sterile dH₂O) was administered proximal to the crush site with a 10 μl Hamilton syringe. An osmotic minipump (Alzet 1004, Charles River, UK) was also implanted between the muscle layers, adjacent to the nerve oriented with the outlet nearest to the crush site, loaded with 1938 solution (100 μM in sterile H₂O) or vehicle (sterile H₂O). Animals were randomly assigned to groups (n=5 per group) and one experimenter was kept blind to condition for conducting functional and histological analyses. Overlying muscle layers were closed using 4/0 sutures (Ethicon) and the skin was closed with wound clips (Clay Adams). Animals were left to recover for 21 days.

### Functional Assessment of Muscle Regeneration

At the end-point of the experiment (21 days), rats were anaesthetised and the sciatic nerve exposed as described above. A reference, ground (Natus) and recording electrode (Ambu Neuroline) were attached into the tail, above the hip bone, and into the tibialis anterior muscle respectively. A microchannel neurointerface (MNI) was placed approximately 2 mm proximal to the injury site and used to stimulate the nerve. The MI was manufactured using a previously documented protocol (Lancashire, H. T. et al. J Neural Eng 13, 034001 (2016). Electrode impedance of the MNI was 27.1±19.8 kΩ at 1k Hz. Compound muscle action potential (CMAP) was obtained by sciatic nerve stimulation with square wave pulses of 100 μsec with intensity from 1-10 mA. Stimulus was increased in 0.2 mA steps until muscle response amplitude no longer increased. CMAP amplitude was measured from peak to peak and recorded in triplicate for both the ipsilateral and contralateral side. The CMAP with the largest amplitude was selected for analysis.

A modified multipoint stimulation technique was used to calculate Motor Unit Number Estimation (MUNE) (Shefner, J. M. et al. Muscle & nerve 34, 603-607 (2006); Jacobsen, A. B. et al. J Vis Exp (2018); Arnold, W. D. et al. J Vis Exp (2015)). Incremental responses were obtained by delivering a submaximal stimulation of 100 μsec duration at a frequency of 1 Hz while increasing the stimulus intensity in increments of 0.02 mA to obtain minimal responses. The initial response was obtained with a stimulus intensity of between 0.21 mA and 0.70 mA. If the initial response did not occur between these stimulus intensities, the stimulating electrode was adjusted to increase or decrease the stimulus intensity as required. Additional Single Motor Unit Potentials (SMUPs) were evoked by stimulation in increments of 0.02 mA to obtain a minimum of four additional increments. The position of the stimulating electrode and the location of the recording electrode was changed to allow the recording of SMUPs from a different site of the muscle. This process was repeated at least three times. The CMAP was divided by the mean magnitude of SMUPs to quantify MUNE.

### Sciatic Nerve Collection and Processing

After electrophysiology recordings, animals were culled with sodium pentobarbital injection according to local regulations. Sciatic nerves, including the common peroneal branch, and tibialis anterior muscles were collected and placed in 4% paraformaldehyde (PFA). Muscles were transferred to phosphate buffered saline (PBS) after 15 min and stored at 4° C. until processing. Nerve samples were fixed overnight in 4% PFA at 4° C. before transferring to PBS. Nerve samples were divided into sciatic nerves including the crush site, and the common peroneal branch for sectioning. Nerve samples were immersed in 30% sucrose overnight at 4° C., then snap frozen in Neg-50 frozen section medium (Thermo Scientific) using liquid nitrogen cooled isopentane. Transverse sections (10 μm) were cut from the distal segment of the common peroneal nerve using a cryostat (HM535, Thermo Scientific). From the sciatic nerve, transverse cryosections (15 μm) were cut from 3 mm and 6 mm distal to the crush site. Sections were adhered to glass slides (Superfrost Plus, Thermo Fisher) for immunofluorescence staining.

For immunofluorescence staining, all washes and dilutions were performed using immunostaining buffer (PBS with 0.002% sodium azide and 0.3% Triton-X 100). Slides were heated to 37° C. for 20 min for antigen retrieval and then blocked with 5% normal horse serum for 40 min. Sections were then incubated in primary antibodies overnight at 4° C., followed by incubation for 45 min at room temperature in secondary antibodies. The following antibodies were used: mouse anti-neurofilament (Biolegend 835604, 1:500), goat anti-choline acetyltransferase (Millipore AB144P, 1:50), DyLight anti-mouse IgG 549 (Vector DI-2549, 1:300) and DyLight anti-goat IgG 488 (Vector DI-1488, 1:300). Slides were coverslipped with Vectashield Hardset mounting medium (Vector, H-1400).

Fluorescence microscopy (Zeiss AxiolabA1, Axiocam Cm1) was carried out for quantification of motor axons (ChAT) in the distal segment of the common peroneal nerve. For analysis of sciatic nerve sections at 3 mm and 6 mm distal to the crush injury, confocal tile scans (Zeiss LSM 710, 20× magnification) were taken of each transverse section. Quantification of all neurofilament-positive axons was performed using Volocity™ software (Perkin Elmer, Waltham, MA).

### Muscle Collection and Processing

Tibialis anterior muscles were fixed in 4% PFA for no longer than 15 min and then embedded in Optimal Cutting Temperature (OCT) and snap-frozen on liquid nitrogen-cooled isopentate or left in immunostaining buffer until ready to be processed. Transverse 20 μm cryosections were taken at 300 μm intervals. A minimum of 20 sections from each sample were obtained from the entire cross-section of muscle and adhered to glass slides for immunofluorescence staining.

All washes and dilutions were performed using immunostaining buffer (PBS containing 0.002% sodium azide and 0.3% Triton-X100). Slides were heated to 42° C. for 30 min with 20 μg/ml proteinase K and then blocked with 10% goat serum for 40 min at room temperature. After washing, the sections were incubated in primary antibody (neurofilament, Biolegend 835604, 1:500), washed, then incubated with DyLight anti-mouse IgG 488 (Vector DI-2488, 1:300) and alpha-bungarotoxin (Alexa 594 conjugate, ThermoFisher Scientific, 1:1000). Sections were mounted using Vectashield Hardset mounting medium.

Fluorescence microscopy (Zeiss AxiolabA1, Axiocam Cm1) was used to determine the proportion of motor endplates (α-bungarotoxin) co-stained with neurofilament to quantify the percentage of reinnervated motor endplates. For each sample, a minimum of 20 non-overlapping regions of the entire muscle cross-section were analysed.

For statistical analyses, data from 1938 and vehicle treated animals were compared by unpaired t=tests (Graphpad Prism 8.0.0).

### Statistical Methods

The statistical methods for the different types of experiments are included in each experimental section above.

### Compound Synthesis

### Experimental

Chemicals and solvents were from commonly used suppliers and were used without further purification. Chromatographic purifications were performed using prepacked SNAP columns using a Biotage Isolera Purification system (Uppsala, Sweden). Microwave assisted reactions were performed using a Biotage Initiator™ microwave synthesiser in sealed vials. Deuterated solvents were obtained from Sigma Aldrich.

NMR spectra were recorded using a Bruker 400 MHz or 500 MHz spectrometer. Chemical shifts are given in ppm relative to the solvent peak and coupling constants (J) are reported in Hz.

LCMS spectra were obtained using one of the following methods:

LCMS Method A: Waters LCMS system (Waters Micromass ZQ Mass Spectrometer attached to an Waters 2000 series HPLC). Analysis performed using a Gemini column (3.0 μM, NX—C18, 110 Å, 50×4.6 mm). Mobile phase A contained 0.1% formic acid in water and mobile phase B contained 0.10% formic acid in HPLC grade acetonitrile. A flow rate of 1.00 mL min-1 was used over a 5.0 min gradient starting with 99% mobile phase A gradually increasing to 100% mobile phase B. The samples were monitored at either 254 nm or 220 nm.

LCMS Method B: Agilent LCMS system (Agilent 6140 series Quadrupole Mass Spectrometer with a multimode source attached to an Agilent 1200 series HPLC). Analysis performed using a Kinetic column (2.6 μM, EVO, C18, 100 Å, 50×2.1 mm). Mobile phase A contained 0.1% formic acid in water and mobile phase B contained 0.1% formic acid in HPLC grade acetonitrile. A flow rate of 1.00 mL min⁻¹ was used over a 5.5 min gradient starting with 99% mobile phase A gradually increasing to 100% mobile phase B. The samples were monitored at either 254 nm or 220 nm.

LCMS Method C: Shimadzu LCMS 2020 system. Analysis performed using a Waters X-Bridge™ column (2.5 μM, MS C18, 100 Å, 50×3.0 mm). Mobile phase A contained 0.1% formic acid in water and mobile phase B contained 0.1% formic acid in HPLC grade acetonitrile. A flow rate of 1.00 mL min⁻¹ was used over a 4.0 min gradient starting with 99% mobile phase A gradually increasing to 100% mobile phase B. The samples were monitored at either 254 nm or 220 nm.

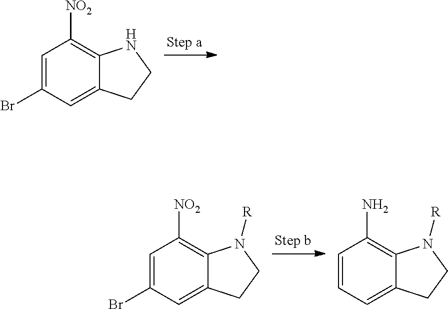

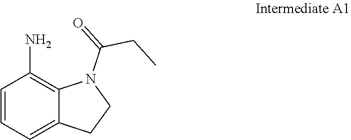

### 1-(7-Aminoindolin-1-yl)propan-1-one

Step a: To propanoyl chloride (0.72 mL, 8.23 mmol) in chloroform (10 mL), triethylamine (0.63 mL, 4.53 mmol) and 5-bromo-7-nitro-indoline (500 mg, 2.06 mmol) were added and the reaction mixture was stirred at rt for 18 h. The reaction mixture was diluted with water and extracted with DCM and the organics were washed with water and then brine before being dried (MgSO₄) and evaporated in vacuo. Purification on the Biotage Isolera gave 1-(5-bromo-7-nitroindolin-1-yl)propan-1-one (571 mg, 92% yield). ¹H NMR (CDCl₃, 500 MHz): 7.77 (s, 1H), 7.52 (s, 1H), 4.23 (t, J=8.1 Hz, 2H), 3.22 (t, J=8.1 Hz, 2H), 2.49 (q, J=7.4 Hz, 2H), 1.22 (t, J=7.4 Hz, 3H).

Step b: Ammonium formate (632 mg, 10.03 mmol) and Pd/C (11 mg, 0.10 mmol) were added to a stirred solution of 1-(5-bromo-7-nitro-indolin-1-yl)propan-1-one (300 mg, 1 mmol) in methanol (4 mL) and the reaction mixture was stirred at rt for 18 h. The reaction mixture was filtered through Celite and the filtrate was reduced in vacuo to give the title compound (176 mg, 92% yield). ¹H NMR (CDCl₃, 500 MHz): 6.97 (t, J=7.7 Hz, 1H), 6.66 (d, J=8.1 Hz, 1H), 6.62 (d, J=8.0 Hz, 1H), 4.79 (br s, 2H), 4.07 (t, J=7.8 Hz, 2H), 3.06 (t, J=7.8 Hz, 2H), 2.58 (q, J=7.4 Hz, 2H), 1.28 (t, J=7.5 Hz, 3H).

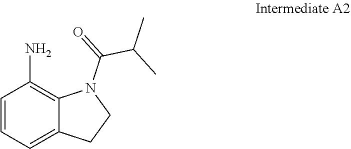

### 1-(7-Aminoindolin-1-yl)-2-methylpropan-1-one

Synthesised using the procedure described for Intermediate A1 using isobutyryl chloride in step a. ¹H NMR (CDCl₃, 400 MHz): 6.97 (t, J=7.4 Hz, 1H), 6.66 (dd, J=7.3 and 1.0 Hz, 1H), 6.60 (dd, J=8.0 and 1.0 Hz, 1H), 4.70 (br s, 2H), 4.12 (t, J=7.7 Hz, 2H), 3.06 (t, J=7.7 Hz, 2H), 2.94 (sept, J=7.0 Hz, 1H), 1.29 (s, 3H), 1.27 (s, 3H).

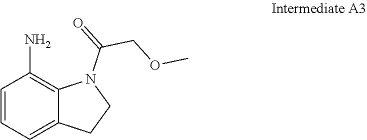

### 1-(7-Aminoindolin-1-yl)-2-methoxyethan-1-one

Synthesised using the procedure described for Intermediate A1 using methoxyacetyl chloride in step a. ¹H NMR (CDCl₃, 400 MHz): 6.97 (t, J=7.7 Hz, 1H), 6.65 (d, J=8.1 Hz, 1H), 6.58 (d, J=8.0 Hz, 1H), 4.26 (s, 2H), 4.03 (t, J=7.7 Hz, 2H), 3.50 (s, 3H), 3.05 (t, J=7.7 Hz, 2H).

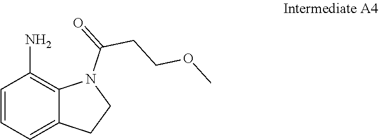 [SMILES: COCCC(=O)N1CCc2cccc(N)c21]

### 1-(7-Aminoindolin-1-yl)-3-methoxypropan-1-one

Synthesised using the procedure described for Intermediate A1 using 3-methoxypropanoyl chloride in step a. ¹H NMR (CDCl₃, 400 MHz): 6.95 (t, J=7.6 Hz, 1H), 6.64 (d, J=7.2 Hz, 1H), 6.58 (d, J=8.0 Hz, 1H), 4.11 (t, J=7.7 Hz, 2H), 3.79 (t, J=6.5 Hz, 2H), 3.38 (s, 3H), 3.03 (t, J=7.7 Hz, 2H), 2.81 (t, J=6.5 Hz, 2H).

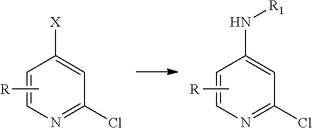

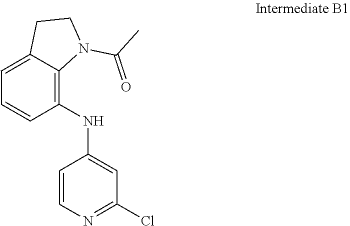 [SMILES: CC(=O)N1CCc2cccc(Nc3ccnc(Cl)c3)c21]

### 1-(7-((2-Chloropyridin-4-yl)amino)indolin-1-yl)ethan-1-one

A mixture of 2-chloro-4-iodopyridine (1 g, 4.2 mmol), 1-(7-aminoindolin-1-yl)ethanone (662 mg, 3.8 mmol), XANTPHOS (145 mg, 0.25 mmol), palladium acetate (38 mg, 0.17 mmol) and cesium carbonate (2.71 g, 8.35 mmol) in 1,4-dioxane (4 mL) was heated in the microwave at 80° C. for 1 h. After cooling to room temperature the mixture was filtered through Celite and washed with ethyl acetate. The filtrate was reduced in vacuo and then triturated with diethyl ether/petrol to give the title compound as a yellow solid (887 mg, 84%). ¹H NMR (CDCl₃, 400 MHz) δ 8.67 (s, 1H), 7.98 (d, J=5.7 Hz, 1H), 7.27 (d, J=8.1 Hz, 1H), 7.18 (t, J=7.7 Hz, 1H), 7.00 (dq, J=7.2, 1.1 Hz, 1H), 6.82 (d, J=2.1 Hz, 1H), 6.70 (dd, J=5.8, 2.1 Hz, 1H), 4.13 (t, J=7.7 Hz, 2H), 3.15 (t, J=7.8 Hz, 2H), 2.35 (s, 3H). LC-MS method A; RT 2.21; m/z [M+H]⁺ 288.0.

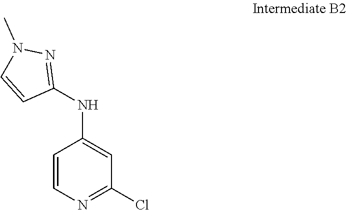

### 2-Chloro-N-(1-methyl-1H-pyrazol-3-yl)pyridin-4-amine

Synthesised using the procedure described for Intermediate B1 using 2-chloro-4-iodopyridine and 3-amino-1-methyl-1H-pyrazole. ¹H NMR (CDCl₃, 400 MHz) δ 8.05 (d, J=5.8 Hz, 1H), 7.29 (d, J=2.3 Hz, 1H), 7.10 (d, J=2.1 Hz, 1H), 6.87 (dd, J=5.8, 2.1 Hz, 1H), 6.58 (s, 1H),_5.98 (d, J=2.3 Hz, 1H), 3.85 (s, 3H).

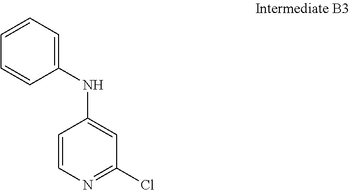 [SMILES: Clc1cc(Nc2ccccc2)ccn1]

### 2-Chloro-N-phenylpyridin-4-amine

Synthesised using the procedure described for Intermediate B1 using 2-chloro-4-iodopyridine and aniline. ¹H NMR (DMSO-d6, 400 MHz) δ 9.08 (s, 1H), 7.98 (d, J=5.8 Hz, 1H), 7.43-7.35 (m, 2H), 7.21 (dd, J=7.5, 1.1 Hz, 2H), 7.11 (td, J=7.3, 1.2 Hz, 1H), 6.85 (dd, J=5.8, 2.1 Hz, 1H), 6.82 (d, J=2.1 Hz, 1H).

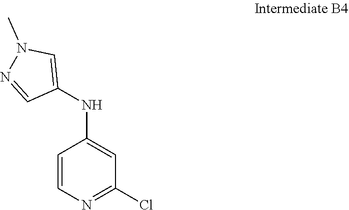

### 2-Chloro-N-(1-methyl-1H-pyrazol-4-yl)pyridin-4-amine

Synthesised using the procedure described for Intermediate B1 using 2-chloro-4-iodopyridine and 1-methylpyrazol-4-amine. ¹H NMR (CDCl₃, 400 MHz) δ 7.95 (d, J=5.8 Hz, 1H), 7.42 (s, 1H), 7.37 (s, 1H), 6.58 (d, J=2.2 Hz, 1H), 6.50 (dd, J=5.8, 2.0 Hz, 1H), 5.92 (s, 1H), 3.92 (s, 3H).

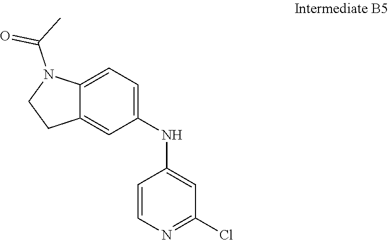 [SMILES: CC(=O)N1CCc2cc(Nc3ccnc(Cl)c3)ccc21]

### 1-(5-((2-Chloropyridin-4-yl)amino)indolin-1-yl)ethan-1-one

Synthesised using the procedure described for Intermediate B1 using 2-chloro-4-iodopyridine and 1-(5-aminoindolin-1-yl)ethanone. ¹H NMR (CDCl₃, 500 MHz) δ 8.24-8.20 (m, 1H), 8.00 (d, J=5.7 Hz, 1H), 7.05-6.96 (m, 3H), 6.90 (q, J=1.9 Hz, 1H), 6.70 (d, J=2.1 Hz, 1H), 6.60 (dd, J=5.8, 2.1 Hz, 1H), 4.11 (t, J=8.5 Hz, 2H), 3.23 (t, J=8.4 Hz, 2H), 2.25 (s, 3H).

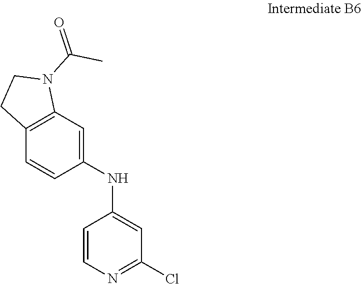 [SMILES: CC(=O)N1CCc2ccc(Nc3ccnc(Cl)c3)cc21]

### 1-(6-((2-Chloropyridin-4-yl)amino)indolin-1-yl)ethan-1-one

Synthesised using the procedure described for Intermediate B1 using 2-chloro-4-iodopyridine and 1-(6-aminoindolin-1-yl)ethanone. ¹H NMR (MeOD, 500 MHz) δ 8.04 (s, 1H), 7.88 (d, J=5.8 Hz, 1H), 7.23 (d, J=7.9 Hz, 1H), 6.89 (d, J=8.0 Hz, 1H), 6.82 (d, J=10.1 Hz, 2H), 4.18 (t, J=8.3 Hz, 2H), 3.19 (d, J=8.5 Hz, 2H), 2.24 (s, 3H).

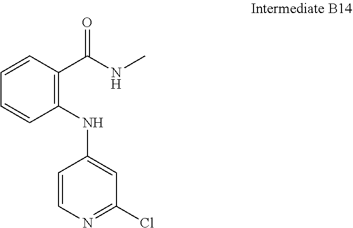 [SMILES: CNC(=O)c1ccccc1Nc1ccnc(Cl)c1]

### 2-((2-chloropyridin-4-yl)amino)-N-methylbenzamide

Synthesised using the procedure described for Intermediate B1 using 2-chloro-4-iodopyridine and 2-amino-N-methyl-benzamide. ¹H NMR (CDCl₃, 400 MHz) δ 9.67 (s, 1H), 8.10 (d, J=5.8 Hz, 1H), 7.54 (dd, J=8.2, 1.2 Hz, 1H), 7.50-7.44 (m, 2H), 7.08-7.02 (m, 2H), 6.87 (dd, J=5.7, 2.1 Hz, 1H), 6.22 (s, 1H), 3.01 (d, J=4.8 Hz, 3H).

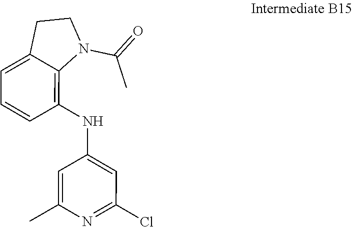 [SMILES: CC(=O)N1CCc2cccc(Nc3cc(C)nc(Cl)c3)c21]

### 1-(7-((2-Chloro-6-methylpyridin-4-yl)amino)indolin-1-yl)ethan-1-one

Synthesised using the procedure described for Intermediate B1 using 2,4-dichloro-6-picoline and 1-(7-aminoindolin-1-yl)ethanone. ¹H NMR (CDCl₃, 400 MHz) δ 9.00 (s, 1H), 7.75 (dd, J=8.2, 1.1 Hz, 1H), 7.14 (dd, J=8.2, 7.3 Hz, 1H), 6.90 (dq, J=7.4, 1.1 Hz, 1H), 6.62-6.58 (m, 1H), 6.56 (d, J=1.5 Hz, 1H), 4.09 (t, J=7.8 Hz, 2H), 3.13-3.06 (m, 2H), 2.39 (s, 3H), 2.36 (s, 3H).

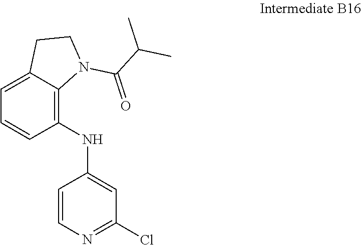 [SMILES: CC(C)C(=O)N1CCc2cccc(Nc3ccnc(Cl)c3)c21]

### 1-(7-((2-Chloropyridin-4-yl)amino)indolin-1-yl)-2-methylpropan-1-one

Synthesised using the procedure described for Intermediate B1 using 2-chloro-4-iodopyridine and Intermediate A2. ¹H NMR (CDCl₃, 400 MHz): 8.50 (s, 1H), 8.00 (d, J=5.8 Hz, 1H), 7.19 (t, J=7.4 Hz, 1H), 7.14 (d, J=7.5 Hz, 1H), 7.03 (dd, J=7.3 and 1.0 Hz, 1H), 6.82 (d, J=2.0 Hz, 1H), 6.70 (dd, J=5.8 and 2.1 Hz, 1H), 4.19 (t, J=7.7 Hz, 2H), 3.15 (t, J=7.7 Hz, 2H), 2.94 (sept, J=6.7 Hz, 1H), 1.29 (s, 3H), 1.27 (s, 3H).

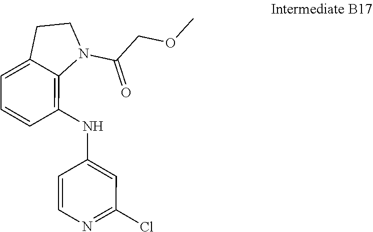 [SMILES: COCC(=O)N1CCc2cccc(Nc3ccnc(Cl)c3)c21]

### 1-(7-((2-Chloropyridin-4-yl)amino)indolin-1-yl)-2-methoxyethan-1-one

Synthesised using the procedure described for Intermediate B1 using 2-chloro-4-iodopyridine and Intermediate A3. ¹H NMR (CDCl₃, 400 MHz): 8.50 (s, 1H), 8.00 (d, J=5.8 Hz, 1H), 7.31 (d, J=7.6 Hz, 1H), 7.19 (t, J=7.4 Hz, 1H), 7.04 (d, J=6.2 Hz, 1H), 6.84 (d, J=2.0 Hz, 1H), 6.73 (dd, J=5.8 and 2.1 Hz, 1H), 4.30 (s, 2H), 4.14 (t, J=7.7 Hz, 2H), 3.50 (s, 3H), 3.17 (t, J=7.8 Hz, 2H)

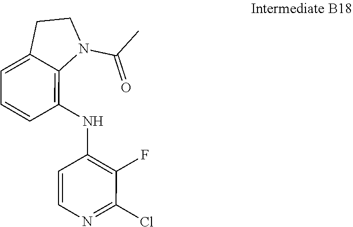 [SMILES: CC(=O)N1CCc2cccc(Nc3ccnc(Cl)c3F)c21]

### 1-(7-((2-Chloro-3-fluoropyridin-4-yl)amino)indolin-1-yl)ethan-1-one

Synthesised using the procedure described for Intermediate B1 using 2-chloro-3-fluoro-4-iodo-pyridine and 1-(7-aminoindolin-1-yl)ethanone. ¹H NMR (CDCl₃, 500 MHz): 9.04 (s, 1H), 7.82 (d, J=5.6 Hz, 1H), 7.27 (d, J=8.1 Hz, 1H), 7.20 (t, J=7.5 Hz, 1H), 7.04 (d, J=8.0 Hz, 1H), 6.97 (t, J=5.8 Hz, 1H), 4.16 (t, J=7.9 Hz, 2H), 3.18 (t, J=7.9 Hz, 2H), 2.38 (s, 3H).

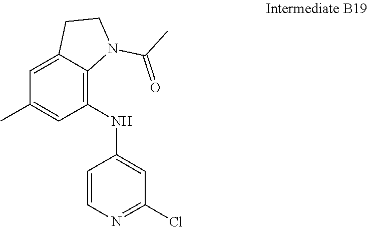 [SMILES: CC(=O)N1CCc2cc(C)cc(Nc3ccnc(Cl)c3)c21]

### 1-(7-((2-Chloropyridin-4-yl)amino)-5-methylindolin-1-yl)ethan-1-one

Synthesised using the procedure described for Intermediate B1 using 2-chloro-4-iodopyridine and 1-(7-amino-5-methyl-indolin-1-yl). ¹H NMR (CDCl₃, 500 MHz): 8.75 (s, 1H), 8.00 (d, J=5.8 Hz, 1H), 7.09 (s, 1H), 6.84-6.83 (m, 2H), 6.72 (dd, J=5.8 and 2.1 Hz, 1H), 4.12 (t, J=7.7 Hz, 2H), 3.11 (t, J=7.8 Hz, 2H), 2.36 (s, 3H), 2.35 (s, 3H).

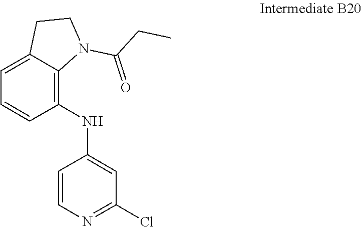 [SMILES: CCC(=O)N1CCc2cccc(Nc3ccnc(Cl)c3)c21]

### 1-(7-((2-Chloropyridin-4-yl)amino)indolin-1-yl)propan-1-one

Synthesised using the procedure described for Intermediate B1 using 2-chloro-4-iodopyridine (and Intermediate A1. ¹H NMR (CDCl₃, 500 MHz): 8.69 (s, 1H), 7.98 (d, J=5.8 Hz, 1H), 7.27 (d, J=5.6 Hz, 1H), 7.16 (t, J=7.5 Hz, 1H), 7.00 (d, J=7.3 Hz, 1H), 6.81 (d, J=1.9 Hz, 1H), 6.70 (dd, J=5.8 and 2.0 Hz, 1H), 4.12 (t, J=7.8 Hz, 2H), 3.13 (t, J=7.8 Hz, 2H), 2.58 (q, J=7.4 Hz, 2H), 1.27 (t, J=7.4 Hz, 3H).

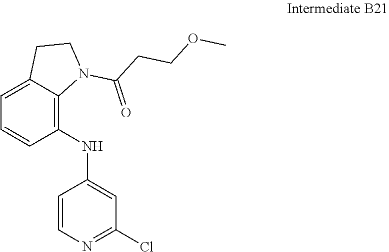 [SMILES: COCCC(=O)N1CCc2cccc(Nc3ccnc(Cl)c3)c21]

### 1-(7-((2-Chloropyridin-4-yl)amino)indolin-1-yl)-3-methoxypropan-1-one

Synthesised using the procedure described for Intermediate B1 using 2-chloro-4-iodopyridine and Intermediate A4. ¹H NMR (CDCl₃, 400 MHz): 8.55 (s, 1H), 8.00 (d, J=5.8 Hz, 1H), 7.29 (d, J=5.8 Hz, 1H), 7.20 (t, J=7.5 Hz, 1H), 7.03 (d, J=8.1 Hz, 1H), 6.83 (d, J=2.0 Hz, 1H), 6.71 (dd, J=5.8 and 2.1 Hz, 1H), 4.22 (t, J=7.8 Hz, 2H), 3.82 (t, J=6.2 Hz, 2H), 3.40 (s, 3H), 3.14 (t, J=7.7 Hz, 2H), 2.84 (t, J=6.2 Hz, 2H).

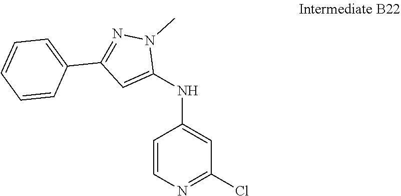 [SMILES: Cn1nc(-c2ccccc2)cc1Nc1ccnc(Cl)c1]

### 2-Chloro-N-(1-ethyl-3-phenyl-1H-pyrazol-5-yl)pyridin-4-amine

Synthesised using the procedure described for Intermediate B1 using 2-chloro-4-iodopyridine and 2-methyl-5-phenyl-pyrazol-3-amine. ¹H NMR (CDCl₃, 400 MHz): 8.07 (d, J=5.7 Hz, 1H), 7.77 (d, J=7.1 Hz, 2H), 7.41 (t, J=7.2 Hz, 2H), 7.33 (t, J=2.0 Hz, 1H), 6.54 (dd, J=5.7 and 2.2 Hz, 1H), 6.45 (s, 1H), 6.06 (br s, 1H), 3.76 (s, 3H).

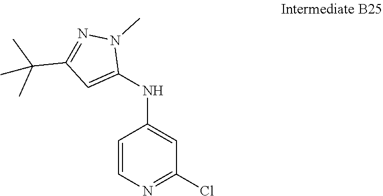 [SMILES: Cn1nc(C(C)(C)C)cc1Nc1ccnc(Cl)c1]

### N-(3-(tert-Butyl)-1-methyl-1H-pyrazol-5-yl)-2-chloropyridin-4-amine

Synthesised using the procedure described for Intermediate B1 using 2-chloro-4-iodopyridine and 5-tert-butyl-2-methyl-pyrazol-3-amine. ¹H NMR (CDCl₃, 400 MHz): 8.06 (d, J=5.7 Hz, 1H), 6.56 (d, J=2.1 Hz, 1H), 6.47 (d, J=2.2 Hz, 1H), 5.99 (s, 1H), 4.75 (s, 1H), 3.66 (s, 3H), 1.31 (s, 9H).

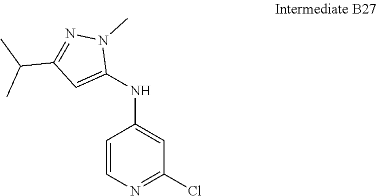 [SMILES: CC(C)c1cc(Nc2ccnc(Cl)c2)n(C)n1]

### 2-Chloro-N-(3-isopropyl-1-methyl-1H-pyrazol-5-yl)pyridin-4-amine

Synthesised using the procedure described for Intermediate B1 using 2-chloro-4-iodopyridine and 5-isopropyl-2-methyl-pyrazol-3-amine. ¹H NMR (CDCl₃, 400 MHz): 8.04 (d, J=5.7 Hz, 1H), 6.56 (d, J=2.0 Hz, 1H), 6.48 (dd, J=5.7 and 2.2 Hz, 1H), 5.95 (s, 1H), 3.65 (s, 3H), 2.94 (sept, J=6.9 Hz, 1H), 1.27 (s, 3H), 1.26 (s, 3H).

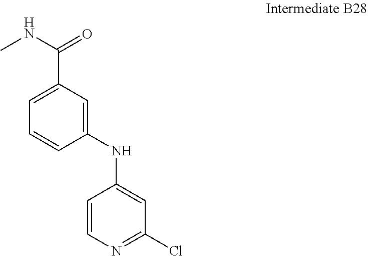 [SMILES: CNC(=O)c1cccc(Nc2ccnc(Cl)c2)c1]

### 3-((2-Chloropyridin-4-yl)amino)-N-methylbenzamide

Synthesised using the procedure described for Intermediate B1 using 2-chloro-4-iodopyridine and 3-amino-N-methylbenzamide. ¹H NMR (MeOD, 400 MHz): 7.96 (d, J=6.6 Hz, 1H), 7.69 (t, J=1.8 Hz, 1H), 7.59-7.57 (m, 1H), 7.50 (t, J=7.7 Hz, 1H), 7.42-7.39 (m, 1H), 6.93-6.91 (m, 2H), 2.95 (s, 3H).

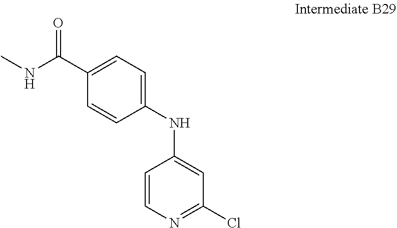 [SMILES: CNC(=O)c1ccc(Nc2ccnc(Cl)c2)cc1]

### 4-((2-Chloropyridin-4-yl)amino)-N-methylbenzamide

Synthesised using the procedure described for Intermediate B1 using 2-chloro-4-iodopyridine and 4-amino-N-methylbenzamide. ¹H NMR (MeOD, 400 MHz): 8.01 (d, J=5.8 Hz, 1H), 7.86 (d, J=8.8 Hz, 2H), 7.30 (d, J=8.8 Hz, 2H), 7.02-6.99 (m, 2H), 2.95 (s, 3H).

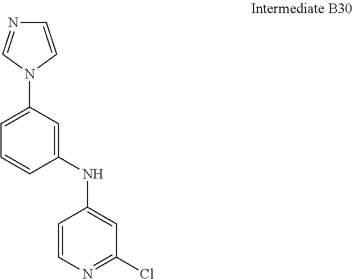 [SMILES: Clc1cc(Nc2cccc(-n3ccnc3)c2)ccn1]

### N-(3-(1H-Imidazol-1-yl)phenyl)-2-chloropyridin-4-amine

Synthesised using the procedure described for Intermediate B1 using 2-chloro-4-iodopyridine and 3-imidazol-1-ylaniline. ¹H NMR (MeOD, 400 MHz): 8.15 (s, 1H), 7.98 (d, J=6.4 Hz, 1H), 7.58 (s, 1H), 7.54 (t, J=8.1 Hz, 1H), 7.38 (t, J=2.0 Hz, 1H), 7.34-7.32 (m, 1H), 7.31-7.28 (m, 1H), 7.16 (s, 1H), 6.97-6.95 (m, 2H).

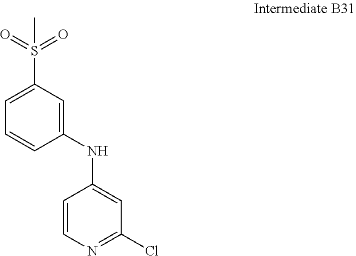 [SMILES: CS(=O)(=O)c1cccc(Nc2ccnc(Cl)c2)c1]

### 2-Chloro-N-(3-(methylsulfonyl)phenyl)pyridin-4-amine

Synthesised using the procedure described for Intermediate B1 using 2-chloro-4-iodopyridine and 3-methylsulfonylaniline. ¹H NMR (MeOD, 400 MHz): 8.01 (d, J=6.6 Hz, 1H), 7.75 (t, J=1.7 Hz, 1H), 7.69-7.67 (m, 1H), 7.64 (t, J=7.7 Hz, 1H), 7.58-7.56 (m, 1H), 6.96-6.95 (m, 2H), 3.15 (s, 3H).

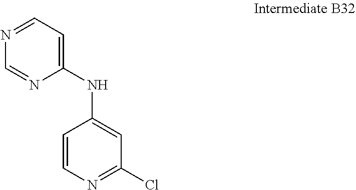 [SMILES: Clc1cc(Nc2ccncn2)ccn1]

### N-(2-Chloropyridin-4-yl)pyrimidin-4-amine

Synthesised using the procedure described for Intermediate B1 using 2-chloro-4-iodopyridine and pyrimidin-4-amine. ¹H NMR (MeOD, 400 MHz): 8.78 (d, J=0.6 Hz, 1H), 8.39 (d, J=6.0 Hz, 1H), 8.16 (d, J=5.8 Hz, 1H), 8.08 (d, J=1.9 Hz, 1H), 7.62 (dd, J=5.8 and 2.0 Hz, 1H), 6.91 (dd, J=6.0 and 1.2 Hz, 1H).

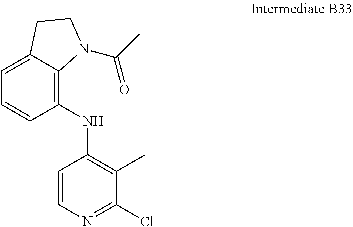 [SMILES: CC(=O)N1CCc2cccc(Nc3ccnc(Cl)c3C)c21]

### 1-(7-((2-Chloro-3-methylpyridin-4-yl)amino)indolin-1-yl)ethan-1-one

Synthesised using the procedure described for Intermediate B1 using 2-chloro-4-iodo-3-methyl-pyridine and 1-(7-aminoindolin-1-yl)ethanone. ¹H NMR (CDCl₃, 400 MHz): 8.65 (s, 1H), 7.87 (d, J=5.7 Hz, 1H), 7.23 (d, J=8.1 Hz, 1H), 7.17 (t, J=7.2 Hz, 1H), 7.00 (dd, J=7.2 and 1.1 Hz, 1H), 6.84 (d, J=5.7 Hz, 1H), 4.14 (t, J=7.8 Hz, 2H), 3.16 (t, J=7.8 Hz, 2H), 2.42 (s, 3H), 2.36 (s, 3H).

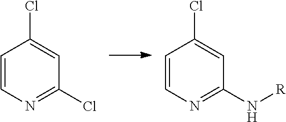

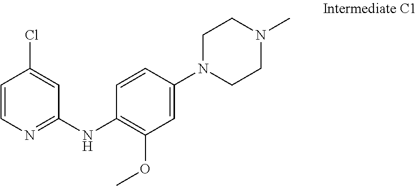 [SMILES: COc1cc(N2CCN(C)CC2)ccc1Nc1cc(Cl)ccn1]

### 4-chloro-N-(2-methoxy-4-(4-methylpiperazin-1-yl)phenyl)pyridin-2-amine

2-Methoxy-4-(4-methylpiperazin-1-yl)aniline (0.60 g, 2.71 mmol), Pd(OAc)₂ (12 mg, 0.054 mmol), cesium carbonate (1.76 g, 5.41 mmol) and XANTPHOS (47 mg, 0.08 mmol) were placed in a 20 mL microwave vial which was evacuated and back filled with argon. A solution of 2,4-dichloropyridine (0.8 g, 5.42 mmol) in 1,4-dioxane (12 mL) was added. The resulting suspension was heated in the microwave at 100° C. for 2 h. The reaction mixture was then poured into water (100 mL) and extracted with DCM (3×50 mL). The combined organics were washed with brine (50 mL), separated, dried (MgSO₄) and reduced in vacuo. Purification on the Biotage Isolera gave the title compound as an off-white solid (0.95 g, 63%). ¹H NMR (CDCl₃, 400 MHz) δ 8.05 (d, J=5.4 Hz, 1H), 7.63-7.56 (m, 1H), 6.72 (s, 1H), 6.69-6.64 (m, 2H), 6.55 (dd, J=6.7, 2.6 Hz, 2H), 3.85 (s, 3H), 3.28-3.20 (m, 4H), 2.76-2.69 (m, 4H), 2.44 (s, 3H). LCMS method A; RT 0.75; m/z [M+H]⁺ 333.2.

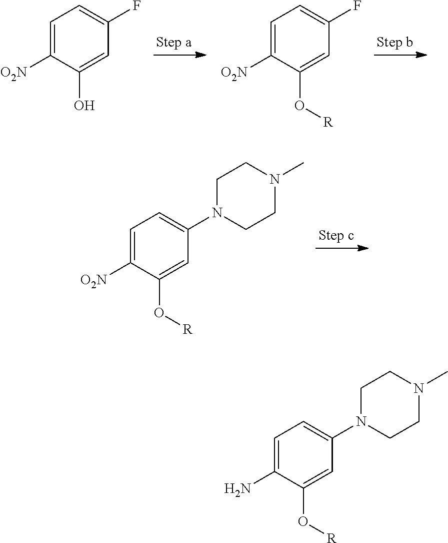

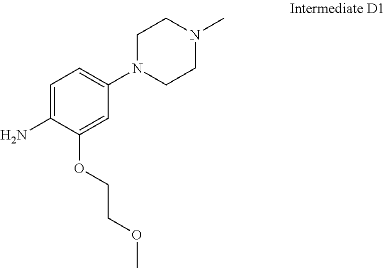 [SMILES: COCCOc1cc(N2CCN(C)CC2)ccc1N]

### 2-(2-Methoxyethoxy)-4-(4-methylpiperazin-1-yl)aniline

Step a: To a stirred solution of 2-bromoethyl methyl ether (0.3 mL, 3.18 mmol) and potassium carbonate (880 mg, 6.37 mmol) in DMF (6 mL), 5-fluoro-2-nitro-phenol (500 mg, 3.18 mmol) was added and the reaction mixture was stirred at rt for 18 h. The reaction mixture was then diluted with water and the resulting precipitate was collected by vacuum filtration to afford 4-fluoro-2-(2-methoxyethoxy)-1-nitro-benzene (210 mg, 31% yield). ¹H NMR (CDCl₃, 400 MHz): 7.96 (dd, J=9.1 and 6.0 Hz, 1H), 6.85 (dd, J=10.3 and 2.5 Hz, 1H), 6.78-6.73 (m, 1H), 4.26 (t, J=4.7 Hz, 2H), 3.83 (t, J=4.7 Hz, 2H), 3.48 (s, 3H)

Step b: To a stirred solution of potassium carbonate (270 mg, 1.95 mmol) and 1-methylpiperazine (0.11 mL, 0.98 mmol) in DMF (5 mL) was added 4-fluoro-2-(2-methoxyethoxy)-1-nitro-benzene (210 mg, 0.98 mmol) and the reaction mixture was stirred at rt for 18 h. The reaction mixture was then diluted with water and the resulting precipitate was collected by vacuum filtration to afford 1-[3-(2-methoxyethoxy)-4-nitro-phenyl]-4-methyl-piperazine (253 mg, 88% yield). ¹H NMR (CDCl₃, 400 MHz): 7.99 (d, J=9.3 Hz, 1H), 6.44 (dd, J=9.3 and 2.6 Hz, 1H), 6.42 (d, J=2.5 Hz, 1H), 4.25 (t, J=4.8 Hz, 2H), 3.84 (t, J=5.0 Hz, 2H), 3.50 (s, 3H), 3.41 (t, J=5.1 Hz, 4H), 2.56 (t, J=5.2 Hz, 4H), 2.37 (s, 3H).

Step c: Ammonium formate (270 mg, 4.28 mmol) and palladium on carbon (9 mg, 0.09 mmol) were added to a stirred solution of 1-[3-(2-methoxyethoxy)-4-nitro-phenyl]-4-methyl-piperazine (253 mg, 0.86 mmol) in methanol (5 mL) and the reaction mixture was stirred at rt for 18 h. The reaction mixture was then filtered through Celite and the filtrate was reduced in vacuo to give the title compound (165 mg, 73% yield). ¹H NMR (CDCl₃, 400 MHz): 6.65 (d, J=8.4 Hz, 1H), 6.54 (d, J=2.5 Hz, 2H), 6.54 (dd, J=8.4 and 2.5 Hz, 1H), 4.13 (t, J=4.7 Hz, 2H), 3.74 (t, J=4.8 Hz, 2H), 3.60 (br s, 2H), 3.07 (t, J=4.9 Hz, 4H), 2.58 (t, J=5.0 Hz, 4H), 2.35 (s, 3H).

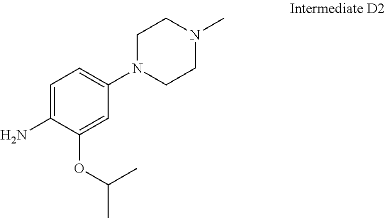 [SMILES: CC(C)Oc1cc(N2CCN(C)CC2)ccc1N]

### 2-Isopropoxy-4-(4-methylpiperazin-1-yl)aniline

Synthesised using the procedure described for Intermediate D1 using 2-iodopropane in step a. ¹H NMR (CDCl₃, 400 MHz): 6.65 (d, J=8.4 Hz, 1H), 6.52 (d, J=2.5 Hz, 1H), 6.42 (dd, J=8.4 and 2.5 Hz, 1H), 4.50 (sept, J=6.1 Hz, 1H), 3.06 (t, J=4.9 Hz, 4H), 2.58 (t, J=5.0 Hz, 4H), 2.34 (s, 3H), 1.35 (s, 3H), 1.33 (s, 3H).

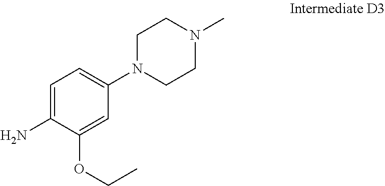 [SMILES: CCOc1cc(N2CCN(C)CC2)ccc1N]

### 2-Ethoxy-4-(4-methylpiperazin-1-yl)aniline

Synthesised using the procedure described for Intermediate D1 using 2-iodoethane in step a. ¹H NMR (CDCl₃, 400 MHz): 6.64 (d, J=8.4 Hz, 1H), 6.51 (d, J=2.4 Hz, 1H), 6.41 (dd, J=8.4 and 2.5 Hz, 1H), 4.04 (qu, J=7.0 Hz, 2H), 3.54 (br s, 2H), 3.07 (t, J=4.9 Hz, 4H), 2.58 (t, J=5.0 Hz, 4H), 2.35 (s, 3H), 1.42 (t, J=7.0 Hz, 3H).

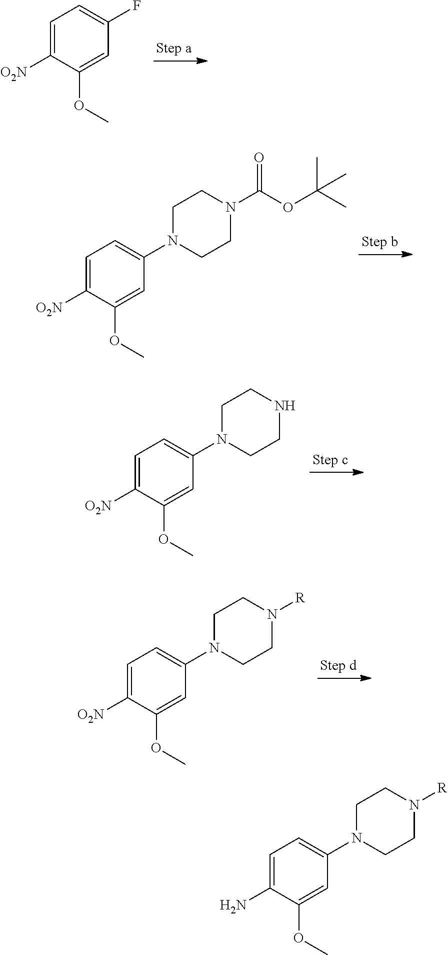

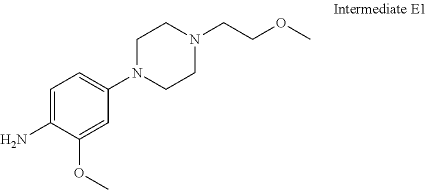 [SMILES: COCCN1CCN(c2ccc(N)c(OC)c2)CC1]

### 2-Methoxy-4-(4-(2-methoxyethyl)piperazin-1-yl)aniline

Step a: To a stirred solution of potassium carbonate (808 mg, 5.84 mmol) and 1-Boc-piperazine (544 mg, 2.92 mmol) in DMF (5 mL) was added 4-fluoro-2-methoxy-1-nitro-benzene (500 mg, 2.92 mmol) and the reaction mixture was stirred at rt for 18 h. The reaction mixture was then diluted with water and the resulting precipitate was collected by vacuum filtration to give tert-butyl 4-(3-methoxy-4-nitro-phenyl)piperazine-1-carboxylate (222 mg, 23% yield). ¹H NMR (CDCl₃, 500 MHz): 8.04 (d, J=9.3 Hz, 1H), 6.44 (dd, J=9.4 and 2.5 Hz, 1H), 6.34 (d, J=2.4 Hz, 1H), 3.98 (s, 3H), 3.63 (t, J=5.0 Hz, 4H), 3.42 (t, J=5.4 Hz, 4H), 1.52 (s, 9H).

Step b: To a solution of tert-butyl 4-(3-methoxy-4-nitro-phenyl)piperazine-1-carboxylate (222 mg, 0.66 mmol) in DCM (10 mL) was added HCL (4M in dioxane) (1 mL, 0.66 mmol) and the reaction mixture was stirred at rt for 18 h. The reaction mixture was then filtered to give 1-(3-methoxy-4-nitro-phenyl)piperazine hydrochloride (178 mg, 0.7234 mmol, 99% yield). ¹H NMR (MeOD, 400 MHz): 7.94 (d, J=9.0 Hz, 1H), 6.67-6.63 (m, 2H), 3.96 (s, 3H), 3.70-3.68 (m, 4H), 3.39-3.37 (m, 4H).

Step c: To a stirred solution of 2-bromoethyl methyl ether (0.08 mL, 0.83 mmol) and potassium carbonate (231 mg, 1.67 mmol) in DMF (3 mL) was added 1-(3-methoxy-4-nitro-phenyl)piperazine (198 mg, 0.83 mmol) and the reaction mixture was stirred at rt for 18 h. The reaction mixture was then diluted with EtOAc and organics were washed with water and brine before being dried (MgSO₄) and reduced in vacuo. The residue was purified by flash silica chromatography to give 1-(2-methoxyethyl)-4-(3-methoxy-4-nitro-phenyl)piperazine (165 mg, 67% yield). ¹H NMR (CDCl₃, 400 MHz): 8.02 (d, J=9.4 Hz, 1H), 6.44 (dd, J=9.4 and 2.6 Hz, 1H), 6.33 (d, J=2.5 Hz, 1H), 3.97 (s, 3H), 3.58 (t, J=5.4 Hz, 2H), 3.45 (t, J=5.1 Hz, 4H), 3.40 (s, 3H), 2.69-2.65 (m, 6H).

Step d: Ammonium formate (176 mg, 2.79 mmol) and palladium on carbon (6 mg, 0.06 mmol) were added to a stirred solution of 1-(2-methoxyethyl)-4-(3-methoxy-4-nitro-phenyl)piperazine (165 mg, 0.56 mmol) in methanol (10 mL) and the reaction mixture was stirred at rt for 18 h. The reaction mixture was then filtered through Celite and the filtrate was reduced in vacuo to give 2-methoxy-4-[4-(2-methoxyethyl)piperazin-1-yl]aniline (107 mg, 72% yield). ¹H NMR (CDCl₃, 400 MHz): 6.64 (d, J=8.3 Hz, 1H), 6.49 (d, J=2.4 Hz, 1H), 6.41 (dd, J=8.3 and 2.5 Hz, 1H), 3.83 (s, 3H), 3.69 (t, J=5.0 Hz, 2H), 3.36 (s, 3H), 3.21 (t, J=4.6 Hz, 4H), 3.07 (t, J=4.7 Hz, 4H), 2.60 (t, J=5.2 Hz, 2H).

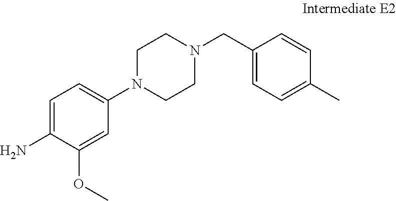 [SMILES: COc1cc(N2CCN(Cc3ccc(C)cc3)CC2)ccc1N]

### 2-Methoxy-4-(4-(4-methylbenzyl)piperazin-1-yl)aniline

Synthesised using the procedure described for Intermediate E1 using 1-(bromomethyl)-4-methylbenzene in step c. ¹H NMR (CDCl₃, 400 MHz): 7.25 (d, J=7.9 Hz, 2H), 7.16 (d, J=7.8 Hz, 2H), 6.65 (d, J=8.3 Hz, 2H), 6.53 (d, J=2.4 Hz, 1H), 6.43 (dd, J=8.4 and 2.5 Hz, 2H), 3.85 (s, 2H), 3.56 (s, 3H), 3.08 (t, J=4.8 Hz, 4H), 2.63 (t, J=5.0 Hz, 4H), 2.37 (s, 3H).

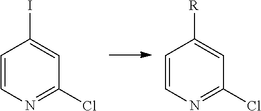

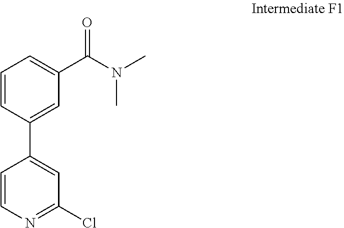 [SMILES: CN(C)C(=O)c1cccc(-c2ccnc(Cl)c2)c1]

### 3-(2-Chloropyridin-4-yl)-N,N-dimethylbenzamide

A mixture of 2-chloro-4-iodopyridine (200 mg, 0.84 mmol), N,N-dimethylbenzamide-3-boronic acid (0.15 mL, 0.75 mmol), bis(triphenylphosphine)palladium(II) dichloride (29 mg, 0.04 mmol) and sodium carbonate (2M aqueous solution) (1.67 mL, 3.34 mmol) in MeCN (3 mL) was heated in the microwave at 120° C. for 45 min. After cooling to rt, the mixture was diluted with ethyl acetate (20 mL) and washed with water (20 mL). The organics were separated, dried, reduced in vacuo and purified on the Biotage Isolera to give the title compound (128 mg, 59% yield). ¹H NMR (CDCl₃, 400 MHz) δ 8.43 (d, J=5.2 Hz, 1H), 7.69-7.61 (m, 2H), 7.56-7.47 (m, 3H), 7.43 (dd, J=5.1, 1.7 Hz, 1H), 3.14 (s, 3H), 3.01 (s, 3H).

### Example 1 (1902)

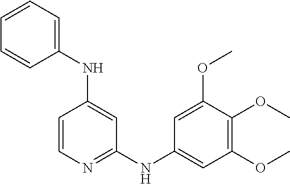 [SMILES: COc1cc(Nc2cc(Nc3ccccc3)ccn2)cc(OC)c1OC]

### N4-phenyl-N2-(3,4,5-trimethoxyphenyl)pyridine-2,4-diamine

A mixture of Intermediate B3 (72 mg, 0.35 mmol), 3,4,5-trimethoxyaniline (64 mg, 0.35 mmol), XANTPHOS (12 mg, 0.02 mmol), palladium acetate (3 mg, 0.01 mmol) and cesium carbonate (226 mg, 0.70 mmol) in DMA (4 mL) was heated in the microwave at 150° C. for 1 h. After cooling to rt the mixture was diluted with ethyl acetate (10 mL) and washed with water (10 mL) and brine (3×10 mL). The organics were separated, dried, reduced in vacuo and purified on the Biotage Isolera to give the title compound. ¹H NMR (MeOD, 500 MHz) a 7.75 (d, J=6.0 Hz, 1H), 7.35-7.28 (m, 2H), 7.21-7.16 (m, 2H), 7.04 (tt, J=7.4, 1.2 Hz, 1H), 6.67 (s, 2H), 6.48 (d, J=2.1 Hz, 1H), 6.38 (dd, J=6.0, 2.1 Hz, 1H), 3.79 (s, 6H), 3.71 (s, 3H). LCMS method A; RT 1.66; m/z [M+H]⁺ 352.1.

### Example 2 (1903)

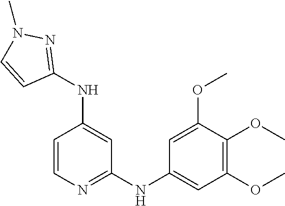 [SMILES: COc1cc(Nc2cc(Nc3ccn(C)n3)ccn2)cc(OC)c1OC]

### N4-(1-methyl-1H-pyrazol-3-yl)-N2-(3,4,5-trimethoxyphenyl)pyridine-2,4-diamine

The title compound was synthesised using the procedure described for Example 1 using Intermediate B2 and 3,4,5-trimethoxyaniline. ¹H NMR (MeOD, 400 MHz) a 7.74 (d, J=6.0 Hz, 1H), 7.44 (d, J=2.3 Hz, 1H), 6.94 (d, J=2.0 Hz, 1H), 6.69 (s, 2H), 6.49 (dd, J=6.0, 2.1 Hz, 1H), 5.92 (d, J=2.3 Hz, 1H), 3.82 (s, 6H), 3.78 (s, 3H), 3.73 (s, 3H). LCMS method A; RT 1.41; m/z [M+H]⁺ 356.1.

### Example 3 (1904)

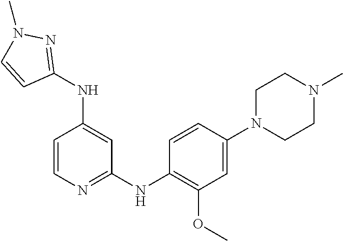 [SMILES: COc1cc(N2CCN(C)CC2)ccc1Nc1cc(Nc2ccn(C)n2)ccn1]

### N2-(2-methoxy-4-(4-methylpiperazin-1-yl)phenyl)-N4-(1-methyl-1H-pyrazol-3-yl)pyridine-2,4-diamine

A mixture of Intermediate B2 (73 mg, 0.35 mmol), 2-methoxy-4-(4-methylpiperazin-1-yl)aniline (77 mg, 0.35 mmol), XANTPHOS (12 mg, 0.02 mmol), palladium acetate (3 mg, 0.01 mmol) and cesium carbonate (226 mg, 0.70 mmol) in DMA (4 mL) was heated in the microwave at 150° C. for 1 h. After cooling to room temperature, the mixture was filtered through an SCX cartridge washing first with DCM and then with ammonia in methanol (7M). The ammonia layer was reduced in vacuo and purified on the Biotage Isolera to give the title compound. ¹H NMR (MeOD, 400 MHz) δ 7.57 (d, J=6.6 Hz, 1H), 7.46 (d, J=2.3 Hz, 1H), 7.24 (d, J=8.6 Hz, 2H), 6.92 (d, J=2.1 Hz, 1H), 6.70 (d, J=2.5 Hz, 1H), 6.60 (dd, J=8.6, 2.6 Hz, 1H), 6.53 (dd, J=6.7, 2.2 Hz, 1H), 3.84 (s, 3H), 3.79 (s, 3H), 3.24 (t, J=5.1 Hz, 4H), 2.64 (t, J=5.1 Hz, 4H), 2.37 (s, 3H). LCMS Method A; RT 0.84; m/z [M+H]⁺ 394.2.

### Example 4 (1905)

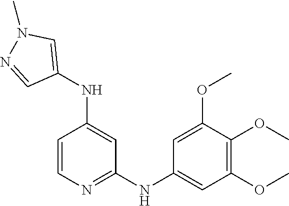 [SMILES: COc1cc(Nc2cc(Nc3cnn(C)c3)ccn2)cc(OC)c1OC]

### N4-(1-methyl-1H-pyrazol-4-yl)-N2-(3,4,5-trimethoxyphenyl)pyridine-2,4-diamine

Synthesised using the procedure described in Example 1 using Intermediate B4 and 3,4,5-trimethoxyaniline. ¹H NMR (MeOD, 400 MHz) δ 7.20 (d, J=6.8 Hz, 2H), 7.00 (d, J=0.9 Hz, 1H), 6.19 (s, 2H), 5.88 (dd, J=6.4, 2.2 Hz, 1H), 5.78 (d, J=2.2 Hz, 1H), 3.45 (s, 3H), 3.38 (s, 6H), 3.31 (s, 3H). LCMS Method A; RT 1.25; m/z [M+H]⁺ 356.1.

### Example 5 (1906)

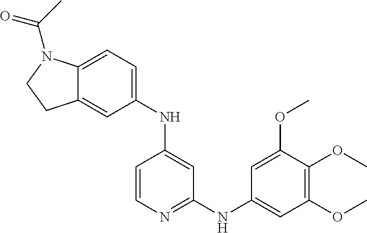 [SMILES: COc1cc(Nc2cc(Nc3ccc4c(c3)CCN4C(C)=O)ccn2)cc(OC)c1OC]

### 1-(5-((2-((3,4,5-trimethoxyphenyl)amino)pyridin-4-yl)amino)indolin-1-yl)ethan-1-one

Synthesised using the procedure described in Example 1 using Intermediate B5 and 3,4,5-trimethoxy aniline. ¹H NMR (CDCl₃, 400 MHz) δ 8.15 (d, J=8.3 Hz, 1H), 7.71 (d, J=6.2 Hz, 1H), 6.99-6.96 (m, 3H), 6.48 (s, 3H), 6.30 (dd, J=6.4, 2.2 Hz, 1H), 6.25 (d, J=2.1 Hz, 1H), 4.07 (t, J=8.4 Hz, 2H), 3.81 (s, 3H), 3.79 (s, 6H), 3.16 (t, J=8.4 Hz, 2H), 2.22 (s, 3H). LCMS Method A; RT 1.62; m/z [M+H]435.1.

### Example 6 (1907)

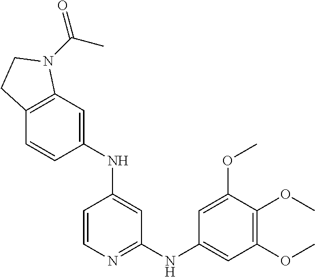 [SMILES: COc1cc(Nc2cc(Nc3ccc4c(c3)N(C(C)=O)CC4)ccn2)cc(OC)c1OC]

### 1-(6-((2-((3,4,5-trimethoxyphenyl)amino)pyridin-4-yl)amino)indolin-1-yl)ethan-1-one

Synthesised using the procedure described in Example 1 using Intermediate B6 and 3,4,5-trimethoxy aniline. ¹H NMR (CDCl₃, 400 MHz) δ 8.04 (d, J=2.0 Hz, 1H), 7.71 (d, J=6.3 Hz, 1H), 7.64 (s, 1H), 7.10 (d, J=7.9 Hz, 1H), 6.87 (dd, J=7.9, 2.1 Hz, 1H), 6.71 (s, 1H), 6.49 (s, 2H), 6.35 (dd, J=6.3, 2.1 Hz, 1H), 6.28 (d, J=2.1 Hz, 1H), 4.09 (t, J=8.4 Hz, 2H), 3.82 (s, 3H), 3.80 (s, 6H), 3.18 (t, J=8.4 Hz, 2H), 2.21 (s, 3H). LCMS Method A; RT 1.60; m/z [M+H]⁺ 435.2.

### Example 7 (1912)

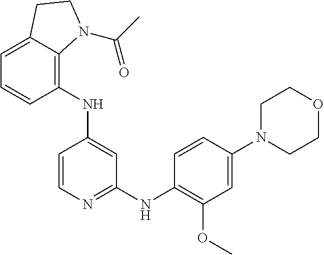 [SMILES: COc1cc(N2CCOCC2)ccc1Nc1cc(Nc2cccc3c2N(C(C)=O)CC3)ccn1]

### 1-(7-((2-((2-Methoxy-4-morpholinophenyl)amino)pyridin-4-yl)amino)indolin-1-yl)ethan-1-one

Synthesised using the procedure described in Example 3 using Intermediate B1 and 2-methoxy-4-morpholinoaniline. ¹H NMR (CDCl₃, 400 MHz) δ 8.78 (s, 1H), 7.94 (s, 1H), 7.64 (d, J=6.5 Hz, 1H), 7.34 (d, J=8.4 Hz, 1H), 7.21 (dd, J=8.2, 1.3 Hz, 1H), 7.12 (dd, J=8.1, 7.2 Hz, 1H), 6.99 (dd, J=7.4, 1.2 Hz, 1H), 6.54-6.42 (m, 2H), 6.29 (dd, J=6.6, 2.2 Hz, 1H), 6.09 (s, 1H), 4.18-4.07 (m, 2H), 3.93-3.84 (m, 4H), 3.83 (s, 3H), 3.19-3.06 (m, 6H), 2.34 (s, 3H). LCMS Method A; RT 1.76; m/z [M+H]⁺ 460.2.

### Example 8 (1934)

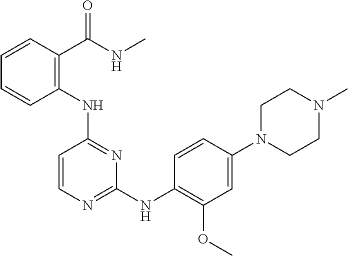 [SMILES: CNC(=O)c1ccccc1Nc1ccnc(Nc2ccc(N3CCN(C)CC3)cc2OC)n1]

### 2-((2-((2-Methoxy-4-(4-methylpiperazin-1-yl)phenyl)amino)pyridin-4-yl)amino)-N-methylbenzamide

Synthesised using the procedure described in Example 3 using Intermediate B14 and 2-methoxy-4-(4-methylpiperazin-1-yl)aniline. ¹H NMR (CDCl₃, 400 MHz) δ 9.71 (s, 1H), 7.67 (s, 1H), 7.48 (d, J=7.8 Hz, 1H), 7.40 (dt, J=15.4, 8.0 Hz, 2H), 7.06 (t, J=7.4 Hz, 1H), 6.53-6.40 (m, 3H), 6.28 (s, 1H), 6.20 (s, 1H), 3.83 (s, 2H), 3.29 (s, 4H), 2.98 (d, J=4.8 Hz, 3H), 2.72 (s, 4H), 2.46 (s, 3H). LCMS Method A; RT 0.90; m/z [M+H]447.1.

### Example 9 (1935)

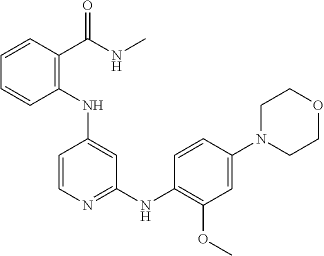 [SMILES: CNC(=O)c1ccccc1Nc1ccnc(Nc2ccc(N3CCOCC3)cc2OC)c1]

### 2-((2-((2-methoxy-4-morpholinophenyl)amino)pyridin-4-yl)amino)-N-methylbenzamide

Synthesised using the procedure described in Example 3 using Intermediate B14 and 2-methoxy-4-morpholinoaniline. ¹H NMR (CDCl₃, 400 MHz) δ 7.91 (d, J=5.8 Hz, 1H), 7.55 (d, J=8.4 Hz, 1H), 7.50 (dd, J=8.4, 1.2 Hz, 1H), 7.44 (dd, J=7.8, 1.6 Hz, 1H), 7.36 (ddd, J=8.5, 7.3, 1.6 Hz, 1H), 6.95 (td, J=7.5, 1.2 Hz, 1H), 6.81 (s, 1H), 6.55-6.46 (m, 3H), 6.38 (d, J=2.0 Hz, 1H), 6.26 (s, 1H), 3.91-3.86 (m, 4H), 3.84 (s, 3H), 3.17-3.10 (m, 4H), 2.98 (d, J=4.8 Hz, 3H). LCMS Method A; RT 1.42; m/z [M+H]434.1.

### Example 10(1936)

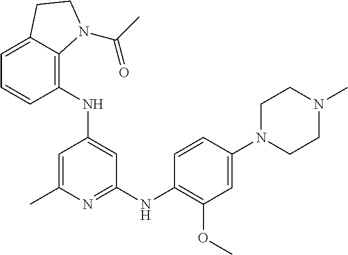 [SMILES: COc1cc(N2CCN(C)CC2)ccc1Nc1cc(Nc2cccc3c2N(C(C)=O)CC3)cc(C)n1]

### 1-(7-((2-((2-Methoxy-4-(4-methylpiperazin-1-yl)phenyl)amino)-6-methylpyridin-4-yl)amino)indolin-1-yl)ethan-1-one

Synthesised using the procedure described in Example 3 using Intermediate B15 and 2-methoxy-4-(4-methylpiperazin-1-yl)aniline. ¹H NMR (MeOD, 400 MHz) δ 7.29 (d, J=8.2 Hz, 1H), 7.10-7.00 (m, 2H), 6.90 (d, J=7.4 Hz, 1H), 6.61 (d, J=2.6 Hz, 1H), 6.49 (dd, J=8.6, 2.5 Hz, 1H), 6.09 (d, J=1.9 Hz, 1H), 5.93 (s, 1H), 4.09 (dd, J=7.3, 3.6 Hz, 2H), 3.78 (s, 3H), 3.35 (d, J=0.9 Hz, 3H), 3.18 (t, J=5.0 Hz, 4H), 3.05 (t, J=7.5 Hz, 2H), 2.61 (q, J=4.3 Hz, 4H), 2.34 (s, 3H), 2.30 (s, 3H). LCMS Method A; RT 1.24; m/z [M+H]⁺ 487.2.

### Example 11 (1937)

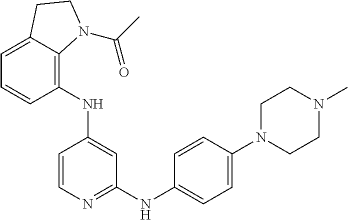 [SMILES: CC(=O)N1CCc2cccc(Nc3ccnc(Nc4ccc(N5CCN(C)CC5)cc4)c3)c21]

### 1-(7-((2-((4-(4-Methylpiperazin-1-yl)phenyl)amino)pyridin-4-yl)amino)indolin-1-yl)ethan-1-one

Synthesised using the procedure described in Example 3 using Intermediate B1 and 4-(4-methylpiperazin-1-yl)aniline. ¹H NMR (CDCl₃, 400 MHz) δ 8.34 (s, 1H), 7.78 (d, J=6.0 Hz, 1H), 7.25 (d, J=8.1 Hz, 1H), 7.21-7.14 (m, 2H), 7.10 (t, J=7.7 Hz, 1H), 6.96-6.87 (m, 4H), 6.34-6.25 (m, 2H), 4.10 (t, J=7.7 Hz, 2H), 3.18 (t, J=5.0 Hz, 4H), 3.11 (t, J=7.6 Hz, 2H), 2.60 (t, J=5.0 Hz, 4H), 2.37 (s, 3H), 2.32 (s, 3H). LCMS Method A; RT 1.20; m/z [M+H]⁺ 443.2.

### Example 12 (1938)

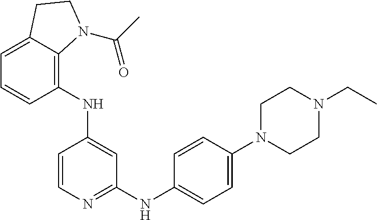 [SMILES: CCN1CCN(c2ccc(Nc3cc(Nc4cccc5c4N(C(C)=O)CC5)ccn3)cc2)CC1]

### 1-(7-(2(4-Ethylpiperazin-1-yl)phenyl)amino)pyridin-4-yl)amino)indolin-1-yl)ethan-1-one

Synthesised using the procedure described in Example 3 using Intermediate B1 and 4-(4-ethylpiperazin-1-yl)aniline. ¹H NMR (CDCl₃, 400 MHz) δ 8.38 (s, 1H), 7.76 (d, J=6.0 Hz, 1H), 7.24 (s, 1H), 7.19-7.13 (m, 2H), 7.13-7.07 (m, 1H), 6.94-6.88 (m, 3H), 6.32-6.24 (m, 2H), 4.12 (q, J=7.4 Hz, 2H), 3.23-3.16 (m, 4H), 3.11 (t, J=7.7 Hz, 2H), 2.64 (dd, J=6.5, 3.5 Hz, 4H), 2.50 (q, J=7.2 Hz, 2H), 2.33 (s, 3H), 1.15 (t, J=7.4, 3H). LCMS Method A; RT 1.31; m/z [M+H]457.1.

### Example 13 (1941)

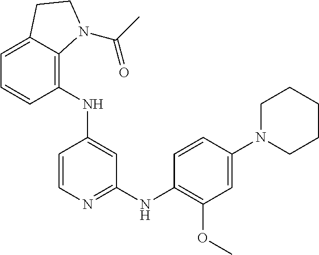 [SMILES: COc1cc(N2CCCCC2)ccc1Nc1cc(Nc2cccc3c2N(C(C)=O)CC3)ccn1]

### 1-(7-((2-((2-Methoxy-4-(piperidin-1-yl)phenyl)amino)pyridin-4-yl)amino)indolin-1-yl)ethan-1-one

Synthesised using the procedure described in Example 3 using Intermediate B1 and 2-methoxy-4-(1-piperidyl)aniline. ¹H NMR (CDCl₃, 400 MHz) δ 8.65 (s, 1H), 7.67 (d, J=6.4 Hz, 1H), 7.59 (s, 1H), 7.33 (d, J=8.6 Hz, 1H), 7.22 (dd, J=8.1, 1.2 Hz, 1H), 7.11 (t, J=7.7 Hz, 1H), 6.96 (dd, J=7.3, 1.2 Hz, 1H), 6.57-6.44 (m, 2H), 6.28 (dd, J=6.4, 2.1 Hz, 1H), 6.13 (d, J=2.1 Hz, 1H), 4.11 (t, J=7.8 Hz, 2H), 3.82 (s, 3H), 3.16-3.08 (m, 6H), 2.33 (s, 3H), 1.73 (p, J=5.7 Hz, 4H), 1.63-1.55 (m, 2H). LCMS Method A; RT 1.52; m/z [M+H]⁺ 458.2.

### Example 14 (1942)

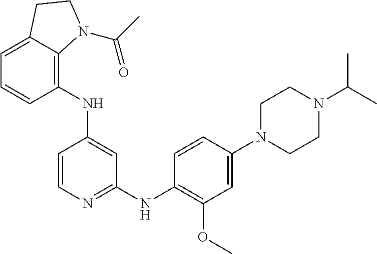 [SMILES: COc1cc(N2CCN(C(C)C)CC2)ccc1Nc1cc(Nc2cccc3c2N(C(C)=O)CC3)ccn1]

### 1-(7-((2-((4-(4-Isopropylpiperazin-1-yl)-2-methoxyphenyl)amino)pyridin-4-yl)amino)indolin-1-yl)ethan-1-one

Synthesised using the procedure described in Example 3 using Intermediate B1 and 4-(4-isopropylpiperazin-1-yl)-2-methoxy-aniline. ¹H NMR (MeOD, 400 MHz) δ 7.53 (d, J=6.9 Hz, 1H), 7.25-7.18 (m, 2H), 7.15 (dd, J=8.1, 5.1 Hz, 2H), 6.71 (d, J=2.5 Hz, 1H), 6.61 (dd, J=8.7, 2.5 Hz, 1H), 6.37 (dd, J=6.9, 2.2 Hz, 1H), 6.11 (s, 1H), 4.19 (t, J=7.8 Hz, 2H), 3.84 (s, 3H), 3.33-3.30 (m, 4H), 3.16 (t, J=7.7 Hz, 2H), 2.93-2.90 (m, 5H), 2.34 (s, 3H), 1.22 (d, J=6.6 Hz, 6H). LCMS Method A; RT 1.15; m/z [M+H]501.2.

### Example 15 (1943)

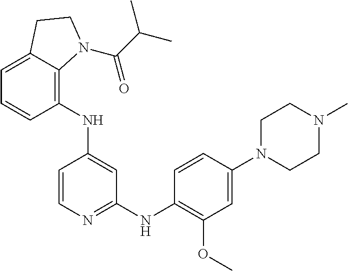 [SMILES: COc1cc(N2CCN(C)CC2)ccc1Nc1cc(Nc2cccc3c2N(C(=O)C(C)C)CC3)ccn1]

### 1-(7-((2-((2-Methoxy-4-(4-methylpiperazin-1-yl)phenyl)amino)pyridin-4-yl)amino)indolin-1-yl)-2-methylpropan-1-one

Synthesised using the procedure described in Example 3 using Intermediate B16 and 2-methoxy-4-(4-methylpiperazin-1-yl)aniline. ¹H NMR (CDCl₃, 500 MHz) δ 8.80 (s, 1H), 8.47 (s, 1H), 7.52 (d, J=6.8 Hz, 1H), 7.16 (dd, J=15.4, 8.3 Hz, 2H), 7.09 (t, J=7.7 Hz, 1H), 7.00 (dd, J=7.2, 1.3 Hz, 1H), 6.50-6.43 (m, 2H), 6.23 (dd, J=6.9, 2.2 Hz, 1H), 5.97 (d, J=2.1 Hz, 1H), 4.15 (t, J=7.7 Hz, 2H), 3.81 (s, 3H), 3.23 (t, J=5.0 Hz, 4H), 3.11 (t, J=7.7 Hz, 2H), 2.89 (h, J=6.7 Hz, 1H), 2.65-2.58 (m, 4H), 2.40 (s, 3H), 1.22 (d, J=6.7 Hz, 6H). LCMS Method A; RT 1.35; m/z [M+H]⁺ 501.3.

### Example 16 (1944)

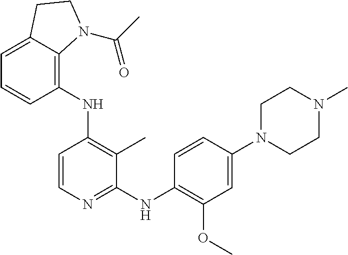 [SMILES: COc1cc(N2CCN(C)CC2)ccc1Nc1nccc(Nc2cccc3c2N(C(C)=O)CC3)c1C]

### 1-(7-((2-((2-Methoxy-4-(4-methylpiperazin-1-yl)phenyl)amino)-3-methylpyridin-4-yl)amino)indolin-1-yl)ethan-1-one

Synthesised using the procedure described in Example 3 using Intermediate B33 and 2-methoxy-4-(4-methylpiperazin-1-yl)aniline. ¹H NMR (CDCl₃, 500 MHz) δ 8.71 (s, 1H), 7.78 (d, J=6.3 Hz, 1H), 7.72 (d, J=9.0 Hz, 1H), 7.17-7.10 (m, 2H), 6.95 (dd, J=6.8, 1.6 Hz, 1H), 6.91 (s, 1H), 6.56-6.51 (m, 3H), 4.12 (t, J=7.7 Hz, 2H), 3.86 (s, 3H), 3.37 (t, J=5.0 Hz, 4H), 3.13 (t, J=7.7 Hz, 2H), 2.93 (t, J=4.9 Hz, 4H), 2.56 (s, 3H), 2.34 (s, 3H), 2.17 (s, 3H). LCMS Method A; RT 1.24; m/z [M+H]⁺ 487.2.

### Example 17 (1950)

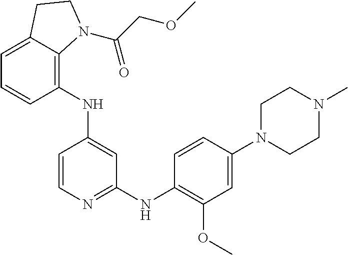 [SMILES: COCC(=O)N1CCc2cccc(Nc3ccnc(Nc4ccc(N5CCN(C)CC5)cc4OC)c3)c21]

### 2-Methoxy-1-(7-((2-((2-methoxy-4-(4-methylpiperazin-1-yl)phenyl)amino)pyridin-4-yl)amino)indolin-1-yl)ethan-1-one

Synthesised using the procedure described in Example 3 using Intermediate B17 and 2-methoxy-4-(4-methylpiperazin-1-yl)aniline. ¹H NMR (CDCl₃, 500 MHz) δ 8.80 (s, 1H), 8.76 (s, 1H), 7.53 (d, J=6.9 Hz, 1H), 7.20-7.09 (m, 3H), 7.03 (dd, J=7.1, 1.3 Hz, 1H), 6.51-6.44 (m, 2H), 6.26 (dd, J=6.9, 2.2 Hz, 1H), 5.97 (d, J=2.2 Hz, 1H), 4.26 (s, 2H), 4.10 (t, J=7.7 Hz, 2H), 3.81 (s, 3H), 3.46 (s, 3H), 3.26 (t, J=5.1 Hz, 4H), 3.13 (t, J=7.7 Hz, 2H), 2.68 (t, J=5.0 Hz, 4H), 2.42 (s, 3H). LCMS Method A; RT 1.10; m/z [M+H]⁺ 503.2.

### Example 18 (1951)

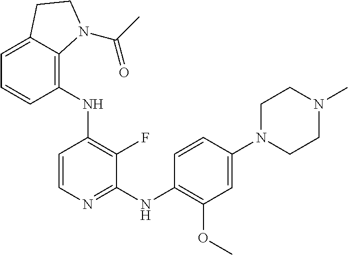 [SMILES: COc1cc(N2CCN(C)CC2)ccc1Nc1nccc(Nc2cccc3c2N(C(C)=O)CC3)c1F]

### 1-(7-((3-Fluoro-2-((2-methoxy-4-(4-methylpiperazin-1-yl)phenyl)amino)pyridin-4-yl)amino)indolin-1-yl)ethan-1-one

Synthesised using the procedure described in Example 3 using Intermediate B18 and 2-methoxy-4-(4-methylpiperazin-1-yl)aniline. ¹H NMR (DMSO-d6, 400 MHz) δ 8.85 (s, 1H), 7.88 (d, J=8.7 Hz, 1H), 7.58 (d, J=5.6 Hz, 1H), 7.18 (d, J=4.2 Hz, 3H), 7.03 (t, J=4.3 Hz, 1H), 6.64 (d, J=2.6 Hz, 1H), 6.49-6.38 (m, 2H), 4.16 (t, J=7.7 Hz, 2H), 3.84 (s, 3H), 3.10 (q, J=5.8 Hz, 6H), 2.47 (d, J=4.9 Hz, 4H), 2.31 (s, 3H), 2.24 (s, 3H). LCMs Method A; RT 1.14; m/z [M+H]⁺ 491.1.

### Example 19 (1958)

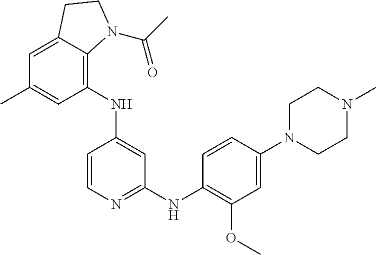

### 1-(7-((2-((2-Methoxy-4-(4-methylpiperazin-1-yl)phenyl)amino)pyridin-4-yl)amino)-5-methylindolin-1-yl)ethan-1-one

Synthesised using the procedure described in Example 3 using Intermediate B19 and 2-methoxy-4-(4-methylpiperazin-1-yl)aniline. ¹H NMR (CDCl₃, 500 MHz) δ 9.15 (s, 1H), 8.91 (s, 1H), 7.50 (d, J=7.0 Hz, 1H), 7.15 (d, J=8.6 Hz, 1H), 6.94 (s, 1H), 6.80 (s, 1H), 6.52-6.45 (m, 2H), 6.22 (dd, J=7.0, 2.2 Hz, 1H), 6.01 (d, J=2.2 Hz, 1H), 4.09 (t, J=7.8 Hz, 2H), 3.82 (s, 3H), 3.24 (t, J=5.0 Hz, 4H), 3.08 (t, J=7.7 Hz, 2H), 2.65 (t, J=5.2 Hz, 4H), 2.41 (s, 3H), 2.31 (s, 3H), 2.28 (s, 3H). LCMS Method A; RT 1.25; m/z [M+H]⁺ 487.2.

### Example 20 (1959)

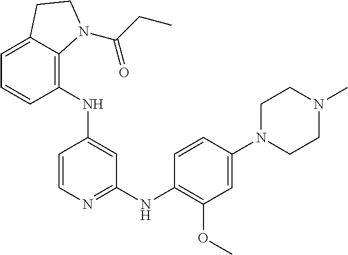 [SMILES: CCC(=O)N1CCc2cccc(Nc3ccnc(Nc4ccc(N5CCN(C)CC5)cc4OC)c3)c21]

### 1-(7-((2-((2-Methoxy-4-(4-methylpiperazin-1-yl)phenyl)amino)pyridin-4-yl)amino)indolin-1-yl)propan-1-one

Synthesised using the procedure described in Example 3 using Intermediate B20 and 2-methoxy-4-(4-methylpiperazin-1-yl)aniline. ¹H NMR (CDCl₃, 500 MHz) δ 9.07 (s, 1H), 8.98 (s, 1H), 7.51 (d, J=6.9 Hz, 1H), 7.16 (dd, J=8.4, 3.4 Hz, 2H), 7.09 (t, J=7.6 Hz, 1H), 7.00 (d, J=7.2 Hz, 1H), 6.50 (d, J=2.5 Hz, 1H), 6.47 (dd, J=8.5, 2.6 Hz, 1H), 6.24 (dd, J=6.9, 2.2 Hz, 1H), 5.96 (d, J=2.3 Hz, 1H), 4.11 (t, J=7.7 Hz, 2H), 3.82 (s, 3H), 3.25 (t, J=5.0 Hz, 4H), 3.12 (t, J=7.7 Hz, 2H), 2.67 (t, J=4.9 Hz, 4H), 2.57 (q, J=7.4 Hz, 2H), 2.42 (s, 3H), 1.28-1.23 (m, 3H). LCMS Method A; RT 1.26; m/z [M+H]⁺ 487.2.

### Example 21 (1961)

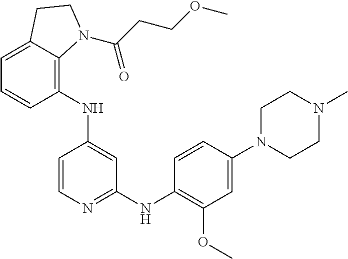 [SMILES: COCCC(=O)N1CCc2cccc(Nc3ccnc(Nc4ccc(N5CCN(C)CC5)cc4OC)c3)c21]

### 3-Methoxy-1-(7-((2-((2-methoxy-4-(4-methylpiperazin-1-yl)phenyl)amino)pyridin-4-yl)amino)indolin-1-yl)propan-1-one

Synthesised using the procedure described in Example 3 using Intermediate B21 and 2-methoxy-4-(4-methylpiperazin-1-yl)aniline. ¹H NMR (CDCl₃, 500 MHz) δ 7.79 (d, J=6.3 Hz, 1H), 7.43 (d, J=8.6 Hz, 1H), 7.11-7.05 (m, 2H), 6.99 (t, J=7.6 Hz, 1H), 6.53 (d, J=2.5 Hz, 1H), 6.49 (dd, J=8.7, 2.5 Hz, 1H), 6.24 (dd, J=6.4, 2.3 Hz, 1H), 6.05 (d, J=2.3 Hz, 1H), 4.18 (t, J=8.6 Hz, 2H), 3.83 (s, 3H), 3.81-3.76 (m, 2H), 3.21 (t, J=5.0 Hz, 4H), 3.13 (t, J=8.6 Hz, 2H), 2.93-2.89 (m, 2H), 2.65 (t, J=5.1 Hz, 4H), 2.40 (s, 3H), 2.18 (s, 3H). LCMS Method A; RT 1.16; m/z [M+H]⁺ 485.1.

### Example 22(1968)

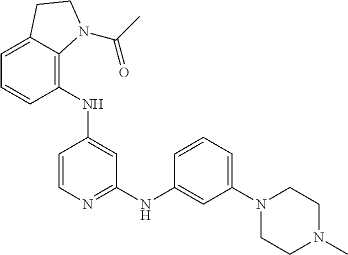 [SMILES: CC(=O)N1CCc2cccc(Nc3ccnc(Nc4cccc(N5CCN(C)CC5)c4)c3)c21]

### 1-(7-((2-((3-(4-Methylpiperazin-1-yl)phenyl)amino)pyridin-4-yl)amino)indolin-1-yl)ethan-1-one

Synthesised using the procedure described in Example 3 using Intermediate B1 and 3-(4-methylpiperazin-1-yl)aniline. ¹H NMR (CDCl₃, 500 MHz) δ 8.83 (s, 1H), 8.03 (s, 1H), 7.68 (d, J=6.5 Hz, 1H), 7.25-7.17 (m, 2H), 7.13 (t, J=7.7 Hz, 1H), 6.99 (dd, J=7.2, 1.2 Hz, 1H), 6.77 (t, J=2.2 Hz, 1H), 6.75-6.70 (m, 1H), 6.67 (dd, J=8.3, 2.4 Hz, 1H), 6.42 (d, J=2.1 Hz, 1H), 6.31 (dd, J=6.5, 2.1 Hz, 1H), 4.12 (t, J=7.8 Hz, 2H), 3.25-3.16 (m, 4H), 3.13 (t, J=7.8 Hz, 2H), 2.61-2.51 (m, 4H), 2.37 (s, 3H), 2.34 (s, 3H). LCMS Method A; RT 1.16; m/z [M+H]⁺ 443.2.

### Example 23 (1971)

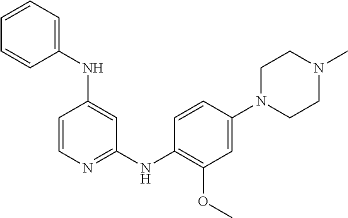 [SMILES: COc1cc(N2CCN(C)CC2)ccc1Nc1cc(Nc2ccccc2)ccn1]

### N2-(2-Methoxy-4-(4-methylpiperazin-1-yl)phenyl)-N4-phenylpyridine-2,4-diamine

A mixture of palladium acetate (2 mg, 0.01 mmol), cesium carbonate (118 mg, 0.36 mmol), Intermediate C1 (60 mg, 0.18 mmol), aniline (0.02 mL, 0.18 mmol) and XANTPHOS (6 mg, 0.01 mmol), in 1,4-dioxane (2 mL) was heated at 100° C. in the microwave for 30 min. After cooling to room temperature the mixture was filtered through an SCX cartridge washing first with DCM and then with ammonia in methanol (7M). The ammonia layer was reduced in vacuo and purified on the Biotage Isolera to give the title compound (20 mg, 28% yield). ¹H NMR (CDCl₃, 400 MHz) δ 8.53 (s, 1H), 7.50 (d, J=6.9 Hz, 1H), 7.34 (t, J=7.8 Hz, 2H), 7.18 (t, J=7.4 Hz, 1H), 7.13 (dd, J=8.1, 2.2 Hz, 3H), 6.85 (s, 1H), 6.47 (d, J=8.1 Hz, 2H), 6.22 (dd, J=6.9, 2.3 Hz, 1H), 5.88 (d, J=2.2 Hz, 1H), 3.80 (s, 3H), 3.28 (t, J=5.1 Hz, 4H), 2.86-2.78 (m, 4H), 2.49 (s, 3H). LCMS Method A; RT 1.09; m/z [M+H]⁺ 390.2.

### Example 24 (1999)

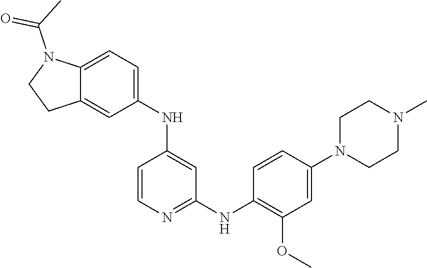 [SMILES: COc1cc(N2CCN(C)CC2)ccc1Nc1cc(Nc2ccc3c(c2)CCN3C(C)=O)ccn1]

### 1-(5-((2-((2-Methoxy-4-(4-methylpiperazin-1-yl)phenyl)amino)pyridin-4-yl)amino)indolin-1-yl)ethan-1-one

Synthesised using the procedure described in Example 1 using Intermediate B5 and 2-methoxy-4-(4-methylpiperazin-1-yl). ¹H NMR (CDCl₃, 400 MHz) δ 8.13 (d, J=8.4 Hz, 2H), 7.47 (d, J=6.8 Hz, 1H), 7.37 (s, 1H), 7.19 (d, J=8.5 Hz, 1H), 7.00-6.92 (m, 2H), 6.51-6.40 (m, 2H), 6.32 (dd, J=6.8, 2.1 Hz, 1H), 5.99 (d, J=2.1 Hz, 1H), 4.07 (t, J=8.5 Hz, 2H), 3.80 (s, 3H), 3.23 (t, J=5.0 Hz, 4H), 3.14 (t, J=8.4 Hz, 2H), 2.66 (q, J=5.4 Hz, 4H), 2.42 (s, 3H), 2.23 (s, 3H). LCMS Method A; RT 1.03; m/z [M+H]⁺ 473.1.

### Example 25 (1992)

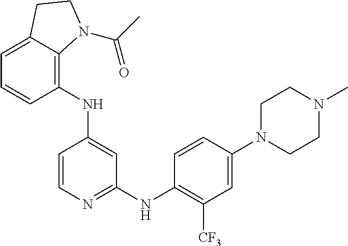 [SMILES: CC(=O)N1CCc2cccc(Nc3ccnc(Nc4ccc(N5CCN(C)CC5)cc4C(F)(F)F)c3)c21]

### 1-(7-((2-((4-(4-Methylpiperazin-1-yl)-2-(trifluoromethyl)phenyl)amino)pyridin-4-yl)amino)indolin-1-yl)ethan-1-one

Synthesised using the procedure described in Example 3 using Intermediate B1 and 4-(4-methylpiperazin-1-yl)-2-(trifluoromethyl)aniline. ¹H NMR (CDCl₃, 400 MHz) δ 8.57 (s, 1H), 7.74 (d, J=6.3 Hz, 1H), 7.46 (d, J=8.9 Hz, 1H), 7.20 (d, J=8.3 Hz, 1H), 7.15 (d, J=2.9 Hz, 1H), 7.09 (t, J=7.7 Hz, 1H), 7.05 (dd, J=8.9, 2.9 Hz, 1H), 6.95 (dd, J=7.2, 1.2 Hz, 1H), 6.35 (dd, J=6.3, 2.1 Hz, 1H), 6.01 (s, 1H), 4.11 (t, J=7.8 Hz, 2H), 3.29-3.19 (m, 4H), 3.12 (t, J=7.7 Hz, 2H), 2.64-2.57 (m, 4H), 2.39 (s, 3H), 2.33 (s, 3H). LCMS Method A; RT 1.20; m/z [M+H]⁺ 511.2.

### Example 26 (1993)

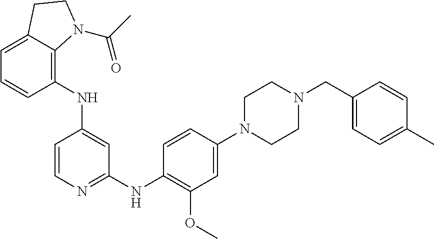 [SMILES: COc1cc(N2CCN(Cc3ccc(C)cc3)CC2)ccc1Nc1cc(Nc2cccc3c2N(C(C)=O)CC3)ccn1]

### 1-(7-((2-((2-Methoxy-4-(4-(4-methylbenzyl)piperazin-1-yl)phenyl)amino)pyridin-4-yl)amino)indolin-1-yl)ethan-1-one

Synthesised using the procedure described in Example 3 using Intermediate B1 and Intermediate E2. ¹H NMR (CDCl₃, 400 MHz) δ 8.85 (s, 1H), 7.59 (d, J=6.6 Hz, 1H), 7.26 (d, J=8.0 Hz, 2H), 7.20-7.14 (m, 3H), 7.10 (t, J=7.7 Hz, 1H), 6.99 (d, J=7.3 Hz, 1H), 6.49 (d, J=2.5 Hz, 1H), 6.46 (dd, J=8.6, 2.5 Hz, 1H), 6.27 (dd, J=6.7, 2.2 Hz, 1H), 6.05 (s, 1H), 4.12 (t, J=7.8 Hz, 2H), 3.81 (s, 3H), 3.56 (s, 2H), 3.22-3.16 (m, 4H), 3.13 (t, J=7.8 Hz, 2H), 2.66-2.58 (m, 4H), 2.36 (s, 3H), 2.33 (s, 3H). LCMS Method A; RT 1.44; m/z [M+H]⁺ 563.3.

### Example 27 (1994)

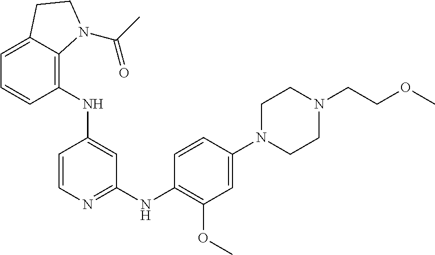 [SMILES: COCCN1CCN(c2ccc(Nc3cc(Nc4cccc5c4N(C(C)=O)CC5)ccn3)c(OC)c2)CC1]

### 1-(7-((2-((2-Methoxy-4-(4-(2-methoxyethyl)piperazin-1-yl)phenyl)amino)pyridin-4-yl)amino)indolin-1-yl)ethan-1-one

Synthesised using the procedure described in Example 3 using Intermediate B1 and Intermediate E1. ¹H NMR (CDCl₃, 400 MHz) δ 8.26 (s, 1H), 7.83 (d, J=5.9 Hz, 1H), 7.56 (d, J=8.5 Hz, 1H), 7.30-7.27 (m, 1H), 7.10 (dd, J=8.2, 7.3 Hz, 1H), 6.98 (s, 1H), 6.91 (dt, J=7.3, 1.1 Hz, 1H), 6.55-6.49 (m, 2H), 6.31 (dd, J=5.9, 2.0 Hz, 1H), 6.26 (d, J=2.0 Hz, 1H), 4.13-4.05 (m, 2H), 3.83 (s, 3H), 3.57 (t, J=5.6 Hz, 2H), 3.39 (s, 3H), 3.22-3.16 (m, 4H), 3.10 (t, J=7.7 Hz, 2H), 2.73-2.62 (m, 6H), 2.32 (s, 3H). LCMS Method A; RT 1.19; m/z [M+H]⁺ 517.2.

### Example 28 (1995)

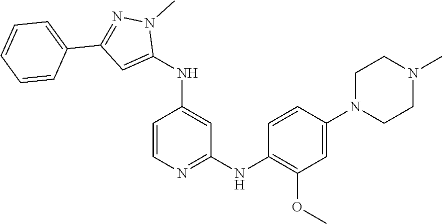 [SMILES: COc1cc(N2CCN(C)CC2)ccc1Nc1cc(Nc2cc(-c3ccccc3)nn2C)ccn1]

### N2-(2-Methoxy-4-(4-methylpiperazin-1-yl)phenyl)-N4-(1-methyl-3-phenyl-1H-pyrazol-5-yl)pyridine-2,4-diamine

Synthesised using the procedure described in Example 3 using Intermediate B22 and 2-methoxy-4-(4-methylpiperazin-1-yl)aniline. ¹H NMR (CDCl₃, 400 MHz) δ 7.76-7.71 (m, 3H), 7.43-7.36 (m, 3H), 7.35-7.28 (m, 1H), 6.72 (s, 1H), 6.49-6.42 (m, 2H), 6.35 (s, 1H), 6.23 (dd, J=6.3, 2.1 Hz, 1H), 5.99 (d, J=2.1 Hz, 1H), 3.79 (s, 3H), 3.75 (s, 3H), 3.16 (t, J=5.1 Hz, 4H), 2.63 (t, J=4.8 Hz, 4H), 2.39 (s, 3H). LCMS Method A; RT 1.20; m/z [M+H]⁺ 470.1.

### Example 79 (1998)

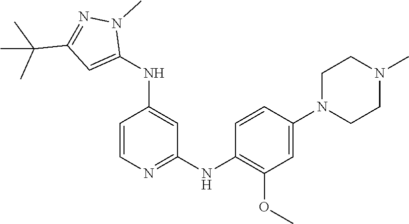 [SMILES: COc1cc(N2CCN(C)CC2)ccc1Nc1cc(Nc2cc(C(C)(C)C)nn2C)ccn1]

### N4-(3-(tert-Butyl)-1-methyl-1H-pyrazol-5-yl)-N2-(2-methoxy-4-(4-methylpiperazin-1-yl)phenyl)pyridine-2,4-diamine

Synthesised using the procedure described in Example 3 using Intermediate B25 and 2-methoxy-4-(4-methylpiperazin-1-yl)aniline. ¹H NMR (CDCl₃, 400 MHz) δ 7.75 (s, 1H), 7.65 (d, J=6.3 Hz, 1H), 7.29 (d, J=8.6 Hz, 1H), 6.88 (s, 1H), 6.48 (d, J=2.5 Hz, 1H), 6.45 (dd, J=8.6, 2.6 Hz, 1H), 6.27 (d, J=6.1 Hz, 1H), 5.94 (d, J=2.1 Hz, 1H), 5.87 (s, 1H), 3.79 (s, 3H), 3.65 (s, 3H), 3.22-3.14 (m, 4H), 2.65-2.60 (m, 4H), 2.39 (s, 3H), 1.26 (s, 9H). LCMS Method A; RT 1.25; m/z [M+H]⁺ 450.2.

### Example 30 (2000)

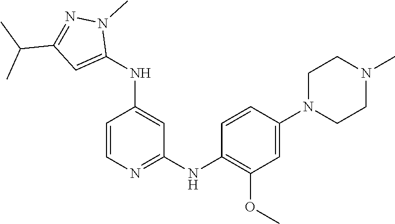 [SMILES: COc1cc(N2CCN(C)CC2)ccc1Nc1cc(Nc2cc(C(C)C)nn2C)ccn1]

### N4-(3-Isopropyl-1-methyl-1H-pyrazol-5-yl)-N2-(2-methoxy-4-(4-methylpiperazin-1-yl)phenyl)pyridine-2,4-diamine

Synthesised using the procedure described in Example 3 using Intermediate B27 and 2-methoxy-4-(4-methylpiperazin-1-yl)aniline. ¹H NMR (CDCl₃, 400 MHz) δ 7.73 (d, J=6.1 Hz, 1H), 7.36 (d, J=8.5 Hz, 1H), 6.53-6.42 (m, 2H), 6.19 (d, J=5.8 Hz, 1H), 5.95 (d, J=1.9 Hz, 1H), 5.86 (s, 1H), 3.81 (s, 3H), 3.65 (s, 3H), 3.20 (t, J=5.0 Hz, 4H), 2.90 (p, J=6.9 Hz, 1H), 2.65 (d, J=6.3 Hz, 4H), 2.40 (s, 3H), 1.26 (s, 6H). LCMS Method A; RT 1.16; m/z [M+H]⁺ 436.2

### Example 31 (2007)

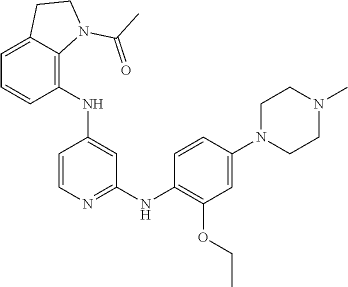 [SMILES: CCOc1cc(N2CCN(C)CC2)ccc1Nc1cc(Nc2cccc3c2N(C(C)=O)CC3)ccn1]

### 1-(7-((2-((2-Ethoxy-4-(4-methylpiperazin-1-yl)phenyl)amino)pyridin-4-yl)amino)indolin-1-yl)ethan-1-one

Synthesised using the procedure described in Example 3 using Intermediate B1 and Intermediate D3. ¹H NMR (CDCl₃, 400 MHz) δ 9.12 (s, 1H), 9.03 (s, 1H), 7.50 (d, J=7.0 Hz, 1H), 7.19-7.07 (m, 3H), 7.05-6.98 (m, 1H), 6.53-6.45 (m, 2H), 6.24 (dd, J=7.0, 2.2 Hz, 1H), 6.00 (d, J=2.2 Hz, 1H), 4.12 (t, J=7.8 Hz, 2H), 4.04 (q, J=7.0 Hz, 2H), 3.27 (t, J=5.1 Hz, 4H), 3.14 (t, J=7.7 Hz, 2H), 2.73-2.71 (m, 4H), 2.45 (s, 3H), 2.33 (s, 3H), 1.39 (t, J=7.0 Hz, 3H). LCMS Method A; RT 1.23; m/z [M+H]⁺ 487.2.

### Example 32 (2008)

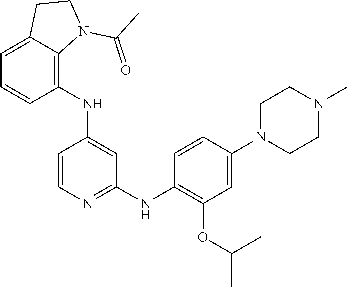 [SMILES: CC(=O)N1CCc2cccc(Nc3ccnc(Nc4ccc(N5CCN(C)CC5)cc4OC(C)C)c3)c21]

### 1-(7-((2-((2-Isopropoxy-4-(4-methylpiperazin-1-yl)phenyl)amino)pyridin-4-yl)amino)indolin-1-yl)ethan-1-one

Synthesised using the procedure described in Example 3 using Intermediate B1 and Intermediate D2. ¹H NMR (CDCl₃, 400 MHz) δ 8.99 (s, 1H), 8.84 (s, 1H), 7.52 (d, J=6.8 Hz, 1H), 7.18 (d, J=8.2 Hz, 2H), 7.11 (t, J=7.6 Hz, 1H), 7.00 (dd, J=7.3, 1.2 Hz, 1H), 6.54-6.45 (m, 2H), 6.25 (dd, J=6.9, 2.2 Hz, 1H), 6.03 (d, J=2.2 Hz, 1H), 4.50 (p, J=6.1 Hz, 1H), 4.12 (t, J=7.8 Hz, 2H), 3.23 (t, J=5.1 Hz, 4H), 3.14 (t, J=7.8 Hz, 2H), 2.67 (s, 4H), 2.42 (s, 3H), 2.33 (s, 3H), 1.31 (d, J=6.1 Hz, 6H). LCMS Method A; RT 1.34; m/z [M+H]⁺ 501.2.

### Example 33 (2009)

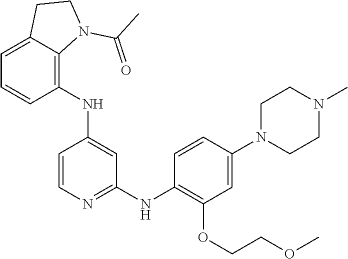 [SMILES: COCCOc1cc(N2CCN(C)CC2)ccc1Nc1cc(Nc2cccc3c2N(C(C)=O)CC3)ccn1]

### 1-(7-((2-((2-(2-Methoxyethoxy)-4-(4-methylpiperazin-1-yl)phenyl)amino)pyridin-4-yl)amino)indolin-1-yl)ethan-1-one

Synthesised using the procedure described in Example 3 using Intermediate B1 and Intermediate D1. ¹H NMR (CDCl₃, 400 MHz) δ 8.93 (s, 1H), 8.47 (s, 1H), 7.57 (d, J=6.8 Hz, 1H), 7.27-7.17 (m, 2H), 7.10 (dd, J=8.1, 7.3 Hz, 1H), 6.99 (dd, J=7.4, 1.1 Hz, 1H), 6.55 (d, J=2.6 Hz, 1H), 6.50 (dd, J=8.7, 2.6 Hz, 1H), 6.26 (dd, J=6.8, 2.2 Hz, 1H), 6.07 (d, J=2.1 Hz, 1H), 4.17-4.05 (m, 4H), 3.78-3.71 (m, 2H), 3.39 (s, 3H), 3.22 (t, J=5.1 Hz, 4H), 3.13 (t, J=7.8 Hz, 2H), 2.66-2.58 (m, 4H), 2.41 (s, 3H), 2.34 (s, 3H). LCMS Method A; RT 1.27; m/z [M+H]⁺ 517.2.

### Example 34 (2010)

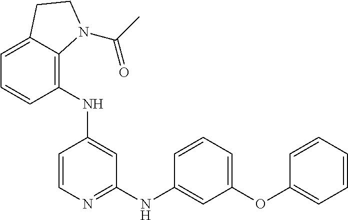 [SMILES: CC(=O)N1CCc2cccc(Nc3ccnc(Nc4cccc(Oc5ccccc5)c4)c3)c21]

### 1-(7-((2-((3-Phenoxyphenyl)amino)pyridin-4-yl)amino)indolin-1-yl)ethan-1-one

Synthesised using the procedure described in Example 1 using Intermediate B1 and 3-phenoxyaniline. ¹H NMR (CDCl₃, 400 MHz) δ 8.38 (s, 1H), 7.86 (d, J=5.8 Hz, 1H), 7.37-7.31 (m, 2H), 7.29 (d, J=1.1 Hz, 1H), 7.23 (t, J=8.1 Hz, 1H), 7.13-7.07 (m, 2H), 7.07-6.97 (m, 4H), 6.92 (dq, J=7.4, 1.1 Hz, 1H), 6.67 (s, 1H), 6.61 (ddd, J=8.1, 2.3, 0.9 Hz, 1H), 6.45 (d, J=2.0 Hz, 1H), 6.37 (dd, J=5.9, 2.0 Hz, 1H), 4.11 (t, J=7.8 Hz, 2H), 3.12 (t, J=7.7 Hz, 2H), 2.33 (s, 3H). LCMS Method A; RT 2.13; m/z [M+H]⁺ 437.2.

### Example 35 (2011)

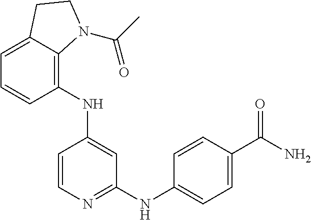 [SMILES: CC(=O)N1CCc2cccc(Nc3ccnc(Nc4ccc(C(N)=O)cc4)c3)c21]

### 4-((4-((1-Acetylindolin-7-yl)amino)pyridin-2-yl)amino)benzamide

Synthesised using the procedure described in Example 1 using Intermediate B1 and 4-aminobenzamide. ¹H NMR (CDCl₃, 400 MHz): 8.53 (br s, 1H), 7.93 (d, J=5.9 Hz, 1H), 7.76 (d, J=8.8 Hz, 2H), 7.37 (d, J=8.8 Hz, 2H), 7.31 (d, J=8.6 Hz, 1H), 7.17 (t, J=7.4 Hz, 1H), 7.02 (br s, 1H), 6.98 (d, J=8.3 Hz, 1H), 6.49 (s, 1H), 6.47 (d, J=5.9 Hz, 1H), 5.75 (br s, 2H), 4.14 (t, J=7.8 Hz, 2H), 3.15 (t, J=7.8 Hz, 2H), 2.36 (s, 3H). LCMS Method A; RT 1.49; m/z [M+H]⁺ 388.2.

### Example 36 (2013)

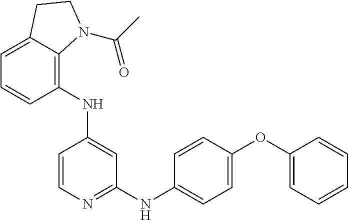 [SMILES: CC(=O)N1CCc2cccc(Nc3ccnc(Nc4ccc(Oc5ccccc5)cc4)c3)c21]

### 1-(7-((2-((4-Phenoxyphenyl)amino)pyridin-4-yl)amino)indolin-1-yl)ethan-1-one

Synthesised using the procedure described in Example 1 using Intermediate B1 and 4-phenoxyaniline. ¹H NMR (CDCl₃, 500 MHz) δ 8.40 (s, 1H), 7.84 (d, J=5.9 Hz, 1H), 7.33 (dd, J=8.5, 7.2 Hz, 2H), 7.27-7.24 (m, 3H), 7.15-7.06 (m, 2H), 7.03-6.96 (m, 4H), 6.94 (d, J=7.2 Hz, 1H), 6.71 (s, 1H), 6.36 (dd, J=6.0, 2.0 Hz, 1H), 6.33 (d, J=2.0 Hz, 1H), 4.12 (td, J=7.4, 3.0 Hz, 2H), 3.12 (t, J=7.7 Hz, 2H), 2.34 (s, 3H). LCMS Method A; RT 2.08; m/z [M+H]⁺ 437.2.

### Example 37 (2014)

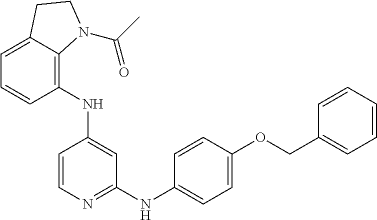 [SMILES: CC(=O)N1CCc2cccc(Nc3ccnc(Nc4ccc(OCc5ccccc5)cc4)c3)c21]

### 1-(7-((2-((4-(Benzyloxy)phenyl)amino)pyridin-4-yl)amino)indolin-1-yl)ethan-1-one

Synthesised using the procedure described in Example 1 using Intermediate B1 and 4-benzyloxyaniline. ¹H NMR (CDCl₃, 400 MHz) δ 8.50 (s, 1H), 7.76 (d, J=6.2 Hz, 1H), 7.47-7.43 (m, 2H), 7.43-7.38 (m, 2H), 7.37-7.31 (m, 1H), 7.23 (dd, J=8.2, 1.1 Hz, 1H), 7.20-7.16 (m, 2H), 7.09 (dd, J=8.1, 7.3 Hz, 2H), 6.97-6.91 (m, 3H), 6.32 (dd, J=6.1, 2.1 Hz, 1H), 6.23 (d, J=2.0 Hz, 1H), 5.06 (s, 2H), 4.14-4.06 (m, 2H), 3.11 (t, J=7.7 Hz, 2H), 2.33 (s, 3H). LCMS Method A; RT 2.09; m/z [M+H]⁺ 451.2.

### Example 38 (2015)

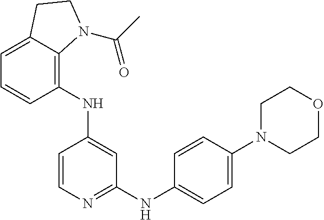 [SMILES: CC(=O)N1CCc2cccc(Nc3ccnc(Nc4ccc(N5CCOCC5)cc4)c3)c21]

### 1-(7-((2-((4-Morpholinophenyl)amino)pyridin-4-yl)amino)indolin-1-yl)ethan-1-one

Synthesised using the procedure described in Example 3 using Intermediate B1 and N-(4-aminophenyl)morpholine. ¹H NMR (CDCl₃, 500 MHz) δ 8.47 (s, 1H), 7.78 (d, J=6.1 Hz, 1H), 7.28-7.24 (m, 1H), 7.23-7.17 (m, 2H), 7.16-7.08 (m, 1H), 6.96 (dd, J=7.3, 1.1 Hz, 2H), 6.93-6.89 (m, 2H), 6.34 (dd, J=6.1, 2.1 Hz, 1H), 6.26 (d, J=2.0 Hz, 1H), 4.13 (t, J=7.7 Hz, 2H), 3.95-3.86 (m, 4H), 3.20-3.10 (m, 6H), 2.35 (s, 3H). LCMS Method A; RT 1.68; m/z [M+H]⁺ 430.1.

### Example 39 (2016)

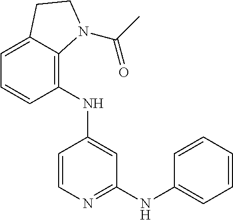 [SMILES: CC(=O)N1CCc2cccc(Nc3ccnc(Nc4ccccc4)c3)c21]

### 1-(7-((2-(Phenylamino)pyridin-4-yl)amino)indolin-1-yl)ethan-1-one

Synthesised using the procedure described in Example 1 using Intermediate B1 and aniline. ¹H NMR (CDCl₃, 400 MHz): 8.38 (br s, 1H), 7.85 (d, J=5.9 Hz, 1H), 7.32-7.27 (m, 3H), 7.25-7.24 (m, 2H), 7.12 (t, J=7.4 Hz, 1H), 7.01 (t, J=7.0 Hz, 1H), 6.92 (dd, J=7.3 and 1.0 Hz, 1H), 6.68 (br s, 1H), 6.43 (d, J=1.8 Hz, 1H), 6.36 (dd, J=5.9 and 2.0 Hz, 1H), 4.10 (t, J=7.6 Hz, 2H), 3.11 (t, J=7.8 Hz, 2H), 2.33 (s, 3H). LCMS Method A; RT 1.76; m/z [M+H]⁺ 345.2.

### Example 40 (2017)

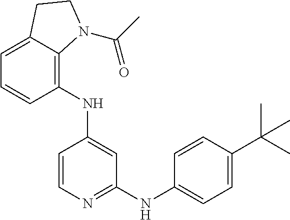 [SMILES: CC(=O)N1CCc2cccc(Nc3ccnc(Nc4ccc(C(C)(C)C)cc4)c3)c21]

### 1-(7-((2-((4-(tert-Butyl)phenyl)amino)pyridin-4-yl)amino)indolin-1-yl)ethan-1-one

Synthesised using the procedure described in Example 1 using Intermediate B1 and 4-(tert-butyl)aniline. ¹H NMR (CDCl₃, 400 MHz): 8.42 (br s, 1H), 7.79 (d, J=6.0 Hz, 1H), 7.32 (d, J=8.7 Hz, 2H), 7.26 (d, J=7.3 Hz, 1H), 7.17 (d, J=8.7 Hz, 2H), 7.11 (t, J=7.4 Hz, 1H), 6.93 (dd, J=7.3 and 1.0 Hz, 1H), 6.90 (m, 1H), 6.38 (d, J=1.9 Hz, 1H), 6.33 (dd, J=6.0 and 2.1 Hz, 1H), 4.11 (t, J=7.8 Hz, 2H), 3.11 (t, J=7.8 Hz, 2H), 2.33 (s, 3H), 1.32 (s, 9H). LCMS Method A; RT 2.13; m/z [M+H]⁺ 401.2.

### Example 41 (2018)

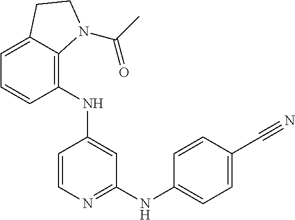 [SMILES: CC(=O)N1CCc2cccc(Nc3ccnc(Nc4ccc(C#N)cc4)c3)c21]

### 4-((4-((1-Acetylindolin-7-yl)amino)pyridin-2-yl)amino)benzonitrile

Synthesised using the procedure described in Example 1 using Intermediate B1 and 4-aminobenzonitrile. ¹H NMR (CDCl₃, 400 MHz): 8.47 (br s, 1H), 7.95 (d, J=5.8 Hz, 1H), 7.52 (d, J=8.9 Hz, 2H), 7.43 (d, J=9.0 Hz, 2H), 7.29 (d, J=7.9 Hz, 1H), 7.15 (t, J=7.4 Hz, 1H), 6.96 (dd, J=7.2 and 1.0 Hz, 1H), 6.60 (br s, 1H), 6.48 (dd, J=5.8 and 2.0 Hz, 1H), 6.39 (d, J=1.8 Hz, 1H), 4.12 (t, J=7.8 Hz, 2H), 3.13 (t, J=7.8 Hz, 2H), 2.34 (s, 3H). LCMS Method A; RT 1.67; m/z [M+H]⁺ 370.2.

### Example 42 (2019)

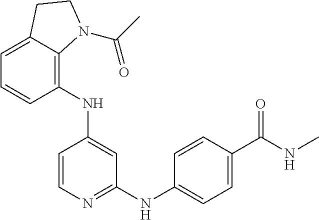 [SMILES: CNC(=O)c1ccc(Nc2cc(Nc3cccc4c3N(C(C)=O)CC4)ccn2)cc1]

### 4-((4-((1-Acetylindolin-7-yl)amino)pyridin-2-yl)amino)-N-methylbenzamide

Synthesised using the procedure described in Example 1 using Intermediate B1 and 4-amino-N-methylbenzamide. ¹H NMR (CDCl₃, 400 MHz): 8.43 (br s, 1H), 7.91 (d, J=5.8 Hz, 1H), 7.68 (d, J=8.8 Hz, 2H), 7.32 (d, J=8.7 Hz, 2H), 7.29 (d, J=7.7 Hz, 1H), 7.14 (t, J=7.4 Hz, 1H), 6.94 (d, J=8.2 Hz, 1H), 6.69 (br s, 1H), 6.45-6.42 (m, 2H), 6.06 (br d, J=4.9 Hz, 1H), 4.11 (t, J=7.8 Hz, 2H), 3.12 (t, J=7.7 Hz, 2H), 3.00 (d, J=4.8 Hz, 3H), 2.33 (s, 3H). LCMS Method A; RT 1.44; m/z [M+H]⁺ 402.2.

### Example 43 (2020)

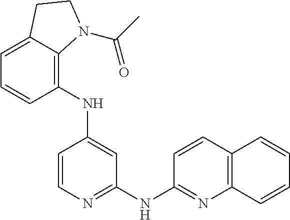 [SMILES: CC(=O)N1CCc2cccc(Nc3ccnc(Nc4ccc5ccccc5n4)c3)c21]

### 1-(7-((2-(Quinolin-2-ylamino)pyridin-4-yl)amino)indolin-1-yl)ethan-1-one

Synthesised using the procedure described in Example 1 using Intermediate B1 and quinolin-2-amine. ¹H NMR (CDCl₃, 400 MHz): 8.85 (br s, 1H), 8.15 (br, s, 1H), 7.99 (d, J=8.8 Hz, 1H), 7.90 (d, J=6.0 Hz, 1H), 7.75 (d, J=8.4 Hz, 1H), 7.69 (d, 8.0 Hz, 1H), 7.63 (t, J=8.4 Hz, 1H), 7.55 (d, J=8.0 Hz, 1H), 7.39-7.30 (m, 4H), 7.06 (d, J=8.2 Hz, 1H), 6.51 (dd, J=6.1 and 2.2 Hz, 1H), 4.16 (t, J=7.8 Hz, 2H), 3.18 (t, J=7.8 Hz, 2H), 2.38 (s, 3H). LCMS Method A; RT 2.04; m/z [M+H]⁺ 396.2.

### Example 44 (2021)

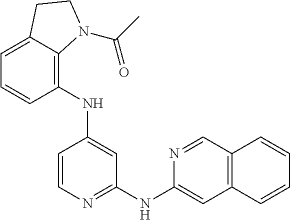 [SMILES: CC(=O)N1CCc2cccc(Nc3ccnc(Nc4cc5ccccc5cn4)c3)c21]

### 1-(7-((2-(Isoquinolin-3-ylamino)pyridin-4-yl)amino)indolin-1-yl)ethan-1-one

Synthesised using the procedure described in Example 1 using Intermediate B1 and isoquinolin-3-amine. ¹H NMR (CDCl₃, 400 MHz): 8.86 (s, 1H), 8.64 (br s, 1H), 7.99 (s, 1H), 7.82 (d, J=6.0 Hz, 1H), 7.75 (d, J=8.9 Hz, 1H), 7.66 (d, J=8.3 Hz, 1H), 7.50 (t, J=6.8 Hz, 1H), 7.33-7.27 (m, 2H), 7.19-7.13 (m, 2H), 6.92 (d, J=7.3 Hz, 2H), 6.38 (dd, J=6.2 and 2.1 Hz, 1H), 4.06 (t, J=7.8 Hz, 2H), 3.07 (t, J=7.8 Hz, 2H), 2.28 (s, 3H). LCMS Method A; RT 2.00; m/z [M+H]⁺ 396.2.

### Example 45 (2022)

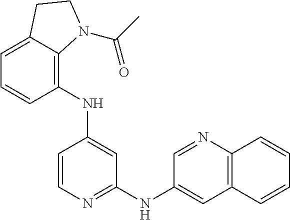 [SMILES: CC(=O)N1CCc2cccc(Nc3ccnc(Nc4cnc5ccccc5c4)c3)c21]

### 1-(7-((2-(Quinolin-3-ylamino)pyridin-4-yl)amino)indolin-1-yl)ethan-1-one

Synthesised using the procedure described in Example 1 using Intermediate B1 and quinolin-3-amine. ¹H NMR (CDCl₃, 400 MHz): 8.69 (d, J=2.7 Hz, 1H), 8.36 (s, 1H), 8.29 (d, J=2.4 Hz, 1H), 7.94-7.90 (m, 2H), 7.66 (d, J=9.4 Hz, 1H), 7.49-7.40 (m, 2H), 7.26 (d, J=8.0 Hz, 1H), 7.08 (t, J=7.4 Hz, 1H), 6.86 (d, J=8.3 Hz, 1H), 6.63 (br s, 1H), 6.40-6.37 (m, 2H), 4.04 (t, J=7.8 Hz, 2H), 3.05 (t, J=7.8 Hz, 2H), 2.26 (s, 3H). LCMS Method A; RT 1.67; m/z [M+H]396.1.

### Example 46 (2023)

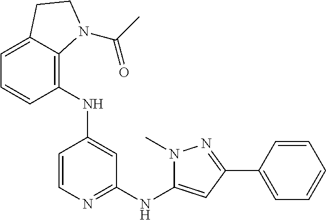 [SMILES: CC(=O)N1CCc2cccc(Nc3ccnc(Nc4cc(-c5ccccc5)nn4C)c3)c21]

### 1-(7-((2-((1-Methyl-3-phenyl-1H-pyrazol-5-yl)amino)pyridin-4-yl)amino)indolin-1-yl)ethan-1-one

Synthesised using the procedure described in Example 1 using Intermediate B1 and 2-methyl-5-phenyl-pyrazol-3-amine. ¹H NMR (CDCl₃, 400 MHz): 8.31 (br s, 1H), 7.78 (d, J=5.9 Hz, 1H), 7.69 (d, J=7.1 Hz, 2H), 7.32 (t, J=7.3 Hz, 1H), 7.23 (d, J=7.4 Hz, 1H), 7.17 (s, 1H), 7.01 (d, J=7.4 Hz, 1H), 6.83 (d, J=7.3 Hz, 1H), 6.34-6.32 (m, 2H), 6.08 (d, J=1.8 Hz, 1H), 5.23 (s, 1H), 4.02 (t, J=7.8 Hz, 2H), 3.72 (s, 3H), 3.03 (t, J=7.7 Hz, 2H), 2.24 (s, 3H). LCMS Method A; RT 1.78; m/z [M+H]⁺ 425.2

### Example 47 (2026)

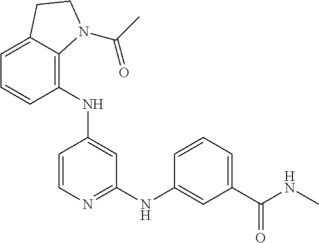 [SMILES: CNC(=O)c1cccc(Nc2cc(Nc3cccc4c3N(C(C)=O)CC4)ccn2)c1]

### 3-((4-((1-Acetylindolin-7-yl)amino)pyridin-2-yl)amino)-N-methylbenzamide

Synthesised using the procedure described in Example 1 using Intermediate B1 and 3-amino-N-methyl-benzamide. ¹H NMR (CDCl₃, 400 MHz): 8.44 (s, 1H), 7.88 (d, J=6.1 Hz, 1H), 7.72 (br s, 1H), 7.44-7.41 (m, 1H), 7.33-7.29 (m, 3H), 7.14 (t, J=7.4 Hz, 1H), 6.93 (d, J=8.3 Hz, 1H), 6.71 (br s, 1H), 6.40-6.38 (m 2H), 6.22 (br d, J=3.4 Hz, 1H), 4.10 (t, J=7.8 Hz, 2H), 3.11 (t, J=7.8 Hz, 2H), 3.01 (d, J=4.8 Hz, 3H), 2.33 (s, 3H).

LCMS Method A; RT 1.48; m/z [M+H]⁺ 402.2.

### Example 48 (2027)

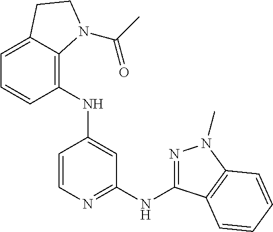 [SMILES: CC(=O)N1CCc2cccc(Nc3ccnc(Nc4nn(C)c5ccccc45)c3)c21]

### 1-(7-((2-((1-Methyl-1H-indazol-3-yl)amino)pyridin-4-yl)amino)indolin-1-yl)ethan-1-one

Synthesised using the procedure described in Example 1 using Intermediate B1 and 1-methylindazol-3-amine. ¹H NMR (CDCl₃, 400 MHz): 8.72 (br s, 1H), 7.82 (d, J=6.2 Hz, 2H), 7.62 (s, 1H), 7.46-7.40 (m, 2H), 7.31 (s, 1H), 7.27 (s, 1H), 7.18 (t, J=7.6 Hz, 1H), 7.12 (t, J=7.9 Hz, 1H), 7.00 (dd, J=7.4 and 0.8 Hz, 1H), 6.41 (dd, J=6.2 and 2.2 Hz, 1H), 4.15 (t, J=7.8 Hz, 2H), 3.96 (s, 3H), 3.16 (t, J=7.7 Hz, 2H), 2.37 (s, 3H). LCMS Method A; RT 1.93; m/z [M+H]⁺ 399.2.

### Example 49 (2028)

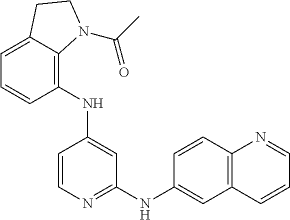 [SMILES: CC(=O)N1CCc2cccc(Nc3ccnc(Nc4ccc5ncccc5c4)c3)c21]

### 1-(7-((2-(Quinolin-6-ylamino)pyridin-4-yl)amino)indolin-1-yl)ethan-1-one

Synthesised using the procedure described in Example 1 using Intermediate B1 and quinolin-6-amine. ¹H NMR (CDCl₃, 400 MHz): 8.68 (dd, J=4.2 and 1.7 Hz, 1H), 8.38 (br s, 1H), 7.96 (d, J=8.4 Hz, 1H), 7.92 (d, J=9.1 Hz, 1H), 7.87 (d, J=5.9 Hz, 1H), 7.77 (d, J=2.5 Hz, 1H), 7.47 (dd, J=9.0 and 2.5 Hz, 1H), 7.28-7.25 (m, 2H), 7.07 (t, J=7.4 Hz, 1H), 6.86 (d, J=7.3 Hz, 1H), 6.79 (br s, 1H), 6.44 (d, J=1.8 Hz, 1H), 6.37 (dd, J=5.9 and 2.0 Hz, 1H), 4.03 (t, J=7.7 Hz, 2H), 3.04 (t, J=7.7 Hz, 2H), 2.26 (s, 3H). LCMS Method A; RT 1.40; m/z [M+H]⁺ 396.2.

### Example 50 (2029)

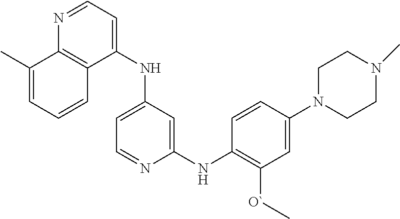

### N2-[2-Methoxy-4-(4-methylpiperazin-1-yl)phenyl]-N4-(4-quinolyl)pyridine-2,4-diamine

Quinolin-4-amine (33 mg, 0.23 mmol), Pd(OAc)₂ (2.7 mg, 0.012 mmol) and XANTPHOS (10 mg, 0.018 mmol) were placed in a microwave vial and then Intermediate C1 (50 mg, 0.15 mmol) in DMA (1 mL) was added. The vial was sealed, evacuated and purged with argon three times. Cesium carbonate (98 mg, 0.30 mmol) in water (0.5 mL) was added (vial evacuated and purged with argon) and the reaction mixture was heated in the microwave at 150° C. for 1 hour. The reaction mixture was then diluted with ethyl acetate, washed with water (3×25 mL) and brine (25 mL). The organic layer was dried over sodium sulfate and the solvent reduced in vacuo. The resulting residue was purified by flash chromatography (0-50% of 90/10/1 DCM/MeOH/NH₄OH). The appropriate fractions were combined and the solvent reduced in vacuo. Further purification by HPLC (XBridge column, 0.1% NH₄OH modifier) followed by evaporation of solvent from the appropriate fractions in a genevac gave the title compound (9 mg, 9%). ¹H NMR (CDCl₃, 400 MHz): 8.70-8.79 (1H, m), 8.04-8.17 (2H, m), 7.88-7.98 (1H, m), 7.69-7.80 (1H, m), 7.50-7.62 (2H, m), 7.31-7.37 (1H, m), 6.45-6.73 (6H, m), 3.87 (3H, s), 3.21 (4H, br. s.), 2.55-2.73 (4H, m), 2.39 (3H, s). LCMS Method B; RT=0.67; m/z [M+H]⁺ 441.2.

### Example 51 (2031)

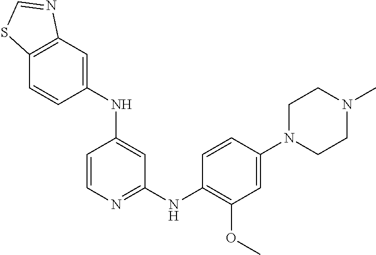 [SMILES: COc1cc(N2CCN(C)CC2)ccc1Nc1cc(Nc2ccc3scnc3c2)ccn1]

### N4-(benzo[d]thiazol-5-yl)-N2-(2-methoxy-4-(4-methylpiperazin-1-yl)phenyl)pyridine-2,4-diamine

Synthesised using the procedure described in Example 50 using Intermediate C1 and benzo[d]thiazol-5-amine. ¹H NMR (CDCl₃, 400 MHz): 8.97-9.07 (1H, m), 7.93-8.00 (2H, m), 7.90 (1H, d, J=8.5 Hz), 7.58-7.70 (1H, m), 6.47-6.63 (3H, m), 6.28-6.43 (2H, m), 6.05 (1H, s), 3.80-3.93 (3H, m), 3.07-3.25 (4H, m), 2.53-2.68 (4H, m), 2.38 (3H, s). LCMS Method B; RT=1.19; m/z [M+H]⁺ 447.2.

### Example 52 (2038)

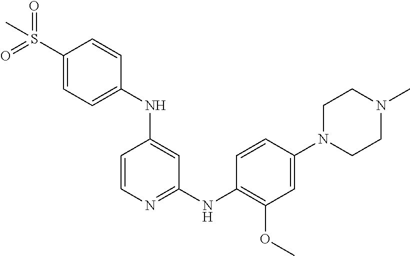

### N2-(2-Methoxy-4-(4-methylpiperazin-1-yl)phenyl)-N4-(4-(methylsulfonyl)phenyl)pyridine-2,4-diamine

Synthesised using the procedure described in Example 50 using Intermediate C1 and 4-(methylsulfonyl)aniline. ¹H NMR (CDCl₃, 400 MHz): 8.00-8.07 (1H, m), 7.80-7.87 (2H, m), 7.51-7.59 (1H, m), 7.16-7.25 (2H, m), 6.48-6.60 (3H, m), 6.41-6.47 (1H, m), 6.37-6.41 (1H, m), 6.25-6.33 (1H, m), 3.85 (3H, s), 3.12-3.28 (4H, m), 3.05 (3H, s), 2.52-2.67 (4H, m), 2.37 (3H, s). LCMS Method B; RT=1.08; m/z [M+H]⁺ 468.2.

### Example 53 (2039)

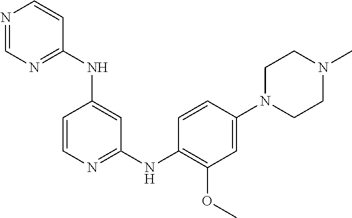 [SMILES: COc1cc(N2CCN(C)CC2)ccc1Nc1cc(Nc2ccncn2)ccn1]

### N2-(2-Methoxy-4-(4-methylpiperazin-1-yl)phenyl)-N4-(pyrimidin-4-yl)pyridine-2,4-diamine

Synthesised using the procedure described in Example 50 using Intermediate C1 and pyrimidin-4-amine. ¹H NMR (CDCl₃, 400 MHz): 8.72-8.80 (1H, m), 8.34-8.41 (1H, m), 8.04-8.11 (1H, m), 7.56-7.63 (1H, m), 7.28-7.34 (1H, m), 6.89-6.94 (1H, m), 6.78-6.83 (1H, m), 6.73-6.77 (1H, m), 6.64-6.69 (1H, m), 6.50-6.57 (2H, m), 3.84 (1H, s), 3.09-3.29 (4H, m), 2.52-2.70 (4H, m), 2.37 (3H, s). LCMS Method B; RT=0.94; m/z [M+H]⁺ 392.2.

### Example 54 (2043)

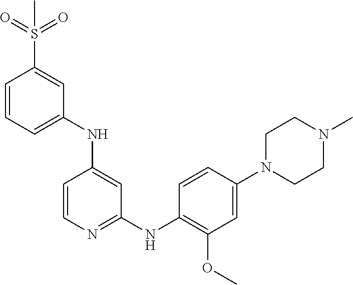 [SMILES: COc1cc(N2CCN(C)CC2)ccc1Nc1cc(Nc2cccc(S(C)(=O)=O)c2)ccn1]

### N2-(2-Methoxy-4-(4-methylpiperazin-1-yl)phenyl)-N4-(3-(methylsulfonyl)phenyl)pyridine-2,4-diamine

Synthesised using the procedure described in Example 50 using Intermediate C1 and 3-methylsulfonylaniline. ¹H NMR (DMSO-d6, 400 MHz): 8.88 (1H, s), 7.81 (1H, d, J=5.5 Hz), 7.50-7.61 (4H, m), 7.40-7.49 (2H, m), 6.60 (1H, d, J=2.3 Hz), 6.45 (1H, dd, J=8.7, 2.4 Hz), 6.31-6.38 (2H, m), 3.79 (3H, s), 3.19 (3H, s), 3.06-3.14 (4H, m), 2.42-2.48 (4H, m), 2.22 (3H, s). LCMS Method B; RT=1.07; m/z [M+H]⁺ 468.2.

### Example 55 (2045)

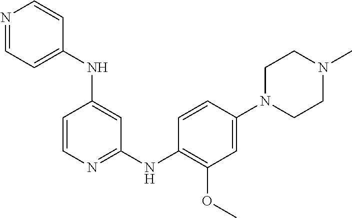 [SMILES: COc1cc(N2CCN(C)CC2)ccc1Nc1cc(Nc2ccncc2)ccn1]

### N2-2-Methoxy-4-(4-methylpiperazin-1-yl)phenyl)-N4-(pyridin-4-yl)pyridine-2,4-diamine

Synthesised using the procedure described in Example 50 using Intermediate C1 and pyridin-4-amine. ¹H NMR (CDCl₃, 400 MHz): 8.37-8.47 (2H, m), 8.07 (1H, d, J=5.8 Hz), 7.50-7.62 (1H, m), 6.92-7.04 (2H, m), 6.52-6.60 (3H, m), 6.49 (1H, dd, J=5.8, 2.0 Hz), 6.43 (1H, d, J=1.8 Hz), 6.19 (1H, s), 3.87 (3H, s), 3.16-3.26 (4H, m), 2.56-2.68 (4H, m), 2.39 (3H, s). LCMS Method C; RT=0.31; m/z [M+H]⁺ 391.1.

### Example 56 (2046)

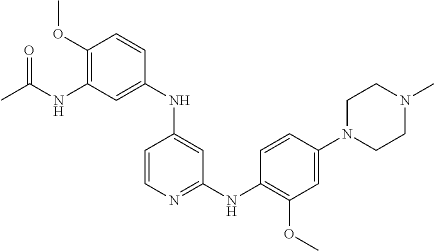 [SMILES: COc1ccc(Nc2ccnc(Nc3ccc(N4CCN(C)CC4)cc3OC)c2)cc1NC(C)=O]

### N-(2-Methoxy-5-((2-((2-methoxy-4-(4-methylpiperazin-1-yl)phenyl)amino)pyridin-4-yl)amino)phenyl)acetamide

Synthesised using the procedure described in Example 50 using Intermediate C1 and N-(5-amino-2-methoxyphenyl)acetamide. ¹H NMR (CDCl₃, 400 MHz): 8.20-8.31 (1H, m), 7.85-7.91 (1H, m), 7.78 (1H, br. s.), 7.59 (1H, d, J=8.5 Hz), 6.91 (1H, dd, J=, 8.7, 2.4 Hz), 6.81-6.87 (1H, m), 6.55-6.64 (2H, m), 6.52 (1H, dd, J=8.5, 2.5 Hz), 6.25 (1H, dd, J=5.9, 1.9 Hz), 6.20 (1H, s), 5.81 (1H, br. s.), 3.91 (3H, s), 3.85 (3H, s), 3.11-3.28 (4H, m), 2.56-2.71 (4H, m), 2.38 (3H, s), 2.23 (3H, s). LCMS Method B; RT=1.16; m/z [M+H]⁺ 477.2.

### Example 57 (2047)

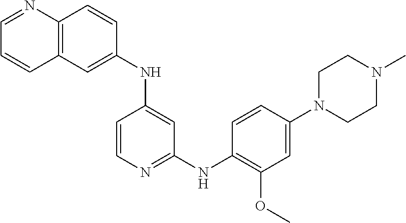 [SMILES: COc1cc(N2CCN(C)CC2)ccc1Nc1cc(Nc2ccc3ncccc3c2)ccn1]

### N2-(2-Methoxy-4-(4-methylpiperazin-1-yl)phenyl)-N4-(quinolin-6-yl)pyridine-2,4-diamine

Synthesised using the procedure described in Example 50 using Intermediate C1 and quinolin-6-amine. ¹H NMR (CDCl₃, 400 MHz): 8.83 (1H, dd, J=4.3, 1.83 Hz), 8.09-7.99 (3H, m), 7.60-7.50 (3H, m), 7.39 (1H, dd, J=8.3, 4.3 Hz), 6.57-6.51 (3H, m), 6.46-6.41 (2H, m), 6.19 (1H, s), 3.86-3.86 (3H, m), 3.21-3.17 (4H, m), 2.63-2.59 (4H, m), 2.39-2.38 (3H, m). LCMS Method B; RT=1.06; m/z [M+H]⁺ 441.2.

### Example 58 (2049)

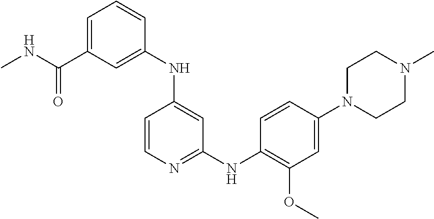

### 3-((2-((2-Methoxy-4-(4-methylpiperazin-1-yl)phenyl)amino)pyridin-4-yl)amino)-N-methylbenzamide

Synthesised using the procedure described in Example 50 using Intermediate C1 and 3-amino-N-methylbenzamide. ¹H NMR (CDCl₃, 400 MHz): 7.97 (1H, d, J=6.0 Hz), 7.59-7.56 (2H, m), 7.39-7.37 (2H, m), 7.29-7.28 (1H, m), 6.57-6.48 (3H, m), 6.35-6.32 (2H, m), 6.14 (1H, d, J=3.8 Hz), 6.01 (1H, s), 3.86-3.85 (3H, m), 3.21-3.17 (4H, m), 3.05-3.03 (3H, m), 2.63-2.59 (4H, m), 2.38 (3H, s). LCMS Method B; RT=1.12; m/z [M+H]⁺ 447.2.

### Example 59 (2052)

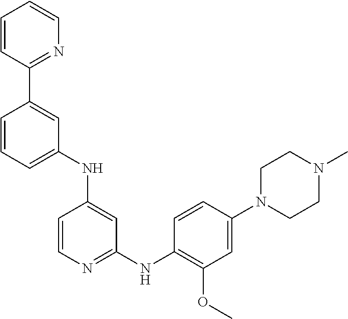 [SMILES: COc1cc(N2CCN(C)CC2)ccc1Nc1cc(Nc2cccc(-c3ccccn3)c2)ccn1]

### N2-(2-ethoxy-4-(4-methylpiperazin-1-yl)phenyl)-N4-(3-(pyridin-2-yl)phenyl)pyridine-2,4-diamine

Synthesised using the procedure described in Example 50 using Intermediate C1 and 3-(pyridin-2-yl)aniline. ¹H NMR (CDCl₃, 400 MHz): 8.73-8.71 (1H, m), 7.97-7.95 (1H, m), 7.88 (1H, t, J=2.0 Hz), 7.81-7.67 (3H, m), 7.58-7.55 (1H, m), 7.44 (1H, t, J=7.9 Hz), 7.28 (1H, s), 6.58-6.54 (2H, m), 6.42-6.34 (3H, m), 6.02 (1H, s), 3.84 (3H, s), 3.17-3.12 (4H, m), 2.62-2.58 (4H, m), 2.38-2.38 (3H, m). LCMS Method B; RT=1.21; m/z [M+H]⁺ 467.2.

### Example 60 (2054)

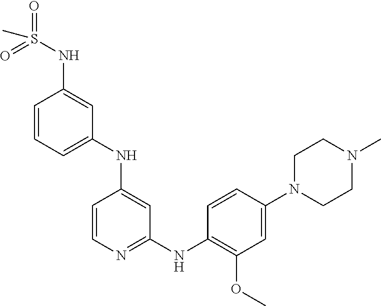

### N-(3-((2-((2-Methoxy-4-(4-methylpiperazin-1-yl)phenyl)amino)pyridin-4-yl)amino)phenyl)methanesulfonamide

Synthesised using the procedure described in Example 50 using Intermediate C1 and N-(3-aminophenyl)methanesulfonamide. ¹H NMR (DMSO-d6, 400 MHz): 9.70 (1H, br. s.), 8.57 (1H, s), 7.75 (1H, d, J=6.3 Hz), 7.57 (1H, d, J=8.5 Hz), 7.39 (1H, s), 7.22 (1H, t, J=8.0 Hz), 7.01 (1H, s), 6.85-6.94 (1H, m), 6.79 (1H, dd, J=8.0, 1.3), 6.60 (1H, d, J=2.5 Hz), 6.45 (1H, dd, J=8.7, 2.4 Hz), 6.24-6.34 (2H, m), 3.78 (3H, s), 3.04-3.15 (4H, m), 2.98 (3H, s), 2.40-2.48 (4H, m), 2.23 (3H, s). LCMS Method B; RT=1.09; m/z [M+H]⁺ 483.2.

### Example 61 (2055)

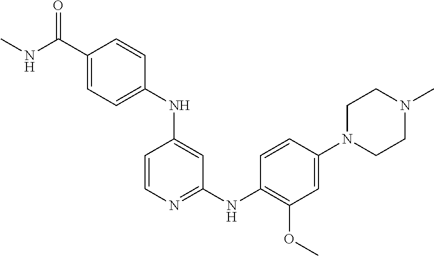

### 4-((2-((2-Methoxy-4-(4-methylpiperazin-1-yl)phenyl)amino)pyridin-4-yl)amino)-N-methylbenzamide

Synthesised using the procedure described in Example 50 using Intermediate C1 and 4-amino-N-methylbenzamide. ¹H NMR (CDCl₃, 400 MHz): 7.99 (1H, d, J=5.5 Hz), 7.72 (2H, d, J=8.8 Hz), 7.56 (1H, d, J=8.5 Hz), 7.14 (2H, d, J=8.8 Hz), 6.56-6.50 (3H, m), 6.39-6.36 (2H, m), 6.08 (2H, m), 3.85 (3H, s), 3.18 (4H, m), 3.02 (3H, 2×s, rotamer), 2.60 (4H, m), 2.37 (3H, s). LCMS Method B; RT=1.15; m/z [M+H]⁺ 447.3.

### Example 62 (2056)

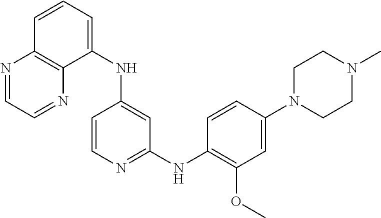 [SMILES: COc1cc(N2CCN(C)CC2)ccc1Nc1cc(Nc2cccc3nccnc23)ccn1]

### N2-(2-Methoxy-4-(4-methylpiperazin-1-yl)phenyl)-N4-(quinoxalin-5-yl)pyridine-2,4-diamine

Synthesised using the procedure described in Example 50 using Intermediate C1 and quinoxalin-5-amine. ¹H NMR (CDCl₃, 400 MHz): 8.90 (1H, d, J=1.8 Hz), 8.73 (1H, d, J=1.8 Hz), 8.20 (1H, br s), 8.08 (1H, d, J=5.8 Hz), 7.72-7.62 (4H, m), 6.70 (1H, dd, J=5.8, 2.0 Hz), 6.65 (1H, d, J=2.0 Hz), 6.61 (1H, br s), 6.58-6.56 (2H, m), 3.87 (3H, s), 3.20 (4H, t, J=5.0 Hz), 2.62 (4H, t, J=5.0 Hz), 2.38 (3H, s). LCMS Method B; RT=1.15; m/z [M+H]⁺ 447.3.

### Example 63 (2057)

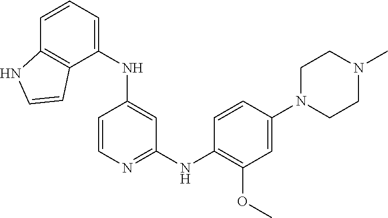 [SMILES: COc1cc(N2CCN(C)CC2)ccc1Nc1cc(Nc2cccc3[nH]ccc23)ccn1]

### N4-(1H-Indol-4-yl)-N2-(2-methoxy-4-(4-methylpiperazin-1-yl)phenyl)pyridine-2,4-diamine

A mixture of Intermediate C1 (75 mg, 0.2 mmol), 1H-indol-4-amine (33 mg, 0.25 mmol), XANTPHOS (8 mg, 0.014 mmol) and cesium carbonate (147 mg, 0.45 mmol) in DMA (1.5 mL) was degassed by bubbling argon for 10 minutes. Then, Pd(OAc)₂ (2 mg, 0.01 mmol) was added and the reaction was heated thermally at 150° C. for 90 minutes. The reaction was diluted with MeOH and loaded directly on to a 5 g SCX cartridge that was eluted with MeOH and then with 2M methanolic ammonia. The basic fractions were combined and reduced in vacuo. Purification by flash chromatography (0-10% MeOH in DCM) gave the title compound as a brown solid (13 mg, 13% yield). ¹H NMR (CDCl₃, 400 MHz): 8.32 (1H, br s), 7.92 (1H, d, J=5.8 Hz), 7.59 (1H, d, J=8.6 Hz), 7.21 (2H, m), 7.16 (1H, t, J=8.0 Hz), 7.03 (1H, m), 6.59-6.53 (2H, m), 6.50-6.47 (2H, m), 6.34 (1H, dd, J=5.8, 2.0 Hz), 6.30 (1H, d, J=1.8 Hz), 6.08 (1H, br s), 3.83 (3H, s), 3.16 (4H, m), 2.60 (4H, m), 2.37 (3H, s). LCMS Method B; RT=1.01; m/z [M+H]⁺ 429.3.

### Example 64 (2061)

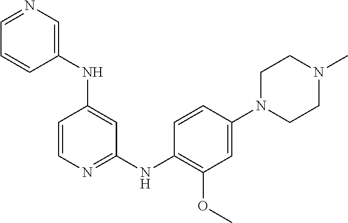 [SMILES: COc1cc(N2CCN(C)CC2)ccc1Nc1cc(Nc2cccnc2)ccn1]

### N2-(2-Methoxy-4-(4-methylpiperazin-1-yl)phenyl)-N4-(pyridin-3-yl)pyridine-2,4-diamine

Synthesised using the procedure described in Example 63 using Intermediate C1 and pyridin-3-amine. ¹H NMR (CDCl₃, 400 MHz): 8.48 (1H, d, J=2.5 Hz), 8.33 (1H, dd, J=4.7, 1.5 Hz), 7.99 (1H, d, J=5.7 Hz), 7.58 (1H, d, J=8.6 Hz), 7.53 (1H, ddd, J=8.3, 2.6, 1.7 Hz), 7.26-7.24 (1H, m), 6.57 (1H, d, J=2.3 Hz), 6.54 (1H, dd, J=8.6, 2.5 Hz), 6.51 (1H, s), 6.33 (1H, dd, J=5.9, 2.1 Hz), 6.28 (1H, d, J=2.0 Hz), 5.88 (1H, s), 3.86 (3H, s), 3.23-3.17 (4H, m), 2.64-2.59 (4H, m), 2.59 (3H, s). LCMS Method B; RT=0.57; m/z [M+H]⁺ 391.0.

### Example 65 (2063)

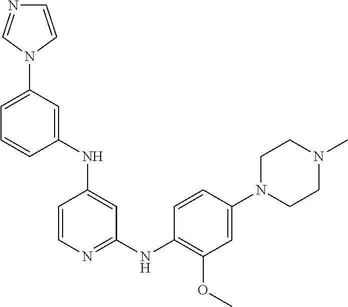 [SMILES: COc1cc(N2CCN(C)CC2)ccc1Nc1cc(Nc2cccc(-n3ccnc3)c2)ccn1]

### N4-(3-(1H-Imidazol-1-yl)phenyl)-N2-(2-methoxy-4-(4-methylpiperazin-1-yl)phenyl)pyridine-2,4-diamine

Synthesised using the procedure described in Example 50 using Intermediate C1 and 3-(1H-imidazol-1-yl)aniline. ¹H NMR (DMSO-d6, 400 MHz) Q: 8.71 (1H, s), 8.23-8.19 (1H, m), 7.78 (1H, d, J=5.7 Hz), 7.68 (1H, t, J=1.4 Hz), 7.55 (1H, d, J=8.7 Hz), 7.48 (1H, s), 7.41 (1H, t, J=8.1 Hz), 7.33 (1H, t, J=2.1 Hz), 7.17 (1H, ddd, J=8.0, 2.3, 0.7 Hz), 7.11 (1H, t, J=1.0 Hz), 7.09 (1H, ddd, J=8.2, 2.0, 0.8 Hz), 6.58 (1H, d, J=2.7 Hz), 6.41 (1H, d, J=2.0 Hz), 6.37 (1H, dd, J=8.6, 2.7 Hz), 6.32 (1H, dd, J=5.7, 2.1 Hz), 3.76 (3H, s), 3.11-3.02 (4H, m), 2.48-2.41 (4H, m), 2.22 (3H, s). LCMS Method C; RT=0.35; m/z [M+H]⁺ 456.2.

### Example 66 (2064)

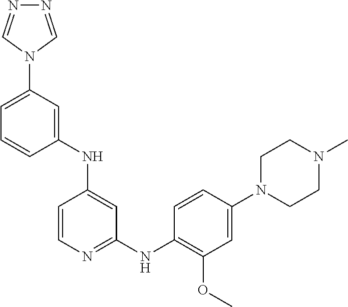 [SMILES: COc1cc(N2CCN(C)CC2)ccc1Nc1cc(Nc2cccc(-n3cnnc3)c2)ccn1]

### N4-(3-(4H-1,2,4-Triazol-4-yl)phenyl)-N2-(2-methoxy-4-(4-methylpiperazin-1-yl)phenyl)pyridine-2,4-diamine

Synthesised using the procedure described in Example 50 using Intermediate C1 and 3-(1,2,4-triazol-4-yl)aniline. ¹H NMR (DMSO-d6, 400 MHz): 9.10 (2H, s), 8.77 (1H, s), 7.79 (1H, d, J=5.7 Hz), 7.58 (1H, d, J=8.7 Hz), 7.49-7.43 (2H, m), 7.42 (1H, t, J=2.2 Hz), 7.21 (1H, ddd, J=8.0, 2.2, 0.7 Hz), 7.15 (1H, ddd, J=8.2, 2.2, 0.8 Hz), 6.57 (1H, d, J=2.7 Hz), 6.43 (1H, d, J=2.0 Hz), 6.38-6.31 (2H, m), 3.76 (3H, s), 3.10-3.02 (4H, m), 2.47-2.41 (4H, m), 2.22 (3H, s). LCMS Method B; RT=1.04; m/z [M+H]⁺ 416.0.

### Example 67 (2066)

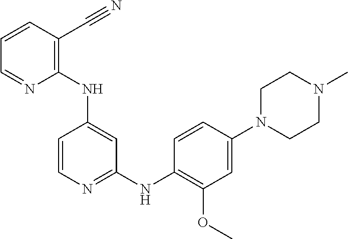 [SMILES: COc1cc(N2CCN(C)CC2)ccc1Nc1cc(Nc2ncccc2C#N)ccn1]

### 2-((2-((2-Methoxy-4-(4-methylpiperazin-1-yl)phenyl)amino)pyridin-4-yl)amino)nicotinonitrile

Synthesised using the procedure described in Example 50 using Intermediate C1 and 2-aminonicotinonitrile. ¹H NMR (CDCl₃, 400 MHz): 8.43-8.54 (1H, m), 8.06-8.17 (1H, m), 7.86 (1H, dd, J=7.8, 1.8 Hz), 7.73 (1H, d, J=9.3 Hz), 7.24 (1H, d, J=1.8 Hz), 6.97-7.05 (2H, m), 6.93 (1H, dd, J=7.5, 5.0 Hz), 6.68 (1H, br. s.), 6.56-6.62 (2H, m), 3.88 (3H, s), 3.15-3.33 (4H, m), 2.59-2.70 (4H, m), 2.40 (3H, s). LCMS Method B; RT=1.10; m/z [M+H]⁺ 416.0.

### Example 68 (2067)

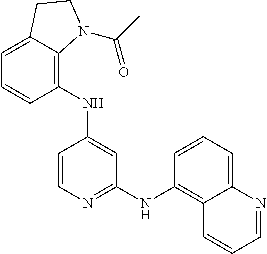 [SMILES: CC(=O)N1CCc2cccc(Nc3ccnc(Nc4cccc5ncccc45)c3)c21]

### 1-(7-((2-(Quinolin-5-ylamino)pyridin-4-yl)amino)indolin-1-yl)ethan-1-one

Synthesised using the procedure described in Example 1 using Intermediate B1 and quinolin-5-amine. ¹H NMR (CDCl₃, 400 MHz): 8.87 (dd, J=4.2 and 1.6 Hz, 1H), 8.36 (d, J=9.2 Hz, 1H), 8.31 (br s, 1H), 7.85 (d, J=8.5 Hz, 1H), 7.78 (d, J=6.0 Hz, 1H), 7.60 (t, J=7.5 Hz, 1H), 7.49 (dd, J=7.5 and 1.0 Hz, 1H), 7.34 (dd, J=8.5 and 4.2 Hz, 1H), 7.07-7.02 (m, 2H), 6.87 (t, J=7.4 Hz, 1H), 6.78 (dd, J=7.2 and 0.9 Hz, 1H), 6.29 (dd, J=6.0 and 2.0 Hz, 1H), 6.07 (d, J=1.9 Hz, 1H), 3.98 (t, J=7.8 Hz, 2H), 2.99 (t, J=7.7 Hz, 2H), 2.21 (s, 3H). LCMS Method A; RT=1.34; m/z [M+H]⁺ 396.2.

### Example 69 (2069)

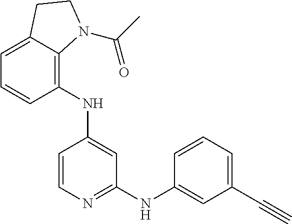 [SMILES: C#Cc1cccc(Nc2cc(Nc3cccc4c3N(C(C)=O)CC4)ccn2)c1]

### 1-(7-((2-((3-Ethynylphenyl)amino)pyridin-4-yl)amino)indolin-1-yl)ethan-1-one

Synthesised using the procedure described in Example 1 using Intermediate B1 and 3-aminophenylacetylene. ¹H NMR (CDCl₃, 400 MHz): 8.54 (br s, 1H), 8.21 (d, J=5.8 Hz, 1H), 7.31 (d, J=8.1 Hz, 1H), 7.17 (t, J=7.4 Hz, 1H), 7.11 (t, J=7.8 Hz, 1H), 7.06 (d, J=2.0 Hz, 1H), 6.99-6.96 (m, 2H), 6.88 (t, J=2.0 Hz, 1H), 6.75 (dd, J=8.2 and 2.4 Hz, 1H), 6.68-6.65 (m, 1H), 4.12 (t, J=7.8 Hz, 2H), 3.68 (br s, 1H), 3.14 (t, J=7.8 Hz, 2H), 2.35 (s, 3H). LCMS Method A; RT=1.59; m/z [M+H]⁺ 369.2.

### Example 70 (2070)

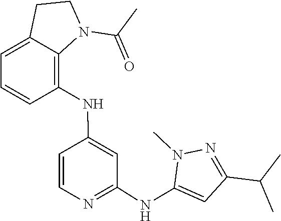 [SMILES: CC(=O)N1CCc2cccc(Nc3ccnc(Nc4cc(C(C)C)nn4C)c3)c21]

### 1-(7-((2-((3-Isopropyl-1-methyl-1H-pyrazol-5-yl)amino)pyridin-4-yl)amino)indolin-1-yl)ethan-1-one

Synthesised using the procedure described in Example 1 using Intermediate B1 and isopropyl-2-methyl-pyrazol-3-amine. ¹H NMR (CDCl₃, 400 MHz): 8.38 (br s, 1H), 7.73 (d, J=5.9 Hz, 1H), 7.14 (d, J=9.0 Hz, 1H), 7.02 (t, J=7.3 Hz, 1H), 6.85 (dd, J=7.3 and 1.0 Hz, 1H), 6.28 (dd, J=6.0 and 2.1 Hz, 1H), 6.04 (d, J=1.8 Hz, 1H), 5.83 (s, 1H), 4.03 (t, J=7.8 Hz, 2H), 3.60 (s, 1H), 3.04 (t, J=7.7 Hz, 2H), 2.25 (s, 3H), 1.19 (s, 3H), 1.17 (s, 3H). LCMS Method A; RT=1.77; m/z [M+H]⁺ 391.2

### Example 71 (2071)

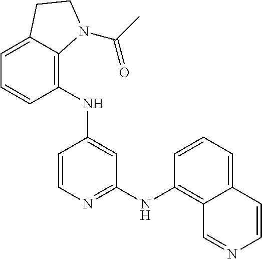 [SMILES: CC(=O)N1CCc2cccc(Nc3ccnc(Nc4cccc5ccncc45)c3)c21]

### 1-(7-((2-(Isoquinolin-8-ylamino)pyridin-4-yl)amino)indolin-1-yl)ethan-1-one

Synthesised using the procedure described in Example 1 using Intermediate B1 and isoquinolin-8-amine. ¹H NMR (CDCl₃, 400 MHz): 9.43 (s, 1H), 8.48 (d, J=5.7 Hz, 1H), 8.30 (br s, 1H), 7.82 (d, J=6.0 Hz, 1H), 7.61-7.54 (m, 3H), 7.48 (d, J=7.6 Hz, 1H), 7.12-7.10 (m, 2H), 6.93 (t, J=7.4 Hz, 1H), 6.80 (d, J=7.3 Hz, 1H), 6.34 (dd, J=6.0 and 2.0 Hz, 1H), 6.20 (d, J=1.9 Hz, 1H), 4.00 (t, J=7.8 Hz, 2H), 3.01 (t, J=7.8 Hz, 2H), 2.22 (s, 3H). LCMS Method A; RT=1.30; m/z [M+H]⁺ 396.1

### Example 72 (2072)

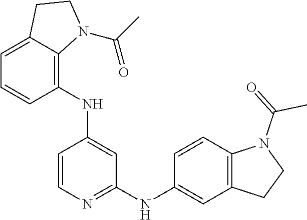 [SMILES: CC(=O)N1CCc2cc(Nc3cc(Nc4cccc5c4N(C(C)=O)CC5)ccn3)ccc21]

### 1-(7-((2-((1-Acetylindolin-5-yl)amino)pyridin-4-yl)amino)indolin-1-yl)ethan-1-one

Synthesised using the procedure described in Example 1 using Intermediate B1 and 1-acetyl-5-amino-2,3-dihydro(1H)indole. ¹H NMR (CDCl₃, 400 MHz): 8.69 (br s, 1H), 8.13 (d, J=8.6 Hz, 1H), 7.70 (d, J=6.2 Hz, 1H), 7.64 (br s, 1H), 7.22 (d, J=8.1 Hz, 1H), 7.15 (s, 1H), 7.12 (t, J=7.4 Hz, 1H), 7.00 (dd, J=8.6 and 2.2 Hz, 1H), 6.96 (d, J=7.3 Hz, 1H), 6.32-6.28 (m, 2H), 4.13-4.05 (m, 4H), 3.20-3.10 (m, 4H), 2.32 (s, 3H), 2.22 (s, 3H). LCMS Method A; RT=1.65; m/z [M+H]⁺ 428.1.

### Example 73 (2073)

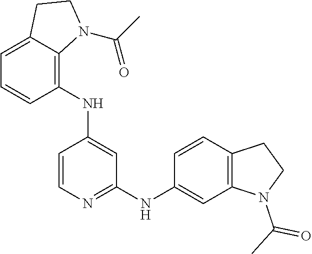 [SMILES: CC(=O)N1CCc2ccc(Nc3cc(Nc4cccc5c4N(C(C)=O)CC5)ccn3)cc21]

### 1-(7-((2-((1-Acetylindolin-6-yl)amino)pyridin-4-yl)amino)indolin-1-yl)ethan-1-one

Synthesised using the procedure described in Example 1 using Intermediate B1 and 1-acetyl-6-amino-2,3-dihydro-1H-indole. ¹H NMR (CDCl₃, 400 MHz): 8.69 (br s, 1H), 8.13 (s, 1H), 7.79-7.69 (m, 2H), 7.32 (d, J=8.2 Hz, 1H), 7.10 (t, J=7.5 Hz, 2H), 6.98 (dd, J=7.9 and 1.5 Hz, 1H), 6.94 (d, J=7.3 Hz, 1H), 6.37 (d, J=1.6 Hz, 1H), 6.30 (dd, J=6.3 and 1.9 Hz, 1H), 4.12-4.06 (m, 4H), 3.18-3.09 (m, 4H), 2.33 (s, 3H), 2.24 (s, 3H). LCMS Method A; RT=1.73; m/z [M+H]⁺ 428.1.

### Example 74 (2079)

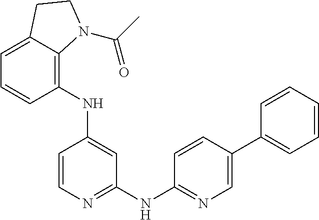 [SMILES: CC(=O)N1CCc2cccc(Nc3ccnc(Nc4ccc(-c5ccccc5)cn4)c3)c21]

### 1-(7-((2-((5-Phenylpyridin-2-yl)amino)pyridin-4-yl)amino)indolin-1-yl)ethan-1-one

Synthesised using the procedure described in Example 1 using Intermediate B1 and 2-amino-5-phenylpyridine. ¹H NMR (CDCl₃, 400 MHz): 9.61 (br s, 1H), 8.42 (s, 1H), 7.87 (dd, J=8.6 and 2.4 Hz, 1H), 7.71 (d, J=8.7 Hz, 1H), 7.63 (d, J=7.0 Hz, 1H), 7.61 (br s, 1H), 7.53 (d, J=7.0 Hz, 2H), 7.47 (t, J=7.2 Hz, 2H), 7.45-7.33 (m, 4H), 7.10 (d, J=7.3 Hz, 1H), 6.47 (dd, J=7.0 and 2.3 Hz, 1H), 4.17 (t, J=7.8 Hz, 2H), 3.18 (t, J=7.8 Hz, 2H), 2.38 (s, 3H). LCMS Method A; RT=2.25; m/z [M+H]⁺ 422.1.

### Example 75 (2101)

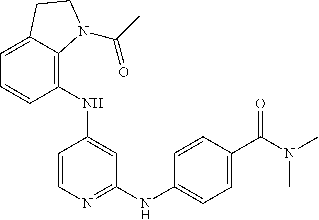 [SMILES: CC(=O)N1CCc2cccc(Nc3ccnc(Nc4ccc(C(=O)N(C)C)cc4)c3)c21]

### 4-((4-((1-Acetylindolin-7-yl)amino)pyridin-2-yl)amino)-N,N-dimethylbenzamide

Synthesised using the procedure described in Example 1 using Intermediate B1 and 4-amino-N,N-dimethyl-benzamide. ¹H NMR (CDCl₃, 400 MHz): 8.50 (s, 1H), 7.86 (d, J=6.0 Hz, 1H), 7.38 (d, J=8.7 Hz, 2H), 7.29 (d, J=8.7 Hz, 2H), 7.26 (s, 1H), 7.13 (t, J=7.4 Hz, 1H), 7.00 (br s, 1H), 6.94 (dd, J=7.3 and 1.0 Hz, 1H), 6.43 (d, J=1.8 Hz, 1H), 6.40 (dd, J=6.0 and 2.0 Hz, 1H), 4.11 (t, J=7.8 Hz, 2H), 3.12 (t, J=7.7 Hz, 2H), 3.06 (br s, 6H), 2.33 (s, 3H). LCMS Method A; RT=1.53; m/z [M+H]⁺ 416.2.

### Example 76-(2102)

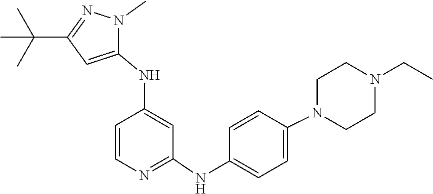 [SMILES: CCN1CCN(c2ccc(Nc3cc(Nc4cc(C(C)(C)C)nn4C)ccn3)cc2)CC1]

### N4-(3-(tert-Butyl)-1-methyl-1H-pyrazol-5-yl)-N2-(4-(4-ethylpiperazin-1-yl)phenyl)pyridine-2,4-diamine

Synthesised using the procedure described in Example 1 using Intermediate B25 and 4-(4-ethylpiperazin-1-yl)aniline. ¹H NMR (CDCl₃, 400 MHz): 7.70 (d, J=5.2 Hz, 1H), 7.17 (d, J=8.9 Hz, 2H), 6.97 (d, J=8.8 Hz, 2H), 6.29-6.27 (m, 1H), 6.06-6.05 (d, J=2.2 Hz, 1H), 5.97 (s, 1H), 3.62 (s, 3H), 3.22 (t, J=5.6 Hz, 4H), 2.79 (t, J=5.0 Hz, 4H), 2.64 (q, J=7.8 Hz, 2H), 1.27 (s, 9H), 1.20 (t, J=7.2 Hz, 3H). LCMS Method A; RT=1.15; m/z [M+H]⁺ 434.2.

### Example 77 (2103)

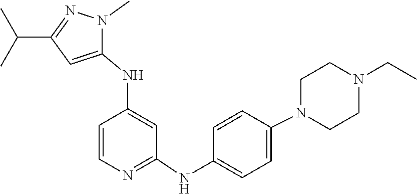 [SMILES: CCN1CCN(c2ccc(Nc3cc(Nc4cc(C(C)C)nn4C)ccn3)cc2)CC1]

### N2-(4-(4-Ethylpiperazin-1-yl)phenyl)-N4-(3-isopropyl-1-methyl-1H-pyrazol-5-yl)pyridine-2,4-diamine

Synthesised using the procedure described in Example 1 using Intermediate B27 and 4-(4-ethylpiperazin-1-yl)aniline. ¹H NMR (MeOD, 400 MHz): 7.69 (d, J=6.2 Hz, 1H), 7.18 (d, J=8.8 Hz, 2H), 6.97 (d, J=8.9 Hz, 2H), 6.27 (dd, J=6.2 and 2.0 Hz, 1H), 6.07 (d, J=2.0 Hz, 1H), 5.94 (s, 1H), 3.22 (t, J=4.8 Hz, 4H), 2.86 (sept, J=8.2 Hz, 1H), 2.79 (t, J=5.0 Hz, 4H), 2.64 (q, J=7.3 Hz, 2H), 1.23 (s, 3H), 1.22 (s, 3H), 1.20 (t, J=7.3 Hz, 3H). LCMS Method A; RT=1.13; m/z [M+H]⁺ 420.2.

### Example 78 (2104)

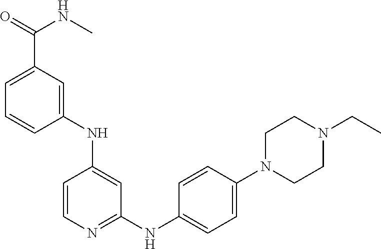 [SMILES: CCN1CCN(c2ccc(Nc3cc(Nc4cccc(C(=O)NC)c4)ccn3)cc2)CC1]

### 3-((2-((4-(4-Ethylpiperazin-1-yl)phenyl)amino)pyridin-4-yl)amino)-N-methylbenzamide

Synthesised using the procedure described in Example 1 using Intermediate B28 and 4-(4-ethylpiperazin-1-yl)aniline. ¹H NMR (MeOD, 400 MHz): 7.71 (t, J=1.8, 1H), 7.62 (d, J=6.7 Hz, 1H), 7.55 (d, J=7.8 Hz, 1H), 7.46 (t, J=7.9 Hz, 1H), 7.36-7.33 (m, 1H), 7.19 (d, J=8.9 Hz, 2H), 7.01 (d, J=9.0 Hz, 2H), 6.45 (dd, J=6.7 and 2.2 Hz, 1H), 6.34 (d, J=2.1 Hz, 1H), 3.25 (t, J=4.9 Hz, 4H), 2.93 (s, 3H), 2.82 (t, J=4.9 Hz, 4H), 2.67 (q, J=7.3 Hz, 2H), 1.21 (t, J=7.2 Hz, 3H). LCMS Method A; RT=0.94; m/z [M+H]⁺ 431.2.

### Example 79 (2105)

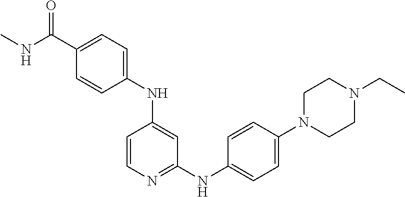 [SMILES: CCN1CCN(c2ccc(Nc3cc(Nc4ccc(C(=O)NC)cc4)ccn3)cc2)CC1]

### 4-((2-((4-(4-Ethylpiperazin-1-yl)phenyl)amino)pyridin-4-yl)amino)-N-methylbenzamide

Synthesised using the procedure described in Example 1 using Intermediate B29 and 4-(4-ethylpiperazin-1-yl)aniline. ¹H NMR (MeOD, 400 MHz): 7.78 (d, J=8.7 Hz, 2H), 7.71 (d, J=6.2 Hz, 1H), 7.22 (dd, J=8.7 and 3.4 Hz, 4H), 6.97 (d, J=9.0 Hz, 2H), 6.47-6.44 (m, 2H), 3.20 (t, J=4.8 Hz, 4H), 2.91 (s, 3H), 2.76 (t, J=5.0 Hz, 4H), 2.61 (q, J=7.3 Hz, 2H), 1.18 (t, J=7.2 Hz, 3H). LCMS Method A; RT=1.13; m/z [M+H]⁺ 431.2.

### Example 80 (2106)

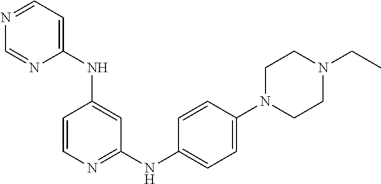 [SMILES: CCN1CCN(c2ccc(Nc3cc(Nc4ccncn4)ccn3)cc2)CC1]

### N2-(4-(4-Ethylpiperazin-1-yl)phenyl)-N4-(pyrimidin-4-yl)pyridine-2,4-diamine

Synthesised using the procedure described in Example 1 using Intermediate B32 and 4-(4-ethylpiperazin-1-yl)aniline. ¹H NMR (MeOD, 400 MHz): 8.66 (s, 1H), 8.29 (d, J=6.0 Hz, 1H), 7.86 (d, J=5.9, 1H), 7.47 (d, J=1.6 Hz, 1H), 7.29 (d, J=8.9 Hz, 2H), 6.98 (d, J=9.1 Hz, 2H), 6.95 (d, J=1.9 Hz, 1H), 6.86 (dd, J=6.0 and 1.1 Hz, 1H), 3.23 (t, J=4.6 Hz, 4H), 2.85 (t, J=5.0 Hz, 4H), 2.69 (q, J=7.3 Hz, 2H), 1.21 (t, J=7.3 Hz, 3H). LCMS Method A; RT=0.90; m/z [M+H]⁺ 376.2.

### Example 81 (2107)

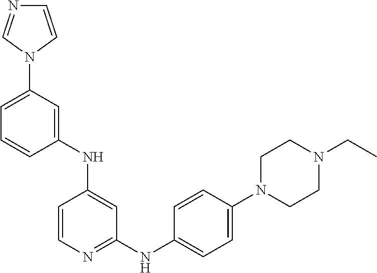 [SMILES: CCN1CCN(c2ccc(Nc3cc(Nc4cccc(-n5ccnc5)c4)ccn3)cc2)CC1]

### N4-(3-(1H-Imidazol-1-yl)phenyl)-N2-(4-(4-ethylpiperazin-1-yl)phenyl)pyridine-2,4-diamine

Synthesised using the procedure described in Example 1 using Intermediate B30 and 4-(4-ethylpiperazin-1-yl)aniline. ¹H NMR (MeOD, 400 MHz): 8.12 (s, 1H), 7.69 (d, J=6.4 Hz, 1H), 7.55 (s, 1H), 7.48 (t, J=8.2 Hz, 1H), 7.40 (t, J=2.1 Hz, 1H), 7.26 (dd, J=8.2 and 2.2 Hz, 1H), 7.20-7.17 (m, 4H), 6.91 (d, J=9.0 Hz, 2H), 3.22 (t, J=4.7 Hz, 4H), 2.85 (t, J=5.0 Hz, 4H), 2.71 (q, J=7.4 Hz, 2H), 1.22 (t, J=7.2 Hz, 3H). LCMS Method A; RT=0.70; m/z [M+H]⁺ 440.2.

### Example 82(2108)

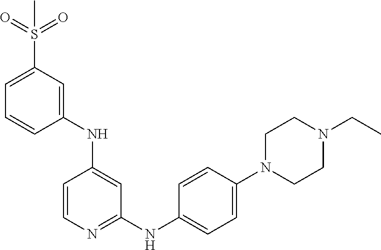 [SMILES: CCN1CCN(c2ccc(Nc3cc(Nc4cccc(S(C)(=O)=O)c4)ccn3)cc2)CC1]

### N2-(4-(4-Ethylpiperazin-1-yl)phenyl)-N4-(3-(methylsulfonyl)phenyl)pyridine-2,4-diamine

Synthesised using the procedure described in Example 1 using Intermediate B31 and 4-(4-ethylpiperazin-1-yl)aniline. ¹H NMR (MeOD, 400 MHz): 7.76-7.73 (m, 2H), 7.59-7.56 (m, 2H), 7.46 (d, J=6.9 Hz, 1H), 7.21 (d, J=8.5 Hz, 2H), 7.00 (d, J=8.8 Hz, 2H), 6.45-6.42 (m, 2H), 3.25 (t, J=4.8 Hz, 4H), 3.10 (s, 3H), 2.86 (t, J=4.9 Hz, 4H), 2.71 (q, J=7.3 Hz, 2H), 1.22 (t, J=7.3 Hz, 3H). LCMS Method A; RT=1.04; m/z [M+H]⁺ 452.1.

### Example 83 (2109)

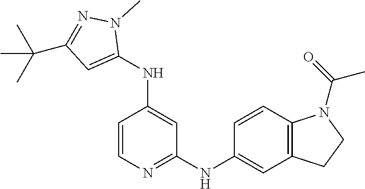 [SMILES: CC(=O)N1CCc2cc(Nc3cc(Nc4cc(C(C)(C)C)nn4C)ccn3)ccc21]

### 1-(5-((4-((3-(tert-Butyl)-1-methyl-1H-pyrazol-5-yl)amino)pyridin-2-yl)amino)indolin-1-yl)ethan-1-one

Synthesised using the procedure described in Example 1 using Intermediate B25 and 1-acetyl-5-amino-2,3-dihydro(1H)indole. ¹H NMR (MeOD, 400 MHz): 8.00 (d, J=8.6 Hz, 1H), 7.74 (d, J=5.8 Hz, 1H), 7.24 (s, 1H), 7.04 (d, J=8.5 Hz, 1H), 6.25 (dd, J=5.9 and 1.7 Hz, 1H), 6.07 (d, J=1.8 Hz, 1H), 5.97 (s, 1H), 4.11 (t, J=8.3 Hz, 2H), 3.62 (s, 3H), 3.17 (t, J=8.4 Hz, 2H), 2.21 (s, 3H), 1.27 (s, 9H). LCMS Method A; RT=1.63; m/z [M+H]⁺ 405.2.

### Example 85 (2110)

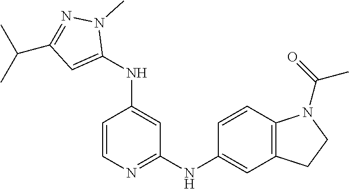 [SMILES: CC(=O)N1CCc2cc(Nc3cc(Nc4cc(C(C)C)nn4C)ccn3)ccc21]

### 1-(5-((4-((3-Isopropyl-1-methyl-1H-pyrazol-5-yl)amino)pyridin-2-yl)amino)indolin-1-yl)ethan-1-one

Synthesised using the procedure described in Example 1 using Intermediate B27 and 1-acetyl-5-amino-2,3-dihydro(1H)indole. ¹H NMR (MeOD, 400 MHz): 7.99 (d, J=8.6 Hz, 1H), 7.74 (d, J=6.0 Hz, 1H), 7.25 (s, 1H), 7.05 (d, J=8.4 Hz, 1H), 6.25 (dd, J=6.0 and 2.0 Hz, 1H), 6.09 (d, J=1.9 Hz, 1H), 5.94 (s, 1H), 4.10 (t, J=8.4 Hz, 2H), 3.62 (s, 3H), 3.16 (t, J=8.4 Hz, 2H), 2.87 (sept, J=6.9 Hz, 1H), 2.20 (s, 3H), 1.24 (s, 3H), 1.22 (s, 3H).

LCMS Method A; RT=1.61; m/z [M+H]⁺ 391.2.

### Example 86 (2111)

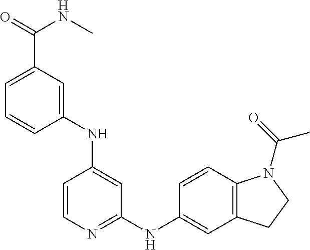 [SMILES: CNC(=O)c1cccc(Nc2ccnc(Nc3ccc4c(c3)CCN4C(C)=O)c2)c1]

### 3-((2-((1-Acetylindolin-5-yl)amino)pyridin-4-yl)amino)-N-methylbenzamide

Synthesised using the procedure described in Example 1 using Intermediate B28 and 1-acetyl-5-amino-2,3-dihydro(1H)indole. ¹H NMR (MeOD, 400 MHz): 8.00 (d, J=8.6 Hz, 1H), 7.73 (d, J=6.1 Hz, 1H), 7.68 (t, J=1.7 Hz, 1H), 7.47 (d, J=7.7 Hz, 1H), 7.41 (t, J=7.8 Hz, 1H), 7.32 (d, J=8.8 Hz, 1H), 7.23 (s, 1H), 7.06 (dd, J=8.6 and 1.9 Hz, 1H), 6.45-6.41 (m, 2H), 4.07 (t, J=8.4 Hz, 2H), 3.14 (t, J=8.4 Hz, 2H), 2.94 (s, 3H), 2.19 (s, 3H).

LCMS Method A; RT=1.46; m/z [M+H]⁺ 402.2.

### Example 87 (2112)

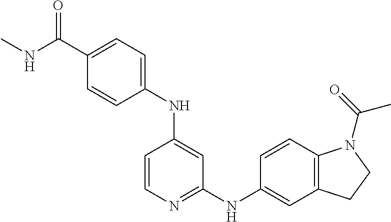 [SMILES: CNC(=O)c1ccc(Nc2ccnc(Nc3ccc4c(c3)CCN4C(C)=O)c2)cc1]

### 4-((2-((1-Acetylindolin-5-yl)amino)pyridin-4-yl)amino)-N-methylbenzamide

Synthesised using the procedure described in Example 1 using Intermediate B29 and 1-acetyl-5-amino-2,3-dihydro(1H)indole. ¹H NMR (MeOD, 400 MHz): 7.99 (d, J=8.6 Hz, 1H), 7.79-7.76 (m, 3H), 7.27 (s, 1H), 7.20 (d, J=8.7 Hz, 2H), 7.06 (dd, J=8.6 and 2.0 Hz, 1H), 6.51 (d, J=1.9 Hz, 1H), 6.45 (dd, J=6.0 and 2.0 Hz, 1H), 4.08 (t, J=8.4 Hz, 2H), 3.15 (t, J=8.3 Hz, 2H), 2.90 s, 3H), 2.19 (s, 3H). LCMS Method A; RT=1.42; m/z [M+H]⁺ 402.2.

### Example 88 (2115)

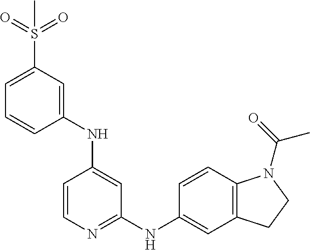 [SMILES: CC(=O)N1CCc2cc(Nc3cc(Nc4cccc(S(C)(=O)=O)c4)ccn3)ccc21]

### 1-(5-((4-((3-(Methylsulfonyl)phenyl)amino)pyridin-2-yl)amino)indolin-1-yl)ethan-1-one

Synthesised using the procedure described in Example 1 using Intermediate B31 and 1-acetyl-5-amino-2,3-dihydro(1H)indole. ¹H NMR (MeOD, 400 MHz): 7.92 (d, J=8.6 Hz, 1H), 7.70 (d, J=6.0 Hz, 1H), 7.62 (s, 1H), 7.46-7.45 (m, 2H), 7.38-7.35 (m, 1H), 7.17 (s, 1H), 6.98 (dd, J=8.6 and 2.1 Hz, 1H), 6.38 (d, J=1.9 Hz, 1H), 6.33 (dd, J=6.0 and 2.0 Hz, 1H), 4.01 (t, J=8.3 Hz, 2H), 3.09 (t, J=8.3 Hz, 2H), 3.00 (s, 3H), 2.11 (s, 3H). LCMS Method A; RT=1.45; m/z [M+H]⁺ 423.1.

### Example 89 (2116)

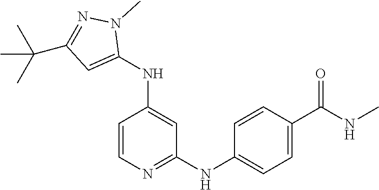 [SMILES: CNC(=O)c1ccc(Nc2cc(Nc3cc(C(C)(C)C)nn3C)ccn2)cc1]

### 4-((4-((3-(tert-Butyl)-1-methyl-1H-pyrazol-5-yl)amino)pyridin-2-yl)amino)-N-methylbenzamide

Synthesised using the procedure described in Example 1 using Intermediate B25 and 4-amino-N-methylbenzamide. ¹H NMR (MeOD, 400 MHz): 7.81 (d, J=5.9 Hz, 1H), 7.67 (d, J=8.8 Hz, 2H), 7.44 (d, J=8.7 Hz, 2H), 6.28 (dd, J=5.9 and 2.0 Hz, 1H), 6.15 (d, J=1.9 Hz, 1H), 5.98 (s, 1H), 3.60 (s, 3H), 2.86 (s, 3H), 1.26 (s, 9H). LCMS Method A; RT=1.48; m/z [M+H]⁺ 379.2.

### Example 90 (2117)

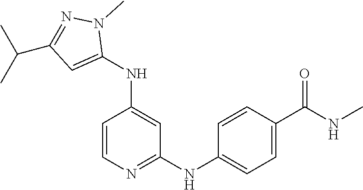 [SMILES: CNC(=O)c1ccc(Nc2cc(Nc3cc(C(C)C)nn3C)ccn2)cc1]

### 4-((4-((3-Isopropyl-1-methyl-1H-pyrazol-5-yl)amino)pyridin-2-yl)amino)-N-methylbenzamide

Synthesised using the procedure described in Example 1 using Intermediate B27 and 4-amino-N-methylbenzamide. ¹H NMR (MeOD, 400 MHz): 7.86 (d, J=5.9 Hz, 1H), 7.72 (d, J=8.7 Hz, 2H), 7.48 (d, J=8.8 Hz, 2H), 6.34 (dd, J=5.9 and 2.0 Hz, 1H), 6.20 (d, J=2.0 Hz, 1H), 5.99 (s, 1H), 3.64 (s, 3H), 2.93-2.85 (m, 4H), 1.26 (s, 3H), 1.25 (s, 3H). LCMS Method A; RT=1.34; m/z [M+H]⁺ 365.2.

### Example 91 (2347)

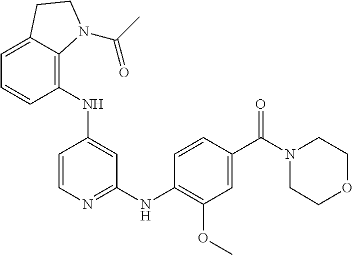 [SMILES: COc1cc(C(=O)N2CCOCC2)ccc1Nc1cc(Nc2cccc3c2N(C(C)=O)CC3)ccn1]

### 1-(7-((2-((2-Methoxy-4-(morpholine-4-carbonyl)phenyl)amino)pyridin-4-yl)amino)indolin-1-yl)ethan-1-one

Synthesised using the procedure described in Example 1 using Intermediate B1 and (4-amino-3-methoxyphenyl)(morpholino)methanone. ¹H NMR (DMSO-d6, 400 MHz) δ 8.82 (bs, 1H), 8.13 (bs, 1H), 7.79 (d, J=6 Hz, 1H), 7.20 (m, J=8 Hz, 2H), 7.03 (s, 2H), 6.95 (dd, J=6 Hz, 1H), 6.48 (bs, 1H), 6.33 (d, J=6 Hz, 1H), 4.14 (t, J=8 Hz, 2H), 3.85 (s, 3H), 3.61 (m, J=5 Hz, 4H), 3.52 (bs, 4H), 3.08 (t, J=8 Hz, 2H), 2.28 (s, 3H). LCMS Method C; RT=2.02; m/z [M+H]⁺ 488.2.

### Example 92 (2348)

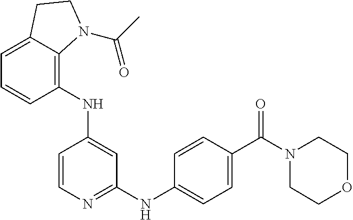 [SMILES: CC(=O)N1CCc2cccc(Nc3ccnc(Nc4ccc(C(=O)N5CCOCC5)cc4)c3)c21]

### 1-(7-((2-((4-(Morpholine-4-carbonyl)phenyl)amino)pyridin-4-yl)amino)indolin-1-yl)ethan-1-one

Synthesised using the procedure described in Example 1 using Intermediate B1 and (4-aminophenyl)(morpholino)methanone. ¹H NMR (CDCl₃, 400 MHz) δ 8.39 (s, 1H), 7.92 (t, J=4 Hz, 1H), 7.38-7.28 (m, 5H), 7.13 (t, J=8 Hz, 1H), 6.95-6.93 (m, 1H), 6.53 (s, 1H), 6.428 (t, J=4 Hz, 2H), 4.11 (t, J=8 Hz, 2H), 3.70-3.66 (br m, 9H), 3.12 (t, J=8 Hz, 2H), 2.33 (s, 3H). LCMS Method C; RT=1.89; m/z [M+H]⁺ 458.2.

### Example 93 (2349)

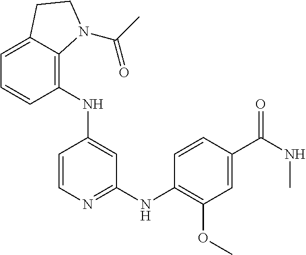 [SMILES: CNC(=O)c1ccc(Nc2cc(Nc3cccc4c3N(C(C)=O)CC4)ccn2)c(OC)c1]

### 4-((4-((1-Acetylindolin-7-yl)amino)pyridin-2-yl)amino)-3-methoxy-N-methylbenzamide

Synthesised using the procedure described in Example 1 using Intermediate B1 and 4-amino-3-methoxy-N-methylbenzamide. ¹H NMR (CDCl₃, 400 MHz) δ 9.44 (s, 1H), 9.07 (s, 1H), 7.53-7.35 (m, 3H), 7.14-7.05 (m, 5H), 6.31 (dd, J=2 Hz, J=2 Hz, 1H), 6.16 (d, J=2 Hz 1H), 4.13 (t, J=8 Hz, 2H), 3.87 (s, 1H), 3.14 (t, J=8 Hz, 2H), 3.02 (d, J=5 3H), 2.34 (s, 3H). LCMS Method C; RT=1.96; m/z [M+H]432.1.

### Example 94 (2350)

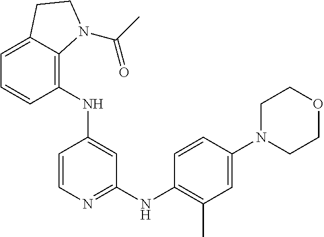 [SMILES: CC(=O)N1CCc2cccc(Nc3ccnc(Nc4ccc(N5CCOCC5)cc4C)c3)c21]

### 1-(7-((2-((2-Methyl-4-morpholinophenyl)amino)pyridin-4-yl)amino)indolin-1-yl)ethan-1-one

Synthesised using the procedure described in Example 1 using Intermediate B1 and 2-methyl-4-morpholinoaniline. ¹H NMR (DMSO-d6, 400 MHz) δ 8.65 (bs, 1H), 7.9 (bs, 1H), 7.64 (d, J=6 Hz, 1H), 7.17-7.10, (m, 3H), 7.00 (d, J=7 Hz, 1H), 6.81 (d, J=3 Hz, 1H), 6.75 (dd, J=6 Hz, 1H), 6.16 (dd, J=4 Hz, 1H), 5.91 (s, 1H), 4.11 (t, J=8 Hz, 2H), 3.73 (m, 3H), 3.06 (m, 7H), 2.25 (s, 4H), 2.13 (s, 3H). LCMS Method C; RT=2.25; m/z [M+H]⁺ 444.2.

### Example 95 (2351)

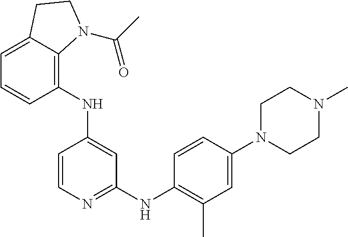 [SMILES: CC(=O)N1CCc2cccc(Nc3ccnc(Nc4ccc(N5CCN(C)CC5)cc4C)c3)c21]

### 1-(7-((2-((2-Methyl-4-(4-methylpiperazin-1-yl)phenyl)amino)pyridin-4-yl)amino)indolin-1-yl)ethan-1-one

Synthesised using the procedure described in Example 1 using Intermediate B1 and 2-methyl-4-(4-methylpiperazin-1-yl)aniline. ¹H NMR (DMSO-d6, 400 MHz) δ 9.80 (bs, 1H), 9.60 (bs, 1H), 9.26 (bs, 1H), 7.56 (d, J=7 Hz, 1H), 7.16 (m, 4H), 7.00 (d, J=2 Hz, 1H), 6.91 (dd, 1H), 6.38 (dd, J=6 hz, 1H), 5.88 (d, J=2 Hz, 1H), 4.11 (t, J=8 Hz, 2H), 3.84 (bs, 4H), 3.51 (bs, 3H), 3.09 (t, J=8 Hz, 2H), 2.87 (s, 3H), 2.22 (s, 3H), 2.13 (s, 3H). LCMS Method C; RT=1.55; m/z [M+H]457.2.

### Example 96 (2353)

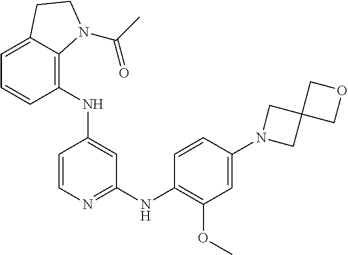 [SMILES: COc1cc(N2CC3(COC3)C2)ccc1Nc1cc(Nc2cccc3c2N(C(C)=O)CC3)ccn1]

### 1-(7-((2-((2-Methoxy-4-(2-oxa-6-azaspiro[3.3]heptan-6-yl)phenyl)amino)pyridin-4-yl)amino)indolin-1-yl)ethan-1-one

Synthesised using the procedure described in Example 1 using Intermediate B1 and 2-methoxy-4-(2-oxa-6-azaspiro[3.3]heptan-6-yl)aniline. ¹H NMR (DMSO-d6, 400 MHz) δ 8.39 (s, 1H), 7.68 (d, J=8 Hz, 1H), 7.40 (d, J=7 Hz, 1H), 7.26 (s, 1H), 7.15-7.09 (m, 2H), 9.96-6.94 (m, 1H), 6.12-6.09 (m, 2H), 6.03 (s, 1H), 6.02-5.96 (m, 1H), 4.72 (s, 4H), 4.11 (t, J=8 Hz, 2H), 3.94 (s, 4H), 3.74 (s, 3H), 3.04 (t, J=8 Hz, 2H), 2.26 (s, 3H). LCMS Method C; RT=2.21; m/z [M+H]⁺ 472.2.

### Example 97 (2354)

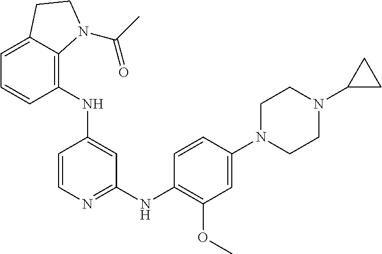 [SMILES: COc1cc(N2CCN(C3CC3)CC2)ccc1Nc1cc(Nc2cccc3c2N(C(C)=O)CC3)ccn1]

### 1-(7-((2-((4-(4-Cyclopropylpiperazin-1-yl)-2-methoxyphenyl)amino)pyridin-4-yl)amino)indolin-1-yl)ethan-1-one

Synthesised using the procedure described in Example 1 using Intermediate B1 and 4-(4-cyclopropylpiperazin-1-yl)-2-methoxyaniline. ¹H NMR (MeOD, 400 MHz) δ 7.49 (d, J=7.3 Hz, 1H), 7.25 (d, J=4.4 Hz, 2H), 7.20 (d, J=4.4 Hz, 1H), 7.16 (d, J=8.6 Hz, 1H), 6.78 (d, J=2.5 Hz, 1H), 6.67 (d, J=8.7 Hz, 1H), 6.43 (dd, J=7.4, 2.2 Hz, 1H), 6.10 (s, 1H), 4.21 (t, J=7.8 Hz, 2H), 3.86 (s, 3H), 3.56 (s, 4H), 3.50 (t, J=5.0 Hz, 2H), 3.41 (t, J=5.1 Hz, 2H), 3.18 (t, J=7.8 Hz, 2H), 2.87 (s, 1H), 2.35 (s, 3H). LCMS Method C; RT=1.59; m/z [M+H]⁺ 499.3.

### Example 98 (2355)

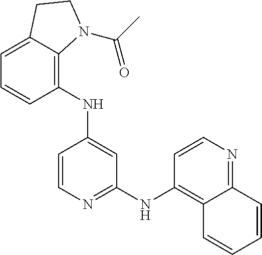 [SMILES: CC(=O)N1CCc2cccc(Nc3ccnc(Nc4ccnc5ccccc45)c3)c21]

### 1-(7-((2-(Quinolin-4-ylamino)pyridin-4-yl)amino)indolin-1-yl)ethan-1-one

Synthesised using the procedure described in Example 1 using Intermediate B1 and quinolin-4-amine. ¹H NMR (CDCl₃, 400 MHz) δ 8.71 (d, J=5 Hz 1H), 8.57 (s, 1H), 8.06 (d, J=8 Hz 1H), 8.02-7.98 (m, 2H), 7.70 (t, J=6 Hz, 1H), 7.55-7.51 (m, 2H), 7.14 (t, J=8 Hz, 2H), 6.96 (d, J=3 Hz 1H), 6.68 (s, 1H), 6.68-6.54 (m, 1H), 4.12 (t, J=8 Hz, 2H), 3.13 (t, J=8 Hz, 2H), 3.01 (s, 2H), 2.94 (s, 2H), 2.34 (s, 3H), 2.08 (s, 3H). LCMS Method C; RT=1.79; m/z [M+H]⁺ 396.1.

### Example 99 (2356)

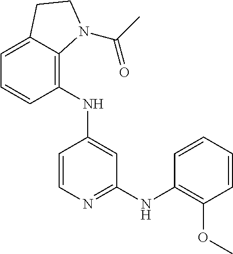 [SMILES: COc1ccccc1Nc1cc(Nc2cccc3c2N(C(C)=O)CC3)ccn1]

### 1-(7-((2-((2-Methoxyphenyl)amino)pyridin-4-yl)amino)indolin-1-yl)ethan-1-one

Synthesised using the procedure described in Example 1 using Intermediate B1 and 2-methoxyaniline. ¹H NMR (CDCl₃, 400 MHz) δ 8.21 (bs, 1H), 7.94-7.91 (m, 2H), 7.32 (d, J=8 Hz, 1H), 7.13 (t, J=8 Hz, 1H), 6.93-6.87 (m, 4H), 6.78 (bs, 1H), 6.38 (s, 1H) 6.37 (d, J=2 Hz, 1H), 4.12-4.08 (m, 2H), 2.33 (d, J=5 Hz, 3H). LCMS Method C; RT=2.13; m/z [M+H]⁺ 375.1.

### Example 100 (2357)

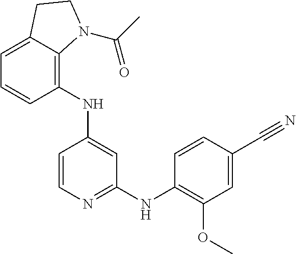 [SMILES: COc1cc(C#N)ccc1Nc1cc(Nc2cccc3c2N(C(C)=O)CC3)ccn1]

### 4-((4-((1-Acetylindolin-7-yl)amino)pyridin-2-yl)amino)-3-methoxybenzonitrile

Synthesised using the procedure described in Example 1 using Intermediate B1 and 4-amino-3-methoxybenzonitrile. ¹H NMR (CDCl₃, 400 MHz) δ 8.46 (bs, 1H), 8.39 (d, J=8 Hz, 1H), 7.98 (d, J=6 Hz, 1H), 7.32 (d, J=7 Hz, 1H), 7.26 (m, 1H), 7.16 (t, J=7 Hz, 1H), 7.09 (bs, 1H), 7.02 (d, J=2 Hz, 1H), 6.96 (dd, J=6 Hz, 1H), 6.49 (dd, 1H), 6.38 (d, 1H), 4.12 (m, 2H), 3.91 (s, 3H), 3.13 (t, J=8 Hz, 2H), 2.34 (s, 3H). LCMS Method C; RT=2.15; m/z [M+H]400.1.

### Example 101 (2358)

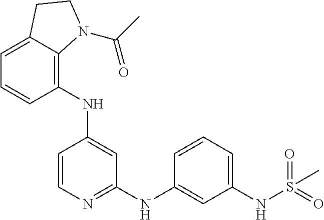 [SMILES: CC(=O)N1CCc2cccc(Nc3ccnc(Nc4cccc(NS(C)(=O)=O)c4)c3)c21]

### N-(3-((4-((1-Acetylindolin-7-yl)amino)pyridin-2-yl)amino)phenyl)methanesulfonamide

Synthesised using the procedure described in Example 1 using Intermediate B1 and N-(3-aminophenyl)methanesulfonamide. ¹H NMR (DMSO-d6, 400 MHz) δ 9.528 (bs, 1H), 8.73-8.57 (bd, 1H), 7.82 (d, J=6 Hz, 1H), 7.47 (t, J=2 Hz, 1H), 7.42 (dd, J=8 Hz, 1H), 7.24-7.11 (m, 3H), 7.01 (dd, J=7 Hz, 1H), 6.93 (t, J=8 Hz, 1H), 6.68 (dd, J=7 Hz, 1H), 6.46 (t, 1H), 6.31 (s, 1H), 6.25 (dd, J=4 Hz, 1H), 4.15 (t, J=8 Hz, 2H), 3.08 (t, J=8 Hz, 2H), 3.08 (s, 3H), 2.28 (s, 3H). LCMS Method C; RT=2.07; m/z [M+H]⁺ 438.1.

### Example 102 (2359)

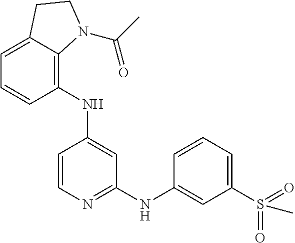 [SMILES: CC(=O)N1CCc2cccc(Nc3ccnc(Nc4cccc(S(C)(=O)=O)c4)c3)c21]

### 1-(7-((2-((3-(Methylsulfonyl)phenyl)amino)pyridin-4-yl)amino)indolin-1-yl)ethan-1-one

Synthesised using the procedure described in Example 1 using Intermediate B1 and 3-(methylsulfonyl)aniline. ¹H NMR (CDCl₃, 400 MHz) δ 8.54 (bs, 1H), 7.95-7.93 (m, 2H), 7.62-7.59 (m, 1H) 7.49-7.46 (m, 2H), 7.43 (d, J=8 Hz, 1H), 7.32 (d, J=8 Hz, 1H), 7.18 (t, J=8 Hz, 1H), 6.94 (d, J=7 Hz, 1H), 6.49 (bs, 1H), 6.45 (dd, J=2 Hz, J=2 Hz, 1H), 6.39 (d, J=2 Hz, 1H), 4.11 (t, J=8 Hz, 2H), 3.12 (t, J=8 Hz, 2H), 3.05 (s, 3H), 2.34 (s, 3H). LCMS Method C; RT=1.93; m/z [M+H]423.1.

### Example 103 (1940)

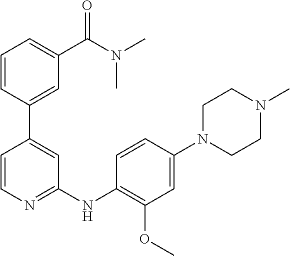 [SMILES: COc1cc(N2CCN(C)CC2)ccc1Nc1cc(-c2cccc(C(=O)N(C)C)c2)ccn1]

### 3-(2-((2-Methoxy-4-(4-methylpiperazin-1-yl)phenyl)amino)pyridin-4-yl)-N,N-dimethylbenzamide

Synthesised using the procedure described in Example 3 using Intermediate F1 and 2-methoxy-4-(4-methylpiperazin-1-yl)aniline. ¹H NMR (CDCl₃, 400 MHz) δ 8.23 (dd, J=5.3, 0.8 Hz, 1H), 7.77-7.71 (m, 1H), 7.61 (dt, J=7.1, 1.6 Hz, 2H), 7.51-7.40 (m, 2H), 6.93-6.86 (m, 2H), 6.69 (s, 1H), 6.57 (d, J=8.1 Hz, 2H), 3.87 (s, 3H), 3.23-3.18 (m, 4H), 3.14 (s, 3H), 3.00 (s, 3H), 2.61 (dd, J=6.2, 3.8 Hz, 4H), 2.37 (s, 3H). LCMS Method A; RT 1.08; m/z [M+H]⁺ 446.2.

1. A compound that (a) is of formula (I): 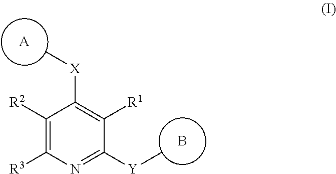
or (b) is a tautomer, N-oxide, pharmaceutically acceptable salt, or solvate thereof; wherein:
X is a bond or NH;
Y is a bond or NH;
with the proviso that at least one of X and Y is NH;
R¹ is H, F or CH₃;
R² is H, F, Cl, Br, —COR⁴, —SO₂R⁵, —SOR⁵, —CN, —NO, —NO₂ or —NR⁶₃⁺;
R³ is H, CH₃, C₂-C₆ alkyl substituted with 0 to 3 R⁷, —COR⁴, —SO₂R⁵, —SOR⁵, —CN, —NO, —NO₂ or —NR⁶₃⁺;
R⁴ is independently selected from H, C₁-C₆ alkyl substituted with 0 to 3 R⁷, —OH, —OR⁸, —NH₂, —NHR⁸ or —NR⁸₂;
R⁵ is independently selected from C₁-C₆ alkyl substituted with 0 to 3 R⁷, —OH, —OR⁸, —NH₂, —NHR⁸ or —NR⁸₂;
R⁶ is independently selected from C₁-C₃ alkyl;
R⁷ is independently selected from O—C₁-C₃ alkyl, F or Cl;
R⁸ is independently selected from C₁-C₆ alkyl substituted with 0 to 3 R⁷;
ring A is selected from a ring within group I, group II, group III, group IV and group V, wherein * denotes attachment to X;
group I is: 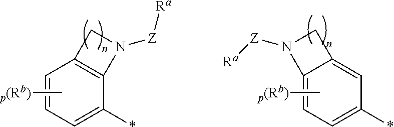
group II is: 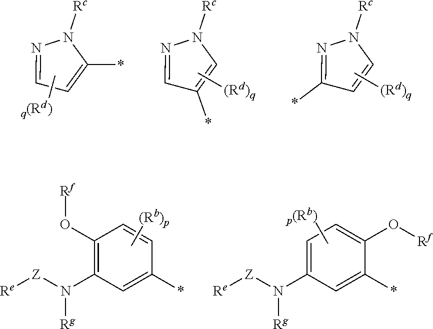
group III is: 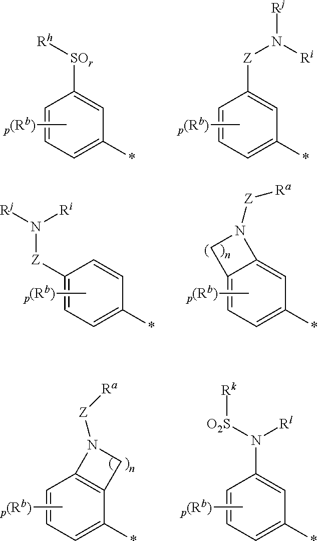
group IV is: 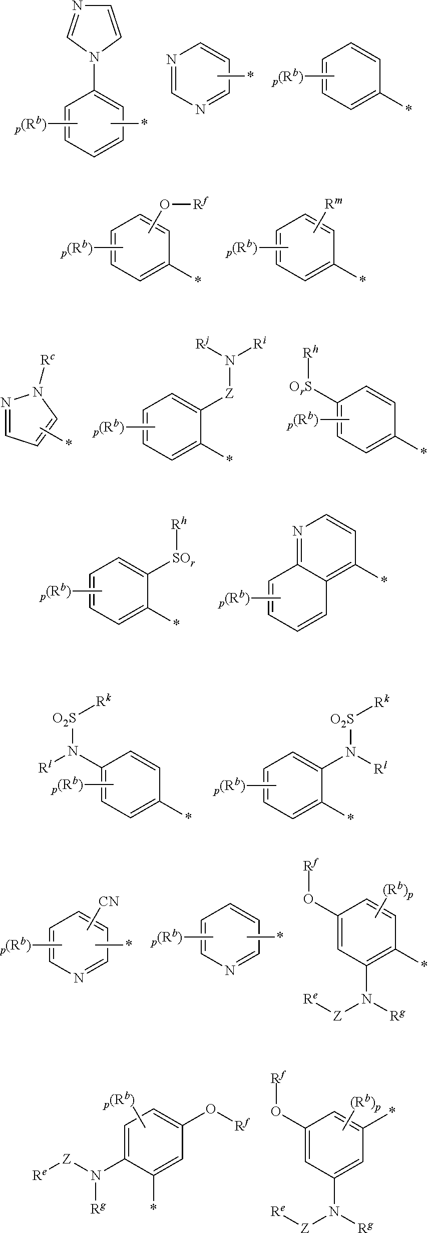 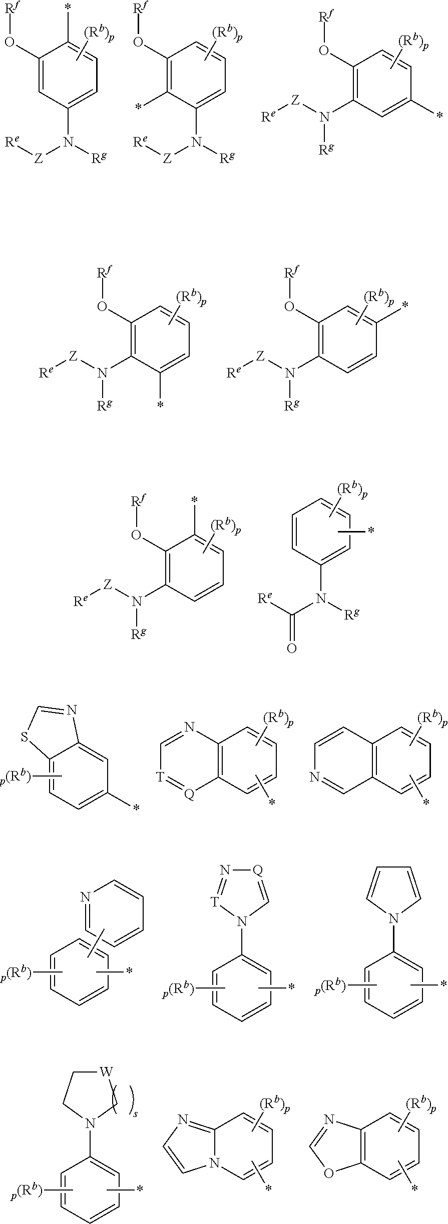 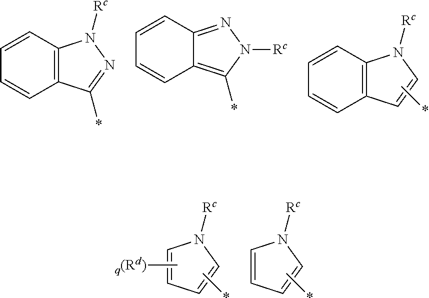
group V is: 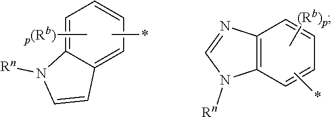
ring B is selected from a ring within group IA, group IIA, group IIA, group IVA, and group VA, wherein $ denotes attachment to Y;
group IA is: 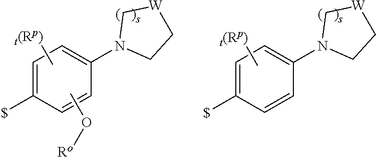
group IIA is: 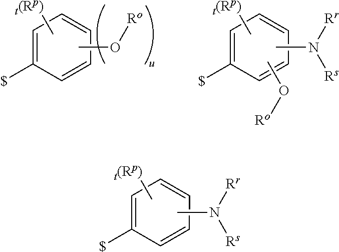
group IIIA is: 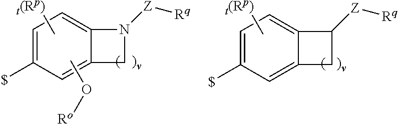
group IVA is: 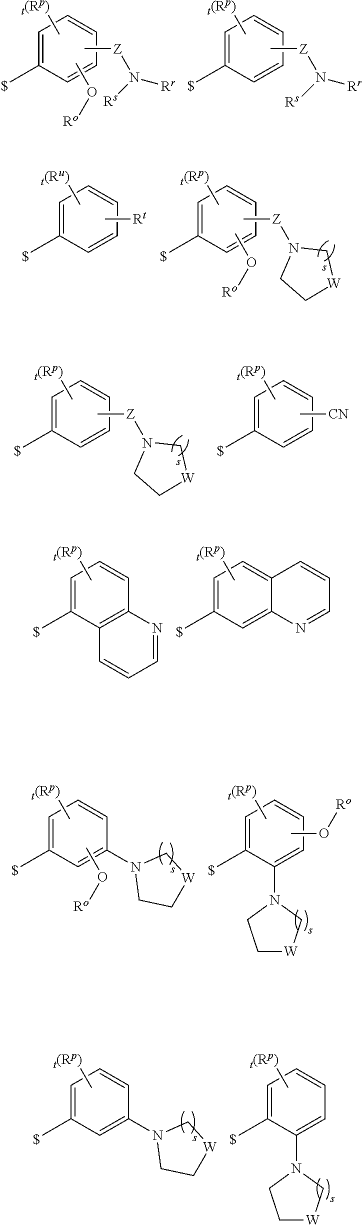
group VA is: 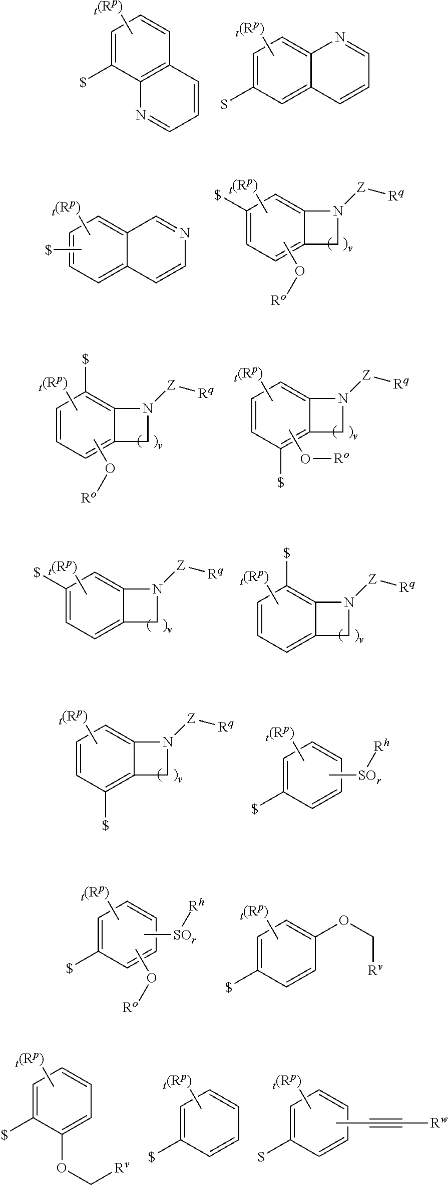 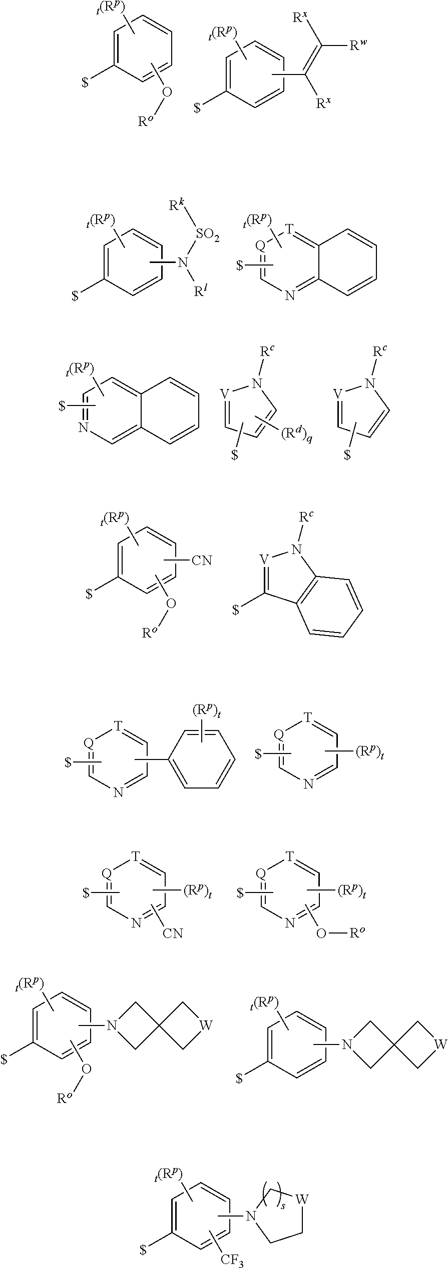
Q and T are each selected from CH or N, with the proviso that at most one of Q and T may be N;
V is CH or N;
W is CH₂, O, NR^{y}, S, S(O) or S(O)₂;
Z is C(O), S(O) or S(O)₂;
R^{a} is C₁-C₆ alkyl substituted with 0 to 3 substituents independently selected from O—C₁-C₃ alkyl, F and Cl;
R^{b} is independently selected from C₁-C₆ alkyl, F and Cl;
R^{c} is C₁-C₆ alkyl substituted with 0 to 3 substituents independently selected from O—C₁-C₃ alkyl, F and Cl;
R^{d} is independently selected from C₁-C₆ alkyl substituted with 0 to 3 substituents independently selected from O—C₁-C₃ alkyl, F and Cl; or phenyl substituted with 0 to 3 substituents independently selected from O—C₁-C₃ alkyl, F and Cl;
R^{e} is C₁-C₆ alkyl substituted with 0 to 3 substituents independently selected from O—C₁-C₃ alkyl, F and Cl;
R^{f} is C₁-C₆ alkyl substituted with 0 to 3 substituents independently selected from O—C₁-C₃ alkyl, F and Cl;
R^{g} is H or C₁-C₃ alkyl;
R^{h} is C₁-C₆ alkyl substituted with 0 to 3 substituents independently selected from O—C₁-C₃ alkyl, F and Cl;
Rⁱ is C₁-C₆ alkyl substituted with 0 to 3 substituents independently selected from O—C₁-C₃ alkyl, F and Cl;
R^{j} is H or C₁-C₃ alkyl
R^{k} is C₁-C₆ alkyl substituted with 0 to 3 substituents independently selected from O—C₁-C₃ alkyl, F and Cl;
R^{l} is H or C₁-C₃ alkyl;
R^{m} is C₁-C₆ alkyl substituted with 0 to 3 substituents independently selected from O—C₁-C₃ alkyl, F and Cl;
Rⁿ is H or C₁-C₃ alkyl;
R^{o} is independently selected from C₁-C₆ alkyl substituted with 0 to 3 substituents independently selected from O—C₁-C₃ alkyl, F and Cl; or phenyl substituted with 0 to 3 substituents independently selected from O—C₁-C₃ alkyl, F and Cl;
R^{p} is independently selected from C₁-C₆ alkyl, F, Cl and Br;
R^{q} is C₁-C₆ alkyl substituted with 0 to 3 substituents independently selected from O—C₁-C₃ alkyl, F and Cl;
R^{r} is H or C₁-C₆ alkyl substituted with 0 to 3 substituents independently selected from O—C₁-C₃ alkyl, F and Cl;
R^{s} is H or C₁-C₃ alkyl;
R^{t} is C₁-C₆ alkyl substituted with 0 to 3 substituents independently selected from O—C₁-C₃ alkyl, F and Cl;
R^{u} is independently selected from C₁-C₆ alkyl, F and Cl;
R^{v} is phenyl substituted with 0-2 substituents selected from C₁-C₆ alkyl, F and Cl;
R^{w} is H or C₁-C₃ alkyl;
R^{x} is independently selected from H or C₁-C₃ alkyl;
R^{y} is H, C₁-C₆ alkyl substituted with 0 to 3 substituents independently selected from O—C₁-C₃ alkyl, F and Cl; benzyl substituted with 0 to 3 substituents independently selected from C₁-C₆ alkyl, F and Cl; or C₃-C₆ cycloalkyl substituted with 0 to 3 substituents independently selected from C₁-C₆ alkyl, O—C₁-C₃ alkyl, F and Cl;
n is 1 to 3;
p is 0 to 2;
q is 1 or 2;
r is 0 to 2;
s is 1 to 3;
t is 0 to 2;
u is 2 to 3;
v is 1 to 3;
and wherein:
when X is NH; Y is NH; R² is H; and ring A is a ring within group I or group II; then: ring B is a ring within group IA, group IIA, group IIIA, group IVA or group VA;
when X is NH; Y is NH; R² is H; and ring A is a ring within group III, then: ring B is a ring within group IA, group IIA or group IIIA;
when X is NH; Y is NH R² is H; and ring A is a ring within group IV, then: ring B is a ring within group IA or group IIA;
when X is NH; Y is NH; R² is H; and ring A is a ring within group V, then: ring B is a ring within group IA;
when X is NH; Y is NH; and R² is F, Cl, Br, —COR⁴, —SO₂R⁵, —SOR⁵, —CN, —NO, —NO₂ or —N⁶₃⁺; then: ring A is a ring within group I or group II and ring B is a ring within group IA;
when X is a bond and Y is NH, then: R² is H; ring A is a ring within group I, group II and group III; and ring B is a ring within group IA; and
when X is NH and Y is a bond, then: R² is H; ring A is a ring within group I, group II and group III; and ring B is a ring within group IA, group IIA, and group IIIA;
and with the proviso that the compound of formula (I) is not (a) a compound selected from the group: 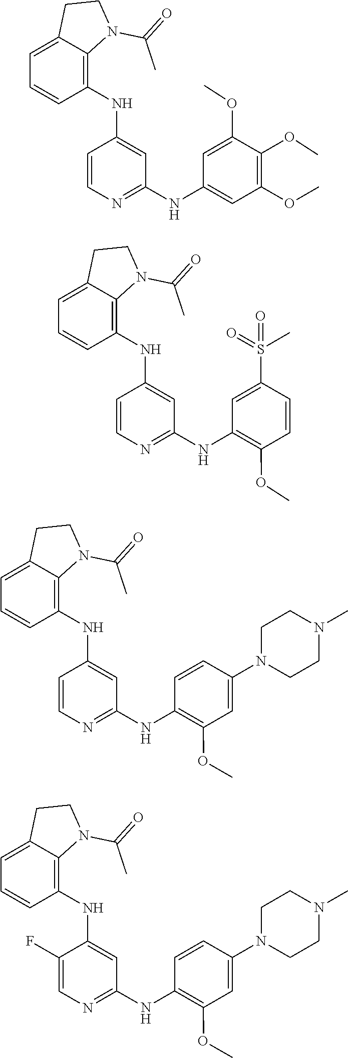
nor (b) a tautomer, N-oxide, pharmaceutically acceptable salt, or solvate thereof.

2. The compound according to claim 1, wherein:
when X is NH; Y is NH; R² is H; and ring A is a ring within group I; then: ring B is a ring within group IA, group IIA, group IIIA, group IVA or group VA; and
when X is NH; Y is NH; R² is H; and ring A is a ring within group II; then: ring B is a ring within group IA, group IIA, group IIIA or group IVA.

3. The compound according to claim 1, wherein:
X is NH;
Y is NH;
R² is H or F;
R³ is H or CH₃;
Q is selected from CH or N;
T is CH;
W is CH₂, O, or NR^{y};
Z is C(O);
R^{a} is C₁-C₃ alkyl substituted with 0 to 1 substituent selected from O—C₁-C₃ alkyl;
R^{b} is independently selected from C₁-C₃ alkyl;
R^{c} is C₁-C₃ alkyl;
R^{d} is independently selected from C₁-C₄ alkyl or is phenyl;
R^{e} is C₁-C₃ alkyl;
R^{f} is C₁-C₃ alkyl;
R^{g} is H;
R^{h} is C₁-C₃ alkyl;
Rⁱ is C₁-C₃ alkyl;
R^{j} is H;
R^{k} is C₁-C₃ alkyl;
R^{l} is H;
R^{m} is C₁-C₄ alkyl;
Rⁿ is H;
R^{o} is independently selected from C₁-C₃ alkyl substituted with 0 to 1 substituent selected from O—C₁-C₃ alkyl; or phenyl;
R^{p} is independently selected from C₁-C₃ alkyl;
R^{q} is C₁-C₃ alkyl substituted with 0 to 1 substituent selected from O—C₁-C₃ alkyl;
R^{r} is H or C₁-C₃ alkyl;
R^{t} is C₁-C₄ alkyl;
R^{u} is independently selected from C₁-C₃ alkyl;
R^{v} is phenyl;
R^{w} is H;
R^{x} is H;
R^{y} is C₁-C₃ alkyl substituted with 0 to 1 substituent selected from O—C₁-C₃ alkyl; or is benzyl substituted with 0 to 1 substituent selected from C₁-C₃ alkyl; or is C₃-C₅ cycloalkyl substituted with 0 to 1 substituent selected from C₁-C₃ alkyl;
n is 2;
p is 0 or 1;
q is 1;
r is 2;
s is 2;
t is 0 or 1; and
v is 2.

4. The compound according to claim 1, wherein:
R¹ is H or F; and/or
R^{y} is C₁-C₃ alkyl substituted with 0 to 1 substituent selected from O—C₁-C₃ alkyl; or is benzyl; or is cyclopropyl.

5. The compound according to claim 4, wherein:
R^{y} is C₁-C₃ alkyl substituted with 0 to 1 substituent selected from O—C₁-C₃ alkyl; or is cyclopropyl.

6. The compound according to claim 1, wherein:
ring A is a ring within group I, group II, group III and group IV; and
ring B is a ring within group IA, group IIA, group IIIA and group IVA; and
when X is NH; Y is NH; R² is H; and ring A is a ring within group I or group II; then: ring B is a ring within group IA, group IIA, group IIIA or group IVA;
when X is NH; Y is NH; R² is H; and ring A is a ring within group III, then: ring B is a ring within group IA, group IIA or group IIIA;
when X is NH; Y is NH; R² is H; and ring A is a ring within group IV, then: ring B is a ring within group IA.

7. The compound according to claim 6, wherein:
when X is NH; Y is NH; R² is H; and ring A is a ring within group I; then: ring B is a ring within group IA, group IIA, group IIIA or group IVA; and
when X is NH; Y is NH; R² is H; and ring A is a ring within group II, then: ring B is a ring within group IA, group IIA or group IIIA.

8. The compound according to claim 1, wherein:
group IV is:

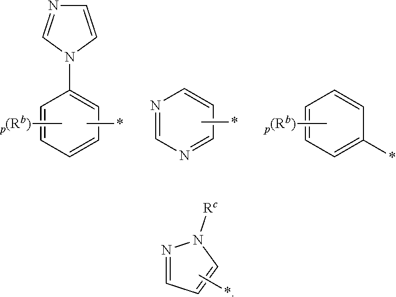

9. The compound according to claim 1 that (a) is of formula (Ia): 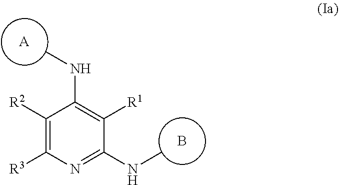
or (b) is a tautomer, N-oxide, pharmaceutically acceptable salt, or solvate thereof; wherein:
R¹ is H or F;
R² is H or F;
R³ is H or CH₃;
ring A is selected from a ring within group I, group II, group III and group IV, wherein * denotes attachment to the NH;
group I is: 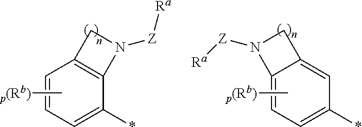
group II is: 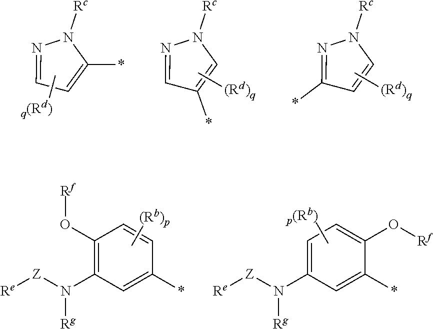
group III is: 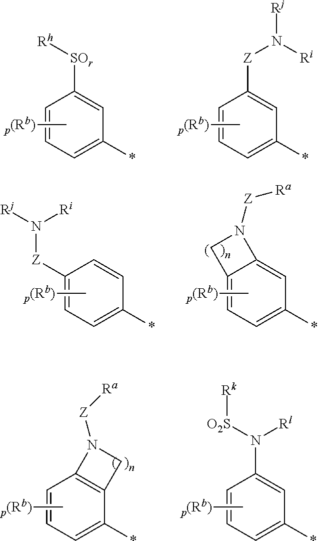
group IV is: 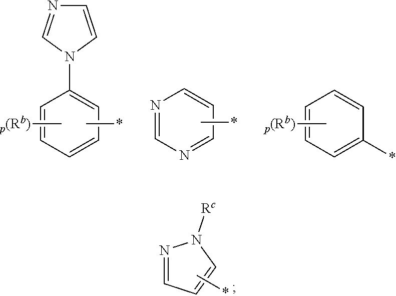
ring B is selected from a ring within group IA, group IIA, group IIA and group IVA, wherein ^{$} denotes attachment to the NH;
group IA is: 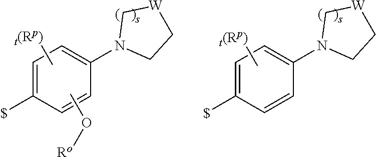
group IIA is: 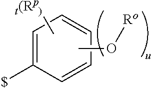
group IIIA is: 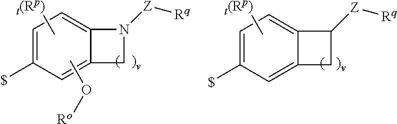
group IIIVA is: 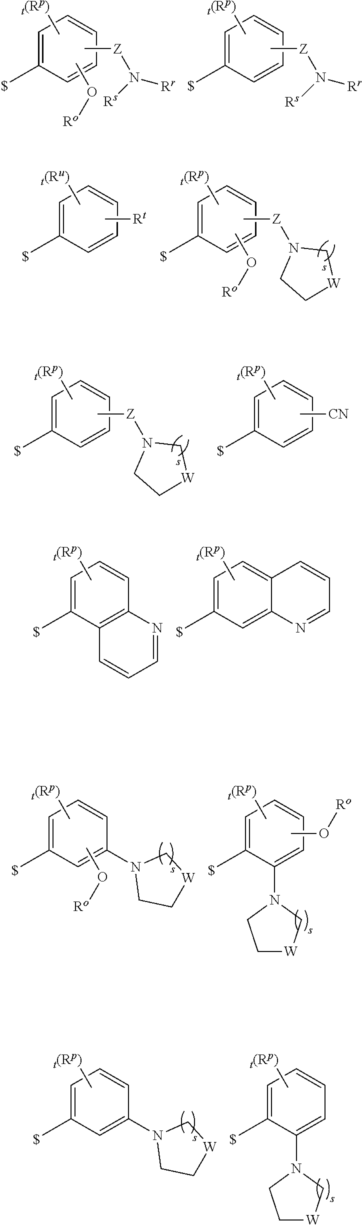
wherein:
W is CH₂, O, or NR^{y};
Z is C(O);
R^{a} is C₁-C₃ alkyl substituted with 0 to 1 substituent selected from O—C₁-C₃ alkyl;
R^{b} is independently selected from C₁-C₃ alkyl;
R^{c} is C₁-C₃ alkyl;
R^{d} is selected from C₁-C₄ alkyl or phenyl;
R^{e} is C₁-C₃ alkyl;
R^{f} is C₁-C₃ alkyl;
R^{g} is H;
R^{h} is C₁-C₃ alkyl;
Rⁱ is C₁-C₃ alkyl;
R^{j} is H;
R^{k} is C₁-C₃ alkyl;
R^{l} is H;
R^{o} is independently selected from C₁-C₃ alkyl substituted with 0 to 1 substituent selected from O—C₁-C₃ alkyl; or phenyl;
R^{p} is independently selected from C₁-C₃ alkyl
R^{q} is C₁-C₃ alkyl substituted with 0 to 1 substituent selected from O—C₁-C₃ alkyl;
R^{r} is H or C₁-C₃ alkyl;
R^{s} is H or C₁-C₃ alkyl;
R^{t} is C₁-C₄ alkyl;
R^{u} is independently selected from C₁-C₃ alkyl;
R^{y} is C₁-C₃ alkyl substituted with 0 to 1 substituent selected from O—C₁-C₃ alkyl; or is cyclopropyl;
n is 2;
p is 0 or 1;
q is 1;
r is 2;
s is 2;
t is 0 or 1;
u is 2 to 3;
v is 2;
and wherein:
when R² is H; and ring A is a ring within group I; then: ring B is a ring within group IA, group IIA, group IIIA or group IVA;
when R² is H; and ring A is a ring within group II, then: ring B is a ring within group IA, group IIA or group IIIA;
when R² is H; and ring A is a ring within group III, then: ring B is a ring within group IA, group IIA or group IIIA;
when R² is H; and ring A is a ring within group IV, then: ring B is a ring within group IA;
when R² is F then: ring A is a ring within group I or group II and ring B is a ring within group IA;
and with the proviso that the compound of formula (Ia) is not (a) a compound selected from the group: 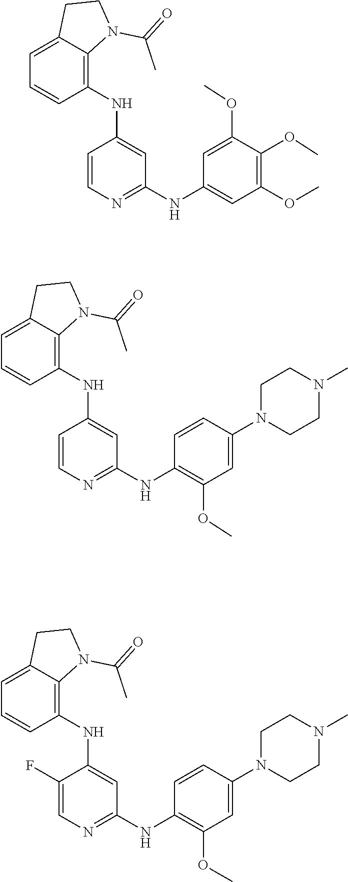 [SMILES: COc1cc(Nc2cc(Nc3cccc4c3N(C(C)=O)CC4)ccn2)cc(OC)c1OC ; COc1cc(N2CCN(C)CC2)ccc1Nc1cc(Nc2cccc3c2N(C(C)=O)CC3)ccn1 ; COc1cc(N2CCN(C)CC2)ccc1Nc1cc(Nc2cccc3c2N(C(C)=O)CC3)c(F)cn1]
nor (b) a tautomer, N-oxide, pharmaceutically acceptable salt, or solvate thereof.

10. The compound according to claim 1, wherein:
group I is: 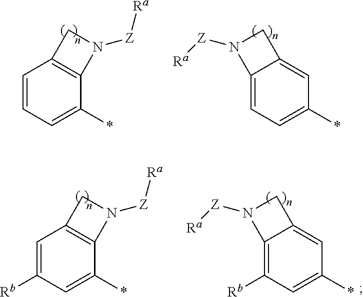
and/or
group II is: 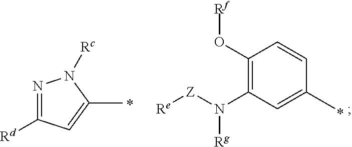
and/or
group III is: 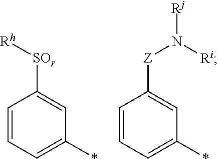
and/or
group IV is: 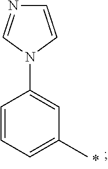
and/or
group IA is: 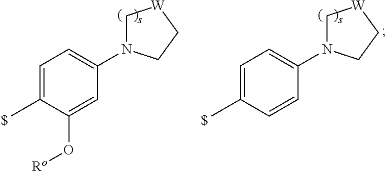
and/or
group IIA is: 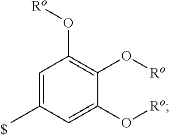
and/or
group IIIA is: 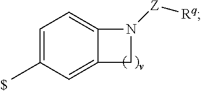
and/or
group IVA is:

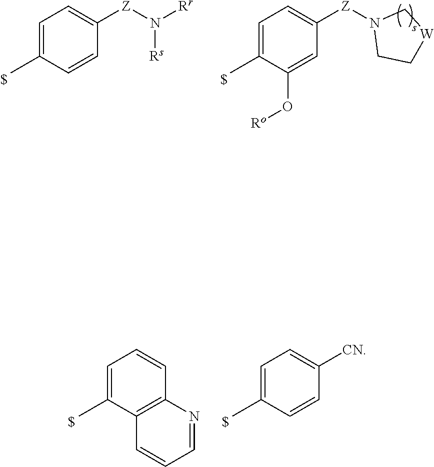

11. The compound according to claim 1, wherein:
R³ is H; and/or
R^{a} is CH₃, CH₂CH₃, or CH₂OCH₃; and/or
R^{d} is CH(CH₃)₂ or C(CH₃)₃; and/or
R^{o} is independently selected from CH₃, CH₂CH₃, or CH(CH₃)₂; and/or
R^{q} is CH₃, CH₂CH₃, or CH₂OCH₃; and/or
R^{r} is C₁-C₃ alkyl; and/or
R^{s} is H; and/or
R^{y} is CH₃, CH₂CH₃, or CH(CH₃)₂.

12. The compound according to claim 1, wherein:
when R² is H; and ring A is a ring within group II, then: ring B is a ring within group IA or group IIA; and
when R² is H; and ring A is a ring within group III, then: ring B is a ring within group IA or group IIA.

13. The compound according to claim 1 that (a) is of formula (Ib): 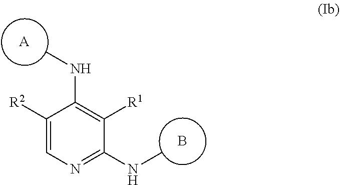
or (b) is a tautomer, N-oxide, pharmaceutically acceptable salt, or solvate thereof; wherein:
R¹ is H or F;
R² is H or F;
ring A is selected from a ring within group I, group II, group III and group IV, wherein * denotes attachment to the NH;
group I is: 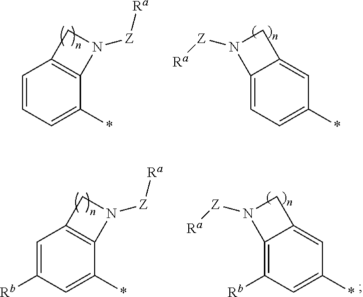
group II is: 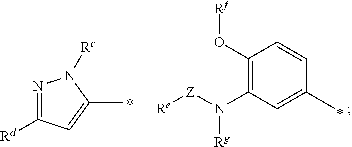
group III is: 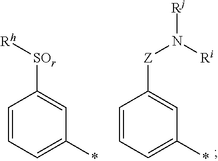
group IV is: 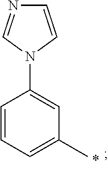
ring B is selected from a ring within group IA, group IIA, group IIA and group IVA, wherein ^{$} denotes attachment to the NH;
group IA is: 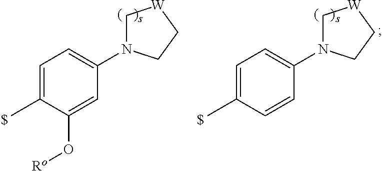
group IIA is: 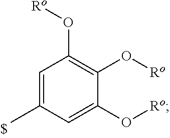
group IIIA is: 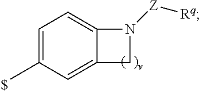
group IIIVA is: 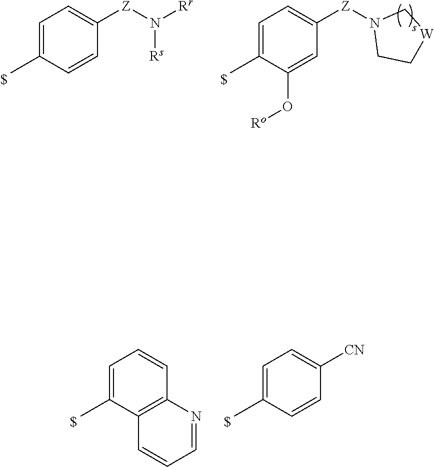
wherein:
W is CH₂, O, or NR^{y};
Z is C(O);
R^{a} is CH₃, CH₂CH₃, or CH₂OCH₃;
R^{b} is C₁-C₃ alkyl;
R^{c} is C₁-C₃ alkyl;
R^{d} is CH(CH₃)₂ or C(CH₃)₃;
R^{e} is C₁-C₃ alkyl;
R^{f} is C₁-C₃ alkyl;
R^{g} is H;
R^{h} is C₁-C₃ alkyl;
Rⁱ is C₁-C₃ alkyl;
R^{j} is H;
R^{o} is independently selected from CH₃, CH₂CH₃, or CH(CH₃)₂;
R^{q} is CH₃, CH₂CH₃, or CH₂OCH₃;
R^{r} is C₁-C₃ alkyl;
R^{s} is H;
R^{y} is CH₃, CH₂CH₃, or CH(CH₃)₂;
n is 2;
r is 2;
s is 2;
v is 2;
and wherein:
when R² is H; and ring A is a ring within group I; then: ring B is a ring within group IA, group IIA, group IIIA or group IVA;
when R² is H; and ring A is a ring within group II, then: ring B is a ring within group IA or group IIA; and
when R² is H; and ring A is a ring within group III, then: ring B is a ring within group IA or group IIA.
when R² is H; and ring A is a ring within group IV, then: ring B is a ring within group IA;
when and R² is F; then: ring A is a ring within group I or group II and ring B is a ring within group IA;
and with the proviso that the compound of formula (Ib) is not (a) a compound selected from the group: 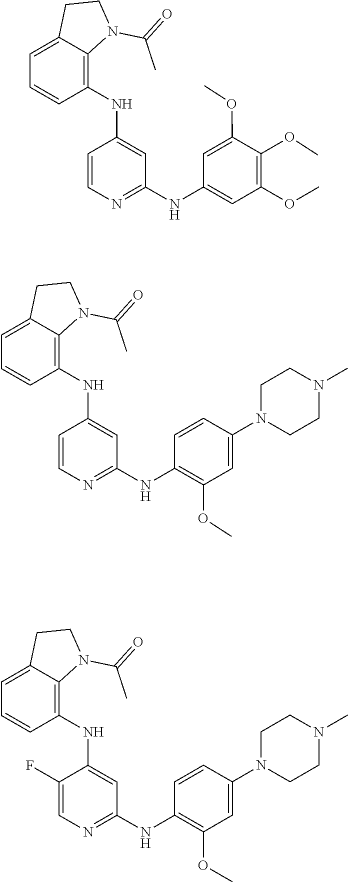 [SMILES: COc1cc(Nc2cc(Nc3cccc4c3N(C(C)=O)CC4)ccn2)cc(OC)c1OC ; COc1cc(N2CCN(C)CC2)ccc1Nc1cc(Nc2cccc3c2N(C(C)=O)CC3)ccn1 ; COc1cc(N2CCN(C)CC2)ccc1Nc1cc(Nc2cccc3c2N(C(C)=O)CC3)c(F)cn1]
nor (b) a tautomer, N-oxide, pharmaceutically acceptable salt, or solvate thereof.

14. The compound according to claim 1, wherein:
group I is: 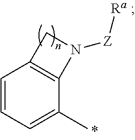
and/or
group II is:

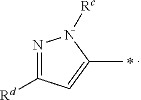

15. The compound according to claim 1, wherein the formula (I) is selected from:

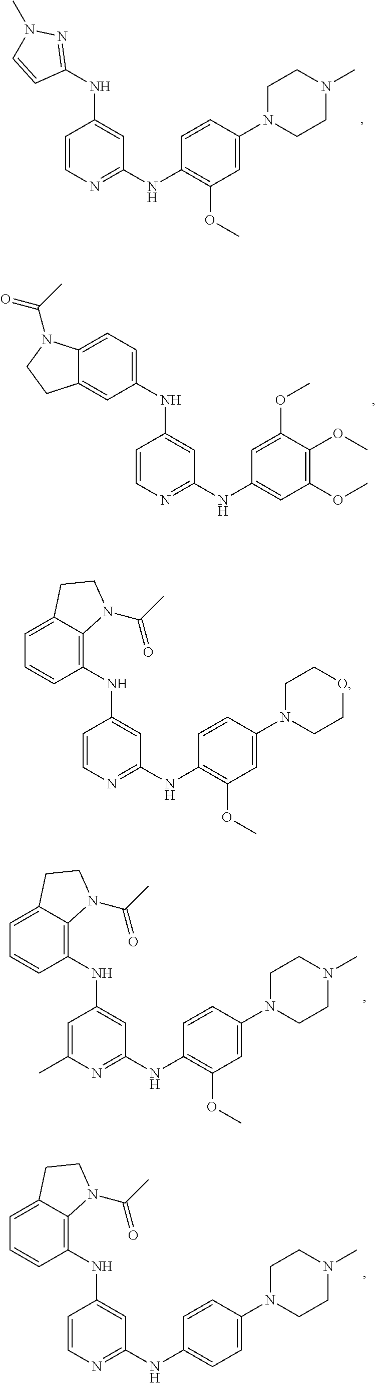 [SMILES: COc1cc(N2CCN(C)CC2)ccc1Nc1cc(Nc2ccn(C)n2)ccn1 ; COc1cc(Nc2cc(Nc3ccc4c(c3)CCN4C(C)=O)ccn2)cc(OC)c1OC ; COc1cc(N2CCOCC2)ccc1Nc1cc(Nc2cccc3c2N(C(C)=O)CC3)ccn1 ; COc1cc(N2CCN(C)CC2)ccc1Nc1cc(Nc2cccc3c2N(C(C)=O)CC3)cc(C)n1 ; CC(=O)N1CCc2cccc(Nc3ccnc(Nc4ccc(N5CCN(C)CC5)cc4)c3)c21]

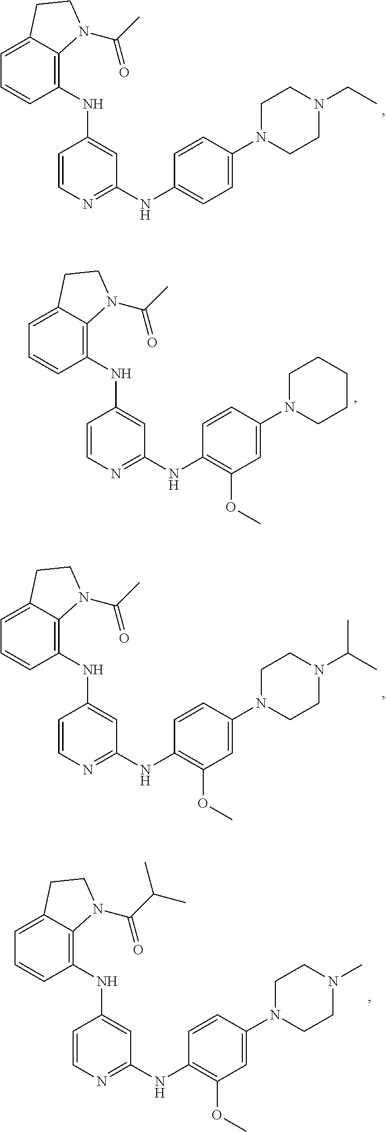 [SMILES: CCN1CCN(c2ccc(Nc3cc(Nc4cccc5c4N(C(C)=O)CC5)ccn3)cc2)CC1 ; COc1cc(N2CCCCC2)ccc1Nc1cc(Nc2cccc3c2N(C(C)=O)CC3)ccn1 ; COc1cc(N2CCN(C(C)C)CC2)ccc1Nc1cc(Nc2cccc3c2N(C(C)=O)CC3)ccn1 ; COc1cc(N2CCN(C)CC2)ccc1Nc1cc(Nc2cccc3c2N(C(=O)C(C)C)CC3)ccn1]

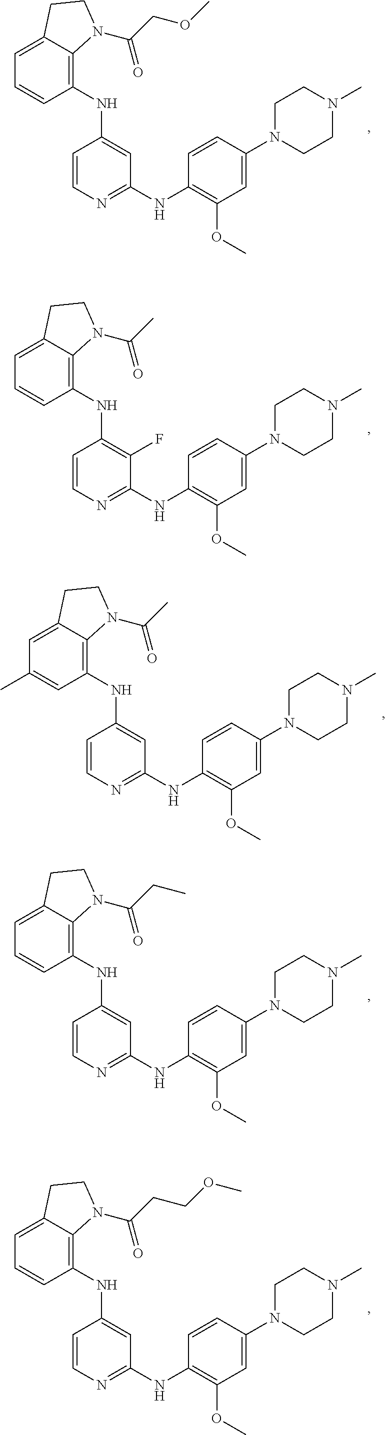

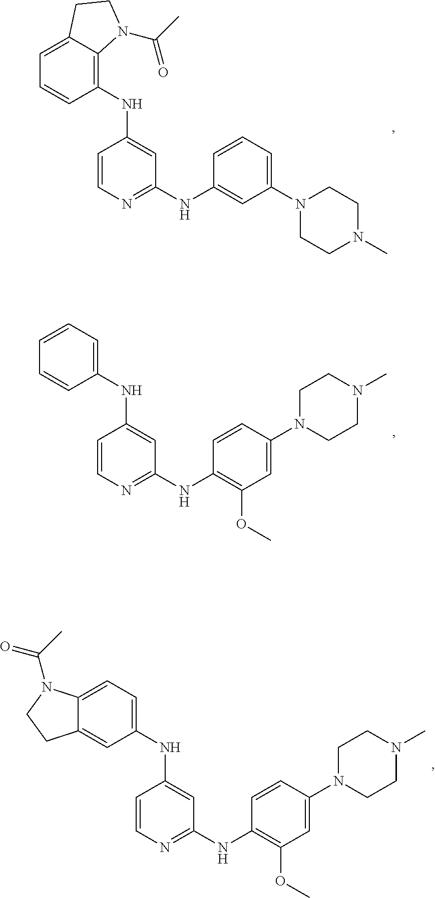 [SMILES: CC(=O)N1CCc2cccc(Nc3ccnc(Nc4cccc(N5CCN(C)CC5)c4)c3)c21 ; COc1cc(N2CCN(C)CC2)ccc1Nc1cc(Nc2ccccc2)ccn1 ; COc1cc(N2CCN(C)CC2)ccc1Nc1cc(Nc2ccc3c(c2)CCN3C(C)=O)ccn1]

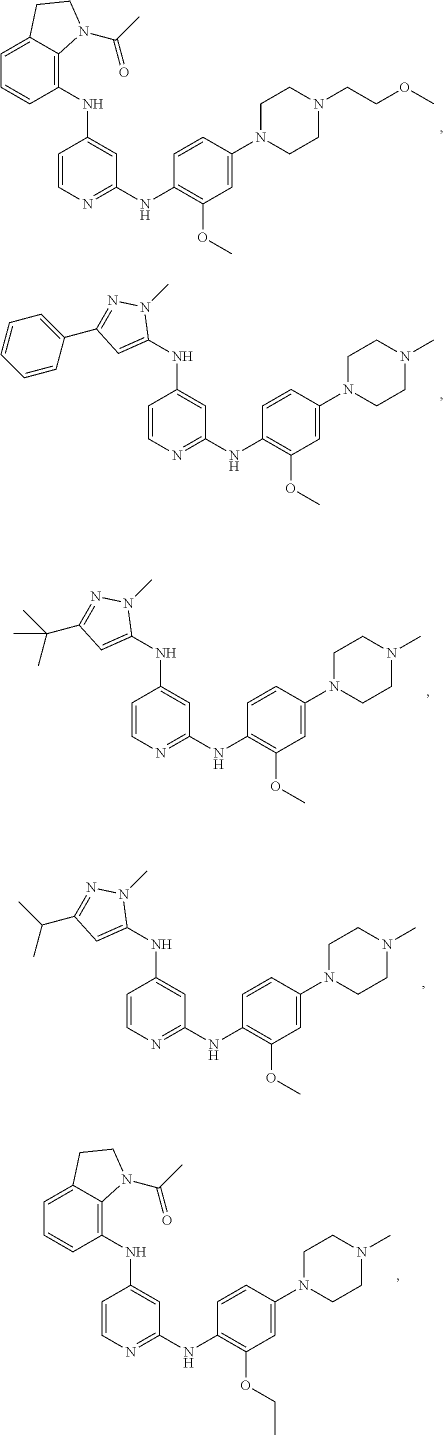

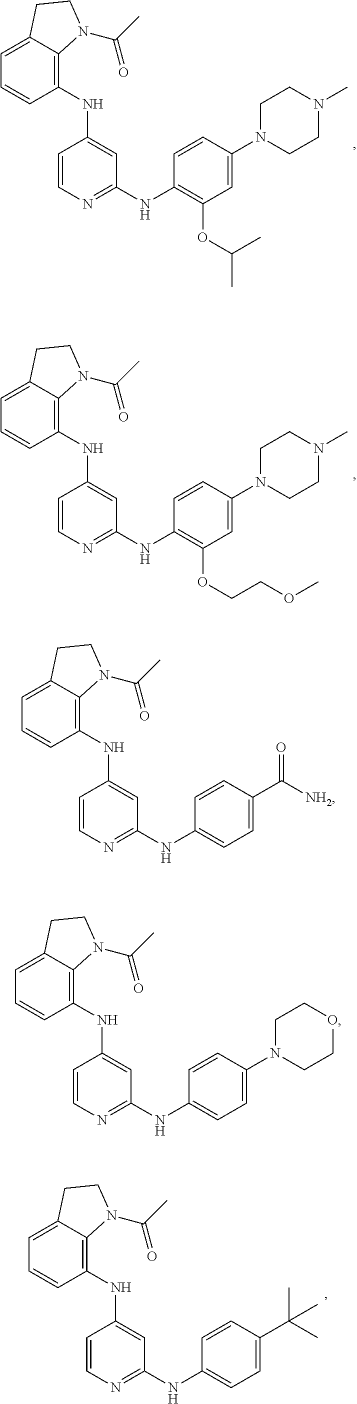 [SMILES: CC(=O)N1CCc2cccc(Nc3ccnc(Nc4ccc(N5CCN(C)CC5)cc4OC(C)C)c3)c21 ; COCCOc1cc(N2CCN(C)CC2)ccc1Nc1cc(Nc2cccc3c2N(C(C)=O)CC3)ccn1 ; CC(=O)N1CCc2cccc(Nc3ccnc(Nc4ccc(C(N)=O)cc4)c3)c21 ; CC(=O)N1CCc2cccc(Nc3ccnc(Nc4ccc(N5CCOCC5)cc4)c3)c21 ; CC(=O)N1CCc2cccc(Nc3ccnc(Nc4ccc(C(C)(C)C)cc4)c3)c21]

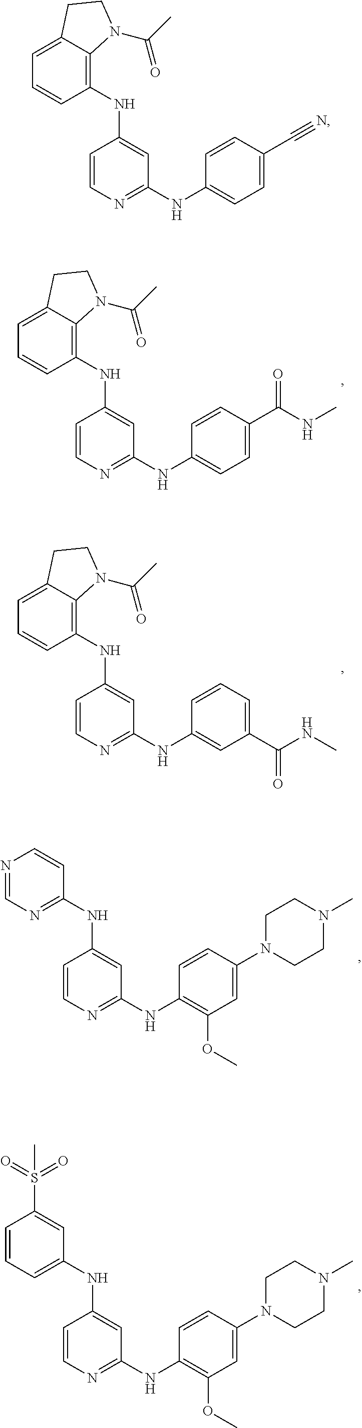 [SMILES: CC(=O)N1CCc2cccc(Nc3ccnc(Nc4ccc(C#N)cc4)c3)c21 ; CNC(=O)c1ccc(Nc2cc(Nc3cccc4c3N(C(C)=O)CC4)ccn2)cc1 ; CNC(=O)c1cccc(Nc2cc(Nc3cccc4c3N(C(C)=O)CC4)ccn2)c1 ; COc1cc(N2CCN(C)CC2)ccc1Nc1cc(Nc2ccncn2)ccn1 ; COc1cc(N2CCN(C)CC2)ccc1Nc1cc(Nc2cccc(S(C)(=O)=O)c2)ccn1]

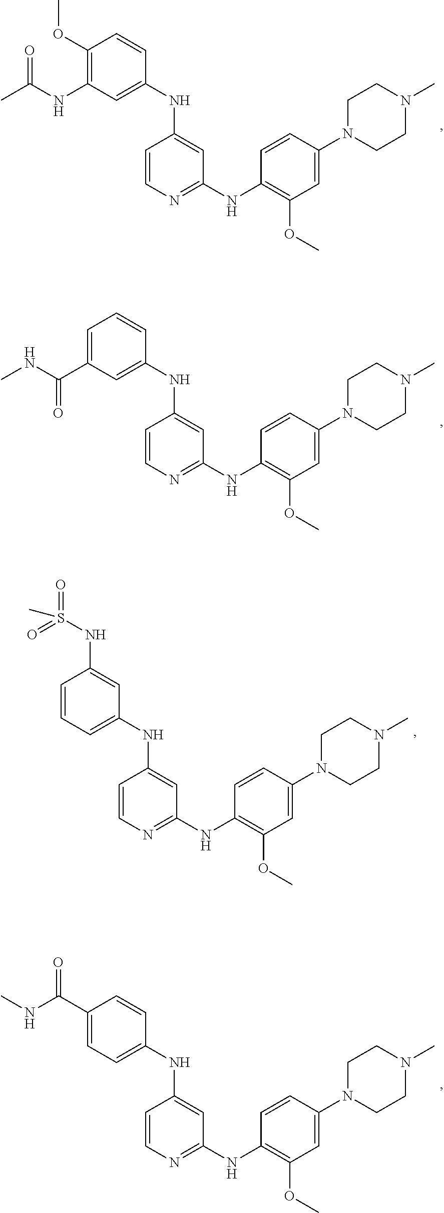

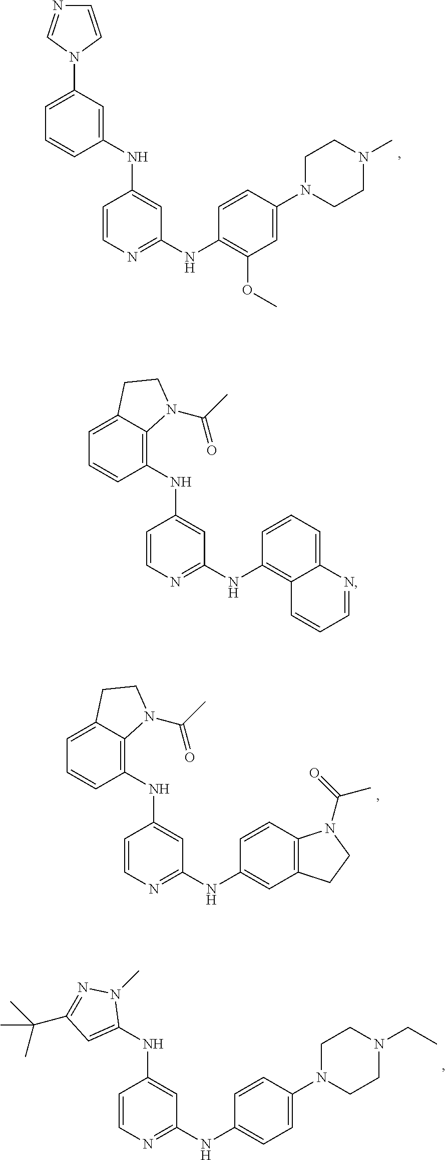

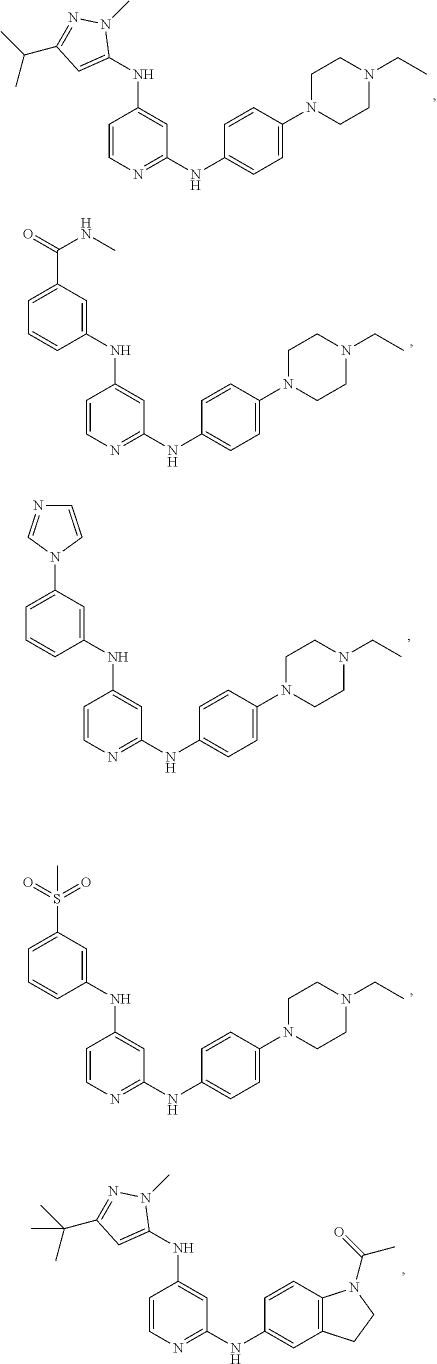

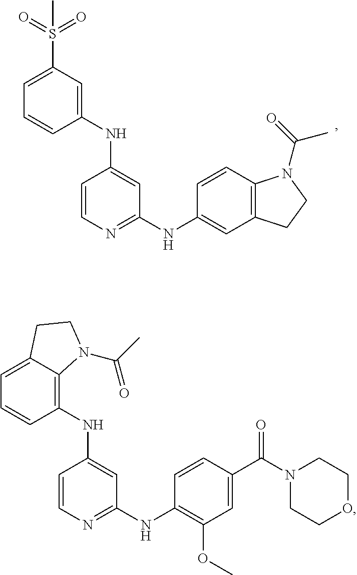 [SMILES: CC(=O)N1CCc2cc(Nc3cc(Nc4cccc(S(C)(=O)=O)c4)ccn3)ccc21 ; COc1cc(C(=O)N2CCOCC2)ccc1Nc1cc(Nc2cccc3c2N(C(C)=O)CC3)ccn1]

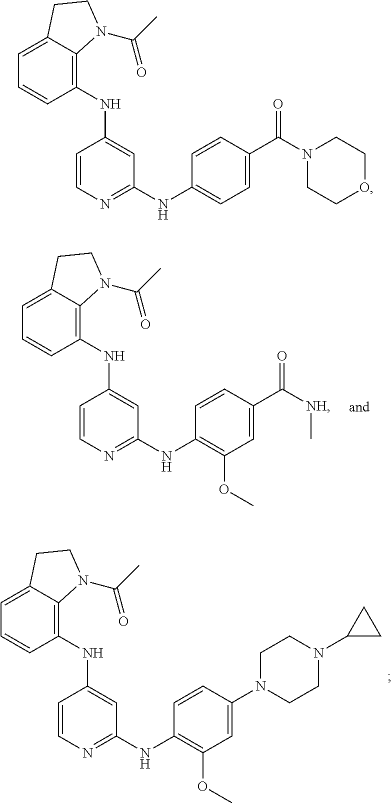 [SMILES: CC(=O)N1CCc2cccc(Nc3ccnc(Nc4ccc(C(=O)N5CCOCC5)cc4)c3)c21 ; CNC(=O)c1ccc(Nc2cc(Nc3cccc4c3N(C(C)=O)CC4)ccn2)c(OC)c1 ; COc1cc(N2CCN(C3CC3)CC2)ccc1Nc1cc(Nc2cccc3c2N(C(C)=O)CC3)ccn1]

16. The compound according to claim 15, wherein the formula (I) is selected from:

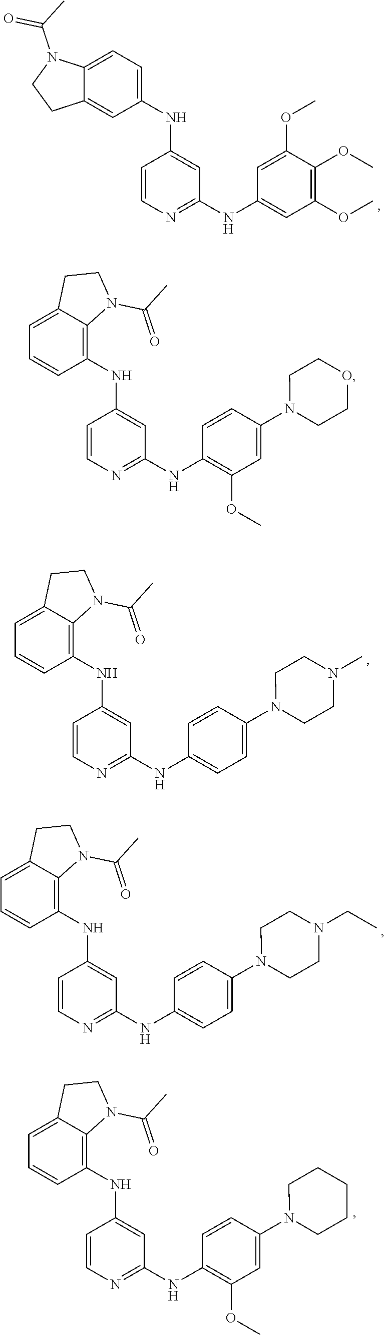 [SMILES: COc1cc(Nc2cc(Nc3ccc4c(c3)CCN4C(C)=O)ccn2)cc(OC)c1OC ; COc1cc(N2CCOCC2)ccc1Nc1cc(Nc2cccc3c2N(C(C)=O)CC3)ccn1 ; CC(=O)N1CCc2cccc(Nc3ccnc(Nc4ccc(N5CCN(C)CC5)cc4)c3)c21 ; CCN1CCN(c2ccc(Nc3cc(Nc4cccc5c4N(C(C)=O)CC5)ccn3)cc2)CC1 ; COc1cc(N2CCCCC2)ccc1Nc1cc(Nc2cccc3c2N(C(C)=O)CC3)ccn1]

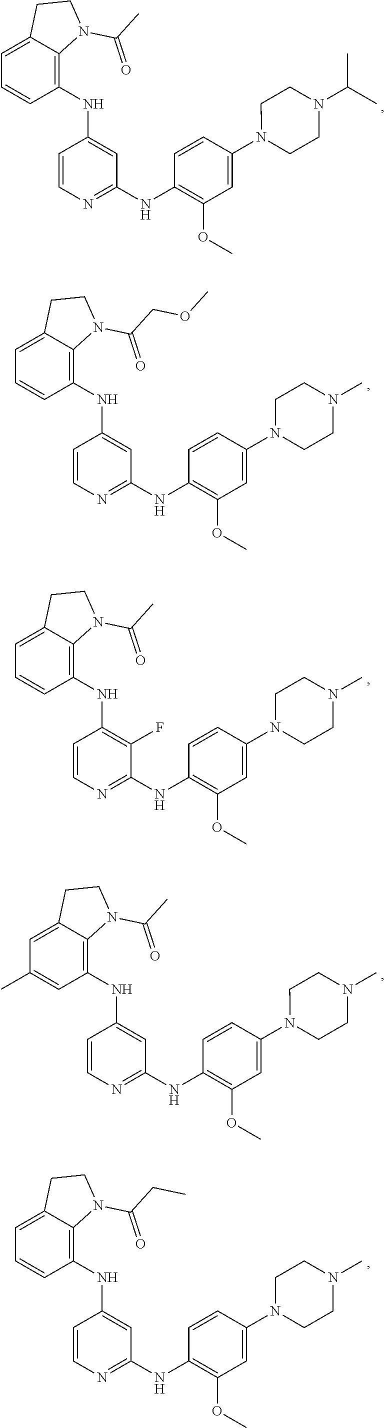

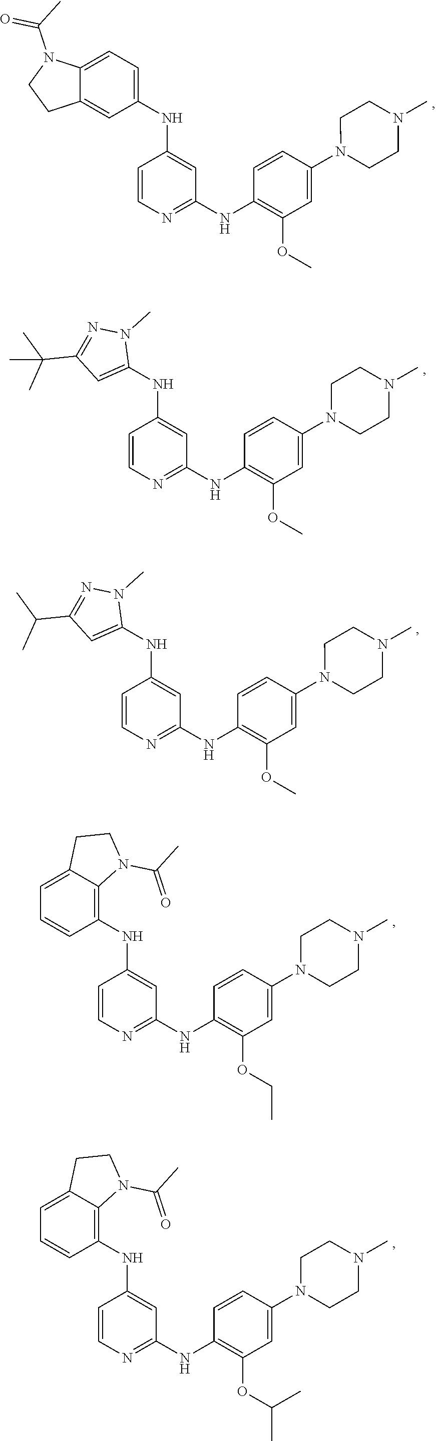

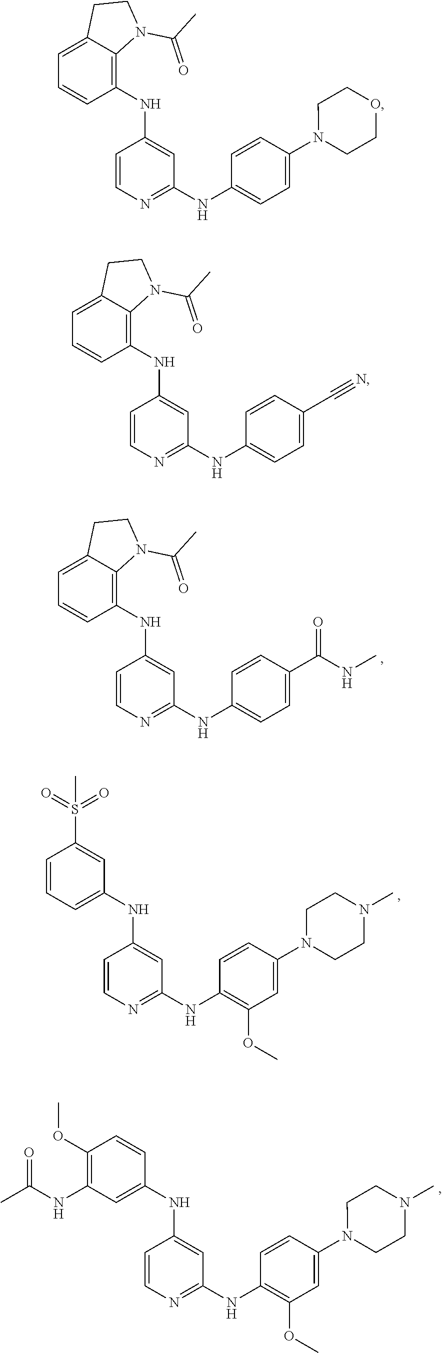

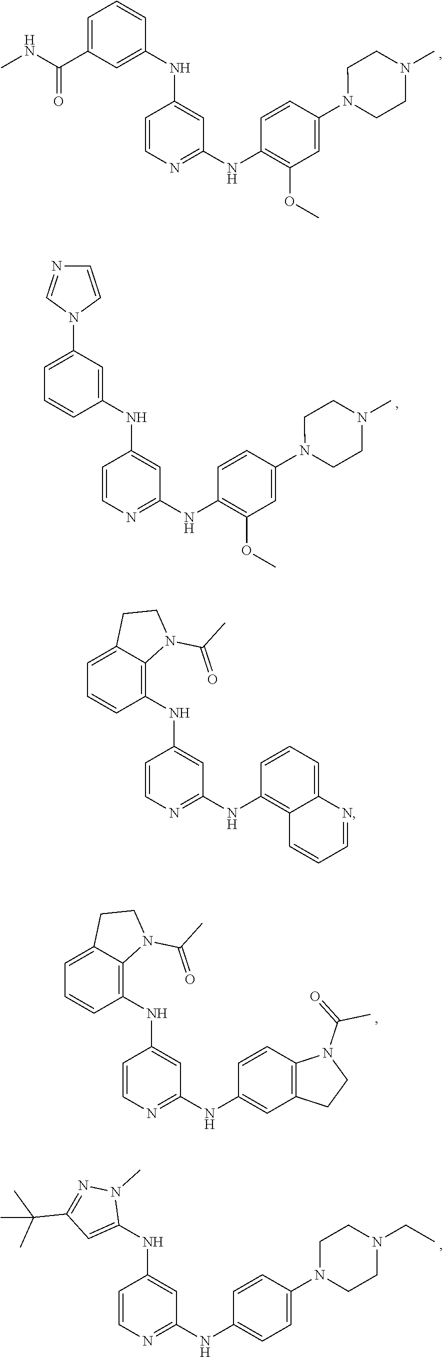

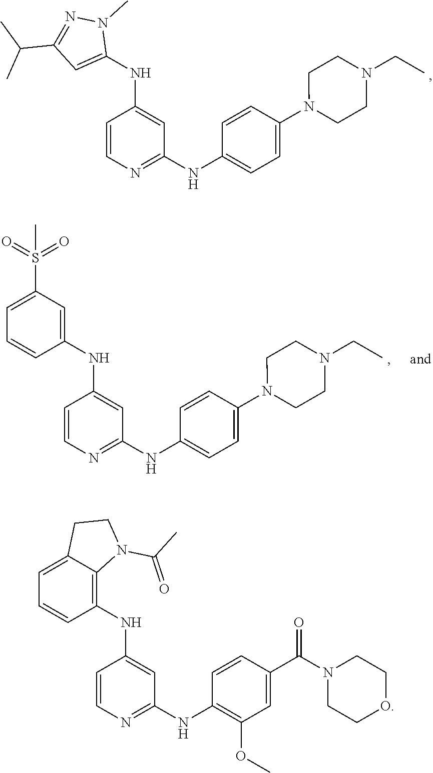 [SMILES: CCN1CCN(c2ccc(Nc3cc(Nc4cc(C(C)C)nn4C)ccn3)cc2)CC1 ; CCN1CCN(c2ccc(Nc3cc(Nc4cccc(S(C)(=O)=O)c4)ccn3)cc2)CC1 ; COc1cc(C(=O)N2CCOCC2)ccc1Nc1cc(Nc2cccc3c2N(C(C)=O)CC3)ccn1]

17. The compound according to claim 16, wherein the formula (I) is:

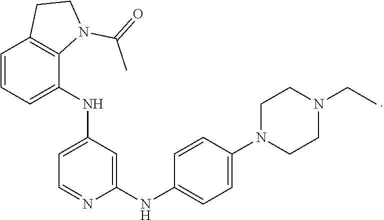 [SMILES: CCN1CCN(c2ccc(Nc3cc(Nc4cccc5c4N(C(C)=O)CC5)ccn3)cc2)CC1]

18. A pharmaceutical composition comprising a compound according to claim 1 in association with one or more pharmaceutically acceptable carriers.

19. (canceled)

20. A method for treating and/or preventing a disorder susceptible to treatment by PI3Kα activation in a patient in need thereof, the method comprising administering a compound according to claim 1 to the patient.

21. A method for treating and/or preventing peripheral nerve injury in a patient in need thereof, the method comprising administering a compound according to claim 1 to the patient.
